SEQUENCE LISTING (254 sequences)

SEQ ID NO: 1 - protein, 8 aa, synthetic construct
GGTFSSYA

SEQ ID NO: 2 - protein, 8 aa, synthetic construct
IIPIFGTA

SEQ ID NO: 3 - protein, 22 aa, synthetic construct
DNLGYCSGGSCYSDYYYYYMDV

SEQ ID NO: 4 - protein, 6 aa, synthetic construct
NIGQQI

SEQ ID NO: 5 - skipped

SEQ ID NO: 6 - protein, 11 aa, synthetic construct
QVWDSGSDHVV

SEQ ID NO: 7 - protein, 13 aa, synthetic construct
TRSSGSIASTYVH

SEQ ID NO: 8 - protein, 7 aa, synthetic construct
EDNQRPS

SEQ ID NO: 9 - protein, 11 aa, synthetic construct
QSYDSSNGSVV

SEQ ID NO: 10 - protein, 13 aa, synthetic construct
TRSSGSIGSDYVH

SEQ ID NO: 11 - protein, 11 aa, synthetic construct
QSYDRSNHEVV

SEQ ID NO: 12 - protein, 7 aa, synthetic construct
EDNRRPS

SEQ ID NO: 13 - protein, 11 aa, synthetic construct
QSYDSTTNHVV

SEQ ID NO: 14 - protein, 13 aa, synthetic construct
TRSSGRIVSDYVH

SEQ ID NO: 15 - protein, 11 aa, synthetic construct
QSYDSSNAYVV

SEQ ID NO: 16 - protein, 11 aa, synthetic construct
EGDNIGQQIVH

SEQ ID NO: 17 - protein, 7 aa, synthetic construct
DGSRRPS

SEQ ID NO: 18 - protein, 11 aa, synthetic construct
QVWDITSDHVV

SEQ ID NO: 19 - protein, 7 aa, synthetic construct
SDSDRPS

SEQ ID NO: 20 - protein, 11 aa, synthetic construct
GGDYIGGESVH

SEQ ID NO: 21 - protein, 7 aa, synthetic construct
DDTHRPS

SEQ ID NO: 22 - protein, 11 aa, synthetic construct
QVWDVSSDHVV

SEQ ID NO: 23 - protein, 11 aa, synthetic construct
GGHNIGSNIVH

SEQ ID NO: 24 - protein, 7 aa, synthetic construct
HNTNRPS

SEQ ID NO: 25 - protein, 11 aa, synthetic construct
QVWDSSSEHVV

SEQ ID NO: 26 - protein, 22 aa, synthetic construct
NFMLTQPRSVSESPGKTVTISC

SEQ ID NO: 27 - protein, 15 aa, synthetic construct
WYQQRPGSSPTTVIY

SEQ ID NO: 28 - protein, 34 aa, synthetic construct
GVPDRFSGSIDSSSNSASLTISGLKTEDEADYYC

SEQ ID NO: 29 - protein, 10 aa, synthetic construct
FGGGTKLTVL

SEQ ID NO: 30 - protein, 30 aa, synthetic construct
QVQLVQSGAEVKKPGSSVKVSCKASGGTFS

SEQ ID NO: 31 - protein, 14 aa, synthetic construct
WVRQAPGQGLEWMG

SEQ ID NO: 32 - protein, 32 aa, synthetic construct
RVTITADESTSTAYMELRSLRSDDTAVYYCAR

SEQ ID NO: 33 - protein, 11 aa, synthetic construct
WGQGTLVTVSS

SEQ ID NO: 34 - protein, 131 aa, synthetic construct
QVQLVQSGAEVKKPGSSVKVSCKASGGTFSSYAISWVRQAPGQGLEWMGGIIPIFGTANYAQKFQGRVTITADESTSTAYMELRSLRSDDTAVYYCARDNLGYCSGGSCYSDYYYYYMDVWGQGTLVTVSS

SEQ ID NO: 35 - protein, 112 aa, synthetic construct
NFMLTQPRSVSESPGKTVTISCTRSSGSIGSDYVHWYQQRPGSSPTTVIYEDNQRPSGVPDRFSGSIDSSSNSASLTISGLKTEDEADYYCQSYDRSNHEVVFGGGTKLTVL

SEQ ID NO: 36 - DNA, 393 bp, synthetic construct
  source 1..393: mol_type = other DNA
caggtgcagctggtgcaatctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgaggagcctgagatctgacgacacggccgtgtattactgtgcgagagacaacctaggatattgtagtggtggtagctgctactctgactactactactactacatggacgtctggggccagggcaccctggtcaccgtctcgagt

SEQ ID NO: 37 - DNA, 336 bp, synthetic construct
  source 1..336: mol_type = other DNA
aattttatgctgactcagccccgctctgtgtcggagtctccggggaagacggtaaccatctcctgcacccgcagcagtggcagcattggcagcgactatgtgcattggtaccagcagcgcccgggcagctcccccaccactgtcatctatgaggataaccaaagaccctctggggtccctgatcggttctctggctccatcgacagctcctccaactctgcctccctcaccatctctggactgaagactgaggacgaggctgactactactgtcagtcttatgatcgcagcaatcatgaagtggtgttcggcggagggaccaagctgaccgtccta

SEQ ID NO: 38 - protein, 25 aa, synthetic construct [sequence omitted]

SEQ ID NO: 39 - protein, 17 aa, synthetic construct
ISWVRQAPGQGLEWMGG

SEQ ID NO: 40 - protein, 40 aa, synthetic construct
NYAQKFQGRVTITADESTSTAYMELRSLRSDDTAVYYCAR

SEQ ID NO: 41 - protein, 22 aa, synthetic construct
SYELTQPPSVSVAPGRTATITC

SEQ ID NO: 42 - protein, 25 aa, synthetic construct
SYELTQPPSVSVAPGRTATITCEGD

SEQ ID NO: 43 - protein, 15 aa, synthetic construct
WYQQKPGQAPVAVIS

SEQ ID NO: 44 - protein, 17 aa, synthetic construct
VHWYQQKPGQAPVAVIS

SEQ ID NO: 45 - protein, 32 aa, synthetic construct
GIPERFSGSNSGNTATLTISRVEAGDEADYYC

SEQ ID NO: 46 - protein, 36 aa, synthetic construct
DRPSGIPERFSGSNSGNTATLTISRVEAGDEADYYC

SEQ ID NO: 47 - protein, 10 aa, synthetic construct
FGGGTKVTVL

SEQ ID NO: 48 - DNA, 20 bp, synthetic construct
  source 1..20: mol_type = other DNA
cgctgcccagccggccatgg

SEQ ID NO: 49 - DNA, 22 bp, synthetic construct
  source 1..22: mol_type = other DNA
ctgaaccgcctccaccactcga

SEQ ID NO: 50 - DNA, 19 bp, synthetic construct
  source 1..19: mol_type = other DNA
ctctggcggtggcgctagc

SEQ ID NO: 51 - DNA, 26 bp, synthetic construct
  source 1..26: mol_type = other DNA
gatggtgatgatgatgtgcggatgcg

SEQ ID NO: 52 - DNA, 32 bp, synthetic construct
  source 1..32: mol_type = other DNA
ggctacgaagacaccccagccggccatggctc

SEQ ID NO: 53 - DNA, 62 bp, synthetic construct
  source 1..62: mol_type = other DNA
ggctacgaagactaccatccactccaaaccttggcccggtgcknnacgaacccagctaatcg

SEQ ID NO: 54 - DNA, 62 bp, synthetic construct
  source 1..62: mol_type = other DNA
ggctacgaagactaccatccactccaaaccknngcccggtgcctgacgaacccagctaatcg

SEQ ID NO: 55 - DNA, 62 bp, synthetic construct
  source 1..62: mol_type = other DNA
ggctacgaagactaccatccactccaaknnttggcccggtgcctgacgaacccagctaatcg

SEQ ID NO: 56 - DNA, 62 bp, synthetic construct
  source 1..62: mol_type = other DNA
ggctacgaagactaccatccactccaaaccttggcccggtgcctgacgaacccagctaatcg

SEQ ID NO: 57 - DNA, 62 bp, synthetic construct
  source 1..62: mol_type = other DNA
ggctacgaagactaccatccactcknnaccttggcccggtgcctgacgaacccagctaatcg

SEQ ID NO: 58 - DNA, 57 bp, synthetic construct
  source 1..57: mol_type = other DNA
ggctacgaagactaatgggtggtattatcccgatttttggtactgctaattatgcgc

SEQ ID NO: 59 - DNA, 52 bp, synthetic construct
  source 1..52: mol_type = other DNA
ggctacgaagactatcacgggcgcagtaataknnagcggtatcatcgctacg

SEQ ID NO: 60 - DNA, 52 bp, synthetic construct
  source 1..52: mol_type = other DNA
ggctacgaagactatcacgggcgcagtaatacacagcggtatcatcgctacg

SEQ ID NO: 61 - DNA, 46 bp, synthetic construct
  source 1..46: mol_type = other DNA
ggctacgaagactagtgataatctgggttattgcagcggcggctcc

SEQ ID NO: 62 - DNA, 49 bp, synthetic construct
  source 1..49: mol_type = other DNA
ggctacgaagactaagacggtgactaaggtaccknnaccccaaacatcc

SEQ ID NO: 63 - DNA, 49 bp, synthetic construct
  source 1..49: mol_type = other DNA
ggctacgaagactaagacggtgactaaggtaccttgaccccaaacatcc

SEQ ID NO: 64 - DNA, 40 bp, synthetic construct
  source 1..40: mol_type = other DNA
ggctacgaagactagtctcgagcggtggtggcggctccgg

SEQ ID NO: 65 - DNA, 59 bp, synthetic construct
  source 1..59: mol_type = other DNA
ggctacgaagactagacggtcgtcggtgacgagcccgggcgknnctgataccaatgcac

SEQ ID NO: 66 - DNA, 52 bp, synthetic construct
  source 1..52: mol_type = other DNA
ggctacgaagactagacggtcgtcggknncgagcccgggcgctgctgatacc

SEQ ID NO: 67 - DNA, 59 bp, synthetic construct
  source 1..59: mol_type = other DNA
ggctacgaagactagacggtcgtcggtgacgagcccgggcgctgctgataccaatgcac

SEQ ID NO: 68 - DNA, 52 bp, synthetic construct
  source 1..52: mol_type = other DNA
ggctacgaagactacgtcatctatgaggacaaccagcgtccgagcggggtgc

SEQ ID NO: 69 - DNA, 45 bp, synthetic construct
  source 1..45: mol_type = other DNA
ggctacgaagactagctttggcagtagtaknncgcctcgtcctcg

SEQ ID NO: 70 - DNA, 45 bp, synthetic construct
  source 1..45: mol_type = other DNA
ggctacgaagactagctttggcagtagtagtccgcctcgtcctcg

SEQ ID NO: 71 - DNA, 68 bp, synthetic construct
  source 1..68: mol_type = other DNA
ggctacgaagactaaagctacgaccgctctaaccacgaagttgtttttggcnnkggtacgaagctgac

SEQ ID NO: 72 - DNA, 55 bp, synthetic construct
  source 1..55: mol_type = other DNA
ggctacgaagactacaactttcaacagtttctgcggccccattcagatcctcttc

SEQ ID NO: 73 - DNA, 61 bp, synthetic construct
  source 1..61: mol_type = other DNA
ggctacgaagactagttgtttagcaaaacctcatacagaaaattcatttactaacgtctgg

SEQ ID NO: 74 - DNA, 37 bp, synthetic construct
  source 1..37: mol_type = other DNA
ggctacgaagaccatgggccgcatagaaaggaacaac

SEQ ID NO: 75 - DNA, 20 bp, synthetic construct
  source 1..20: mol_type = other DNA
tggaaaaacgccagcaacgc

SEQ ID NO: 76 - DNA, 19 bp, synthetic construct
  source 1..19: mol_type = other DNA
ccctcatagttgcgtaacg

SEQ ID NO: 77 - protein, 129 aa, synthetic construct
QVQLVQSGAEVKKPGSSVKVSCKASGGTFSSYAISWVRQAPGQGLEWMGGIIPIFGTANYAQKFQGRVTITADESTSTAYMELRSLRSDDTAVYYCARDNLGYCSGGSCYSDYYYYYMDVWGQGTLVTV

SEQ ID NO: 78 - protein, 129 aa, synthetic construct
HVQLVQSGAEVKKPGSSVKVSCKASGGTFSSYAISWVRQAPGQTLEWMGGIIPIFGTANYAQKFQGRVTITADESTSTAYMELRSLRSDDTAVYYCARDNLGYCSGGSCYSDYYYYYMDVWGQGTLVTV

SEQ ID NO: 79 - protein, 129 aa, synthetic construct
QVQLVQSGAEVKKPGSSVKVSCKASGGTFSSYAISWVRSAPGQGLEWMGGIIPIFGTANYAQKFQGRVTITADESTSTAYMELRSLRSDDTAVYYCARDNLGYCSGGSCYSDYYYYYMDVWGVGTLVTV

SEQ ID NO: 80 - protein, 129 aa, synthetic construct
QVQLVQSGAEVKKPGSSVKVSCKASGGTFSSYAISWVRQAPGQGLEWMGGIIPIFGTANYAQKFKGRVTITADESTSTAYMELRSLRSDDTADYYCARDNLGYCSGGSCYSDYYYYYMDVWGQGTLVTV

SEQ ID NO: 81 - protein, 129 aa, synthetic construct
QVQLVQSGAEVKKPGSSVKVSCKASGGTFSSYAISWVRQAPGQRLEWMGGIIPIFGTANYAQKFQGRVTITADESTSTAYMELRSLRSDDTAQYYCARDNLGYCSGGSCYSDYYYYYMDVWGQGTLVTV

SEQ ID NO: 82 - protein, 129 aa, synthetic construct
QVQLVQSGAEVKKPGSSVKVSCKASGGTFSSYAISWVRRAPGQGLEWMGGIIPIFGTANYAQKFQGRVTITADESTSTAYMELRSLRSDDTAQYYCARDNLGYCSGGSCYSDYYYYYMDVWGQGTLVTV

SEQ ID NO: 83 - protein, 129 aa, synthetic construct
QVQLVQSGAEVKKPGSSVKVSCKASGGTFSSYAISWVRQAPGQNLEWMGGIIPIFGTANYAQKFQGRVTITADESTSTAYMELRSLRSDDTAVYYCARDNLGYCSGGSCYSDYYYYYMDVWGQGTLVTV

SEQ ID NO: 84 - protein, 129 aa, synthetic construct
QVQLVQSGAEVKKPGSSVKVSCKASGGTFSSYAISWVRTAPGQGLEWMGGIIPIFGTANYAQKFQGRVTITADESTSTAYMELRSLRSDDTATYYCARDNLGYCSGGSCYSDYYYYYMDVWGQGTLVTV

SEQ ID NO: 85 - protein, 129 aa, synthetic construct
QVQLVQSGAEVKKPGSSVKVSCKASGGTFSSYAISWVRQAPGQYLEWMGGIIPIFGTANYAQKFQGRVTITADESTSTAYMELRSLRSDDTAVYYCARDNLGYCSGGSCYSDYYYYYMDVWGQGTLVTV

SEQ ID NO: 86 - protein, 129 aa, synthetic construct
QFQLVQSGAEVKKPGSSVKVSCKASGGTFSSYAISWVRQAPGQGLEWMGGIIPIFGTANYAQKFQGRVTITADESTSTAYMELRSLRSDDTATYYCARDNLGYCSGGSCYSDYYYYYMDVWGQGTLVTV

SEQ ID NO: 87 - protein, 129 aa, synthetic construct
QVQLVQSGAEVKKPGSSVKVSCKASGGTFSSYAISWVREAPGQGLEWMGGIIPIFGTANYAQKFQGRVTITADESTSTAYMELRSLRSDDTAIYYCARDNLGYCSGGSCYSDYYYYYMDVWGQGTLVTV

SEQ ID NO: 88 - protein, 112 aa, synthetic construct
NFMLTQPRSVSESPGKTVTISCTRSSGSIGSDYVHWYQQRPGSRPTTVIYEDNQRPSGVPDRFSGSIDSSSNSASLTISGLKTEDEADYYCQSYDRSNHEVVFGGGTKLTVL

SEQ ID NO: 89 - protein, 112 aa, synthetic construct
NFMLTQPRSVSESPGKTVTISCTRSSGSIGSDYVHWYQQRPGSQPTTVIYEDNQRPSGVPDRFSGSIDSSSNSASLTISGLKTEDEATYYCQSYDRSNHEVVFGVGTKLTVL

SEQ ID NO: 90 - protein, 112 aa, synthetic construct
NFMLTQPRSVSESPGKTVTISCTRSSGSIGSDYVHWYQQRPGSKPTTVIYEDNQRPSGVPDRFSGSIDSSSNSASLTISGLKTEDEADYYCQSYDRSNHEVVFGGGTKLTVL

SEQ ID NO: 91 - protein, 112 aa, synthetic construct
NFMLTQPRSVSESPGKTVTISCTRSSGSIGSDYVHWYQQRPGSSPTTVIYEDNQRPSGVPDRFSGSIDSSSNSASLTISGLKTEDEADYYCQSYDRSNHEVVFGWGTKLTVL

SEQ ID NO: 92 - protein, 112 aa, synthetic construct
NFMLTQPRSVSESPGKTVTISCTRSSGSIGSDYVHWYQFRPGSSPTTVIYEDNQRPSGVPDRFSGSIDSSSNSASLTISGLKTEDEADYYCQSYDRSNHEVVFGGGTKLTVL

SEQ ID NO: 93 - protein, 112 aa, synthetic construct
NFMLTQPRSVSESPGKTVTISCTRSSGSIGSDYVHWYQQRPGSSPTTVIYEDNQRPSGVPDRFSGSIDSSSNSASLTISGLKTEDEAAYYCQSYDRSNHEVVFGGGTKLTVL

SEQ ID NO: 94 - protein, 112 aa, synthetic construct
NFMLTQPRSVSESPGKTVTISCTRYSGSIGSDYVHWYQVRPGSSPTTVIYEDNQRPSGVPDRFSGSIDSSSNSASLTISGLKTEDEAHYYCQSYDRSNHEVVFGHGTKLTVL

SEQ ID NO: 95 - protein, 131 aa, synthetic construct
XVQLVQSXAEVKKPGSXVKVSCKASGGTFSSYAISWVRXAPGQXXEXMGGIIPIFGTANYAQKFQGRVTITADXSTSTAYMXLXSLRSDDTAXYXCARDNLGYCSGGSCYSDYYYYYMDVXGXGTLVTVSS

SEQ ID NO: 96 - protein, 129 aa, synthetic construct
SSGGGGSGGGGSGGGASNFMLTQPRSVSESPGKTVTISCTRSXGSIGSDXVHWXQXRPGSXXTXVIXEDNQRPSGVPDRFSGSIDSSSNSASLTISGLKTEDEAXYXCQSYDRSNHEVVXGXGTKLTVX

SEQ ID NO: 97 - protein, 30 aa, synthetic construct
XVQLVQSXAEVKKPGSXVKVSCKASGGTFS

SEQ ID NO: 98 - protein, 14 aa, synthetic construct
WVRXAPGQXXEXMG

SEQ ID NO: 99 - protein, 32 aa, synthetic construct
RVTITADXSTSTAYMXLXSLRSDDTAXYXCAR

SEQ ID NO: 100 - protein, 11 aa, synthetic construct
XGXGTLVTVSS

SEQ ID NO: 101 - protein, 15 aa, synthetic construct
WXQXRPGSXXTXVIX

SEQ ID NO: 102 - protein, 34 aa, synthetic construct
GVPDRFSGSIDSSSNSASLTISGLKTEDEAXYXC

SEQ ID NO: 103 - protein, 10 aa, synthetic construct
XGXGTKLTVX

SEQ ID NO: 104 - protein, 13 aa, synthetic construct
TRSXGSIGSDXVH

SEQ ID NO: 105 - protein, 133 aa, synthetic construct
QVQLVQSGAEVKKPGSSVKVSCKASGGTFSSYAISWVRQAPGQGLEWMGGIIPIFGTANYAQKFQGRVTITADESTSTAYMELRSLRSDDTAVYYCARDNLGYCSGGSCYSDYYYYYMDVWGQGTLVTVSSGG

SEQ ID NO: 106 - protein, 133 aa, synthetic construct
QVQLVQSGAEVKKPGSYVKVSCKASGGTFSSYAISWVRSAPGQGLEWMGGIIPIFGTANYAQKFQGRVTITADESTSTAYMELRSLRSDDTAVYYCARDNLGYCSGGSCYSDYYYYYMDVWGQGTLVTVSSGG

SEQ ID NO: 107 - protein, 133 aa, synthetic construct
QVQLVQSGAEVKKPGSSVKVSCKASGGTFSSYAISWVRSAPGQGLEWMGGIIPIFGTANYAQKFQGRVTITADESTSTAYMELRSLRSDDTAVYYCARDNLGYCSGGSCYSDYYYYYMDVWGQGTLVTVSSGG

SEQ ID NO: 108 - protein, 132 aa, synthetic construct
QVQLVQSGAEVKKPGSSVKVSCKASGGTFSSYAISWVRQAPGQGLEWMGGIIPIFGTANYAQKFQGRVTITADESTSTAYMLRSLRSDDTAVYYCARDNLGYCSGGSCYSDYYYYYMDVWGQGTLVTVSSGG

SEQ ID NO: 109 - protein, 133 aa, synthetic construct
QVQLVQSGAEVKKPGSSVKVSCKASGGTFSSYAISWVRQAPGQGLEWMGGIIPIFGTANYAQKFQGRVTITADESTSTAYMELRSLRSDDTAVYYCARDNLGYCSGGSCYSDYYYYYMDVWGTGTLVTVSSGG

SEQ ID NO: 110 - protein, 132 aa, synthetic construct
QVQLVQSAEVKKPGSSVKVSCKASGGTFSSYAISWVRQAPGQGLEWMGGIIPIFGTANYAQKFQGRVTITADESTSTAYMELRSLRSDDTAVYYCARDNLGYCSGGSCYSDYYYYYMDVWGRGTLVTVSSGG

SEQ ID NO: 111 - protein, 133 aa, synthetic construct
QVQLVQSGAEVKKPGSSVKVSCKASGGTFSSYAISWVRQAPGQGLEWMGGIIPIFGTANYAQKFQGRVTITADESTSTAYMELRSLRSDDTATYYCARDNLGYCSGGSCYSDYYYYYMDVWGTGTLVTVSSGG

SEQ ID NO: 112 - protein, 133 aa, synthetic construct
QVQLVQSGAEVKKPGSSVKVSCKASGGTFSSYAISWVRQAPGQPLEWMGGIIPIFGTANYAQKFQGRVTITADESTSTAYMELRSLRSDDTAVYYCARDNLGYCSGGSCYSDYYYYYMDVWGRGTLVTVSSGG

SEQ ID NO: 113 - protein, 133 aa, synthetic construct
QVQLVQSGAEVKKPGSSVKVSCKASGGTFSSYAISWVRQAPGQRLEWMGGIIPIFGTANYAQKFQGRVTITADESTSTAYMELRSLRSDDTANYYCARDNLGYCSGGSCYSDYYYYYMDVWGQGTLVTVSSGG

SEQ ID NO: 114 - protein, 133 aa, synthetic construct
QVQLVQSGAEVKKPGSSVKVSCKASGGTFSSYAISWVRQAPGQNLEWMGGIIPIFGTANYAQKFQGRVTITADESTSTAYMELRSLRSDDTAIYYCARDNLGYCSGGSCYSDYYYYYMDVWGQGTLVTVSSGG

SEQ ID NO: 115 - protein, 132 aa, synthetic construct
QVQLVQSGAEVKKPGSSVKVSCKASGGTFSSYAISWVRQAPGQSLEWMGGIIPIFGTANYAQKFQGRVTITADSTSTAYMELRSLRSDDTAQYYCARDNLGYCSGGSCYSDYYYYYMDVWGQGTLVTVSSGG

SEQ ID NO: 116 - protein, 133 aa, synthetic construct
QVQLVQSGAEVKKPGSSVKVSCKASGGTFSSYAISWVRQAPGQGLEWMGGIIPIFGTANYAQKFQGRVTITADESTSTAYMELRSLRSDDTAVYYCARDNLGYCSGGSCYSDYYYYYMDVWGKGTLVTVSSGG

SEQ ID NO: 117 - protein, 133 aa, synthetic construct
QVQLVQSGAEVKKPGSSVKVSCKASGGTFSSYAISWVRQAPGQKLEWMGGIIPIFGTANYAQKFQGRVTITADESTSTAYMELRSLRSDDTAVYYCARDNLGYCSGGSCYSDYYYYYMDVWGPGTLVTVSSGG

SEQ ID NO: 118 - protein, 133 aa, synthetic construct
HVQLVQSGAEVKKPGSSVKVSCKASGGTFSSYAISWVREAPGQGLEWMGGIIPIFGTANYAQKFQGRVTITADESTSTAYMELRSLRSDDTAVYYCARDNLGYCSGGSCYSDYYYYYMDVWGDGTLVTVSSGG

SEQ ID NO: 119 - protein, 133 aa, synthetic construct
HVQLVQSGAEVKKPGSSVKVSCKASGGTFSSYAISWVRQAPGQQLEWMGGIIPIFGTANYAQKFQGRVTITADESTSTAYMELRSLRSDDTATYYCARDNLGYCSGGSCYSDYYYYYMDVWGPGTLVTVSSGG

SEQ ID NO: 120 - protein, 133 aa, synthetic construct
HVQLVQSGAEVKKPGSSVKVSCKASGGTFSSYAISWVRQAPGQQLEWMGGIIPIFGTANYAQKFQGRVTITADESTSTAYMELRSLRSDDTALYYCARDNLGYCSGGSCYSDYYYYYMDVWGKGTLVTVSSGG

SEQ ID NO: 121 - protein, 133 aa, synthetic construct
QVQLVQSGAEVKKPGSSVKVSCKASGGTFSSYAISWVRQAPGQALEWMGGIIPIFGTANYAQKFQGRVTITADESTSTAYMELRSLRSDDTAAYYCARDNLGYCSGGSCYSDYYYYYMDVWGPGTLVTVSSGG

SEQ ID NO: 122 - protein, 133 aa, synthetic construct
QVQLVQSGAEVKKPGSSVKVSCKASGGTFSSYAISWVRQAPGQKLEWMGGIIPIFGTANYAQKFQGRVTITADESTSTAYMELRSLRSDDTAKYYCARDNLGYCSGGSCYSDYYYYYMDVWGIGTLVTVSSGG

SEQ ID NO: 123 - protein, 133 aa, synthetic construct
QVQLVQSGAEVKKPGSSVKVSCKASGGTFSSYAISWVRQAPGQTLEWMGGIIPIFGTANYAQKFQGRVTITADESTSTAYMELRSLRSDDTAVYYCARDNLGYCSGGSCYSDYYYYYMDVWGTGTLVTVSSGG

SEQ ID NO: 124 - protein, 133 aa, synthetic construct
QVQLVQSGAEVKKPGSSVKVSCKASGGTFSSYAISWVRAAPGQGLEWMGGIIPIFGTANYAQKFQGRVTITADESTSTAYMELKSLRSDDTAVYYCARDNLGYCSGGSCYSDYYYYYMDVWGPGTLVTVSSGG

SEQ ID NO: 125 - protein, 114 aa, synthetic construct
ASNFMLTQPRSVSESPGKTVTISCTRSSGSIGSDYVHWYQQRPGSSPTTVIYEDNQRPSGVPDRFSGSIDSSSNSASLTISGLKTEDEADYYCQSYDRSNHEVVFGGGTKLTVL

SEQ ID NO: 126 - protein, 114 aa, synthetic construct
ASNFMLTQPRSVSESPGKTVTISCTRSSGSIGSDYVHWYQLRPGSSPTTVIYEDNQRPSGVPDRFSGSIDSSSNSASLTISGLKTEDEADYYCQSYDRSNHEVVFGGGTKLTVL

SEQ ID NO: 127 - protein, 114 aa, synthetic construct
ASNFMLTQPRSVSESPGKTVTISCTRSSGSIGSDYVHWYQYRPGSSPTTVIYEDNQRPSGVPDRFSGSIDSSSNSASLTISGLKTEDEADYYCQSYDRSNHEVVFGGGTKLTVL

SEQ ID NO: 128 - protein, 114 aa, synthetic construct
ASNFMLTQPRSVSESPGKTVTISCTRSSGSIGSDYVHWYQSRPGSSPTTVIYEDNQRPSGVPDRFSGSIDSSSNSASLTISGLKTEDEASYYCQSYDRSNHEVVFGGGTKLTVL

SEQ ID NO: 129 - protein, 114 aa, synthetic construct
ASNFMLTQPRSVSESPGKTVTISCTRSSGSIGSDYVHWYQQRPGSSPTTVIYEDNQRPSGVPDRFSGSIDSSSNSASLTISGLKTEDEANYYCQSYDRSNHEVVFGGGTKLTVL

SEQ ID NO: 130 - protein, 114 aa, synthetic construct
ASNFMLTQPRSVSESPGKTVTISCTRSSGSIGSDYVHWYQIRPGSSPTTVIYEDNQRPSGVPDRFSGSIDSSSNSASLTISGLKTEDEATYYCQSYDRSNHEVVFGGGTKLTVL

SEQ ID NO: 131 - protein, 114 aa, synthetic construct
ASNFMLTQPRSVSESPGKTVTISCTRSSGSIGSDYVHWYQTRPGSSPTTVIYEDNQRPSGVPDRFSGSIDSSSNSASLTISGLKTEDEAFYYCQSYDRSNHEVVFGGGTKLTVL

SEQ ID NO: 132 - protein, 114 aa, synthetic construct
ASNFMLTQPRSVSESPGKTVTISCTRSSGSIGSDYVHWYQQRPGSSPTTVIYEDNQRPSGVPDRFSGSIDSSSNSASLTISGLKTEDEADYYCQSYDRSNHEVVFGSGTKLTVL

SEQ ID NO: 133 - protein, 114 aa, synthetic construct
ASNFMLTQPRSVSESPGKTVTISCTRSSGSIGSDYVHWYQQRPGSSPTTVIYEDNQRPSGVPDRFSGSIDSSSNSASLTISGLKTEDEADYYCQSYDRSNHEVVFGGGTKLTVI

SEQ ID NO: 134 - protein, 114 aa, synthetic construct
ASNFMLTQPRSVSESPGKTVTISCTRSSGSIGSDYVHWYQARPGSSPTTVIYEDNQRPSGVPDRFSGSIDSSSNSASLTISGLKTEDEADYYCQSYDRSNHEVVFGGGTKLTVL

SEQ ID NO: 135 - protein, 114 aa, synthetic construct
ASNFMLTQPRSVSESPGKTVTISCTRSSGSIGSDYVHWYQRRPGSSPTTVIYEDNQRPSGVPDRFSGSIDSSSNSASLTISGLKTEDEADYYCQSYDRSNHEVVFGGGTKLTVL

SEQ ID NO: 136 - protein, 114 aa, synthetic construct
ASNFMLTQPRSVSESPGKTVTISCTRSSGSIGSDYVHWYQQRPGSPPTTVIYEDNQRPSGVPDRFSGSIDSSSNSASLTISGLKTEDEADYYCQSYDRSNHEVVFGGGTKLTVL

SEQ ID NO: 137 - protein, 114 aa, synthetic construct
ASNFMLTQPRSVSESPGKTVTISCTRSYGSIGSDYVHWYQQRPGSTPTTVIYEDNQRPSGVPDRFSGSIDSSSNSASLTISGLKTEDEADYYCQSYDRSNHEVVFGGGTKLTVL

SEQ ID NO: 138 - protein, 114 aa, synthetic construct
ASNFMLTQPRSVSESPGKTVTISCTRSSGSIGSDYVHWYQQRPGSTPTTVIYEDNQRPSGVPDRFSGSIDSSSNSASLTISGLKTEDEADYYCQSYDRSNHEVVFGYGTKLTVL

SEQ ID NO: 139 - protein, 114 aa, synthetic construct
ASNFMLTQPRSVSESPGKTVTISCTRSSGSIGSDYVHWYQQRPGSSPTTVIYEDNQRPSGVPDRFSGSIDSSSNSASLTISGLKTEDEAVYYCQSYDRSNHEVVFGGGTKLTVL

SEQ ID NO: 140 - protein, 114 aa, synthetic construct
ASNFMLTQPRSVSESPGKTVTISCTRSSGSIGSDYVHWYQQRPGSSPTTVIYEDNQRPSGVPDRFSGSIDSSSNSASLTISGLKTEDEALYYCQSYDRSNHEVVFGGGTKLTVL

SEQ ID NO: 141 - protein, 114 aa, synthetic construct
ASNFMLTQPRSVSESPGKTVTISCTRSSGSIGSDYVHWYQQRPGSSPTTVIYEDNQRPSGVPDRFSGSIDSSSNSASLTISGLKTEDEASYYCQSYDRSNHEVVFGGGTKLTVL

SEQ ID NO: 142 - protein, 114 aa, synthetic construct
ASNFMLTQPRSVSESPGKTVTISCTRSSGSIGSDYVHWYQQRPGSAPTTVIYEDNQRPSGVPDRFSGSIDSSSNSASLTISGLKTEDEASYYCQSYDRSNHEVVFGGGTKLTVL

SEQ ID NO: 143 - protein, 114 aa, synthetic construct
ASNFMLTQPRSVSESPGKTVTISCTRSSGSIGSDYVHWYQQRPGSVPTTVIYEDNQRPSGVPDRFSGSIDSSSNSASLTISGLKTEDEAAYYCQSYDRSNHEVVFGLGTKLTVL

SEQ ID NO: 144 - protein, 114 aa, synthetic construct
ASNFMLTQPRSVSESPGKTVTISCTRSSGSIGSDYVHWYQVRPGSSPTTVIYEDNQRPSGVPDRFSGSIDSSSNSASLTISGLKTEDEARYYCQSYDRSNHEVVFGGGTKLTVL

SEQ ID NO: 145 - protein, 164 aa, synthetic construct
XXQVQLVESGGGLVQPGGSLRLSCAASGFTFSSGSYAMSWVRQAPGKGLEWVGGISPKTSGGWNTYYADSVKGRFTISRDNSKNFDTFTLYLQMNSLRAEDTAVYYCATXRRDQLYGXXPLXYGGGGGGGGYYYYGSGXEXLYDAFDYDPQYSWGQGTLVTVSS

SEQ ID NO: 146 - protein, 115 aa, synthetic construct
QSVLTQPPSVSGAPGQTVTISCXGSSSNIGSYYDVSWYQQLPGTAPKLLIYDDSNRPSGVPDRFSGSXDXKSGNTASLTISGLQAEDEADYYCQSWDSSLSGXVVFGGGTKLTVL

SEQ ID NO: 147 - protein, 117 aa, synthetic construct
DIVMTQSPSSLSASVGDRVTITCRASQSILYSSSSKSYLAWYQQKPGQAPKLLIYGASSRASGVPSRFSGSGSGTDFTLTISSLQPEDFATYYCQQYNSWVPPLXFTFGQGTKVEIK

SEQ ID NO: 148 - protein, 256 aa, synthetic construct
MAEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGASDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKAAASAHHHHHH

SEQ ID NO: 149 - protein, 258 aa, synthetic construct
MAEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGASDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKAAASAHHHHHH

SEQ ID NO: 150 - protein, 254 aa, synthetic construct
MAQVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIFGVVTNFDNWGQGTLVTVSSGGGGSGGGGSGGGASDIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKVEIKAAASAHHHHHH

SEQ ID NO: 151 - protein, 23 aa, synthetic construct
DIQMTQSPSSLSASVGDRVTITC

SEQ ID NO: 152 - protein, 15 aa, synthetic construct
WYQQKPGKAPKLLIY

SEQ ID NO: 153 - protein, 32 aa, synthetic construct
GVPSRFSGSGSGTDFTLTISSLQPEDVATYYC

SEQ ID NO: 154 - protein, 10 aa, synthetic construct
FGQGTKVEIK

SEQ ID NO: 155 - protein, 30 aa, synthetic construct
EVQLVESGGGLVQPGRSLRLSCAASGFTFD

SEQ ID NO: 156 - protein, 14 aa, synthetic construct
WVRQAPGKGLEWVS

SEQ ID NO: 157 - protein, 32 aa, synthetic construct
RFTISRDNAKNSLYLQMNSLRAEDTAVYYCAK

SEQ ID NO: 158 - protein, 15 aa, synthetic construct
WYQQKPGKAPKVLIY

SEQ ID NO: 159 - protein, 32 aa, synthetic construct
GVPSRFSGSGSGTDFTLTISSLQPEDFATYYC

SEQ ID NO: 160 - protein, 30 aa, synthetic construct
EVQLVESGGGLVQPGGSLRLSCAASGYTFT

SEQ ID NO: 161 - protein, 14 aa, synthetic construct
WVRQAPGKGLEWVG

SEQ ID NO: 162 - protein, 32 aa, synthetic construct
RFTFSLDTSKSTAYLQMNSLRAEDTAVYYCAK

SEQ ID NO: 163 - protein, 23 aa, synthetic construct
DIQMTQSPSSLSASAGDRVTITC

SEQ ID NO: 164 - protein, 15 aa, synthetic construct
WYQQKPGKAPKRLIY

SEQ ID NO: 165 - protein, 32 aa, synthetic construct
GVPSRFSGSGSGTEFTLTISSLQPEDLASYYC

SEQ ID NO: 166 - protein, 30 aa, synthetic construct
QVQLVQSGAEVKKPGASVKVSCKVSGFTLT

SEQ ID NO: 167 - protein, 14 aa, synthetic construct
WVRQAPGKGLEWMG

SEQ ID NO: 168 - protein, 32 aa, synthetic construct
RVTMTEDTSTDTAYMELTSLRSEDTAVYYCST

SEQ ID NO: 169 - protein, 11 aa, synthetic construct
RASQGIRNYLA

SEQ ID NO: 170 - protein, 7 aa, synthetic construct
AASTLQS

SEQ ID NO: 171 - protein, 9 aa, synthetic construct
QRYNRAPYT

SEQ ID NO: 172 - protein, 11 aa, synthetic construct
SASQDISNYLN

SEQ ID NO: 173 - protein, 7 aa, synthetic construct
FTSSLHS

SEQ ID NO: 174 - protein, 9 aa, synthetic construct
QQYSTVPWT

SEQ ID NO: 175 - protein, 11 aa, synthetic construct
RASQAIRNDLG

SEQ ID NO: 176 - protein, 7 aa, synthetic construct
AAFNLQS

SEQ ID NO: 177 - protein, 9 aa, synthetic construct
QQYNRYPWT

SEQ ID NO: 178 - protein, 5 aa, synthetic construct
DYAMH

SEQ ID NO: 179 - protein, 17 aa, synthetic construct
AITWNSGHIDYADSVEG

SEQ ID NO: 180 - protein, 12 aa, synthetic construct
VSYLSTASSLDY

SEQ ID NO: 181 - protein, 5 aa, synthetic construct
NYGMN

SEQ ID NO: 182 - protein, 17 aa, synthetic construct
WINTYTGEPTYAADFKR

SEQ ID NO: 183 - protein, 14 aa, synthetic construct
YPHYYGSSHWYFDV

SEQ ID NO: 184 - protein, 5 aa, synthetic construct
ELSIH

SEQ ID NO: 185 - protein, 17 aa, synthetic construct
GFDPEDGETIYAQKFQG

SEQ ID NO: 186 - protein, 10 aa, synthetic construct
IFGVVTNFDN

SEQ ID NO: 187 - protein, 14 aa, synthetic construct
WVRXAPGKXXEXVS

SEQ ID NO: 188 - protein, 32 aa, synthetic construct
RFTISRDXAKNSLYLXMXSLRAEDTAXYXCAK

SEQ ID NO: 189 - protein, 11 aa, synthetic construct
XGXGTLVTVSS

SEQ ID NO: 190 - protein, 15 aa, synthetic construct
WXQXKPGKXXKXLIX

SEQ ID NO: 191 - protein, 32 aa, synthetic construct
GVPSRFSGSGSGTDFTLTISSLQPEDVAXYXC

SEQ ID NO: 192 - protein, 10 aa, synthetic construct
XGXGTKVEIX

SEQ ID NO: 193 - protein, 14 aa, synthetic construct
WVRQAPGKXLEWVS

SEQ ID NO: 194 - protein, 32 aa, synthetic construct
RFTISRDNAKNSLYLQMNSLRAEDTAXYYCAK

SEQ ID NO: 195 - protein, 11 aa, synthetic construct
WGXGTLVTVSS

SEQ ID NO: 196 - protein, 15 aa, synthetic construct
WYQXKPGKAPKLLIY

SEQ ID NO: 197 - protein, 14 aa, synthetic construct
WVRXAPGKXXEXVG

SEQ ID NO: 198 - protein, 32 aa, synthetic construct
RFTFSLDXSKSTAYLXMXSLRXEDTAXYXCAK

SEQ ID NO: 199 - protein, 32 aa, synthetic construct
GVPSRFSGSGSGTDFTLTISSLQPEDFAXYXC

SEQ ID NO: 200 - protein, 14 aa, synthetic construct
WVRQAPGKXLEWVG

SEQ ID NO: 201 - protein, 32 aa, synthetic construct
RFTFSLDTSKSTAYLQMNSLRXEDTAXYYCAK

SEQ ID NO: 202 - protein, 15 aa, synthetic construct
WYQXKPGKAPKVLIY

SEQ ID NO: 203 - protein, 14 aa, synthetic construct
WVRXAPGKXXEXMG

SEQ ID NO: 204 - protein, 32 aa, synthetic construct
RVTMTEDXSTDTAYMXLXSLRSEDTAXYXCST

SEQ ID NO: 205 - protein, 32 aa, synthetic construct
GVPSRFSGSGSGTEFTLTISSLQPEDLAXYXC

SEQ ID NO: 206 - protein, 14 aa, synthetic construct
WVRQAPGKXLEWMG

SEQ ID NO: 207 - protein, 32 aa, synthetic construct
RVTMTEDTSTDTAYMELTSLRSEDTAXYYCST

SEQ ID NO: 208 - protein, 15 aa, synthetic construct
WYQXKPGKAPKRLIY

SEQ ID NO: 209 - protein, 302 aa, synthetic construct
MKYLLPTAAAGLLLLAAQPAMAEVQLVESGGGLVQPGGSLRLSCAASGYTFTNYGMNWVRQAPGKXLEWVGWINTYTGEPTYAADFKRRFTFSLDTSKSTAYLQMNSLRAEDTAXYYCAKYPHYYGSSHWYFDVWGXGTLVTVSSGGGGSGGGGSGGGASDIQMTQSPSSLSASVGDRVTITCSASQDISNYLNWYQXKPGKAPKVLIYFTSSLHSGVPSRFSGSGSGTDFTLTISSLQPEDFATYYCQQYSTVPWTFGQGTKVEIKAAASAHHHHHHKLDYKDHDGDYKDHDIDYKDDDDK

SEQ ID NO: 210 - protein, 302 aa, synthetic construct
MKYLLPTAAAGLLLLAAQPAMAEVQLVESGGGLVQPGGSLRLSCAASGYTFTNYGMNWVRQAPGKALEWVGWINTYTGEPTYAADFKRRFTFSLDTSKSTAYLQMNSLRAEDTAAYYCAKYPHYYGSSHWYFDVWGTGTLVTVSSGGGGSGGGGSGGGASDIQMTQSPSSLSASVGDRVTITCSASQDISNYLNWYQFKPGKAPKVLIYFTSSLHSGVPSRFSGSGSGTDFTLTISSLQPEDFATYYCQQYSTVPWTFGQGTKVEIKAAASAHHHHHHKLDYKDHDGDYKDHDIDYKDDDDK

SEQ ID NO: 211 - protein, 302 aa, synthetic construct
MKYLLPTAAAGLLLLAAQPAMAEVQLVESGGGLVQPGGSLRLSCAASGYTFTNYGMNWVRQAPGKALEWVGWINTYTGEPTYAADFKRRFTFSLDTSKSTAYLQMNSLRAEDTATYYCAKYPHYYGSSHWYFDVWGDGTLVTVSSGGGGSGGGGSGGGASDIQMTQSPSSLSASVGDRVTITCSASQDISNYLNWYQLKPGKAPKVLIYFTSSLHSGVPSRFSGSGSGTDFTLTISSLQPEDFATYYCQQYSTVPWTFGQGTKVEIKAAASAHHHHHHKLDYKDHDGDYKDHDIDYKDDDDK

SEQ ID NO: 212 - protein, 302 aa, synthetic construct
MKYLLPTAAAGLLLLAAQPAMAEVQLVESGGGLVQPGGSLRLSCAASGYTFTNYGMNWVRQAPGKPLEWVGWINTYTGEPTYAADFKRRFTFSLDTSKSTAYLQMNSLRAEDTAIYYCAKYPHYYGSSHWYFDVWGSGTLVTVSSGGGGSGGGGSGGGASDIQMTQSPSSLSASVGDRVTITCSASQDISNYLNWYQSKPGKAPKVLIYFTSSLHSGVPSRFSGSGSGTDFTLTISSLQPEDFATYYCQQYSTVPWTFGQGTKVEIKAAASAHHHHHHKLDYKDHDGDYKDHDIDYKDDDDK

SEQ ID NO: 213 - protein, 302 aa, synthetic construct
MKYLLPTAAAGLLLLAAQPAMAEVQLVESGGGLVQPGGSLRLSCAASGYTFTNYGMNWVRQAPGKSLEWVGWINTYTGEPTYAADFKRRFTFSLDTSKSTAYLQMNSLRTEDTATYYCAKYPHYYGSSHWYFDVWGSGTLVTVSSGGGGSGGGGSGGGASDIQMTQSPSSLSASVGDRVTITCSASQDISNYLNWYQTKPGKAPKVLIYFTSSLHSGVPSRFSGSGSGTDFTLTISSLQPEDFATYYCQQYSTVPWTFGQGTKVEIKAAASAHHHHHHKLDYKDHDGDYKDHDIDYKDDDDK

SEQ ID NO: 214 - protein, 302 aa, synthetic construct
MKYLLPTAAAGLLLLAAQPAMAEVQLVESGGGLVQPGGSLRLSCAASGYTFTNYGMNWVRQAPGKTLEWVGWINTYTGEPTYAADFKRRFTFSLDTSKSTAYLQMNSLRAEDTALYYCAKYPHYYGSSHWYFDVWGPGTLVTVSSGGGGSGGGGSGGGASDIQMTQSPSSLSASVGDRVTITCSASQDISNYLNWYQIKPGKAPKVLIYFTSSLHSGVPSRFSGSGSGTDFTLTISSLQPEDFATYYCQQYSTVPWTFGQGTKVEIKAAASAHHHHHHKLDYKDHDGDYKDHDIDYKDDDDK

SEQ ID NO: 215 - protein, 300 aa, synthetic construct
MKYLLPTAAAGLLLLAAQPAMAEVQLVESGGGLVQPGRSLRLSCAASGFTFDDYAMHWVRQAPGKXLEWVSAITWNSGHIDYADSVEGRFTISRDNAKNSLYLQMNSLRAEDTAXYYCAKVSYLSTASSLDYWGXGTLVTVSSGGGGSGGGGSGGGASDIQMTQSPSSLSASVGDRVTITCRASQGIRNYLAWYQXKPGKAPKLLIYAASTLQSGVPSRFSGSGSGTDFTLTISSLQPEDVATYYCQRYNRAPYTFGQGTKVEIKAAASAHHHHHHKLDYKDHDGDYKDHDIDYKDDDDK

SEQ ID NO: 216 - protein, 300 aa, synthetic construct
MKYLLPTAAAGLLLLAAQPAMAEVQLVESGGGLVQPGRSLRLSCAASGFTFDDYAMHWVRQAPGKYLEWVSAITWNSGHIDYADSVEGRFTISRDNAKNSLYLQMNSLRAEDTAYYYCAKVSYLSTASSLDYWGSGTLVTVSSGGGGSGGGGSGGGASDIQMTQSPSSLSASVGDRVTITCRASQGIRNYLAWYQSKPGKAPKLLIYAASTLQSGVPSRFSGSGSGTDFTLTISSLQPEDVATYYCQRYNRAPYTFGQGTKVEIKAAASAHHHHHHKLDYKDHDGDYKDHDIDYKDDDDK

SEQ ID NO: 217 - protein, 300 aa, synthetic construct
MKYLLPTAAAGLLLLAAQPAMAEVQLVESGGGLVQPGRSLRLSCAASGFTFDDYAMHWVRQAPGKGLEWVSAITWNSGHIDYADSVEGRFTISRDNAKNSLYLQMNSLRAEDTADYYCAKVSYLSTASSLDYWGQGTLVTVSSGGGGSGGGGSGGGASDIQMTQSPSSLSASVGDRVTITCRASQGIRNYLAWYQRKPGKAPKLLIYAASTLQSGVPSRFSGSGSGTDFTLTISSLQPEDVATYYCQRYNRAPYTFGQGTKVEIKAAASAHHHHHHKLDYKDHDGDYKDHDIDYKDDDDK

SEQ ID NO: 218 - protein, 300 aa, synthetic construct
MKYLLPTAAAGLLLLAAQPAMAEVQLVESGGGLVQPGRSLRLSCAASGFTFDDYAMHWVRQAPGKRLEWVSAITWNSGHIDYADSVEGRFTISRDNAKNSLYLQMNSLRAEDTAFYYCAKVSYLSTASSLDYWGTGTLVTVSSGGGGSGGGGSGGGASDIQMTQSPSSLSASVGDRVTITCRASQGIRNYLAWYQLKPGKAPKLLIYAASTLQSGVPSRFSGSGSGTDFTLTISSLQPEDVATYYCQRYNRAPYTFGQGTKVEIKAAASAHHHHHHKLDYKDHDGDYKDHDIDYKDDDDK

SEQ ID NO: 219 - protein, 300 aa, synthetic construct; identical to SEQ ID NO: 218

SEQ ID NO: 220 - protein, 300 aa, synthetic construct
MKYLLPTAAAGLLLLAAQPAMAEVQLVESGGGLVQPGRSLRLSCAASGFTFDDYAMHWVRQAPGKTLEWVSAITWNSGHIDYADSVEGRFTISRDNAKNSLYLQMNSLRAEDTASYYCAKVSYLSTASSLDYWGPGTLVTVSSGGGGSGGGGSGGGASDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKAAASAHHHHHHKLDYKDHDGDYKDHDIDYKDDDDK

SEQ ID NO: 221 - DNA, 28 bp, synthetic construct
  source 1..28: mol_type = other DNA
gtaaccaccacacccgccgcgcttaatg

SEQ ID NO: 222 - DNA, 26 bp, synthetic construct
  source 1..26: mol_type = other DNA
caccatacccacgccgaaacaagcgc

SEQ ID NO: 223 - DNA, 18 bp, synthetic construct
  source 1..18: mol_type = other DNA
ggtctgctgctcctcgct

SEQ ID NO: 224 - DNA, 35 bp, synthetic construct
  source 1..35: mol_type = other DNA
tatggtctcactaacccattccaaknntttacctg

SEQ ID NO: 225 - DNA, 35 bp, synthetic construct
  source 1..35: mol_type = other DNA
tatggtctcattagtgcaattacctggaactctgg

SEQ ID NO: 226 - DNA, 32 bp, synthetic construct
  source 1..32: mol_type = other DNA
tatggtctcaccttggcacagtagtaknnagc

SEQ ID NO: 227 - DNA, 33 bp, synthetic construct
  source 1..33: mol_type = other DNA
tatggtctcaaaggtttcttacttgtccaccgc

SEQ ID NO: 228 - DNA, 33 bp, synthetic construct
  source 1..33: mol_type = other DNA
tatggtctcacagtgaccaaagtaccknnaccc

SEQ ID NO: 229 - DNA, 37 bp, synthetic construct
  source 1..37: mol_type = other DNA
tatggtctcaactgtcagcagtggtggtggtggtagc

SEQ ID NO: 230 - DNA, 34 bp, synthetic construct
  source 1..34: mol_type = other DNA
tatggtctcatggagccttacccggtttknnttg

SEQ ID NO: 231 - DNA, 32 bp, synthetic construct
  source 1..32: mol_type = other DNA
tatggtctcatccaaagctgctgatttacgct

SEQ ID NO: 232 - DNA, 23 bp, synthetic construct
  source 1..23: mol_type = other DNA
gtgatggtgatgatgatgtgcgg

SEQ ID NO: 233 - DNA, 33 bp, synthetic construct
  source 1..33: mol_type = other DNA
tatggtctcagacccattccaaknncttacccg

SEQ ID NO: 234 - DNA, 36 bp, synthetic construct
  source 1..36: mol_type = other DNA
tatggtctcaggtcggttggattaatacctacaccg

SEQ ID NO: 235 - DNA, 37 bp, synthetic construct
  source 1..37: mol_type = other DNA
tatggtctcaacttagcacaatagtaknnagcagtgt

SEQ ID NO: 236 - DNA, 34 bp, synthetic construct
  source 1..34: mol_type = other DNA
tatggtctcaaagtacccacactattacggttcc

SEQ ID NO: 237 - DNA, 30 bp, synthetic construct
  source 1..30: mol_type = other DNA
tatggtctcaccaaagtaccknnaccccaa

SEQ ID NO: 238 - DNA, 31 bp, synthetic construct
  source 1..31: mol_type = other DNA
tatggtctcattggttactgtgagttccggt

SEQ ID NO: 239 - DNA, 35 bp, synthetic construct
  source 1..35: mol_type = other DNA
tatggtctcactttacctggcttknnttggtacca

SEQ ID NO: 240 - DNA, 39 bp, synthetic construct
  source 1..39: mol_type = other DNA
tatggtctcaaaagccccgaaagttttgatttatttcac

SEQ ID NO: 241 - DNA, 22 bp, synthetic construct
  source 1..22: mol_type = other DNA
tgatggtgatgatgatgtgcgg

SEQ ID NO: 242 - DNA, 33 bp, synthetic construct
  source 1..33: mol_type = other DNA
tatggtctcagggagggatcatccctatctttg

SEQ ID NO: 243 - DNA, 31 bp, synthetic construct
  source 1..31: mol_type = other DNA
tatggtctcactctcgcacamnnatacacgg

SEQ ID NO: 244 - DNA, 38 bp, synthetic construct
  source 1..38: mol_type = other DNA
tatggtctcaagagacaacctaggatattgtagtggtg

SEQ ID NO: 245 - DNA, 35 bp, synthetic construct
  source 1..35: mol_type = other DNA
tatggtctcacgctgccaatgctgccactgctgcg

SEQ ID NO: 246 - DNA, 33 bp, synthetic construct
  source 1..33: mol_type = other DNA
tatggtctagcgactatgtgcattggnnkcagc

SEQ ID NO: 247 - DNA, 30 bp, synthetic construct
  source 1..30: mol_type = other DNA
tatggtctcagatgacmnnggtmnnggagc

SEQ ID NO: 248 - DNA, 34 bp, synthetic construct
  source 1..34: mol_type = other DNA
tatggtctcacatcnnkgaggataaccaaagacc

SEQ ID NO: 249 - DNA, 39 bp, synthetic construct
  source 1..39: mol_type = other DNA
tatggtctcactgcgatcataagactgacamnngtagtc

SEQ ID NO: 250 - DNA, 35 bp, synthetic construct
  source 1..35: mol_type = other DNA
tatggtctcagcagcaatcatgaagtggtgnnkgg

SEQ ID NO: 251 - DNA, 22 bp, synthetic construct
  source 1..22: mol_type = other DNA
tgcggatgcggccgcgggctga

SEQ ID NO: 252 - DNA, 29 bp, synthetic construct
  source 1..29: mol_type = other DNA
tatggtctcatcccatmnnctcmnnccct

SEQ ID NO: 253 - DNA, 24 bp, synthetic construct
  source 1..24: mol_type = other DNA
ggctttgttagcagccggatctca

SEQ ID NO: 254 - protein, 260 aa, synthetic construct
MAQVQLVQSGAEVKKPGSSVKVSCKASGGTFSSYAISWVRQAPGQGLEWMGGIIPIFGTANYAQKFQGRVTITADESTSTAYMELRSLRSDDTAVYYCARDNLGYCSGGSCYSDYYYYYMDVWGQGTLVTVSSGGGGSGGGGSGGGASNFMLTQPRSVSESPGKTVTISCTRSSGSIGSDYVHWNQQRPGSSPTTVIYEDNQRPSGVPDRFSGSIDSSSNSASLTISGLKTEDEADYYCQSYDRSNHEVVFGGGTKLTVL

(19) United States
(12) Patent Application Publication
(10) Pub. No.: US 2026/0317413 A1
(43) Pub. Date: Oct. 15, 2026

(54) PH-SENSITIVE ANTIBODIES

(71) Applicant: Danmarks Tekniske Universitet, Kongens Lyngby (DK)

(72) Inventors: Jack Wade, Kongens Lyngby (DK); Andreas Hougaard Laustsen-Kiel, Kongens Lyngby (DK); Christoffer Vinther Sørensen, Kongens Lyngby (DK)

(21) Appl. No.: 19/481,587

(22) PCT Filed: May 8, 2024
(86) PCT No.: PCT/EP2024/062776

§ 371 Date: Nov. 5, 2025

(30) Foreign Application Priority Data
May 9, 2023 (EP) 23172448.5
Nov. 9, 2023 (EP) 23208932.6

(51) Int. Cl. C07K 16/24; C07K 16/22

(52) U.S. Cl. CPC C07K 16/241; C07K 16/22; C07K 2317/565; C07K 2317/567; C07K 2317/622; C07K 2317/92; C07K 2317/94

(57) ABSTRACT

A major challenge in the field of antibody-based therapeutics is the development of antibodies with enhanced duration of action, prolonged half-life and which can be delivered at lower dose to patients. Current approaches to achieve this rely on engineering of pH-dependent antigen binding properties to antibodies which are laborious, and typically require further optimization of pH-sensitive interactions in a low-throughput, case-to-case manner as they largely rely on introduction of histidines mutations to the specific antibody complementarity-determining regions (CDRs). In the present invention, the inventors provide antibodies and other antigen-binding proteins carrying mutations outside of the paratope conferring universal pH sensitive binding properties to antibodies. The invention also relates to methods of isolating and generating said antigen-binding proteins, compositions and medical uses thereof.

### TECHNICAL FIELD

The present invention relates to antigen-binding proteins, for example antibodies, comprising amino acid mutations conferring pH-dependent antigen binding properties to said antigen-binding proteins, methods of obtaining thereof, and medical uses thereof.

### BACKGROUND

Antigen-binding proteins, such as antibodies, are an attractive class of therapeutics due to their typically high specificity and affinity of their variable domain for their target, together with the ability to engage effector functions.

Antibody-mediated target degradation is an example of an attractive strategy to neutralize antigens due to the possibilities of achieving therapeutic efficacy at very low doses and modulating the function of a target without binding to an inhibitory epitope. A classical, antibody-based approach to degrade extracellular proteins using non-stoichiometric drug: target ratios is via the antibody recycling neonatal Fc receptor (FcRn). The FcRn, which binds pH-dependently to the antibody Fc domain, has been employed for degrading plasma antigens (Ref. 1). Antibodies that have low affinity for their antigen at endosomal acidic pH selectively release the target for lysosomal degradation during recycling, whereas the antibody itself is returned back to the plasma unbound. This allows the antibody to neutralize multiple target molecules (non-stoichiometric neutralization) and theoretically be administered at a lower dose than a conventional antibody (Ref. 1)

Fc variants with differential pH dependencies to FcRn have been developed, however, the mechanisms behind how antibodies can accommodate pH-dependent antigen-binding are not well understood. The current approaches for engineering of pH-dependent antigen binding properties are laborious, and typically require further optimization of pH-sensitive interactions in a low-throughput, case-to-case manner as they rely on introduction of histidines mutations to the specific antibody complementarity-determining regions (CDRs).

Methods and determinants conferring universal pH-sensitive antigen-binding properties to antibodies are lacking, thereby limiting the development of therapeutic antibodies with improved pharmacokinetic properties.

### SUMMARY

The invention is defined in the attached claims.

In the field of antibody engineering, approaches taking advantage of FcRn recycling to augment the circulating half-life of antibodies have been described in the art (Ref. 2). The FcRn receptor naturally has a high affinity for the Fc domain of antibodies at acidic pH and low affinity at neutral pH. This allows the antibody to bind FcRn in the acidic endosome and be rescued from lysosomal transfer and degradation. Upon being returned to the cell surface, lower affinity at neutral pH subsequently causes the antibody to be released from FcRn into the plasma.

This principle has been used in specific applications, where monoclonal antibodies have been engineered to have weaker antigen binding at acidic pH, and be recycled back into circulation, ready to target more venom toxin antigens.

By engineering the antibody Fc domain to interact with a higher affinity to FcRn at neutral pH, the internalization rate, and therefore the antigen degradation rate of recycling antibodies, is increased. More specifically, recycling IgG antibodies typically bind their cognate antigen with high affinity at about neutral pH (7.4) and low affinity at acidic pH (<6.0), allowing for the release of the antigen in the endosome, and subsequent lysosomal degradation of the antigen, whilst the antibody is rescued by FcRn-mediated pathways (Ref. 1).

On the other hand, the process of engineering antibodies with pH-dependent antigen binding properties typically requires multiple rounds of discovery, multiple engineering steps, cumbersome design and analysis of individual mutants to selectively tune the antibody affinity at both neutral and acidic pH.

Many of these approaches have focused on the use of histidine residues due to the change in the charge state of this amino acid at endosomal acidic pH. Histidine residues can either be introduced into the CDRs of antibodies in a low-throughput fashion by creating rationally designed panels of antibody mutants that can be tested on-by-one, (Ref. 1) or they can be introduced semi-randomly at scale into the CDRs of synthetic antibody libraries allowing for higher throughput discovery (Ref. 3) e.g., via phage display selection (Ref. 4). More specifically, the engineering of pH-sensitive binding in the antibody variable domains is typically achieved by either introducing histidine mutations sequentially into CDR loops (histidine walking) or combinatorial histidine-scanning approaches using in vitro display technologies (Refs.1, 5).

As histidine residues exhibit a change in charge within the physiological pH range of the antibody recycling pathway, these have successfully been introduced into the complementarity-determining regions (CDRs) of antibodies to engineer pH-switches into non-pH-dependent antibodies based on said computational design or in vitro display technologies (Ref. 6). In an effort to discover antibodies with pH-dependent antigen binding properties a priori using phage display, histidine-doped libraries have been created in which the CDRs of the antibodies have been enriched with histidine residues. This approach suffers however from limitations due to the introduction of mutations in regions of the antigen-binding proteins which are engaging in antigen recognition, and therefore impact antibody function. Furthermore, due to the very high variability of the CDR regions of antibodies, such mutations are specific for each specific antibody.

Understanding how antibody binding can be affected by changes in pH would expand the possibilities for engineering pH-dependent antibodies, in particular for pH-determinants located outside the antibody paratope, which would be beneficial for example when pH-dependent interactions are not able to be accommodated in the antibody paratope without a significant loss of antibody function.

In the present invention, the inventors provide antibodies and other antigen-binding proteins carrying mutations outside of the paratope having the pH sensitive binding properties. Such antibodies were for example identified by investigating the molecular basis for antibody neutralization and the relationship between the antibody paratope, epitope, and paratope independent factors on pH-dependent antigen binding in a panel of light chain shuffled cross-reactive antibodies specific to long-chain α-neurotoxins. The antibodies having the pH-sensitive binding properties are exemplified by antibodies binding different targets, such as antibodies specific to long-chain α-neurotoxins of snake venoms, antibodies specific to tumor necrosis factor-alpha (TNF-alpha), such as Adalimumab, vascular endothelial growth factor (VEGF) such as Bevacizumab, supporting that the principles provided herein are universal, because the mutations are located outside the paratope, and thus can be introduced into virtually any antibody.

The optimized light chains for these antibodies can thus introduce pH-dependent antigen binding. A pool of antibodies has been screened for pH-dependent binding properties to different long-chain α-neurotoxins using biolayer interferometry, which identified one antibody able to bind pH-dependently to all long-chain α-neurotoxins tested. To investigate the mechanism for the pH-dependent binding properties of this antibody to its antigen, X-ray crystallography was chosen to determine whether pH-dependent binding was facilitated by the antibody paratope or epitope.

Structural studies revealed that cross-reactivity was achieved by exploiting a conserved functional constraint in long-chain α-neurotoxins required for their inhibition of the nAChR, determined by the antibody heavy chain paratope. In addition to being cross-reactive, one light chain shuffled antibody, 2555_01_A01, was able to bind pH-dependently to all long-chain α-neurotoxins tested. Structural characterization of 2555_01_A01 bound to α-cbtx at different pH suggest that broad pH-dependent binding was conferred away from the paratope-epitope interface, in the light-heavy chain interface. Further, the inventors reported the crystal structure of this antibody bound to a long-chain α-neurotoxin, determined at 1.6 Å, and elucidated the basis for the neutralization mechanism of this lineage of antibodies. Through the antibody heavy chain complementarity determining region 3, these antibodies mimicked conserved interactions between long-chain α-neurotoxins and the acetylcholine receptor to neutralize long-chain α-neurotoxins and achieve broad cross-reactivity. This antibody also bound pH-dependently to all long-chain α-neurotoxins tested, initiating further structural studies to investigate the pH-dependent binding mechanism.

Importantly, determining the structures of the antibody bound to long-chain a neurotoxin at different pH identified a network of residues that respond in concert to low pH in the antibody structure, located at the interface between the antibody heavy and light chain. In addition, the antibody paratope and epitope analysed were shared with other antibodies that were non-pH dependent, indicating that the pH-dependent binding mechanism for this antibody resided outside the paratope and epitope, and pre-empted further structural studies.

Based on these findings, the inventors found pH-dependent binding conferred by the antibody framework itself, specifically mediated by amino acid residues located in the light-heavy chain interface, which could provide a universal method for introducing pH-dependent antigen binding properties into antibodies.

An important benefit of this approach is that pH-dependent antigen binding could, be less dependent of the CDRs, thereby dramatically expanding the range of compatible paratopes for recycling antibodies. In turn, this strategy could improve the rapidity and precision of discovery campaigns seeking to find pH-dependent antibodies against different targets. Without being bound by theory, inventors believe that the framework interface facilitates antibody-antigen binding by modulating the orientation of the heavy and light chains. Because the heavy-light chain orientation can be engineered to conformationally change dependent on the pH, this may serve as a universal approach to lower binding affinity at a given pH and enable the design of in vitro display libraries consisting of pH-dependent antibodies bearing this synthetic framework.

The present invention also provides methods to improve the discovery of pH-dependent antibodies using in vitro display technology. As the inventors observed that pH-dependent antigen binding could be encoded exclusively away from the paratope, by using a minimal set of mutations not affecting antigen binding within the antibody framework, potentially in the heavy-light chain interface, an approach to engineer a generic pH switch into the antibody variable domain, using a library of antigen-binding proteins variable light and heavy chains with one or more mutations among the set identified.

A phage display library targeting the antibody heavy-light chain interface framework region was therefore designed and validated to introduce a generic pH switch into the antibody variable domain. With pH-dependent binding engineered as a pre-determined feature into antibodies, the invention provides a consistent, high throughput approach to discovering and/or engineering pH-dependent antibodies against different targets using in vitro display technologies a priori in a single discovery campaign.

All in all, the inventors therefore describe pH-dependent antigen-binding proteins comprising a minimal set of mutations outside of the paratope conferring them said pH-dependent antigen-binding properties, and an approach to engineer thereof by targeting the antibody framework region (FWR).

In addition, the inventors have identified further framework residue mutations associated with increased pH sensitivity of antigen binding proteins using point mutations.

The inventors have also identified further framework residues relevant for pH-sensitivity of antigen-binding protein based on in silico analysis of interaction points of heavy and light chain interfaces with the conservation of amino acids determined by alignment of 328 therapeutic antibodies.

The present invention may also further improve the pharmacology of antibodies that suffer from target-mediated clearance, improve their cost effectiveness by having a longer duration of action and a lower administration dose, and have the potential for resulting in therapies with better patient compliance (due to less frequent dosing). Another example includes the design of specialized in vitro display antibody libraries with pre-established pH-dependent binding properties that may be used in a generic fashion to discover recycling antibodies and/or antibodies that engage with their target antigen at exact anatomical locations at the right time.

In one aspect, the invention relates to a method for isolating antigen-binding proteins having a pH-dependent scaffold, wherein the scaffold consists of the regions of the variable regions not being part of the paratope, said method comprising the steps of:
providing a library comprising antigen-binding proteins, each comprising an antibody light chain variable region (VL), wherein said VLs comprise one or more mutations positioned outside of the paratope of the antigen-binding protein, wherein said library comprises a plurality of antigen-binding proteins containing different mutations in the VL;
selecting antigen-binding proteins that display higher binding affinity in acidic pH than neutral pH, or lower binding affinity in acidic pH than neutral pH from said library, thereby isolating antigen-binding proteins having a pH-dependent scaffold.

In a second aspect, the invention relates to a method of generating a pH-dependent antigen-binding protein binding a specific epitope, said method comprising the steps of:
isolating an antigen-binding protein having a pH dependent scaffold according to the method described herein;
providing an antigen-binding protein binding said specific epitope;
exchanging the paratope of said antigen-binding protein having a pH dependent scaffold for the paratope of said antigen-binding protein binding said epitope, thereby generating a pH dependent antigen-binding protein binding said specific epitope.

A third aspect of the invention relates to a pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein the pH-dependent antigen-binding protein has a higher binding affinity in acidic pH than neutral pH, or lower binding affinity in acidic pH than neutral pH, and wherein said pH-dependent antigen-binding protein does not comprise heavy chain complementarity-determining regions 1, 2 and 3 of SEQ. ID NOs 1, 2, 3 respectively and light chain complementarity-determining region 1, 2 and 3 of SEQ. ID NOs 4, 5, 6 respectively.

A fourth aspect of the invention relates to a pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein one or more mutations are introduced in residue positions selected from the group consisting of: VH 39, VH 44, VH 89, VH 105, VL 38, VL 43, VL 50, VL 85 and VL 100 according to Kabat compared to a parental FWR1, FWR2, FWR2′, FWR3 and FWR4, and wherein the pH-dependent antigen-binding protein has higher binding affinity in acidic pH than neutral pH, or lower binding affinity in acidic pH than neutral pH, and wherein said pH-dependent antigen-binding protein does not comprise heavy chain complementarity-determining regions 1, 2 and 3 of SEQ. ID NOs 1, 2, 3 respectively and light chain complementarity-determining region 1, 2 and 3 of SEQ. ID NOs 4, 5, 6 respectively.

A fifth aspect of the invention relates to a method for producing a pH-dependent antigen-binding protein directed to an antigen of interest, said method comprising the steps of:
providing an antigen-binding protein binding an antigen of interest;
introducing in said antigen-binding protein one or more mutations in residue positions selected from the group consisting of: VH 39, VH 44, VH 45, VH 47, VH 89, VH 91, VH 103, VH 105, VL 32, VL 36, VL 38, VL 43, VL 44, VL 46, VL49, VL 50, VL 85 VL 87, VL 98 and VL 100 according to Kabat,
thereby producing a pH-dependent antigen-binding protein targeted to said antigen of interest.

As described herein, in some embodiments of the antigen-binding proteins, pH-dependent antigen binding proteins, and methods described herein, one or more mutations are introduced in the antigen-binding protein to confer pH-dependent binding properties to said antigen-binding. In some embodiments, mutations to specific amino acid residues at specific residue positions of the VH and VL of said antigen-binding proteins confer pH-dependent binding properties to said antigen-binding proteins.

Thus, an aspect of the invention relates to an antigen-binding protein comprising an antibody light chain variable region (VL) and an antibody heavy chain variable region (VH), wherein one or more mutations are introduced at residue positions selected from the group consisting of: VH 39, VH 44, VH 45, VH 47, VH 89, VH 91, VH 103, VH 105, VL 32, VL36, VL 38, VL 43, VL 44, VL 46, VL49, VL 50, VL 85, VL 87, VL 98 and VL 100 according to Kabat numbering, wherein
the VH contains one or more, such as two, such as three, such as four, of the following amino acid residues:
   an S, E, R, T, or A at position 39,
   a P, R, N, S, K, Q, A, Y, or T at position 44,
   a T, N, I, Q, A, L, Y, D, F, S, or K at position 89, and/or
   a T, R, K, P, D, I, S, or T at position 105.
and/or
wherein the VL contains one or more, such as two, such as three, such as four, such as five, such as six, such as seven, of the following amino acid residues:
   an N at position 36,
   a L, Y, S, I, T, A, R, F, or V at position 38,
   a P, T, A, R, Q, K, or V at position 43
   an A, or S at position 46,
   an A, H, or S at position 49,
   an S, N, T, F, V, L, S, A, H, or R at position 85, and/or
   an S, Y, W, or L at position 100,
wherein all positions are indicated according to Kabat numbering.

A sixth aspect of the present invention relates to a composition comprising the pH-dependent antigen-binding protein as described herein and a pharmaceutically acceptable excipient.

A seventh aspect of the present invention relates to a pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein the pH-dependent antigen-binding protein has a higher binding affinity in acidic pH than neutral pH, or a lower binding affinity in acidic pH than neutral pH, or the composition as described herein, for use in a method of treatment of cancer, autoimmune diseases, metabolic diseases, or haematological diseases in a patient in need thereof.

An eighth aspect of the present invention relates to a method of treating cancer, autoimmune diseases, metabolic diseases, or haematological diseases, comprising administering to a patient in need thereof a pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein the pH-dependent antigen-binding protein has a higher binding affinity in acidic pH than neutral pH, or lower binding afffinity in acidic pH than neutral pH, or the composition as described herein.

A ninth aspect of the present invention relates to the use of a pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein the pH-dependent antigen-binding protein has a higher binding affinity in acidic pH than neutral pH, or lower binding affinity in acidic pH than neutral pH, or the composition as described herein, in the manufacture of a medicament for the treatment of cancer, autoimmune diseases, metabolic diseases, or haematological diseases.

A tenth aspect of the present invention relates to the use of a pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein the pH-dependent antigen-binding protein has higher binding affinity in acidic pH than neutral pH, or lower binding affinity in acidic pH than neutral pH, or the composition as described herein, in an in vitro method for detection and/or diagnosis of cancer.

An eleventh aspect of the present invention relates to an in-vitro antigen detection method comprising:
providing a pH-dependent antigen-binding protein as described herein;
contacting the pH-dependent antigen-binding protein with the antigen it binds to; and
detecting the contact between the pH-dependent antigen-binding protein and its antigen,
thereby detecting the antigen.

A twelfth aspect of the present invention relates to an in-vitro antigen purification method comprising:
providing a pH-dependent antigen-binding protein as described herein;
contacting the pH-dependent antigen-binding protein with the antigen it binds to in a complex mixture; and
separating the pH-dependent antigen-binding protein/antigen complex from the complex mixture,
thereby purifying the antigen.

### DESCRIPTION OF DRAWINGS

FIG. 1: Binding characterization and in vivo neutralization of long-chain α-neurotoxins.

(A) Light chain complementarity determining region loop sequences for the antibodies used in the study, numbered according to the Kabat scheme (CDRL1 spans from residues 22 to 34, CDRL2 spans from residues 50 to 56 and CDRL3 spans from residues 89 to 97 including positions 95, 95A, and 95B). The properties of amino acid residues are in different grey shades and described beneath the sequences (charged: R, E, D, H; Polar: T, S, Q, N; Non-polar: G, I, A, Y, V, W, P). (B) Affinity of anti-long chain neurotoxin clones to α-bgtx at pH 7.4, data points are shaped according to the antibody light chain germline. (C-E) pH-sensitive binding fold differences of anti-long chain neurotoxin clones to α-cbtx, α-eptx and α-bgtx. The fold difference for the IGVLA and parent clones binding to to α-bgtx was measured as a product of their affinity difference under steady-state conditions at pH 5.5 and pH 7.4, all other fold differences were measured as the change in dissociate rate between pH 5.5 and 7.4.

FIG. 2. 2555_01_A01 antibody bound to α-cbtx.

(A) 2555_01_A01 scFv bound to α-cbtx. The antibody heavy chain (HC, dark grey) recognizes the Finger II of α-cbtx (Finger II, light grey) through the CDRH3, conformationally stabilized by an intramolecular disulfide bridge (pointed at by black arrow). The antibody light chain (LC) is colored in medium grey. (B) SEC chromatogram overlay of 2555_01_A01 unbound and in complex with α-cbtx.

FIG. 3. Neutralization of α-cbtx by 2555_01_A01 antibody through receptor mimicry.

(A) From left to right: Structure of the Torpedo nAChR bound to α-bgtx (6uwz). Finger II of α-bgtx (top structure, Bgtx) is bound at the interface between the gamma (left structure, light grey, gamma) and alpha (right structure, medium grey, alpha) domains of nAChR, R33 on finger II is shown in stick format. The key response element loop C (dark grey, left dotted circle) is depicted on the alpha domain of the nAChR. An overlay of tyrosine residues on loop C and CDRH3 (dark grey cyclic structure, middle dotted circle) coordinating to R33 and R36 on long-chain α-neurotoxins, illustrating mimicry of interactions between nAChR and 2555_01_A01. Right: 2555_01_A01 bound to α-cbtx. (B) Core interactions between the CDRH3 and Finger II of α-cbtx, targeting conserved residues in long-chain α-neurotoxins (R33, R36, D26). (C) Opposing view of interactions to the backbone and C-Terminal of α-cbtx by CDRH3 and CDRL3 loops.

FIG. 4. Light chain effects on pH-sensitive binding for 2555_01_A01 antibody. (A) Rear view of the CDRL3 (dark grey) interface with α-cbtx (light grey, R70) at pH 6.0. Hydrogen bonds shown by dotted lines. (B) Remodeled H95b hydrogen bond following rotamer change at pH 4.5 indicated by the black arrow. (C) Effect of D95a and H95b on pH-dependent binding by a double mutation into a non-pH dependent HE motif seen in the non-pH-dependent 2554_01_D11 antibody. (D) Proposed residue network involved in pH-dependent binding for 2555_01_A01 as seen in the pH 4.5 model. (E) Fo-Fo subtraction of proposed residues using refined pH 5.5 and pH 4.5 data sets. The pH 4.5 model is shown and highlights the electron density above the phenyl ring at pH 5.5. Heavy chain and associated residues are shown in dark grey (e.g D95, Y1001), light chain in medium grey (e.g H34, S50) and α-cbtx in light grey (e.g. R33), respectively. Fourier maps contoured at 3 σ.

FIG. 5. Binding characterization of the 2554_01_D11 antibody by SPR and framework library design.

(A) Sensorgrams for the monovalent 2554_01_D11 Fab binding to immobilized α-cobratoxin measured at pH 7.4 and (B) pH 5.1. The order of the curves from top to bottom on each graph correspond to the decreasing concentrations of the antibody tested as indicated on the right panel. (C) Library design highlighting sites selected for library creation (VH: Q39, G44, V89, Q105, VL: Q38, S43, D85, G100), numbered using the Kabat scheme. (D) The location of the residues is shown on the variable domain of the 2555_01_A01 antibody (lower part of the VH and VL, in stick format), which is a light chain variant of 2554_01_D11 (2555_01_A01) clone.

FIG. 6. Enrichment of phages from two rounds of selection using the binding framework interface library.

Enrichment was calculated by fold-difference between test and no-antigen control selections.

FIG. 7. Up-scaling of inserts following golden gate assembly.

(A) Up-scaling of individual heavy (HC) and light chains (LC). Two PCRs were carried out with the heavy chain as the reverse primer contained a mutation site. (B) PCR optimization of assembled scFv library following gel extraction. Different input amounts of extracted scFv and concentrations of each primer were tested. The scFv reference was generated from the purchased scFv vector used to build the library. The 1 ng scFv+0.1 μM primer concentration showed the purest product and was taken forward to electroporation.

FIG. 8. Cloning efficiency of the scFv library into pSANG4 phagemid.

(A) Agarose gel analysis of colony PCR amplicons from individual transformants using the pSANG5th_For and gpilll primers. A reference amplicon was generated from the template pSANG4 phagemid used in the library creation. (B) Restriction digestion of amplicons derived from 6 library transformants and the reference using Bpil endonuclease. The reference scFv had Bpil restriction sites and was included as control.

FIG. 9. Monoclonal DELFIA (dissociation-enhanced lanthanide fluorescence immunoassay) of 92 randomly picked framework-shifted scFvs from the second round of antibody phage display selection

The binding signal was measured as time-resolved fluorescence (TRF) with excitation at 320 nm and emission at 615 nm in a Victor Nivo Multimode Microplate reader. The x-axis represents the baseline binding affinity of the clones to the target antigen with higher signal corresponding to higher binding. The y-axis is the ratio of the DELFIA signals obtained at pH 7.4 and pH 5.8 for each clone, also referred to as pH sensitivity. “Parent antibody” refers to the parent antibody used to generate the chain-interface library. “pH-sensitive antibody” refers to a positive control antigen binding protein displaying very high pH sensitivity in the range 10-11.

FIG. 10. Amino acid sequences alignments of the scFvs (Heavy-chains) with a pH-sensitive DELFIA signal (pH 7.4 DELFIA/pH 5.8 DELFIA) above 1.25.

Grey areas highlight the residues that are mutated in the framework-mutated library used for the phage display discovery. Dots correspond to identical amino acids as clone 2554_01_D11, “*” indicates deleted residues compared to the clone 2554_01_D11.

FIG. 11. Amino acid sequences alignments of the scFvs (Light-chains) with a pH-sensitive DELFIA signal (pH 7.4 DELFIA/pH 5.8 DELFIA) above 1.25.

Grey areas highlight the residues that are mutated in the framework-mutated library. Dots correspond to identical amino acids as clone 2554_01_D11,

FIG. 12. Point mutations and associated pH sensitivity

The figure shows different point mutations and the level of pH-sensitivity they resulted in. pH-sensitivity was calculated by dividing the average DELFIA signal at pH 7.4 by the average signal at pH 5.8 (pH7.4/pH5.8). AVR: Average, Pos CTR: positive control.

FIG. 13. Alignment of antibody sequences from the Therapeutic Structural Antibody Database (Thera-SAbDab)

Therapeutic Structural Antibody Database (Thera-SAbDab) (https://opig.stats.ox.ac.uk/webapps/sabdab-sabpred/therasabdab/search/). The complete Heavy chain, Lambda light chain, and Kappa light chain consensus sequences are each split over 2 rows labelled “1/2” and “2/2”. Split sequences 1/2 and 2/2 are directly consecutive for each chain, starting with the sequence “1/2”. “+” symbols may be any amino acid. Hash symbols (#) are positions the inventors have mutated in the framework-mutated library and observed increased pH sensitivity compared to the parental antibody. Asterisks (*) highlight amino acids (AAs) denoted as interface interacting AAs calculated as the buried solvent-accessible surface area using the ChimeraX function: interface. Triangles (▴) highlight additional residues identified by point mutations and associated with increased pH sensitivity of scFvs. Circles (●) highlight additional residues relevant for pH-sensitivity of antigen-binding proteins identified based on interface interaction and conservation.

FIG. 14. Structure-guided engineering in D11 light chain improves pH-dependent binding to α-cobratoxin

Screening the effect of light chain mutations on capacity to bind pH-dependently to cobratoxin by DELFIA. Binding assessed in a pH 7.4 (dark bars) or pH 5.8 (grey bars) pH-shift regiment.

FIG. 15. The framework mutations identified are identical in blockbuster antibodies

A. DELFIA immunoassay results showing that mutagenesis of 8 strategic amino acid residues located in the heavy-light chain interface increases the pH-dependence of the antibody-antigen interaction of the model antigen (α-cobratoxin) and antibody (2554_01_D11-graphed as grey dot), while the binding signal is retained, thus demonstrating the utility of the universal pH-switch of the present invention. B. Representations of the conserved similarity in the four key amino acid residues identified in the mutated D11 (2554_01_D11) (dots circled in panel A) antibody when comparing to the two blockbuster mAbs, bevacizumab (Avastin) and adalimumab (Humira). Dark ribbons: Heavy chain, Grey ribbons: Light chain, Dark spheres: Four positions where mutations induce pH-dependent antigen binding. These models indicate that the frame mutations that provide 2554_01_D11 with pH-dependent antigen binding properties are conserved in other (blockbuster) mAbs.

FIG. 16. Site-directed mutagenesis of Bevacizumab and Adalimumab in scFv formats induces an increase in pH-dependent antigen binding

Site-directed mutagenesis of amino acid residues located in the heavy-light-chain interface of Adalimumab and Bevacizumab (bottom and top panel respectively) in scFv formats (black bars) induces an increase in pH-dependent antigen binding as measured by DELFIA immunoassay. Parent antibody (non-mutated Bevacizumab, top panel, non-mutated Adalimumab, bottom panel) are shown in grey.

FIG. 17. Sequences alignments of top 5 Bevacizumab and Adalimumab candidates from FIG. 16.

A. Sequencing of top 5 Bevacizumab candidates (1, 2, 3, 4, 5 sequences shaded in grey) show mutations in the 4 identified residue positions. B. Sequencing of top 5 Adalimumab candidates (1, 2, 3, 4, 5 sequences shaded in grey) show mutations in the 4 identified residue positions. Light residue positions highlight the residues that were mutated, which are labelled as “X” in the parent antibody consensus sequence above the 5 mutants sequences.

FIG. 18. Site-directed mutagenesis of antibody 2554_01_D11 in ScFv format induces an increase in pH-dependent antigen binding

DELFIA immunoassay showing that mutagenesis of the VH 45, VH 47, VH 91, VL 36, VL 44, VL 46, VL49, VL 87, and/or VL98 strategic amino acid residues located in the heavy-light chain interface increases the pH-dependence of the antibody-antigen interaction of the model antigen (α-cobratoxin) and antibody (2554_01_D11). D11 scFv clone labelled with a star “*” is of sequence SEQ ID NO: 254.

### DETAILED DESCRIPTION

### Definitions

As used herein, the singular forms “a”, “an” and “the” include plural referents unless the context clearly states otherwise. Thus, for example, reference to “an antigen-binding protein” includes a plurality of such antigen-binding proteins.

As used herein, the term “antigen-binding protein” includes antibodies, antibody fragments and other antigen-binding protein constructs. The term encompasses intact antibodies that comprise at least two full-length heavy chains and two full-length light chains, as well as derivatives, variants, fragments, and mutations thereof, examples of which include Fab fragments, Fab′ fragments, F(ab′)2 fragments, Fv fragments, domain antibodies, single chain antibodies, scFv, or bi-specific or multi-specific antigen-binding proteins. Preferably, the antigen-binding protein is an antibody or an antigen binding fragment thereof. The antibodies according to the present invention are in general human monoclonal antibodies.

As used herein, the term “amino acid” refers to any of the twenty naturally occurring amino acids that are normally used in the formation of polypeptides, or analogs or derivatives of those amino acids or any non-naturally occurring amino acid, preferably the term “amino acid” refers to any of the twenty naturally occurring amino acids that are normally used in the formation of polypeptides.

Unless otherwise indicated amino acids are abbreviated and mentioned by their conventional nomenclature known to the person skilled in the art, such as the conventional one-letter amino acid code according to the “nomenclature and symbolism for amino acids and peptides” by the international union of pure and applied chemistry (IUPAC) (www.iupac.org).

As used herein the term “complementarity-determining regions” (CDRs) refers to the three hypervariable regions of a light chain (LCDR1 or CDRL1, LCDR2 or CDRL2, and LCDR3 or CDRL3) and/or the three hypervariable regions of a heavy chain (HCDR1 or CDRH1, HCDR2 or CDRH2 and HCDR3 or CDRH3) set within four more highly conserved framework regions of an antigen-binding protein. These hypervariable regions are primarily responsible for antigen recognition. In an antigen-binding protein, the CDRs are disposed relative to each other in three dimensional space to form an antigen binding surface.

As used herein the term “pH-dependent antigen binding-protein” or “pH-sensitive antigen binding-protein” relates to an antigen-binding protein capable to bind to an antigen at a first pH condition, for instance neutral pH, including herein near-neutral physiological human body pH (7.35-7.45), for example physiological plasma pH (7.4), and to dissociate from said antigen at another pH condition, for instance acidic pH defined as pH<7, preferably pH<6, for example acidic conditions encountered in cellular endosomes, more preferably pH in the range of pH 5 to pH 6, such as pH 5.8, such as pH 5.5. The opposite is also comprised in the term. It is preferred that at least one order of magnitude difference in the binding affinity, for instance as measured by the Kd, is observed between the two pH conditions. Unless otherwise indicated “higher binding affinity” or “lower binding affinity” as used herein relate to the binding affinity of the antigen-binding proteins to its antigen. In some embodiments herein the pH-dependence or pH sensitivity is measured as the ratio between two pH conditions of DELFIA TRF signals (Dissociation-Enhanced Lanthanide Fluorescence Immunoassay Time-Resolved-Fluorescence) obtained from an antigen-binding protein binding to its antigen, for example the ratio between DELFIA signals obtained from the antigen-binding protein binding to its antigen at neutral pH such as pH 7.4, and DELFIA signals obtained from the antigen-binding protein binding to its antigen at acidic pH such as pH 5.8 or pH 5.5.

A “paratope” as used herein refers to the antigen-binding site(s) of the antigen-binding protein which engages in binding to the antigen epitope.

As used herein the term framework region (FWR) refers to the regions of the variable region of antibodies or other fragments thereof which are interspersed with the three hypervariable regions (complementarity-determining regions (CDRs)) on each of the variable light and/or heavy chain. The FWR numbering order used herein for instance for a variable region of a full-length IgG comprising 3 CDRs and 4 FWRs on each variable region is as follows from N-terminal to C-terminal ending of said variable region: FWR1, CDR1, FWR2, CDR2, FWR3, CDR3, FWR4. Further, FWRX′ as used herein refers to the residue position directly C-terminally adjacent to the last residue of said FWRX, wherein X can be 1, 2, 3 or 4.

As used herein the term “scaffold” refers to the regions of the variable regions of an antigen-binding protein not being part of the paratope. The term scaffold thus encompasses antibody framework regions (FWR) and/or complementarity-determining regions (CDRs) which are not engaging in epitope binding.

As used herein, “Kabat numbering” refers to the Kabat antibody amino acid residue numbering system. The scheme defines specific positions where insertions and gaps may occur in CDRs and FWRs. In the system, additional amino acid insertions are annotated with letters as described in Kabat E A, Te Wu T, Foeller C, Perry H M, Gottesman K S. Sequences of Proteins of Immunological Interest. (1991). U.S. Department of Health and Human Services, Public Health Service, National Institutes of Health.

As used herein “VH FWRX” and “VL FWRX” refer to the framework region number X of the variable region of the heavy chain (VH) and variable region of the light chain (VL) respectively, of an antigen binding protein, wherein X can be 1, 2, 3 or 4.

As used herein “VH XX” or “VL XX” refer to the residue at position “XX” of the sequence of the variable region of the heavy chain (VH), or variable region of the light chain (VL), respectively e,g, VH 39 refers to the residue at position 39 of an antibody heavy chain variable region (VH). In preferred embodiments the residue numbering is according to Kabat as described hereinabove.

As used herein specific amino acids at specific residue positions in sequences are indicated by the conventional one-letter abbreviation of that amino acid attached to the residue position at which the amino acid is found. For example “L45” refers to amino acid leucin found at residue position 45. In order to specify the type of chain (variable region of light chain or variable region of the heavy chain) on which said specific amino acid at said specific position is found “VL” or “VH” may be further added. For example “VH L45” refers to amino acid leucin found at residue position 45 of the variable region of the heavy chain.

As used herein, the term “variant” refers to either a naturally occurring variation of a given peptide or a recombinantly prepared variation of a given peptide or protein in which one or more amino acid residues have been modified by amino acid substitution, addition, or deletion. The term “variant” may also define either a naturally occurring genetic mutant of a DNA sequence or its encoded RNA or protein product, or a recombinantly prepared variation of a DNA sequence or its encoded RNA or protein product.

As used herein, “K_{d}” or “KD” refers to the equilibrium dissociation constant for a ligand-receptor complex. The K_{d} value is expressed in Molar units (M) and is obtained by dividing the dissociation rate (K_{off}) by the association rate (kₒₙ). The association rate, the dissociation rate and the equilibrium dissociation constant are used to represent the binding affinity of an antibody to an antigen. The binding affinity may be compared between two different conditions such as two different pH conditions in order to compare for instance the binding affinity of an antibody to its antigen in said two conditions.

As used herein, the “K_{off}” or “K_{Dis}” or “dissociation rate constant”, of a binding protein (e.g., an antibody) from an association complex (e.g., an antibody/antigen complex) is as is known in the art. This value indicates the dissociation rate of an antibody from its target antigen or separation of Ab-Ag complex over time into free antibody and antigen.

As used herein the term “developability” refers to the suitability of the antigen-binding protein clones for future antibody development. It can be investigated by characterizing biophysical properties that are indicative of how well the antibodies can be developed and manufactured in large scale without aggregating, precipitating, denaturing, or having suboptimal pharmacokinetics in vivo.

Methods for Isolating and/or Generating Antigen-Binding Proteins Having a pH-Dependent Scaffold

The present invention enables the discovery of pH-dependent antibodies directly from mutant libraries, such as phage display libraries, by engineering a pH-sensitive antigen-binding protein scaffold, enabling the design of phage display libraries with pH-dependent binding properties pre-determined into antigen-binding proteins. This is achieved by selecting for and/or introducing pH-dependent binding, for instance in a non-pH-dependent antibody, selectively through the variable regions not being part of the paratope.

In one aspect, the present invention relates to a method for isolating antigen-binding proteins having a pH-dependent scaffold, wherein the scaffold consists of the regions of the variable regions not being part of the paratope, said method comprising the steps of:
providing a library comprising antigen-binding proteins, each comprising an antibody light chain variable region (VL), wherein said VLs comprise one or more mutations positioned outside of the paratope of the antigen-binding protein, wherein said library comprises a plurality of antigen-binding proteins containing different mutations in the VL;
selecting antigen-binding proteins that display higher binding affinity in acidic pH than neutral pH, or lower binding affinity in acidic pH than neutral pH from said library, thereby isolating antigen-binding proteins having a pH-dependent scaffold.

In a second aspect, the invention relates to a method of generating a pH-dependent antigen-binding protein binding a specific epitope, said method comprising the steps of:
isolating an antigen-binding protein having a pH dependent scaffold as described herein;
providing an antigen-binding protein binding said specific epitope;
exchanging the paratope of said antigen-binding protein having a pH dependent scaffold for the paratope of said antigen-binding protein binding said epitope,
   thereby generating a pH dependent antigen-binding protein binding said specific epitope.

In some embodiments of the invention, the one or more antigen-binding proteins of the library further comprise a heavy chain comprising a variable region (VH), wherein said VHs comprise one or more mutations positioned outside of the paratope of the antigen-binding protein.

In other embodiments, the one or more mutations are in residue positions located at the interface between the VL and the VH of said antigen binding protein.

In further embodiments, the parental sequence of the VLs and/or the parental sequence of the VHs are sequences of an antigen binding protein, which does not have pH-dependent antigen-binding. In yet further embodiments, the parental sequence of the VLs and/or the parental sequence of the VHs are sequences of an antigen binding protein with lower pH-dependent antigen-binding relative to the antigen-binding protein comprising the one or more mutations as described herein.

In other embodiments, the one or more mutations are not positioned in the complementarity-determining-regions (CDRs).

The skilled person will appreciate that classical approaches to modulate the pH-dependence of antigen-binding proteins is through engineering of CDR regions, in particular mutations to and/or from Histidine residues. Herein, the inventors provide an approach comprising engineering one or more antibody framework regions (FWRs) in the antigen-binding protein variable region, located beneath the CDRs, and composed of beta sheets and hairpin loops.

Thus, in preferred embodiments of the invention, the one or more mutations are in one or more of the frameworks regions (FWRs).

In yet other embodiments, the antigen-binding proteins of the library comprise an antibody light chain variable region (VL), wherein said VL comprises 3 complementarity-determining regions, CDR1, CDR2 and CDR3 surrounded by 4 framework regions FWR1, FWR2, FWR3 and FWR4, wherein at least one of said FWR1, FWR2, FWR3 and FWR4 contains one or more mutations compared to a parental FWR1, FWR2, FWR3 and FWR4.

The FWR1, FWR2, FWR3 and FWR4 may be framework regions of any antibody, e.g. from any of the types of antibodies described herein.

In some embodiments, the antigen-binding proteins of the library comprise a VL FWR1 of SEQ. ID NO: 26, except that 1 to 2 amino acids are mutated.

In some embodiments, the antigen-binding proteins of the library comprise a VL FWR2 of SEQ. ID NO: 27, except that 1 to 2 amino acids are mutated.

In other embodiments, the antigen-binding proteins of the library comprise a VL FWR3 of SEQ. ID NO: 28, except that 1 to 2 amino acids are mutated.

In yet other embodiments, the antigen-binding proteins of the library comprise a VL FWR4 of SEQ. ID NO: 29, except that 1 to 2 amino acids are mutated.

In some embodiments, the antigen-binding proteins of the library comprise a VL FWR1 of SEQ. ID NO: 26, SEQ. ID NO: 151, SEQ. ID NO: 157 or SEQ. ID NO: 163, except that 1 to 2 amino acids are mutated.

In some embodiments, the antigen-binding proteins of the library comprise a VL FWR2 of SEQ. ID NO: 27, SEQ. ID NO: 152, SEQ. ID NO: 158 or SEQ. ID NO: 164, except that 1 to 2 amino acids are mutated.

In some embodiments, the antigen-binding proteins of the library comprise a VL FWR3 of SEQ. ID NO: 28, SEQ. ID NO: 153, SEQ. ID NO: 159 or SEQ. ID NO: 165, except that 1 to 2 amino acids are mutated.

In some embodiments, the antigen-binding proteins of the library comprise a VL FWR4 of SEQ. ID NO: 29 or SEQ. ID NO: 154, except that 1 to 2 amino acids are mutated.

In some embodiments, the antigen-binding proteins of the library have a VL sequence of SEQ. ID NO: 35 In further embodiments, the VL sequence is encoded by the sequence of SEQ. ID NO: 37.

In some embodiments of the present invention, the antigen-binding proteins of the library comprise an antibody heavy chain variable region (VH), wherein said VH comprises 3 complementarity-determining regions, CDR1, CDR2 and CDR3 surrounded by 4 framework regions FWR1, FWR2, FWR3 and FWR4, wherein at least one of said FWR1, FWR2, FWR3 and FWR4 contains one or more mutations compared to a parental FWR1, FWR2, FWR3 and FWR4

In other embodiments, the antigen-binding proteins of the library comprise a VH FWR1 of SEQ. ID NO: 30, except that 1 to 2 amino acids are mutated.

In other embodiments, the antigen-binding proteins of the library comprise a VH FWR2 of SEQ. ID NO: 31, except that 1 to 2 amino acids are mutated.

In further embodiments, the antigen-binding proteins of the library comprise a VH FWR3 of SEQ. ID NO: 32, except that 1 to 2 amino acids are mutated.

In yet other embodiments, the antigen-binding proteins of the library comprise a VH FWR4 of SEQ. ID NO: 33, except that 1 to 2 amino acids are mutated.

In some embodiments, the antigen-binding proteins of the library comprise a VH FWR1 of SEQ. ID NO: 30, SEQ. ID NO: 155, SEQ. ID NO: 160 or SEQ. ID NO: 166, except that 1 to 2 amino acids are mutated.

In some embodiments, the antigen-binding proteins of the library comprise a VH FWR2 of SEQ. ID NO: 31, SEQ. ID NO: 156, SEQ. ID NO: 161 or SEQ. ID NO: 167, except that 1 to 2 amino acids are mutated.

In some embodiments, the antigen-binding proteins of the library comprise a VH FWR3 of SEQ. ID NO: 32, SEQ. ID NO: 157, SEQ. ID NO: 162 or SEQ. ID NO: 168, except that 1 to 2 amino acids are mutated.

In some embodiments, the antigen-binding proteins of the library comprise a VH FWR4 of SEQ. ID NO: 33 except that 1 to 2 amino acids are mutated.

In some embodiments, the antigen-binding proteins of the library have a VH sequence of SEQ. ID NO: 34. In further embodiments, the VH sequence is encoded by the sequence of SEQ. ID NO: 36.

In preferred embodiments, the one or more mutations are not at residue positions occupied by Histidine residues.

In other preferred embodiments, the one or more mutations are not mutations to Histidine residues.

In yet other embodiments, the one or more mutations are at residue positions at least 1 amino acid away from an histidine residue, such as at least 2 amino acids, for example at least 5 amino acids, such as at least 8 amino acids, for example at least 10 amino acids, such as at least 15 amino acids, for example at least 20 amino acids, such as at least 25 amino acids, for example at least 50 amino acids away from an histidine residue.

The inventors have identified specific FWR residue positions particularly useful in conferring pH-dependent binding to antigen-binding proteins. In some embodiments, the positions are irrespective of the FWR sequences per se.

In some embodiments, the one or more mutations are at VH residues selected from the group consisting of: 39, 44, 89, and 105 according to Kabat numbering.

In other embodiments, the one or more mutations are at VL residue positions selected from the group consisting of: 38, 43, 85, and 100, according to Kabat numbering.

In some embodiments, the antigen-binding proteins of the library comprise:
a) a VH FWR2 of SEQ ID NO:31
b) a VH FWR3 of SEQ ID NO:32
c) a VH FWR4 of SEQ ID NO:33
and
d) a VL FWR2 of SEQ. ID NO: 27
e) a VL FWR3 of SEQ. ID NO: 28
f) a VL FWR4 of SEQ. ID NO: 39,
   wherein one or more mutations are introduced in residue position VH 39, VH 44, VH 89, VH 105, VL 38, VL 43, VL 85 and/or VL 100 according to Kabat numbering.

In some applications, it may be beneficial that the antigen-binding proteins to which the one or more mutations are introduced display specific framework region combinations, Thus, in some embodiments of the antigen-binding proteins described herein, the one or more mutations are introduced in a VL region of the antigen-binding protein comprising the framework regions:
i. FWR1 of SEQ. ID NO: 26,
ii. FWR2 of SEQ. ID NO: 27,
iii. FWR3 of SEQ. ID NO: 28, and
iv. FWR4 of SEQ. ID NO: 29;
   or
i. FWR1 of SEQ. ID NO: 151,
ii. FWR2 of SEQ. ID NO: 152,
iii. FWR3 of SEQ. ID NO: 153, and
iv. FWR4 of SEQ. ID NO: 154;
   or
i. FWR1 of SEQ. ID NO: 151,
ii. FWR2 of SEQ. ID NO: 158,
iii. FWR3 of SEQ. ID NO: 159, and
iv. FWR4 of SEQ. ID NO: 154;
   or
i. FWR1 of SEQ. ID NO: 163,
ii. FWR2 of SEQ. ID NO: 164,
iii. FWR3 of SEQ. ID NO: 165, and
iv. FWR4 of SEQ. ID NO: 154;
and in a VH region of the antigen-binding protein comprising the framework regions:
i. FWR1 of SEQ. ID NO: 30,
ii. FWR2 of SEQ. ID NO: 31,
iii. FWR3 of SEQ. ID NO: 32, and
iv. FWR4 of SEQ. ID NO: 33;
   or
i. FWR1 of SEQ. ID NO: 155,
ii. FWR2 of SEQ. ID NO: 156,
iii. FWR3 of SEQ. ID NO: 157, and
iv. FWR4 of SEQ. ID NO: 33;
   or
i. FWR1 of SEQ. ID NO: 160,
ii. FWR2 of SEQ. ID NO: 161,
iii. FWR3 of SEQ. ID NO: 162, and
iv. FWR4 of SEQ. ID NO: 33;
   or
i. FWR1 of SEQ. ID NO: 166,
ii. FWR2 of SEQ. ID NO: 167,
iii. FWR3 of SEQ. ID NO: 168, and
iv. FWR4 of SEQ. ID NO: 33.

In other embodiments, the antigen-binding proteins of the library comprise:
a) a VH FWR2 of SEQ ID NO: 156
b) a VH FWR3 of SEQ ID NO: 157
c) a VH FWR4 of SEQ ID NO:33
and
d) a VL FWR2 of SEQ. ID NO: 152
e) a VL FWR3 of SEQ. ID NO: 153
f) a VL FWR4 of SEQ. ID NO: 154,
wherein one or more mutations are introduced in residue position VH 39, VH 44, VH 45, VH 47, VH 89, VH 91, VH 103, VH 105, VL 32, VL 36, VL 38, VL 43, VL 44, VL 46, VL49, VL 50, VL 85 VL 87, VL 98 and/or VL 100 according to Kabat numbering, preferably wherein one or more mutations are introduced in residue position VH 44, VH 89, VH 105, and/or VL 38 according to Kabat numbering.

In further embodiments, the antigen-binding proteins of the library comprise:
a) a VH FWR2 of SEQ ID NO: 161
b) a VH FWR3 of SEQ ID NO: 162
c) a VH FWR4 of SEQ ID NO:33
and
d) a VL FWR2 of SEQ. ID NO: 158
e) a VL FWR3 of SEQ. ID NO: 159
f) a VL FWR4 of SEQ. ID NO: 154,
wherein one or more mutations are introduced in residue position VH 39, VH 44, VH 45, VH 47, VH 89, VH 91, VH 103, VH 105, VL 32, VL 36, VL 38, VL 43, VL 44, VL 46, VL49, VL 50, VL 85 VL 87, VL 98 and/or VL 100 according to Kabat numbering, preferably wherein one or more mutations are introduced in residue position VH 44, VH 89, VH 105, and/or VL 38 according to Kabat numbering.

In some embodiments, the antigen-binding proteins of the library comprise:
a) a VH FWR2 of SEQ ID NO: 164
b) a VH FWR3 of SEQ ID NO: 165
c) a VH FWR4 of SEQ ID NO:33
and
d) a VL FWR2 of SEQ. ID NO: 167
e) a VL FWR3 of SEQ. ID NO: 168
f) a VL FWR4 of SEQ. ID NO: 154,
wherein one or more mutations are introduced in residue position VH 39, VH 44, VH 45, VH 47, VH 89, VH 91, VH 103, VH 105, VL 32, VL 36, VL 38, VL 43, VL 44, VL 46, VL49, VL 50, VL 85 VL 87, VL 98 and/or VL 100 according to Kabat numbering, preferably wherein one or more mutations are introduced in residue position VH 44, VH 89, VH 105, and/or VL 38 according to Kabat numbering.

In preferred embodiments, the step of selecting antigen-binding proteins that display lower KD in acidic pH than neutral pH, or higher KD in acidic pH than neutral pH, from said library comprises using an in vitro display technology.

The step of selection may be for example performed as described in Example 6.

In other preferred embodiments, the in vitro display technology is selected from the group consisting of: phage display, ribosome display, yeast display, bacterial display, mammalian display, and CIS display.

In some embodiments of the method as described herein, the antigen-binding proteins of the library comprise or consist of the pH-dependent antigen-binding proteins as described herein or the antigen binding proteins as described herein.

pH-Dependent Antigen-Binding Proteins

A third aspect of the invention relates to a pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein the pH-dependent antigen-binding protein has higher binding affinity in acidic pH than neutral pH, or lower binding affinity in acidic pH than neutral pH, and wherein said pH-dependent antigen-binding protein does not comprise heavy chain complementarity-determining regions 1, 2 and 3 of SEQ. ID NOs 1, 2, 3 respectively and light chain complementarity-determining region 1, 2 and 3 of SEQ. ID Nos 4, 5, 6 respectively.

Another aspect of the invention relates to a pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein the pH-dependent antigen-binding protein has higher binding affinity in acidic pH than neutral pH, or lower binding affinity in acidic pH than neutral pH, and wherein said pH-dependent antigen-binding protein does not comprise heavy chain complementarity-determining regions 1, 2 and 3 of any of:
SEQ. ID NOs 1, 2, 3 respectively,
SEQ. ID NOs 178, 179, 180 respectively,
SEQ. ID NOs 181, 182, 183 respectively, and
SEQ. ID NOs 184, 185, 186 respectively,
and light chain complementarity-determining region 1, 2 and 3 of any of:
SEQ. ID NOs 4, 5, 6 respectively,
SEQ. ID NOs 169, 170, 171 respectively,
SEQ. ID NOs 172, 173, 174 respectively, and
SEQ. ID NOs 175, 176, 177 respectively.

In preferred embodiments, the pH-dependent antigen binding protein is as defined hereinabove.

### Residue Mutations—Locations

In some embodiments, the one or more mutations of the pH-dependent antigen-binding protein are at residue positions located at the interface between the VL and the VH of the pH-dependent antigen binding protein.

In other embodiments, the one or more mutations of the pH-dependent antigen-binding protein are not at residue positions occupied by Histidine residues.

In further embodiments, the one or more mutations of the pH-dependent antigen-binding protein are not mutations to Histidine residues.

In other embodiments, the one or more mutations of the pH-dependent antigen-binding protein are at residue positions at least 1 amino acid away from an histidine residue, such as at least 2 amino acids, for example at least 5 amino acids, such as at least 8 amino acids, for example at least 10 amino acids, such as at least 15 amino acids, for example at least 20 amino acids, such as at least 25 amino acids, for example at least 50 amino acids away from an histidine residue.

In some embodiments, the one or more mutations of the pH-dependent antigen-binding protein are at VH residues selected from the group consisting of: 39, 44, 89, and 105 according to Kabat numbering, preferably wherein the mutations are at VH residues selected from the group consisting of 44, 89, and 105.

In some embodiments, the one or more mutations are at VH residues selected from the group consisting of: 45, 47, 91, and 103 according to Kabat numbering.

In some embodiments, the one or more mutations are at VH residues selected from the group consisting of: L45, W47, Y91, and W103

In some embodiments, the one or more mutations of the pH-dependent antigen-binding protein are at VL residue positions selected from the group consisting of: 38, 43, 85, and 100, according to Kabat numbering, preferably wherein the mutation is at VL residue position 38.

In some embodiments, the one or more mutations are at VL residue positions selected from the group consisting of: 32, 46, and 49 according to Kabat numbering.

In other embodiments, the one or more mutations are at VL residue positions selected from the group consisting of: 46 and 49 according to Kabat numbering.

In some embodiments, the one or more mutations are at VL residue positions selected from the group consisting of: 36, 44, 87 and 98 according to Kabat numbering.

In some embodiments, the one or more mutations are at VL residues selected from the group consisting of: Y32, T46, and Y49 according to Kabat numbering.

In other embodiments, the one or more mutations are at VL residues selected from the group consisting of: T46, and Y49 according to Kabat numbering.

In some embodiments, the one or more mutations are at VL residues selected from the group consisting of: Y36, P44, Y87, and F98 according to Kabat numbering.

In other embodiments, the one or more mutations are at VH residues selected from the group consisting of: 39, 44, 45, 47, 89, 91, 103 and 105 according to Kabat numbering.

In some embodiments, the one or more mutations are at VL residues selected from the group consisting of: 32, 36, 38, 43, 44, 46, 49, 85, 87, 98 and 100 according to Kabat numbering.

In some embodiments, the one or more mutations are at VL residues selected from the group consisting of: 36, 38, 43, 44, 46, 49, 85, 87, 98 and 100 according to Kabat numbering.

In some embodiments, the one or more mutations are at VH residues selected from the group consisting of: Q39, G44, L45, W47, V89, Y91, W103 and Q105 according to Kabat numbering.

In some embodiments, the one or more mutations are at VL residues selected from the group consisting of: Y32, Y36, Q38, D43, P44, T46, Y49, D85, Y87, F98 and G100 according to Kabat numbering.

In other embodiments, the one or more mutations are at VL residues selected from the group consisting of: Y36, Q38, D43, P44, T46, Y49, D85, Y87, F98 and G100 according to Kabat numbering.

In preferred embodiments, the antigen-binding protein comprises:
a) a VH FWR2 of SEQ ID NO:31
b) a VH FWR3 of SEQ ID NO:32
c) a VH FWR4 of SEQ ID NO:33
and
d) a VL FWR2 of SEQ. ID NO: 27
e) a VL FWR3 of SEQ. ID NO: 28
f) a VL FWR4 of SEQ. ID NO: 39
   wherein one or more mutations are introduced in residue positions selected from the group consisting of: VH 39, VH 44, VH 89, VH 105, VL 38, VL 43, VL 85 and VL 100 according to Kabat numbering.

In other embodiments, the antigen-binding protein further comprises a VH FWR1 of SEQ ID NO:30 and a VL FWR1 of SEQ. ID NO: 26.

In other embodiments, the antigen-binding protein comprises:
a) a VH FWR2 of SEQ ID NO:31
b) a VH FWR3 of SEQ ID NO:32
c) a VH FWR4 of SEQ ID NO:33
and
d) a VL FWR2 of SEQ. ID NO: 27
e) a VL FWR3 of SEQ. ID NO: 28
f) a VL FWR4 of SEQ. ID NO: 29
wherein one or more mutations are introduced in residue positions selected from the group consisting of: VH 45, VH 47, VH 91, VH 103, VL 36, VL 44, VL 87, VL 98 according to Kabat numbering.

In yet other embodiments, the antigen-binding protein comprises:
a) a VH FWR2 of SEQ ID NO:31
b) a VH FWR3 of SEQ ID NO:32
c) a VH FWR4 of SEQ ID NO:33
and
d) a VL FWR2 of SEQ. ID NO: 27
e) a VL FWR3 of SEQ. ID NO: 28
f) a VL FWR4 of SEQ. ID NO: 29
wherein one or more mutations are introduced in residue positions selected from the group consisting of: VL 32, VL 46, VL 49 according to Kabat numbering.

In some embodiments, the antigen-binding protein comprises:
a) a VH FWR2 of SEQ ID NO:31
b) a VH FWR3 of SEQ ID NO:32
c) a VH FWR4 of SEQ ID NO:33
and
d) a VL FWR2 of SEQ. ID NO: 27
e) a VL FWR3 of SEQ. ID NO: 28
f) a VL FWR4 of SEQ. ID NO: 29
wherein one or more mutations are introduced in residue positions selected from the group consisting of: VL 46, VL 49 according to Kabat numbering.

In other embodiments, the antigen-binding protein comprises:
a) a VH FWR2 of SEQ ID NO:31
b) a VH FWR3 of SEQ ID NO:32
c) a VH FWR4 of SEQ ID NO:33
and
d) a VL FWR2 of SEQ. ID NO: 27
e) a VL FWR3 of SEQ. ID NO: 28
f) a VL FWR4 of SEQ. ID NO: 29
wherein one or more mutations are introduced in residue positions selected from the group consisting of: VH 39, VH 44, VH 45, VH 89, VH 91, VH 103, VH 105, VL 32, VL 36, VL 38, VL 43, VL 46, VL 49, VL 85, VL 87, VL 98 and VL 100 according to Kabat numbering.

In some embodiments, the antigen-binding protein comprises:
a) a VH FWR2 of SEQ ID NO:31
b) a VH FWR3 of SEQ ID NO:32
c) a VH FWR4 of SEQ ID NO:33
and
d) a VL FWR2 of SEQ. ID NO: 27
e) a VL FWR3 of SEQ. ID NO: 28
f) a VL FWR4 of SEQ. ID NO: 29
   wherein one or more mutations are introduced in residue positions selected from the group consisting of: VH 39, VH 44, VH 45, VH 89, VH 91, VH 103, VH 105, VL 36, VL 38, VL 43, VL 46, VL 49, VL 85, VL 87, VL 98 and VL 100 according to Kabat numbering.

In other embodiments, the antigen-binding protein comprises:
a) a VH FWR2 of SEQ ID NO: 156
b) a VH FWR3 of SEQ ID NO: 157
c) a VH FWR4 of SEQ ID NO:33
and
d) a VL FWR2 of SEQ. ID NO: 152
e) a VL FWR3 of SEQ. ID NO: 153
f) a VL FWR4 of SEQ. ID NO: 154,
wherein one or more mutations are introduced in residue position VH 39, VH 44, VH 45, VH 47, VH 89, VH 91, VH 103, VH 105, VL 32, VL 36, VL 38, VL 43, VL 44, VL 46, VL49, VL 50, VL 85 VL 87, VL 98 and/or VL 100 according to Kabat numbering, preferably wherein one or more mutations are introduced in residue position VH 44, VH 89, VH 105, and/or VL 38 according to Kabat numbering.

In some embodiments, the antigen-binding protein comprises:
a) a VH FWR2 of SEQ ID NO: 161
b) a VH FWR3 of SEQ ID NO: 162
c) a VH FWR4 of SEQ ID NO:33
and
d) a VL FWR2 of SEQ. ID NO: 158
e) a VL FWR3 of SEQ. ID NO: 159
f) a VL FWR4 of SEQ. ID NO: 154,
wherein one or more mutations are introduced in residue position VH 39, VH 44, VH 45, VH 47, VH 89, VH 91, VH 103, VH 105, VL 32, VL 36, VL 38, VL 43, VL 44, VL 46, VL49, VL 50, VL 85 VL 87, VL 98 and/or VL 100 according to Kabat numbering, preferably wherein one or more mutations are introduced in residue position VH 44, VH 89, VH 105, and/or VL 38 according to Kabat numbering.

In further embodiments, the antigen-binding protein comprises:
a) a VH FWR2 of SEQ ID NO: 164
b) a VH FWR3 of SEQ ID NO: 165
c) a VH FWR4 of SEQ ID NO:33
and
d) a VL FWR2 of SEQ. ID NO: 167
e) a VL FWR3 of SEQ. ID NO: 168
f) a VL FWR4 of SEQ. ID NO: 154,
wherein one or more mutations are introduced in residue position VH 39, VH 44, VH 45, VH 47, VH 89, VH 91, VH 103, VH 105, VL 32, VL 36, VL 38, VL 43, VL 44, VL 46, VL49, VL 50, VL 85 VL 87, VL 98 and/or VL 100 according to Kabat numbering, preferably wherein one or more mutations are introduced in residue position VH 44, VH 89, VH 105, and/or VL 38 according to Kabat numbering.

As described herein, in some embodiments of the antigen-binding proteins, pH-dependent antigen binding proteins, and methods described herein, one or more mutations are introduced in the antigen-binding protein to confer pH-dependent binding properties to said antigen-binding. In some embodiments, mutations to specific amino acid residues at specific residue positions of the VH and VL of said antigen-binding proteins confer pH-dependent binding properties to said antigen-binding proteins.

Thus, an aspect of the invention relates to an antigen-binding protein comprising an antibody light chain variable region (VL) and an antibody heavy chain variable region (VH), wherein one or more mutations are introduced at residue positions selected from the group consisting of: VH 39, VH 44, VH 45, VH 47, VH 89, VH 91, VH 103, VH 105, VL 32, VL36, VL 38, VL 43, VL 44, VL 46, VL49, VL 50, VL 85, VL 87, VL 98 and VL 100 according to Kabat numbering, wherein
the VH contains one or more, such as two, such as three, such as four, of the following amino acid residues:
   an S, E, R, T, or A at position 39,
   a P, R, N, S, K, Q, A, Y, or T at position 44,
   a T, N, I, Q, A, L, Y, D, F, S, or K at position 89, and/or
   a T, R, K, P, D, I, S, or T at position 105.
and/or
wherein the VL contains one or more, such as two, such as three, such as four, such as five, such as six, such as seven, of the following amino acid residues:
   an N at position 36,
   a L, Y, S, I, T, A, R, F, or V at position 38,
   a P, T, A, R, Q, K, or V at position 43
   an A, or S at position 46,
   an A, H, or S at position 49,
   an S, N, T, F, V, L, S, A, H, or R at position 85, and/or
   an S, Y, W, or L at position 100,
wherein all positions are indicated according to Kabat numbering.

In some embodiments, the VH contains one or more, such as two, such as three, such as four, of the following amino acid residues:
an S, E, or A at position 39,
a P, R, N, S, K, Q, A, or T at position 44,
a T, N, I, Q, A, L, Y, D, F, S, or K at position 89, and/or
a T, R, K, P, D, I, S, or T at position 105, wherein all positions are indicated according to Kabat numbering.

In some embodiments the VL contains one or more, such as two, such as three, such as four, such as five, such as six, such as seven, of the following amino acid residues:
an N at position 36,
a L, Y, S, I, T, A, R, F, or V at position 38,
a P, T, A, or V at position 43
an A, or S at position 46,
an A, H, or S at position 49,
an S, N, T, F, V, L, S, A, or R at position 85, and/or
an S, Y, or L at position 100, wherein all positions are indicated according to Kabat numbering.

In preferred embodiments, the antigen-binding protein comprises:

a) a VH FWR2 of
(SEQ ID NO: 98)
WRX₁APGQX₂X₃EX₄MG
b) a VH FWR3 of
(SEQ ID NO: 99)
RVTITADX₅STSTAYMX₆LX₇SLRSDDTAX₈YX₉CAR
c) a VH FWR4 of
(SEQ ID NO: 100)
X₁₀GX₁₁GTLVTVSS,

wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
X₁ is any amino acid except Q, preferably wherein X₁ is S, E, or A, and/or
X₂ is any amino acid except G, preferably wherein X₂ is P, R, N, S, K, Q, A, or T, and/or
X₈ is any amino acid except V, preferably wherein X₈ is T, N, I, Q, A, L, or K, and/or
X₁₁ is any amino acid except Q, more preferably wherein X₁₁ is T, R, K, P, D, I, or T;
and

d) a light chain CDR1 of
(SEQ. ID NO: 104)
TRSX GSIGSDX₂VH
e) a VL FWR2 of
(SEQ. ID NO: 101)
WX₃QX₄RPGSX₅X₆TX₇VIX₈
f) a VL FWR3 of
(SEQ. ID NO: 102)
GVPDRFSGSIDSSSNSASLTISGLKTEDEAX₉YX₁₀C
g) a VL FWR4 of
(SEQ. ID NO: 103)
X₁₁GX₁₂GTKLTVX₁₃,

wherein each of X₁, X₂, X₃, X₄, X₅, X₆, X₇, X₈, X₉, X₁₀, X₁₁, X₁₂, X₁₃ may be any amino acid, with the proviso that at least one of X₂, X₄, X₅, X₇, X₈, X₉, X₁₂ is selected from:
X₂ is any amino acid except Y, preferably wherein X₂ is A, S, T, or H, and/or
X₄ is any amino acid except Q, preferably wherein X₄ is L, Y, S, I, T, A, R, or V, and/or
X₅ is any amino acid except S, preferably wherein X₅ is P, T, A, or V, and/or
X₇ is any amino acid except T preferably wherein X₇ is A, or S, and/or
X₈ is any amino acid except Y, preferably wherein X₈ is A, H, or S, and/or
X₉ is any amino acid except D, preferably wherein X₉ is S, N, T, F, V, L, S, A or R, and/or
X₁₂ is any amino acid except G, preferably wherein X₁₂ is S, Y, or L.

In further embodiments, the antigen-binding protein comprises:

a) a VH FWR2 of
(SEQ ID NO: 98)
WVRX₁APGQX₂X₃EX₄MG
b) a VH FWR3 of
(SEQ ID NO: 99)
RVTITADX₅STSTAYMX₆LX₇SLRSDDTAX₈YX₉CAR
c) a VH FWR4 of
(SEQ ID NO: 100)
X₁₀GX₁₁GTLVTVSS,

wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
X₁ is any amino acid except Q, preferably wherein X₁ is S, E, or A, and/or
X₂ is any amino acid except G, preferably wherein X₂ is P, R, N, S, K, Q, A, or T, and/or
X₈ is any amino acid except V, preferably wherein X₈ is T, N, I, Q, A, L, or K, and/or
X₁₁ is any amino acid except Q, preferably wherein X₁₁ is T, R, K, P, D, I, or T;
and

d) a VL FWR2 of
(SEQ. ID NO: 101)
WX₁QX₂RPGSX₃X₄TX₅VIX₆
e) a VL FWR3 of
(SEQ. ID NO: 102)
GVPDRFSGSIDSSSNSASLTISGLKTEDEAX₆YX₈C
f) a VL FWR4 of
(SEQ. ID NO: 103)
X₉GX₁₀GTKLTVX₁₁,

wherein each of X₁, X₂, X₃, X₄, X₅, X₆, X₇, X₈, X₉, X₁₀, X₁₁ may be any amino acid, with the proviso that at least one of X₂, X₃, X₅, X₆, X₇, X₁₀ is selected from:
X₂ is any amino acid except Q, preferably wherein X₂ is L, Y, S, I, T, A, R, or V, and/or
X₃ is any amino acid except S, preferably wherein X₃ is P, T, A, or V, and/or
X₅ is any amino acid except T preferably wherein X₅ is A, or S, and/or
X₆ is any amino acid except Y, preferably wherein X₆ is A, H, or S, and/or
X₇ is any amino acid except D, preferably wherein X₇ is S, N, T, F, V, L, S, A or R, and/or
X₁₀ is any amino acid except G, preferably wherein X₁₀ is S, Y, or L.

In other embodiments, the antigen-binding protein comprises:

a) a VH FWR2 of
(SEQ ID NO: 187)
WVRX₁APGKX₂X₃EX₄VS
b) a VH FWR3 of
(SEQ ID NO: 188)
RFTISRDX₅AKNSLYLX₆MX₇SLRAEDTAX₈YX₉CAK
c) a VH FWR4 of
(SEQ ID NO: 100)
X₁₀GX₁₁GTLVTVSS,

wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
X₁ is any amino acid except Q, preferably wherein X₁ is S, E, or A, and/or
X₂ is any amino acid except G, preferably wherein X₂ is P, R, N, S, K, Q, A, Y or T, even more preferably wherein X₂ is Y, R, or T, and/or
X₈ is any amino acid except V, preferably wherein X₈ is T, N, I, Q, A, L, Y, D, F, S or K, even more preferably wherein X₈ is Y, D, F, or S and/or
X₁₁ is any amino acid except Q, preferably wherein X₁₁ is T, R, K, P, D, I, S or T, even more preferably wherein X₁₁ is T, S, or P;
and

d) a VL FWR2 of
(SEQ. ID NO: 190)
WX₁QX₂KPGKX₃X₄KX₅LIX₆
e) a VL FWR3 of
(SEQ. ID NO: 191)
GVPSRFSGSGSGTDFTLTISSLQPEDVAX₇YX₈C
f) a VL FWR4 of
(SEQ. ID NO: 192)
X₉GX₁₀GTKVEIX₁₁,

wherein each of X₁, X₂, X₃, X₄, X₅, X₆, X₇, X₈, X₉, X₁₀, X₁₁ may be any amino acid, with the proviso that at least one of X₁, X₂, X₃, X₅, X₆, X₇, X₁₀ is selected from:
X₁ is any amino acid except Y, preferably wherein X₁ is N, and/or
X₂ is any amino acid except Q, preferably wherein X₂ is L, Y, S, I, T, A, R, F, or V, even more preferably wherein X₂ is S, R, or L, and/or
X₃ is any amino acid except A, preferably wherein X₃ is P, T, or V, and/or
X₅ is any amino acid except L preferably wherein X₅ is A, or S, and/or
X₆ is any amino acid except Y, preferably wherein X₆ is A, H, or S, and/or
X₇ is any amino acid except T, preferably wherein X₇ is N, F, V, L, S, A or R, and/or
X₁₀ is any amino acid except Q, preferably wherein X₁₀ is S, Y, or L.

In some embodiments, the antigen-binding protein comprises:

a) a VH FWR2 of
(SEQ ID NO: 193)
WVRQAPGKX₁LEWVS
b) a VH FWR3 of
(SEQ ID NO: 194)
RFTISRDNAKNSLYLQMNSLRAEDTAX₂YYCAK
c) a VH FWR4 of
(SEQ ID NO: 195)
WGX₃GTLVTVSS,
and
d) a VL FWR2 of
(SEQ. ID NO: 196)
WYQX₄KPGKAPKLLIY

wherein each of X₁, X₂, X₃, X₄ may be any amino acid, with the proviso that at least one of X₁, X₂, X₃, X₄ is selected from:
X₁ is any amino acid except G, preferably wherein X₁ is P, R, N, S, K, Q, A, Y or T, even more preferably wherein X₁ is Y, R, or T, and/or
X₂ is any amino acid except V, preferably wherein X₂ is preferably wherein X₂ is T, N, I, Q, A, L, Y, D, F, S or K, even more preferably wherein X₂ is Y, D, F or S, and/or
X₃ is any amino acid except Q, preferably wherein X₃ is T, R, K, P, D, I, S or T, even more preferably wherein X₃ is S, T or P, and/or
X₄ is any amino acid except Q, preferably wherein X₄ is L, Y, S, I, T, A, R, F, or V, even more preferably wherein X₄ is R, S, or L.

In further embodiments, the antigen-binding protein comprises:

a) a VH FWR2 of
(SEQ ID NO: 197)
WVRX₁APGKX₂X₃EX₄VG
b) a VH FWR3 of
(SEQ ID NO: 198)
RFTFSLDX₅SKSTAYLX₆MX₇SLRX₈EDTAX₉YX₁₀CAK
c) a VH FWR4 of
(SEQ ID NO: 189)
X₁₁GX₁₂GTLVTVSS,

wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:

X₁ is any amino acid except Q, preferably wherein X₁ is S, E, or A, and/or
X₂ is any amino acid except G, preferably wherein X₂ is P, R, N, S, K, Q, A, or T, even more preferably wherein X₂ is A, P, S, or T, and/or
X₈ is any amino acid except A, preferably wherein X₈ is T, and/or
X₆ is any amino acid except V, preferably wherein X₈ is T, N, I, Q, A, L, or K, even more preferably wherein X₈ is A, T, I, or L, and/or
X₁₂ is any amino acid except Q, preferably wherein X₁₁ is T, R, K, P, D, I, S, or T, even more preferably wherein X₁₁ is T, D, S, or P;
and

d) a VL FWR2 of
(SEQ. ID NO: 190)
WX₁QX₂KPGKX₃X₄KX₅LIX₆
e) a VL FWR3 of
(SEQ. ID NO: 199)
GVPSRFSGSGSGTDFTLTISSLQPEDFAX₇YX₈C
f) a VL FWR4 of
(SEQ. ID NO: 192)
X₉GX₁₀GTKVEIX₁₁,

wherein each of X₁, X₂, X₃, X₄, X₅, X₆, X₇, X₈, X₉, X₁₀, X₁₁ may be any amino acid, with the proviso that at least one of X₁, X₂, X₃, X₅, X₆, X₇, X₁₀ is selected from:
X₁ is any amino acid except Y, preferably wherein X₁ is N, and/or
X₂ is any amino acid except Q, preferably wherein X₂ is L, Y, S, I, T, A, R, F, or V, even more preferably wherein X₂ is F, L, S, T, or I, and/or
X₃ is any amino acid except A, preferably wherein X₃ is P, T, or V, and/or
X₅ is any amino acid except V preferably wherein X₅ is A, or S, and/or
X₆ is any amino acid except Y, preferably wherein X₆ is A, H, or S, and/or
X₇ is any amino acid except T, preferably wherein X₇ is S, N, F, V, L, A or R, and/or
X₁₀ is any amino acid except Q, preferably wherein X₁₀ is S, Y, or L.

In some embodiments, the antigen-binding protein comprises:

a) a VH FWR2 of
(SEQ ID NO: 200)
WVRQAPGKX₁LEWVG
b) a VH FWR3 of
(SEQ ID NO: 201)
RFTFSLDTSKSTAYLQMNSLRX₂EDTAX₃YYCAK
c) a VH FWR4 of
(SEQ ID NO: 195)
WGX₄GTLVTVSS,
and
d) a VL FWR2 of
(SEQ. ID NO: 202)
WYQX₅KPGKAPKVLIY

wherein each of X₁, X₂, X₃, X₄, may be any amino acid, with the proviso that at least one of X₁, X₂, X₃, X₄, is selected from:
X₁ is any amino acid except G, preferably wherein X₁ is P, R, N, S, K, Q, A, Y or T, even more preferably wherein X₁ is A, P, S, or T, and/or
X₂ is any amino acid except A, preferably wherein X₈ is T, and/or
X₃ is any amino acid except V, preferably wherein X₈ is T, N, I, Q, A, L, Y, D, F, S or K preferably wherein X₂ is A, T, I or L, and/or
X₄ is any amino acid except Q, preferably wherein X₃ is T, R, K, P, D, I, S or T preferably wherein X₃ is T, D, S, or P, and/or
X₅ is any amino acid except Q, preferably wherein X₄ is L, Y, S, I, T, A, R, F or V, even more preferably wherein X₄ is F, L, S, T or I.

In other embodiments, the antigen-binding protein comprises:

a) a VH FWR2 of
(SEQ ID NO: 203)
WRX₁APGKX₂X₃EX₄MG
b) a VH FWR3 of
(SEQ ID NO: 204)
RVTMTEDX₅STDTAYMX₆LX₇SLRSEDTAX₈YX₉CST
c) a VH FWR4 of
(SEQ ID NO: 189)
X₁₀GX₁₁GTLVTVSS,

wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
X₁ is any amino acid except Q, preferably wherein X₁ is S, E, or A, and/or
X₂ is any amino acid except G, preferably wherein X₂ is P, R, N, S, K, Q, A, or T, and/or
X₈ is any amino acid except V, preferably wherein X₈ is T, N, I, Q, A, L, or K, and/or
X₁₁ is any amino acid except Q, preferably wherein X₁₁ is T, R, K, P, D, I, S, or T;
and

d) a VL FWR2 of
(SEQ. ID NO: 190)
WX₁QX₂KPGKX₃X₄KX₅LIX₆
e) a VL FWR3 of
(SEQ. ID NO: 205)
GVPSRFSGSGSGTEFTLTISSLQPEDLAX₇YX₈C
f) a VL FWR4 of
(SEQ. ID NO: 192)
X₉GX₁₀GTKVEIX₁₁,

wherein each of X₁, X₂, X₃, X₄, X₅, X₆, X₇, X₈, X₉, X₁₀, X₁₁ may be any amino acid, with the proviso that at least one of X₁, X₂, X₃, X₅, X₆, X₇, X₁₀ is selected from:
X₁ is any amino acid except Y, preferably wherein X₁ is N, and/or
X₂ is any amino acid except Q, preferably wherein X₂ is L, Y, S, I, T, A, R, F, or V, and/or
X₃ is any amino acid except A, preferably wherein X₃ is P, T, or V, and/or
X₅ is any amino acid except R, preferably wherein X₅ is A, or S, and/or
X₆ is any amino acid except Y, preferably wherein X₆ is A, H, or S, and/or
X₇ is any amino acid except S, preferably wherein X₇ is N, F, V, L, A or R, and/or
X₁₀ is any amino acid except Q, preferably wherein X₁₀ is S, Y, or L.

In preferred embodiments of the antigen-binding proteins of the present invention, variable light chain framework regions (VL FWRs) are interspersed with 3 light chain complementarity-determining regions (CDRLs) and heavy chain framework regions (VH FWRs) are interspersed with 3 heavy chain complementarity-determining regions (CDRHs). In more preferred embodiment of the antigen-binding proteins of the present invention, 4 variable light chain framework regions (VL FWRs) regions are interspersed with 3 light chain complementarity-determining regions (VL CDRs) consecutively, under the format VL FWR1-CDRL1-VL FWR2-CDRL2-VL FWR3-CDRL3-VL FWR4, and 4 variable heavy chain framework regions (VH FWRs) regions are interspersed with 3 heavy chain complementarity-determining regions (VH CDRs) consecutively, under the format VH FWR1-CDRH1-VH FWR2-CDRH2-VH FWR3-CDRH3-VH FWR4.

In other embodiments, the antigen-binding protein comprises:
a) a VH FWR2 of SEQ ID NO:39
b) a VH FWR3 of SEQ ID NO:40
c) a VH FWR4 of SEQ ID NO:33
and
d) a VL FWR2 of SEQ. ID NO: 43
e) FWR2′ is S50 according to Kabat numbering
f) a VL FWR3 of SEQ. ID NO: 45
g) a VL FWR4 of SEQ. ID NO: 47

In other embodiments, the antigen-binding protein further comprises a VH FWR1 of SEQ ID NO:38 and a VL FWR1 of SEQ. ID NO: 41.

In some embodiments, the VL FWR1 of the 2555_01_A01 antibody is of SEQ ID NO: 41. In other embodiments, the VL FWR2 of the 2555_01_A01 antibody is of SEQ ID NO: 42.

In some embodiments, the VL FWR2 of the 2555_01_A01 antibody is of SEQ ID NO: 43. In other embodiments, the VL FWR2 of the 2555_01_A01 antibody is of SEQ ID NO: 44.

In some embodiments, the VL FWR3 of the 2555_01_A01 antibody is of SEQ ID NO: 45. In other embodiments, the VL FWR3 of the 2555_01_A01 antibody is of SEQ ID NO: 46.

In further embodiments, the antigen-binding proteins comprise a VH sequence selected from the group consisting of: SEQ. ID NOs: 77 to 87. In other embodiments, the antigen-binding proteins comprise a VL sequence selected from the group consisting of: SEQ. ID NOs: 35 and 88 to 94.

In some embodiments, the antigen-binding proteins comprise a VH sequence selected from the group consisting of: SEQ. ID NOs: 105 to 124 and a VL sequence selected from the group consisting of: SEQ. ID NOs: 125 to 144.

In yet other embodiments, the antigen-binding proteins comprise a VH sequence selected from the group consisting of: SEQ. ID NOs: 77 to 87 and 105 to 124 and a VL sequence selected from the group consisting of: SEQ. ID NOs: 35, 88 to 94 and 125 to 144.

A fourth aspect of the present invention relates to a pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein one or more mutations are introduced in residue positions selected from the group consisting of: VH 39, VH 44, VH 45, VH 89, VH 91, VH 103, VH 105, VL 32, VL 36, VL 38, VL 43, VL 46, VL 49, VL50, VL 85, VL 87, VL 98 and VL 100 according to Kabat compared to a parental FWR1, FWR2, FWR2′, FWR3 and FWR4, and wherein the pH-dependent antigen-binding protein has higher binding affinity in acidic pH than neutral pH, or lower binding affinity in acidic pH than neutral pH, and wherein said pH-dependent antigen-binding protein does not comprise heavy chain complementarity-determining regions 1, 2 and 3 of SEQ. ID NOs 1, 2, 3 respectively and light chain complementarity-determining region 1, 2 and 3 of SEQ. ID NOs 4, 5, 6 respectively.

An important advantage of the invention is that the methods described herein can in turn allow the pH-dependent antibodies subsequently discovered from the library, to be used as generic scaffolds to generate new synthetic libraries of antibodies with pH-dependent binding pre-determined by their framework region.

Importantly, the pH-dependent antigen binding being ‘pre-determined’ in each clone alleviates the need for histidine doping or other low-throughput engineering approaches for the discovery of recycling antibodies. Moreover, the use of such library leads to the identification of amino acid substitutions in the heavy/light chain interface that robustly endow antibodies with pH-sensitivity, and that the substitutions can be routinely introduced into monoclonal antibodies on a case-by-case basis by rational design.

Therefore, a fifth aspect of the present invention relates to a method for producing a pH-dependent antigen-binding protein directed to an antigen of interest, said method comprising the steps of:
providing an antigen-binding protein binding an antigen of interest;
introducing in said antigen-binding protein one or more mutations in residue positions selected from the group consisting of: VH 39, VH 44, VH 45, VH 47, VH 89, VH 91, VH 103, VH 105, VL 32, VL 36, VL 38, VL 43, VL 44, VL 46, VL49, VL 50, VL 85 VL 87, VL 98 and VL 100 according to Kabat numbering,
   thereby producing a pH-dependent antigen-binding protein targeted to said antigen of interest.

In some embodiments of the method, the one or more mutations are introduced in residue positions selected from the group consisting of: VH 39, VH 44, VH 45, VH 47, VH 89, VH 91, VH 103, VH 105, VL 36, VL 38, VL 43, VL 44, VL 46, VL 49, VL 50, VL 85, VL 87, VL 98 and VL 100 according to Kabat numbering

In other embodiments of the method, the one or more mutations are substitution of an amino acid selected from the group consisting of: VH Q39, VH G44, VH L45, VH W47, VH V89, VH Y91, VH W103, VH Q105, VL Y32, VL Y36, VL Q38, VL S43, VL P44, VL T46, VL Y49, VL E50, VL D85, VL Y87, VL F98, VL G100 according to Kabat numbering for another amino acid.

In further embodiments of the method, the one or more mutations are substitution of an amino acid selected from the group consisting of: VH Q39, VH G44, VH L45, VH W47, VH V89, VH Y91, VH W103, VH Q105, VL Y36, VL Q38, VL S43, VL P44, VL T46, VL Y49, VL E50, VL D85, VL Y87, VL F98, VL G100 according to Kabat numbering for another amino acid.

In yet other embodiments of the method, the one or more mutations are introduced in residue positions from the group consisting of: VH 39, VH 44, VH 89, VH 105, VL 38, VL 43, VL 50, VL 85 and VL 100 according to Kabat numbering for another amino acid.

In some embodiments of the method, the one or more mutations are substitution of an amino acid selected from the group consisting of: VH Q39, VH G44, VH V89, VH Q105, VL Q38, VL S43, VL E50, VL D85 and VL G100 according to Kabat numbering for another amino acid.

In some embodiments of the method, the one or more mutations are introduced in residue positions selected from the group consisting of VH 45, VH 47, VH 91, and VH 103 according to Kabat numbering.

In other embodiments of the method, the one or more mutations are introduced in residue positions selected from the group consisting of VH L45, VH W47, VH Y91, and VH W103 according to Kabat numbering.

In other embodiments of the method, the one or more mutations are introduced in residue positions selected from the group consisting of VL 32, VL 46 and VL 49 according to Kabat numbering.

In further embodiments of the method, the one or more mutations are introduced in residue positions selected from the group consisting of VL 46 and VL 49 according to Kabat numbering.

In preferred embodiments of the method, the one or more mutations are introduced in residue positions selected from the group consisting of VL Y32, VL T46 and VL Y49 according to Kabat numbering.

In other embodiments of the method, the one or more mutations are introduced in residue positions selected from the group consisting of VL T46 and VL Y49 according to Kabat numbering.

In other embodiments of the method, the one or more mutations are introduced in residue positions selected from the group consisting of VL 36, VL 44, VL 87 and VL 98 according to Kabat numbering.

In some embodiments of the method, the one or more mutations are introduced in residue positions selected from the group consisting of VL Y36, VL P44, VL Y87 and VL F98 according to Kabat numbering

In preferred embodiments, the step of introducing the one or more mutations further comprises testing that the antigen-binding protein comprising the one or more mutations has a higher binding affinity in acidic pH than neutral pH, or a lower binding affinity in acidic pH than neutral pH.

It may be beneficial to ensure that the antigen-binding proteins comprising the one or more mutations have a similar or better developability as antibodies comprising the parental residues, in particular it may be beneficial to select the antibodies generated or isolated which present the greatest thermal stability.

Thus, in some embodiments, the method further comprises a step of selecting the antibodies displaying highest thermal stability, such as the highest Fab fragment melting temperature.

### Residue Mutations—Properties of Mutated Amino Acids or Mutations to Said Amino Acids

The skilled person will appreciate that certain amino acids residues have a side chain which can be charged. At neutral pH (7), Aspartic acid and Glutamic acid are for instance negatively charged (acidic side chains) while Lysine, Arginine and Histidine are positively charged (basic side chains).

In some embodiments, the one or more mutations are at residue positions occupied by charged residues.

In further embodiments, the one or more mutations are mutations to charged residues.

In other embodiments, the one or more mutations are at residue positions occupied by residues which can engage in hydrogen bonding.

In yet further embodiments, the one or more mutations are mutations to residues which can engage in hydrogen bonding.

The amino acids (AA) residues mutated in the methods or the pH-dependent antigen-binding proteins of the present invention are preferably AAs having hydrogen donor or acceptor atoms on their side chains to engage in hydrogen bonding. The skilled person will appreciate that hydrogen bonding involves the interaction of an hydrogen atom located between a pair of other atoms having high affinity for electrons. The donor atom of the pair is typically a nitrogen, oxygen or fluorine covalently bonded to an hydrogen atom as —NH, —OH, or —FH. The acceptor atom is typically a nitrogen, oxygen or fluorine with an unshared electron pair. Typically, polar amino acids are capable of forming hydrogen bonds via their side chains.

Thus in some embodiments, the one or more mutations are at residue positions occupied by residues selected from the group consisting of: Arginine, Asparagine, Aspartic acid, Glutamine, Glutamic acid, Histidine, Lysine, Serine, Threonine, Tryptophan, and Tyrosine.

In other embodiments, the one or more mutations are mutations to residues selected from the group consisting of: Arginine, Asparagine, Aspartic acid, Glutamine, Glutamic acid, Histidine, Lysine, Serine, Threonine, Tryptophan, and Tyrosine.

In further embodiments, the one or more mutations are at residue positions occupied by residues selected from the group consisting of: Arginine, Asparagine, Aspartic acid, Glutamine, Glutamic acid, Lysine, Serine, Threonine, Tryptophan, and Tyrosine.

In yet further embodiments, the one or more mutations are mutations to residues selected from the group consisting of: Arginine, Asparagine, Aspartic acid, Glutamine, Glutamic acid, Lysine, Serine, Threonine, Tryptophan, and Tyrosine.

The AAs mutated of the present invention may also preferably be charged AAs.

Thus in some embodiments, the one or more mutations are at residue positions occupied by residues selected from the group consisting of: Arginine, Aspartic acid, Glutamic acid, Histidine, and Lysine.

In other embodiments, the one or more mutations are mutations to residues selected from the group consisting of: Arginine, Aspartic acid, Glutamic acid, Histidine, and Lysine.

In further embodiments, the one or more mutations are at residue positions occupied by residues selected from the group consisting of: Arginine, Aspartic acid, Glutamic acid, Histidine, and Lysine.

In yet further embodiments, the one or more mutations are mutations to residues selected from the group consisting of: Arginine, Aspartic acid, Glutamic acid, Histidine, and Lysine.

pH-Dependence—Kd

In some embodiments, the pH-dependent antigen-binding protein has a lower Kd value to its antigen at acidic pH compared to the Kd value at neutral pH.

In preferred embodiments, the Kd value at acidic pH is decreased by a factor 2, such as a factor 5, for instance a factor 10, such as a factor 25, for instance a factor 50, such as a factor 75, for instance a factor 100, such as a factor 125, for instance a factor 250, such as a factor 500, for instance a factor 750, such as a factor 1000 compared to neutral pH.

In other embodiments, the pH-dependent antigen-binding protein has a higher Kd value to its antigen at acidic pH compared to the Kd value at neutral pH.

In preferred embodiments, the Kd value at acidic pH is increased by a factor 2, such as a factor 5, for instance a factor 10, such as a factor 25, for instance a factor 50, such as a factor 75, for instance a factor 100, such as a factor 125, for instance a factor 250, such as a factor 500, for instance a factor 750, such as a factor 1000 compared to neutral pH.

### Types of Antibodies and Possible Modifications

In some embodiments, the pH-dependent antigen-binding protein is selected from the group consisting of a full-length antibody, a Fab fragment, a F(ab′) fragment, a F(ab′)2 fragment, an scFv, a diabody, and a triabody.

In other embodiments, the pH-dependent antigen-binding protein comprises an immunoglobulin constant region.

In further embodiments, the pH-dependent antigen-binding protein immunoglobulin constant region is selected from the group consisting of IgG, IgM, IgA, IgD, and IgE.

In yet further embodiments, the pH-dependent antigen-binding protein immunoglobulin constant region is selected from the group consisting of IgG and IgA.

In some embodiments, the pH-dependent antigen-binding protein immunoglobulin constant region is selected from the group consisting of IgG1, IgA1, and IgA2.

In other embodiments, the pH-dependent antigen-binding protein immunoglobulin constant region is selected from the group consisting of IgG1, IgG2, IgG3, and IgG4.

In preferred embodiments, the pH-dependent antigen-binding protein is a monoclonal antibody. In further embodiments, the pH-dependent antigen-binding protein is a human antibody or a chimeric antibody.

Chimeric antibodies retains CDRs from its original species incorporated into the antibody from another species, such as humans. Other chimeric antibodies retain the variable regions from its original species fused to constant regions of another species, such a humans. Advantages of chimeric antibodies are a reduced immunogenicity in humans compared to murine antibodies and a cheaper production compared to what is required for generating a fully humanized antibody.

The antibodies may be a multispecific antibodies (e.g. bispecific antibodies) formed from at least two different antibodies, and/or antibody fragments.

In some embodiment, the antigen-binding protein is a human antibody or an antibody based on human scaffold. The antibody may also be a humanised antibody comprising

CDR regions (and possibly a few other residues) transferred from another species having the desired specificity, affinity, and capacity. Humanised antibodies may also comprise synthetic CDR regions, e.g. from a synthetic antibody library.

The generation of antibodies may be achieved by any standard methods in the art for producing antibodies.

Recombinant antibodies may be isolated from libraries of genes encoding fragments of antibodies. The fragments of antibodies can for example be any of the aforementioned antibody fragments, such as Fab, Fv fragments, single chain fragment of heavy and light chain variable domains or single domain antibodies, such as polypeptides comprising or consisting of VH or VL domains. The antigen-binding protein may be single chain antibodies comprising heavy and light chain variable domains linked to each other, e.g. the antigen-binding protein may be a scFv. The libraries of genes may be obtained from natural sources, as in the case of naïve or immunised libraries, or they may be created by synthetic means. Isolation of specific antibodies from the libraries can be mediated by panning of phage displayed antibody libraries on specific antigens or complex mixtures. Libraries may be obtained by panning of phage displayed antibody libraries on a specific antigen from several species in several rounds. The phage displayed antibody libraries may be screened by successive selection on the antigen or a fragment thereof from humans followed by panning the phage displayed antibody libraries on antigens from mice or a fragment of murine antigens corresponding to the fragment from the corresponding human antigen. These pannings may be performed in either order. Alternatively, methods such as yeast display, bacterial display, ribosome display, etc. can be applied in the selection of monoclonal recombinant antibodies.

The antibody may be a human single domain antibody or a single domain antibody based on human sequences, wherein diversity has been artificially generated. Several different libraries of useful human single domain antibodies are available

The skilled person will appreciate that antibodies can beneficially be labelled for certain applications. In some embodiments of the present invention, the pH-dependent antigen-binding protein comprises a detection label.

In further embodiments, the detection label is selected from the group consisting of a colorimetric, a fluorescent, a luminescent, a magnetic, and a paramagnetic label.

In other embodiments, the detection label is biotin.

In yet other embodiments, the detection label is a gold nanoparticle.

### Composition

A sixth aspect of the present invention relates to a composition comprising the pH-dependent antigen-binding protein as described herein and a pharmaceutically acceptable excipient.

### Medical Uses and Methods of Treatments

A seventh aspect of the present invention relates to a pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein the pH-dependent antigen-binding protein has a higher binding affinity in acidic pH than neutral pH, or lower binding affinity in acidic pH than neutral pH, or the composition as described herein, for use in a method of treatment of cancer, autoimmune diseases, metabolic diseases, or haematological diseases in a patient in need thereof.

In preferred embodiments, the cancer is a solid-tumor cancer.

In even preferred embodiments, the pH-dependent antigen-binding protein is the pH-dependent antigen-binding protein is as described herein.

An eighth aspect of the present invention relates to a method of treating cancer, autoimmune diseases, metabolic diseases, or haematological diseases, comprising administering to a patient in need thereof a pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein the pH-dependent antigen-binding protein has higher binding affinity in acidic pH than neutral pH, or lower binding affinity in acidic pH than neutral pH, or the composition as described herein.

An ninth aspect of the present invention relates to the use of a pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein the pH-dependent antigen-binding protein has higher binding affinity in acidic pH than neutral pH, or lower binding affinity in acidic pH than neutral pH, or the composition as described herein in the manufacture of a medicament for the treatment of cancer, autoimmune diseases, metabolic diseases, or haematological diseases.

### In Vitro Methods

A tenth aspect of the present invention relates to the use of a pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein the pH-dependent antigen-binding protein has higher binding affinity in acidic pH than neutral pH, or lower binding affinity in acidic pH than neutral pH, or the composition as described herein in an in vitro method for detection and/or diagnosis of cancer.

The pH sensitive antigen-binding properties may be of different nature, typically binding the antigen at physiological pH while allowing release of the antigen at lower pH (e.g. in endosomes) and enabling antibody recycling to the circulation. This approach could be useful in different applications requiring clearance of circulating proteins (e.g. toxins, cytokines . . . ). On the other hand, the pH sensitive antigen-binding properties may on the contrary allow binding at low-pH and release of the antigen in high pH environments. This property could see applications in targeted treatment of acidic environments e.g. tumor microenvironment.

In preferred embodiments, the plasma recycling of the pH-dependent antigen-binding protein is improved compared to the antigen-binding protein comprising the parental FWR1, FWR2, FWR2′, FWR3 and FWR4.

In other embodiments, the pH-dependent antigen-binding improves the clearance of its antigen from the plasma compared to the antigen-binding protein comprising the parental FWR1, FWR2, FWR2′, FWR3 and FWR4.

In yet other embodiments, the pH-dependent antigen-binding improves the release from its antigen in endosomes compared to the antigen-binding protein comprising the parental FWR1, FWR2, FWR2′, FWR3 and FWR4.

In further embodiments, the intracellular uptake of the pH-dependent antigen-binding protein is improved in acidic microenvironments compared to the antigen-binding protein comprising the parental FWR1, FWR2, FWR2′, FWR3 and FWR4.

An eleventh aspect of the present invention relates to an in-vitro antigen detection method comprising:
providing a pH-dependent antigen-binding protein as described herein
contacting the pH-dependent antigen-binding protein with the antigen it binds to;
and
detecting the contact between the pH-dependent antigen-binding protein and its antigen,
   thereby detecting the antigen.

A twelfth aspect of the present invention relates to an in-vitro antigen purification method comprising:
providing a pH-dependent antigen-binding protein as described herein;
contacting the pH-dependent antigen-binding protein with the antigen it binds to in a complex mixture; and
separating the pH-dependent antigen-binding protein/antigen complex from the complex mixture,
   thereby purifying the antigen.

In preferred embodiments of the antigen-binding protein as described herein, each of the VL and VH comprise 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein said pH-dependent antigen-binding protein does not comprise heavy chain complementarity-determining regions 1, 2 and 3 of SEQ. ID NOs 1, 2, 3 respectively and light chain complementarity-determining region 1, 2 and 3 of SEQ. ID NOs 4, 5, 6 respectively

In even preferred embodiments, the 3 complementarity-determining regions, CDR1, CDR2 and CDR3, the 4 framework regions FWR1, FWR2, FWR3 and FWR4, and the FWR2′ of the antigen binding protein as described herein are as described for the pH-dependent-antigen binding proteins as described herein.

In some embodiments of the method as described herein, the one or more mutations are introduced in residue positions selected from the group consisting of: VH 39, VH 44, VH 45, VH 47, VH 89, VH 91, VH 103, VH 105, VL 36, VL 38, VL 43, VL 44, VL 46, VL 49, VL 85, VL 87, VL 98 and VL 100 according to Kabat numbering.

In other embodiments of the method as described herein, the one or more mutations are substitution of an amino acid selected from the group consisting of: VH Q39, VH G44, VH L45, VH W47, VH V89, VH Y91, VH W103, VH Q105, VL Y32, VL Y36, VL Q38, VL S43, VL P44, VL T46, VL Y49, VL D85, VL Y87, VL F98, VL G100 according to Kabat numbering for another amino acid.

In yet other embodiments of the method as described herein the one or more mutations are substitution of an amino acid selected from the group consisting of: VH Q39, VH G44, VH L45, VH W47, VH V89, VH Y91, VH W103, VH Q105, VL Y36, VL Q38, VL S43, VL P44, VL T46, VL Y49, VL D85, VL Y87, VL F98, VL G100 according to Kabat numbering for another amino acid.

In other embodiments of the method as described herein, the one or more mutations are introduced in residue positions from the group consisting of: VH 39, VH 44, VH 89, VH 105, VL 38, VL 43, VL 85 and VL 100 according to Kabat numbering for another amino acid.

In preferred embodiments of the method, the pH-dependent antigen-binding protein, the antigen binding protein, the composition or the use as described herein, said higher binding affinity in acidic pH than neutral pH, or lower binding affinity in acidic pH than neutral pH, is a lower KD or a higher KD, respectively.

In some embodiments of the method, the pH-dependent antigen-binding protein, the antigen binding protein, the composition or the use as described herein, said higher binding affinity in acidic pH than neutral pH, or lower binding affinity in acidic pH than neutral pH is measured as a ratio of DELFIA signals in acidic pH and neutral pH.

In preferred embodiments of the method, the pH-dependent antigen-binding protein, the antigen binding protein, the composition or the use as described herein, said higher binding affinity in acidic pH than neutral pH, or lower binding affinity in acidic pH than neutral pH is by at least 1.25-fold, such as at least 1.3-fold, such as at least 1.5-fold, such as at least 1.6-fold, such as at least 1.7-fold, such as at least 1.8-fold, such as at least 1.9-fold, such as at least 2-fold, such as at least 2.2-fold, such as at least 2.5-fold, such as at least 5-fold, such as at least 6-fold, such as at least 10-fold, such as at least 10.5-fold.

### EXAMPLES

### Example 1: Profiling Cross-Reactivity and pH-Dependent Binding for Light Chain Shuffled Antibodies

### Material and Methods

### SuperTEV Expression

SuperTEV endoprotease was expressed using the pET39-mCherry-superTEV expression vector in BL21 (DE3) cells (New England Biolabs, NEB-C2527H) as described previously (Ref. 11).

### Expression of Antibody Formats

Full-length human IgGs were produced using mammalian expression as described previously (Ref. 7). Fabs were produced in two batches, the first by Human Embryonic Kidney cells, the second by Chinese Hamster Ovary cells (Ref. 8).

To produce scFv for crystallography, C-term TEV-His-tagged scFv was produced in BL21 (DE3) cells (New England Biolabs, NEB-C2527H) and purified by Nickel affinity purification as described previously, 18 with the exception that Tunair™ shake flasks were used to increase expression. Following Nickel affinity purification, the scFv was buffer-exchanged into 20 mM Tris, 50 mM NaCl, 5 mM EDTA, pH 8.0 buffer using PD-columns (Merck, GE17-0851-01), and up-concentrated to 10 mg/ml using 10 kDa MWCO membranes (Fisher Scientific, 10781543). The C-terminal tag was removed by incubation with the superTEV endoprotease overnight at 4° C. using a 1:20 molar ratio of superTEV:scFv.

Once the C-terminal tag was removed, monomeric scFv was purified by size exclusion chromatography using an NGC Quest™ 10 Plus Chromatography system and a Superdex 75 10/60 HiLoad column (Cytiva, 28989333), which was run using 5 mM Tris, 20 mM NaCl, pH 8.0 buffer at 4° C. as eluent. The concentration of scFv was estimated based on the predicted A280 absorbance of 1 mg/ml of protein using the Expasy ProtParam tool.

Octet Screening of pH-Sensitive Antibodies

An Octet RED96 system (ForteBio) was used to characterise the affinities and pH-sensitivity of antibody Fab fragments. All reagents were transferred into black 96-well plates (Greiner Bio-One, 655209) before starting the assay, which was run at 24° C. using a 1000 rpm shake speed. Streptavidin biosensor tips (Sartorius, 18-5136) were equilibrated in kinetics buffer (Sartorius, 18-1105), prepared in PBS (137 mM NaCl, 3 mM KCl, 8 mM Na2HPO4.2H2O, 1.4 mM KH2PO4, pH 7.4) for 10 min in the dark before the start of the assay. The long chain α-neurotoxin was biotinylated as described previously (Ref. 8) and diluted in kinetics buffer to a concentration of 0.4 μg/mL. Equilibrated biosensors were dipped into biotinylated long chain α-neurotoxin wells for 120 s to allow for sufficient loading, and a no-toxin coated biosensor was used as a reference. Biosensors were then transferred into HEPES-MES (10 mM HEPES 50 mM MES-NaCl 0.05% P20, pH 7.4) running buffer and equilibrated for 30 s before being primed in 10 mM glycine, 2 M NaCl, pH 2.0 regeneration buffer for 3 cycles. Each cycle consisted of 10 s of regeneration and 10 s of neutralization in kinetics buffer. Primed biosensors were transferred into running buffer for 60 s to obtain a stable baseline before being transferred into Fab-containing wells for 120 s. The antibody Fab fragments were prepared in running buffer in a 3-fold titration series in a concentration range spanning from 10-fold lower to 10-fold higher than the expected KD. Dissociation of bound Fab in running buffer was given 1000 s for sufficient dissociation for high-affinity interactions. In the case of affinities and dissociation rates measured at acidic pH, the running buffer was adjusted to pH 5.5. Data was processed in the Octet evaluation software (version 12.2.2.4). The reference was subtracted from the binding curves, and a 1:1 binding model with a global fit was used to fit all curves. The KD values were determined either by a product of the kinetic rates (kd/ka) or by steady-state analysis. Flow cells were regenerated by 5×10 s cycles of regeneration solution followed by neutralization.

### Results

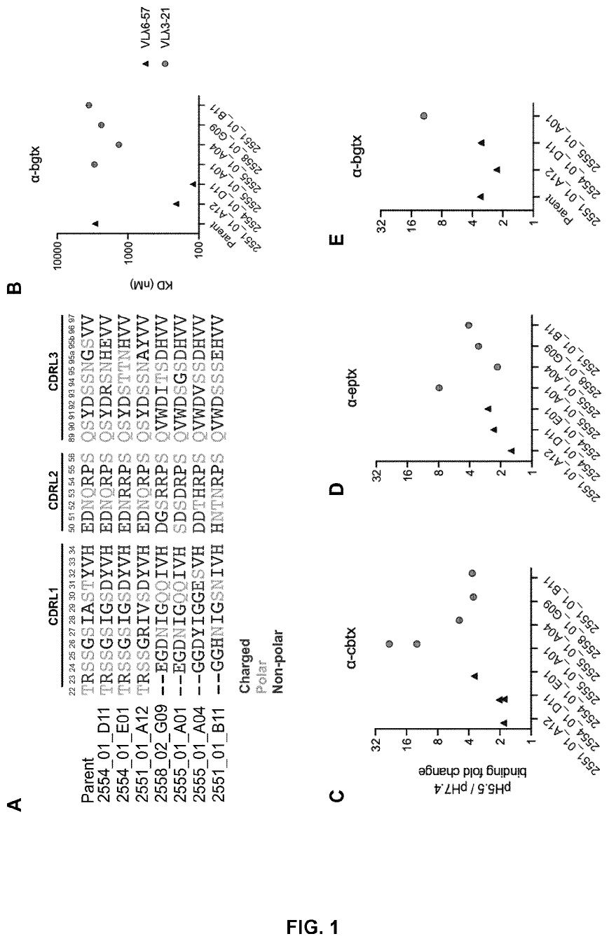

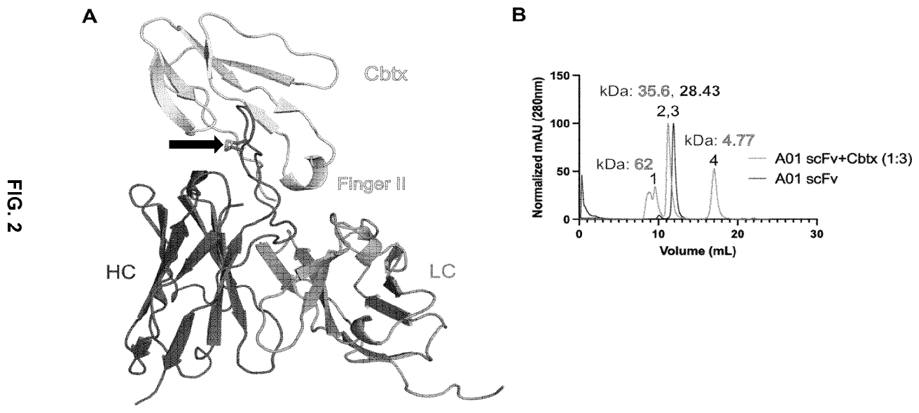

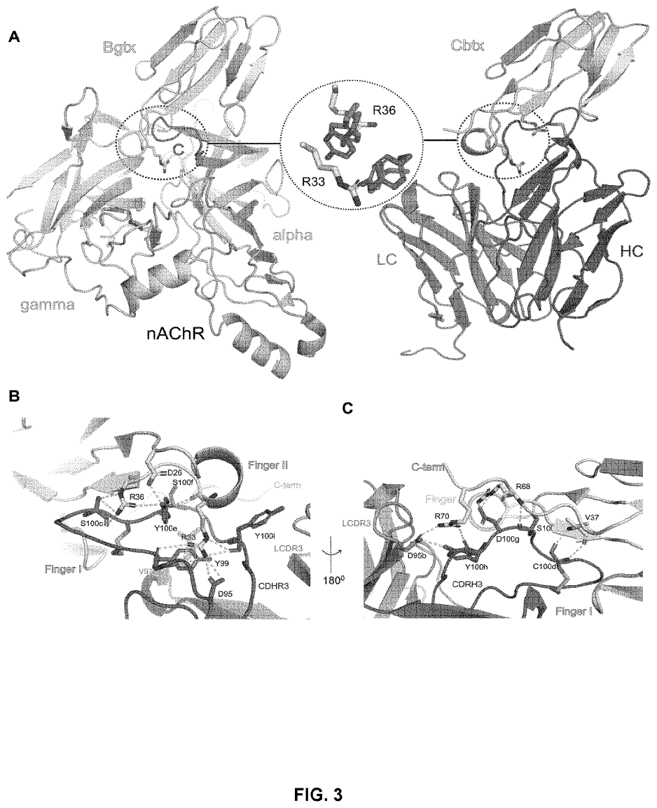

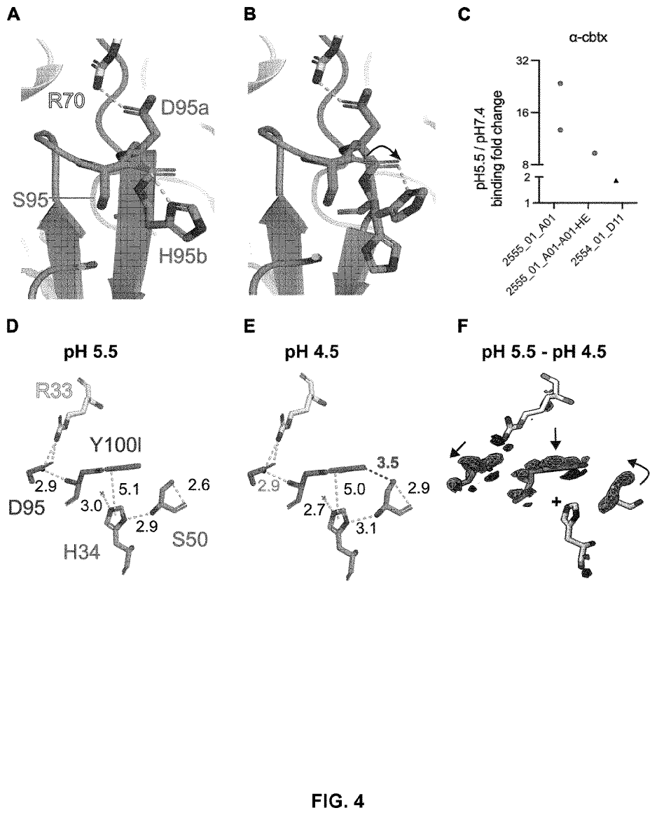

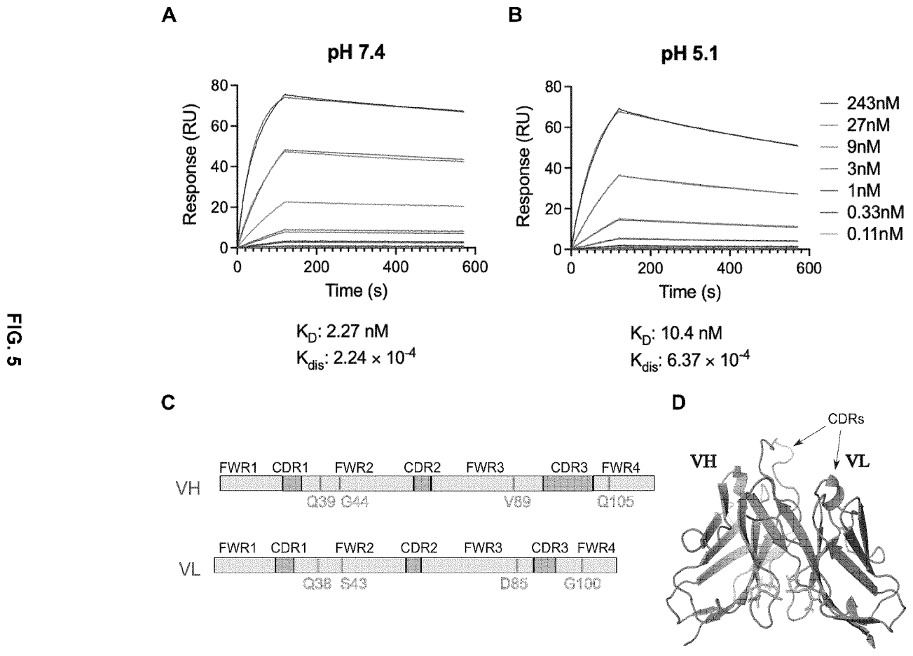

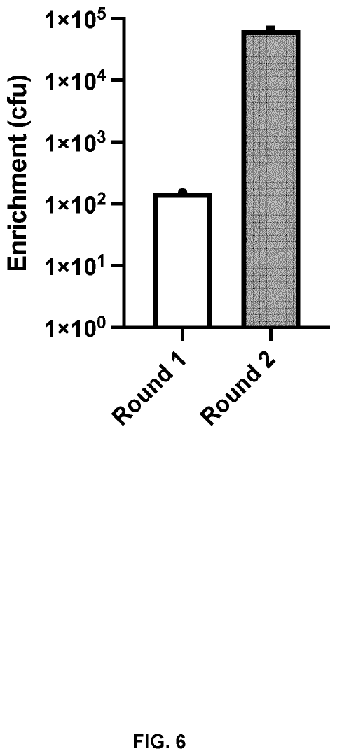

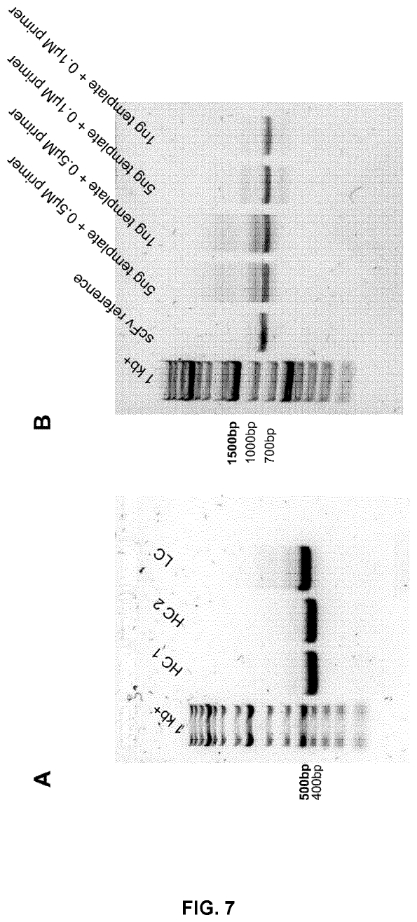

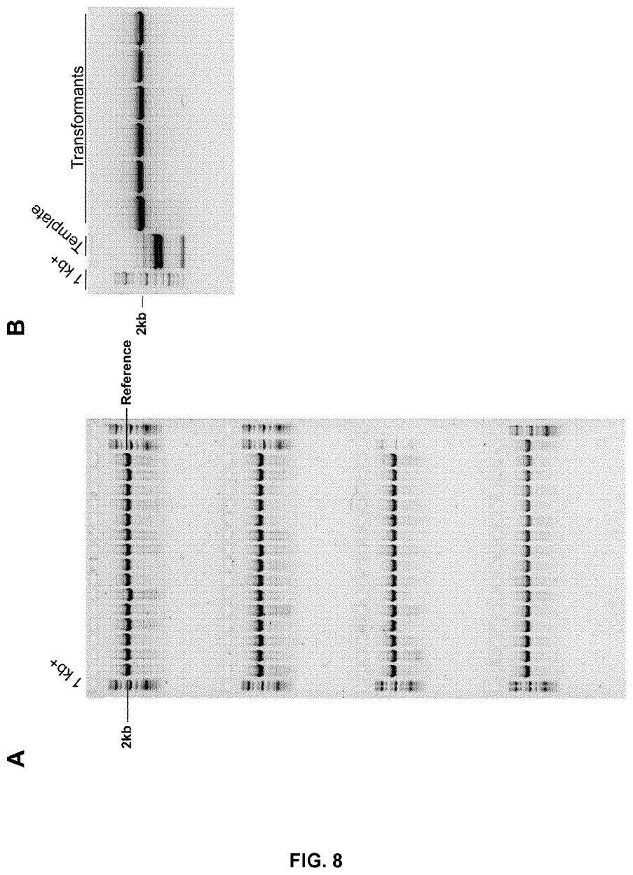

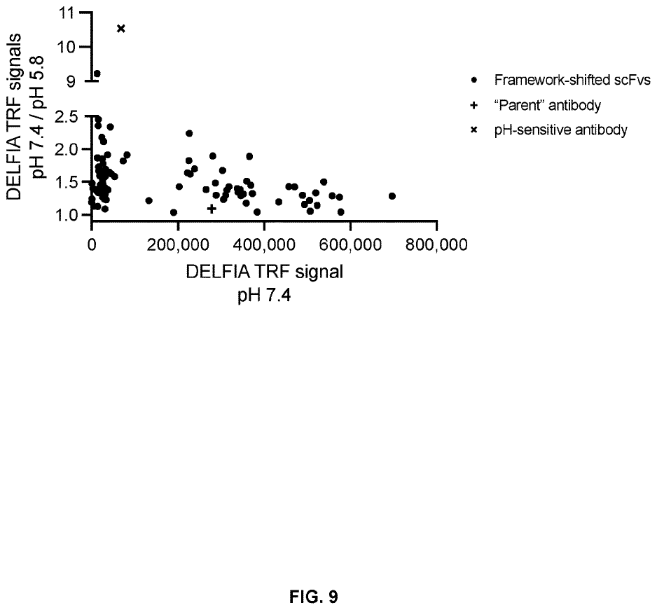

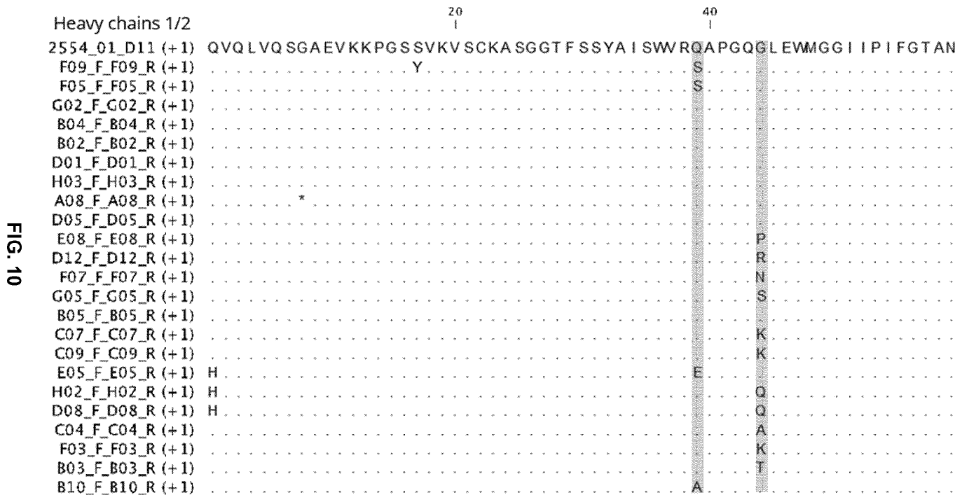

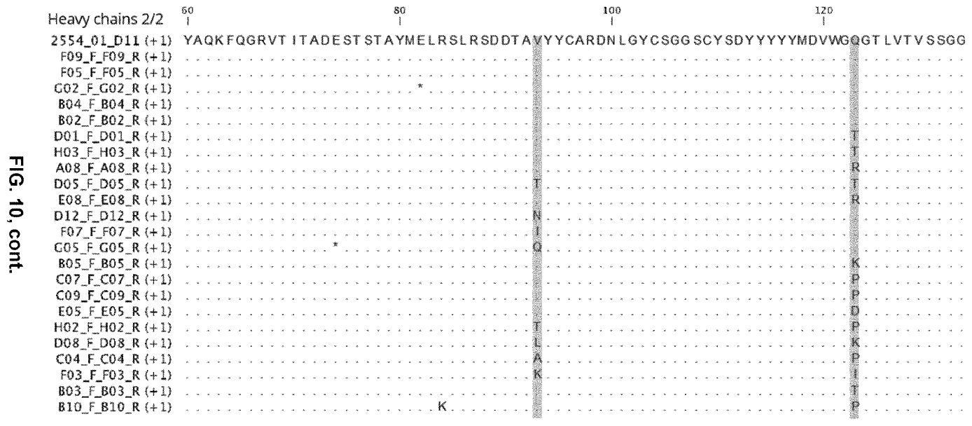

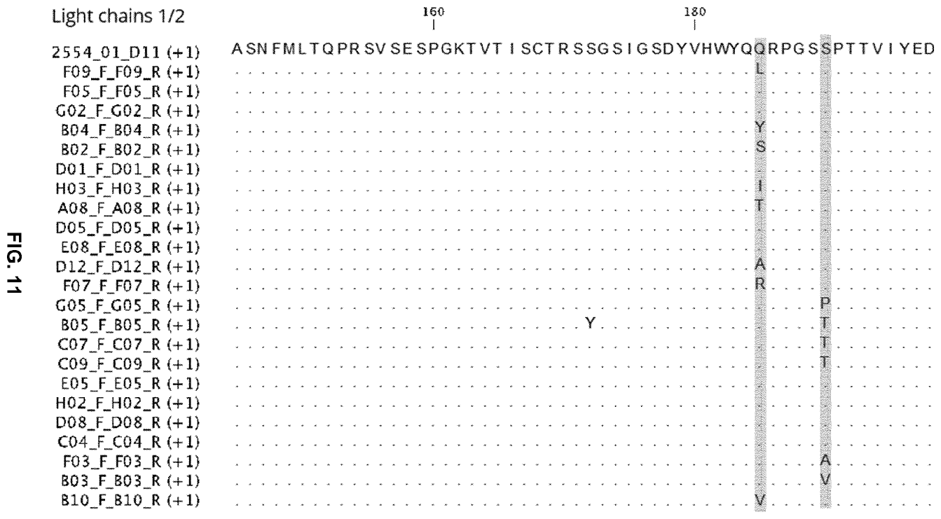

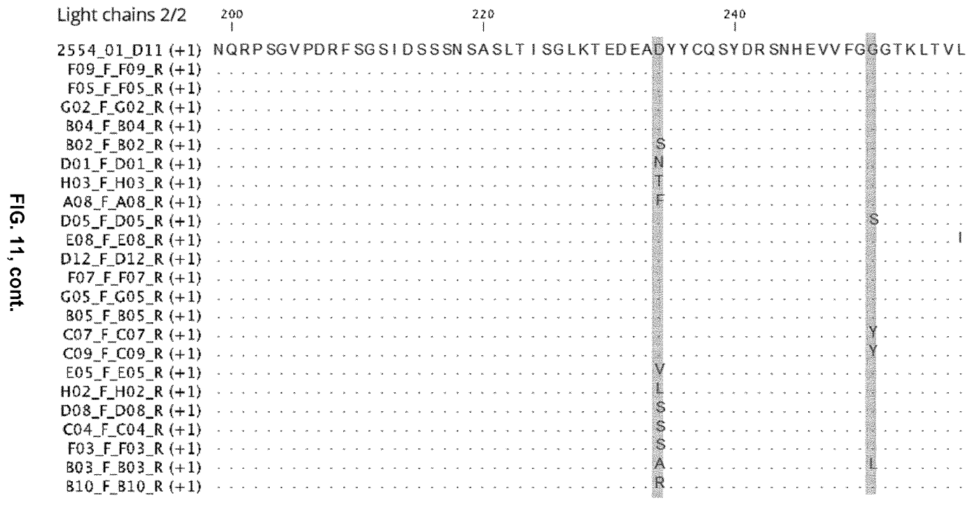

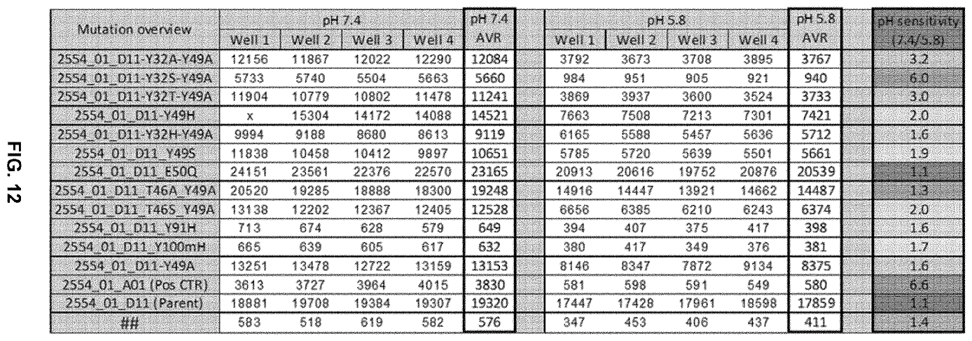

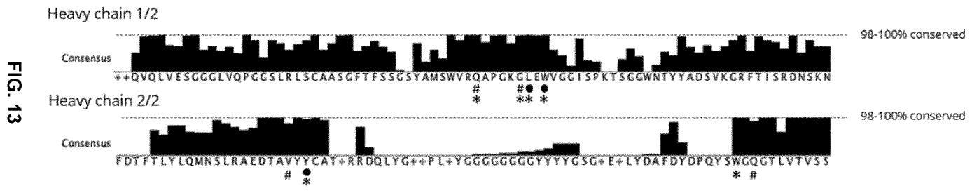

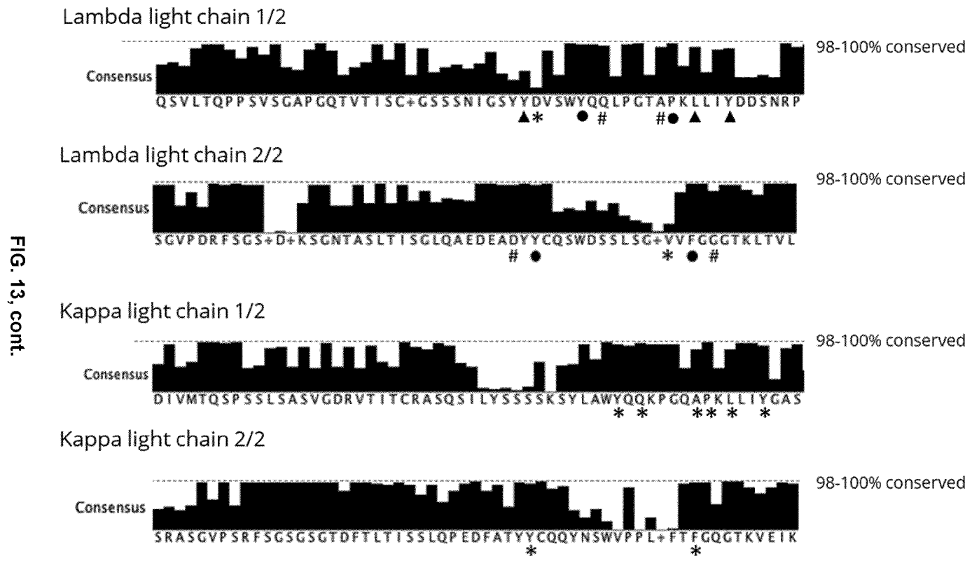

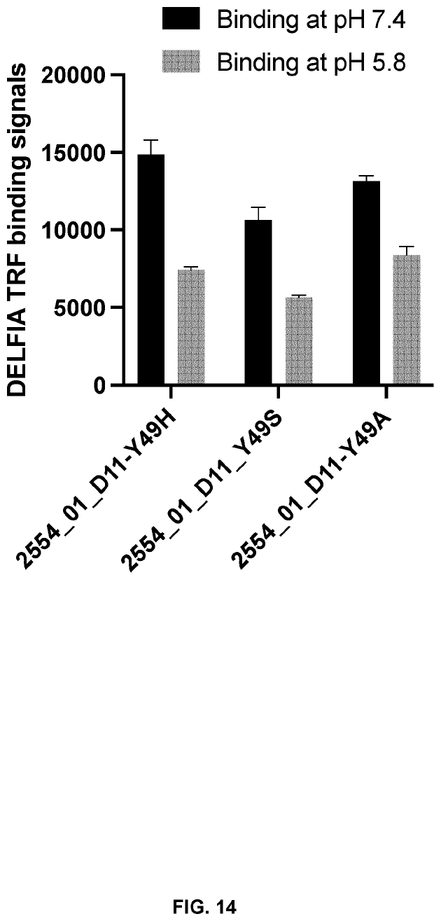

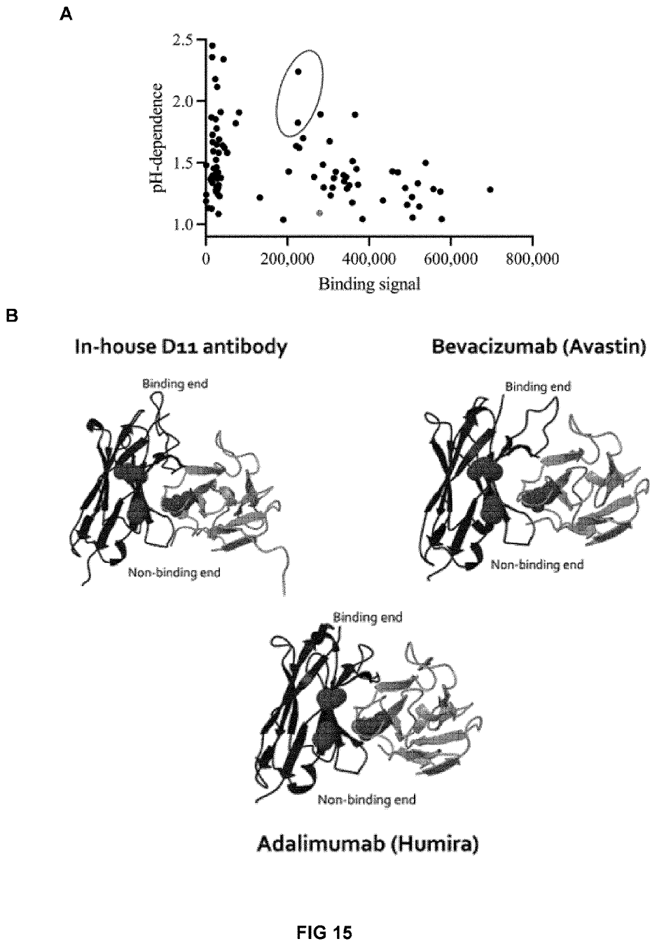

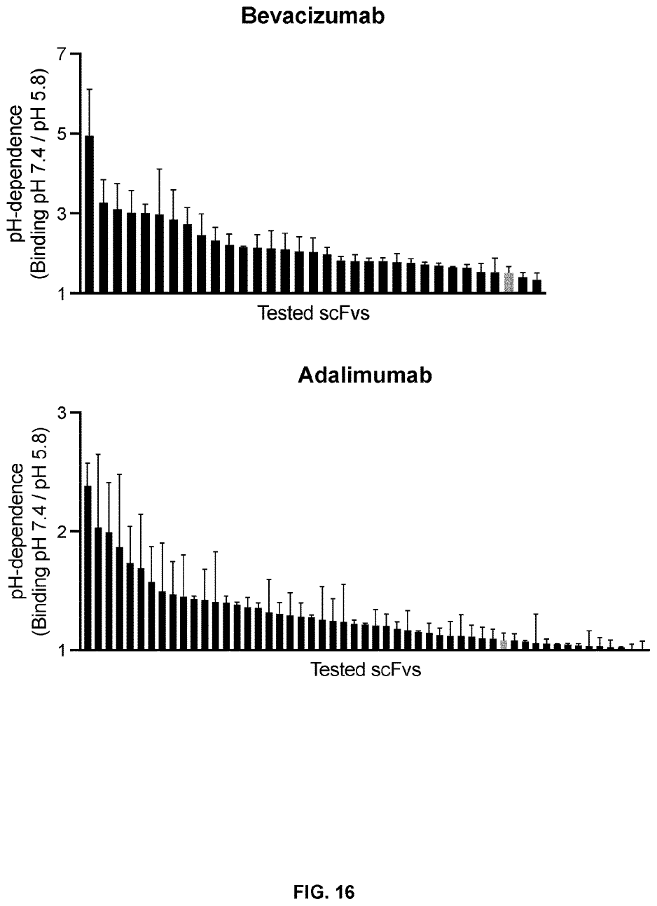

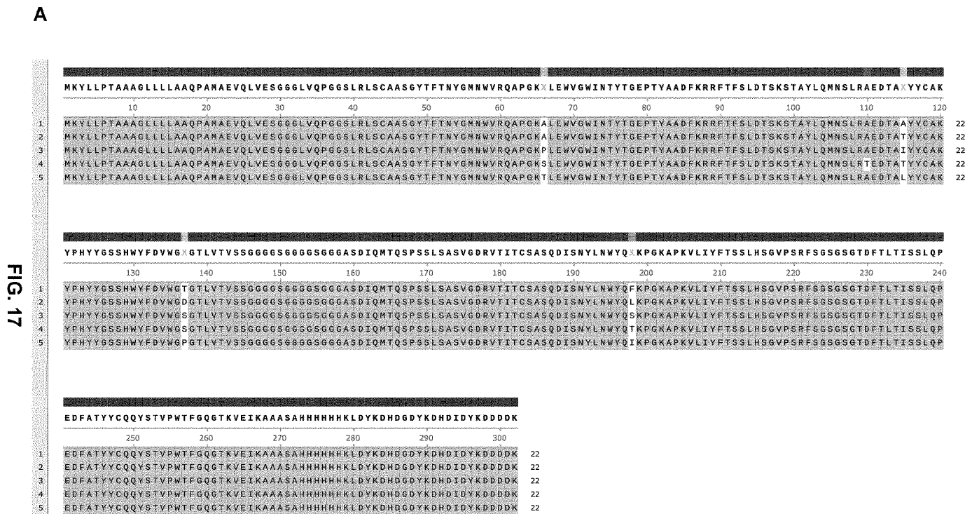

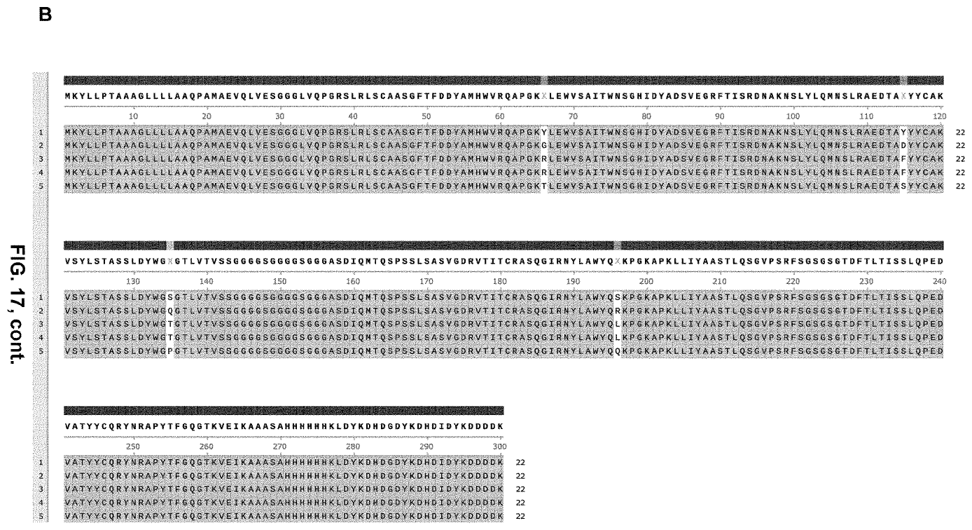

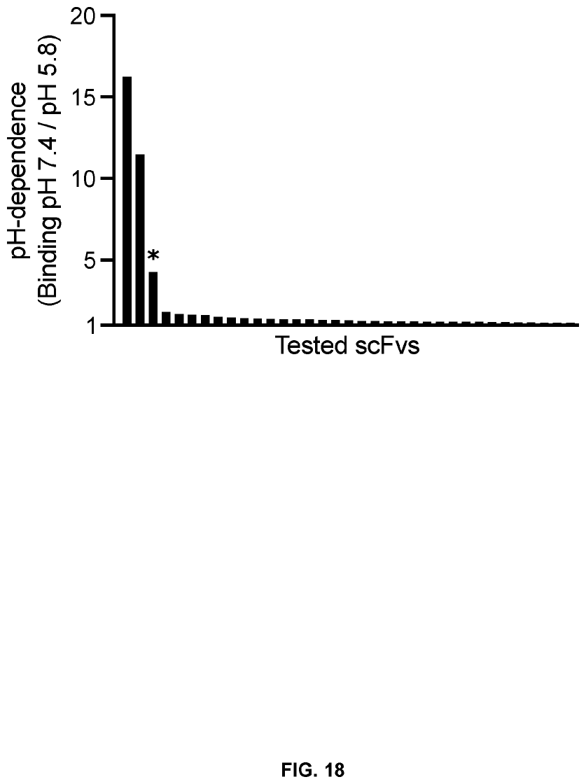

To determine whether cross reactivity and pH-sensitivity could be coupled, the inventors screened a panel of antibodies for pH-sensitive binding to three long-chain α-neurotoxins: N. kaouthia (α-cbtx), D. polylepis (α-eptx) and B. multicinctus (α-bgtx) using biolayer interferometry (BLI). The parent antibody was discovered from a naïve antibody phage display library against α-cbtx, and had been affinity matured by light chain shuffling using α-cbtx and α-eptx antigens to improve cross-reactivity. 1 In total, 7 affinity matured, light chain shuffled clones were chosen for further characterization based on their binding signals to α-cbtx and α-eptx (Table. 2) and their light chain CDR sequence diversity. Clones were grouped into two light chain germlines, IGVL3-23: 2558_02_G09, 2555_01_A01, 2555_01_A04, 2551_01_B11 and IGLV6-57: 2554_01_D11, 2554_01_E01, 2551_01_A12 and the parent antibody. Clones from the same germline had similar CDR sequences, and all clones had the same CDRL3 and CDRL2 loop length. The LCDR1 loop varied most both in terms of length and sequence diversity between the germlines (FIG. 1A).

The affinities of antibodies to α-cbtx and α-eptx were in agreement with the previously reported SPR values. Values ranged between 33.8-2.9 nM for α-cbtx, and clones displayed slightly higher affinities to α-eptx ranging from 0.89-0.44 nM (Tables. 1 and 2). To assess whether light chain shuffling yielded improvements in cross-reactivity, α-bgtx was included due to not being used in the discovery process and having a low sequence identity to α-cbtx (58%). All clones bound with substantially lower affinity to α-bgtx, with KD values ranging from 3 μM-122 nM (FIG. 1C, Tables 3 and 4). The effect of the light chain on cross-reactivity was most pronounced for the light chain shuffled clones from the IGLV6-57 germline, which exhibited an order of magnitude higher binding affinity to α-bgtx in comparison to both the parent antibody and clones from the IGVL3-23 germline (FIG. 1B). This suggests that affinity-matured clones bearing light chains from the IGLV6-57 germline had improved interactions with conserved regions of long-chain α-neurotoxins, possibly to the conserved finger II region, which is central in their inhibition of the nAChR.

Antibodies classified as being pH-dependent typically have a minimum of 4-fold difference in affinity or dissociation rate between pH 7.4 and PH<6.0.1,2 Based on this, one antibody originating from the IGVL3-23 germline, 2555_01_A01, exhibited pH-dependent binding against all three long-chain α-neurotoxins (FIG. 1C-E). 2555_01_A01 was most pH-sensitive to α-cbtx, having an average of 19-fold difference in dissociation rate between pH 7.4 and pH 5.5, followed by α-bgtx (11.7-fold) and α-eptx (7.90). By contrast, the parent antibody and the remaining affinity matured clones were only moderately pH-sensitive to each of the long-chain alpha neurotoxins, ranging in pH-sensitivity from 1.82-4.94 fold. The least pH-dependent antibodies originated from the IGVL6-57 germline, but were also the more cross-reactive, highlighted most clearly for the broadly neutralizing 2554_01_D11 antibody which had a pH-dependent binding fold-difference of 1.82-3.35 between pH 5.5 and pH 7.4, but had the highest affinity to α-bgtx. The most pH-sensitive antibody, 2555_01_A01, was also able to neutralize long-chain α-neurotoxin in a pre-incubation in vivo experiment (data not shown).

To investigate the basis for the broad cross-reactivity of this lineage of antibodies, and the role of the light chain in facilitating pH-dependent binding, the 2555_01_A01 was chosen for structural characterization by X-ray crystallography.

TABLE 1
Affinity kinetics of antibodies binding to α-cobratoxin

| | KD | ka | kdis | kdis | kdis |
|---|---|---|---|---|---|
| | pH7.4 | pH7.4 | pH7.4 | pH 5.5 | (5.5/ |
| ANTIBODY | (M) | (1/Ms) | (1/s) | (1/s) | 7.4) |
| Parent | 8.96E−08 | 1.83E+04 | 1.64E−02 | N/D | N/D |
| 2551_01_A12 | 4.19E−09 | 9.19E+04 | 3.85E−04 | 7.07E−04 | 1.84 |
| 2554_01_D11 | 2.91E−09 | 7.48E+04 | 2.18E−04 | 3.96E−04 | 1.82 |
| 2554_01_E01 | 7.75E−09 | 3.50E+04 | 2.71E−04 | 9.65E−04 | 3.56 |
| 2555_01_A04 | 3.19E−08 | 1.55E+04 | 4.92E−04 | 2.43E−03 | 4.94 |
| 2555_01_A01 | 3.38E−08 | 1.44E+04 | 4.86E−04 | 6.18E−03 | 12.70 |
| 2551_01_B11 | 6.85E−09 | 6.76E+04 | 4.62E−04 | 1.71E−03 | 3.71 |
| 2558_02_G09 | 3.41E−09 | 1.10E+05 | 3.73E−04 | 1.35E−03 | 3.62 |
| A01-HE | 5.40E−08 | | 2.36E−03 | 2.20E−02 | 9.32 |
| 2555_01_A01 | 1.46E−08 | 4.27E+04 | 6.26E−04 | 1.68E−02 | 26.83 |
| (n = 2) | | | | | |
| 2554_01_D11 | 2.90E−09 | | 1.6E−04 | 3.27E−04 | 2.04 |
| (n = 2) | | | | | |

TABLE 2
Affinity kinetics of antibodies binding to α-elapitoxin

| | KD | Ka | Kdis | Kdis | kdis |
|---|---|---|---|---|---|
| | pH 7.4 | 7.4 | pH 7.4 | ph 5.5 | (5.5/ |
| ANTIBODY | (M) | (1/Ms) | (1/s) | (1/s) | 7.4) |
| 2551_01_A12 | 2.108E−07 | 1.504E04 | 3.171E−03 | 7.333E−03 | 2.31 |
| 2554_01_D11 | 1.22401E−07 | 9.46E+03 | 1.16E−03 | 3.88E−03 | 3.35 |

TABLE 3
Affinity kinetics of antibodies binding to α-bungaratoxin

| | KD | KD | KD |
|---|---|---|---|
| | pH 7.4 | pH 5.5 | (5.5/ |
| ANTIBODY | (M) | (M) | 7.4) |
| Parent | 2.93E−06 | 9.65E−06 | 3.29 |
| 2555_01_A04 | 1.35E−06 | N/D | N/D |
| 2555_01_A01 | 2.99E−06 | 3.50E−05 | 11.69 |
| 2551_01_B11 | 3.585E+09 | N/D | N/D |
| 2558_02_G09 | 2.39E−06 | N/D | N/D |

TABLE 4
Steady state kinetics of antibodies binding to α-bungaratoxin

| | KD | ka | kdis | kdis | kdis |
|---|---|---|---|---|---|
| | pH 7.4 | pH 7.4 | pH 7.4 | pH 5.5 | (5.5/ |
| ANTIBODY | (M) | (1/Ms) | (1/s) | (1/s) | 7.4) |
| Parent | 1.43E−08 | 6.47E+04 | 9.25E−04 | | |
| 2551_01_A12 | 5.58E−10 | 2.15E+05 | 1.20E−04 | 1.91E−04 | 1.59 |
| 2554_01_D11 | 7.83E−10 | 1.81E+05 | 1.42E−04 | 3.37E−04 | 2.37 |
| 2554_01_E01 | 8.90E−10 | 1.75E+05 | 1.56E−04 | 4.18E−04 | 2.68 |
| 2555_01_A04 | 4.38E−10 | 2.64E+05 | 1.15E−04 | 2.48E−04 | 2.15 |
| 2555_01_A01 | 8.94E−10 | 2.35E+05 | 2.10E−04 | 1.66E−03 | 7.90 |
| 2551_01_B11 | | | 1.95E−04 | 8.00E−04 | 4.10 |
| 2558_02_G09 | 6.20E−10 | 2.34E+05 | 1.45E−04 | 4.76E−04 | 3.28 |

### Conclusions

In this example, the inventors have analysed how cross-reactive antibodies against long-chain α-neurotoxins can accommodate both antibody function and pH-dependent binding properties. The inventors report on the neutralization mechanism of a lineage of cross-reactive, light chain-shuffled human monoclonal antibodies discovered through phage display technology, targeting long-chain α-neurotoxins. Insights into the pH-dependent binding mechanism of one neutralizing antibody, 2555_01_A01 are also reported, which displayed both pH-dependent binding and cross-reactivity, thus showcasing that these two therapeutically relevant properties are not mutually exclusive. In addition to developability aspects, the inventors propose another parameter that might influence the ability of these antibodies to neutralize long-chain α-neurotoxins, namely the dissociation rate of the antibody as opposed to the overall affinity. This hypothesis is supported by a comparison of the dissociation rates of the three antibodies to α-cbtx. In contrast to the slow dissociation rate of 2554_01_D11 (2.18×10-4/s) from α-cbtx, both the 2555_01_A01 and 2552_02_B02 antibodies have faster dissociation rates of 4.86×10-4/s, 6.26×10-4/s (2555_01_A01, duplicate measurements) and 5.5×10-4/s (2552_02_B02, single measurement), respectively (Table 1). These faster dissociation rates may allow α-cbtx to be released from the α-cbtx-antibody complex over time and accumulate in the neuromuscular junction, even when the affinity is higher as determined for 2552_02_B02. It is conceivable that once α-cbtx accumulates in the neuromuscular junctions, it will bind strongly to nAChRs, from which it does not dissociate, causing complete neuromuscular blockage, leading to the observed delayed lethality. These observations and this hypothesis highlight the relevance of extending the observation period of monoclonal IgGs during in vivo assessment to discriminate between antibody candidates.

### Example 2: Overall Structure

### Material and Methods

Antibody formats were expressed and prepared as described in Example 1.

### Crystallography: Sample Preparation, Data Collection, and Model Building

Lyophilized long chain α-neurotoxin α-cbtx (Latoxan) was re-suspended in Tris-NaCl (5 mM Tris, 20 mM NaCl, pH 8.0) buffer at a concentration between 5-10 mg/mL. Freshly prepared scFv was added in a 1:3 molar ratio of scFv:α-cbtx and incubated overnight at 4° C. to allow binding. The complex was purified using an NGC Quest™ 10 Plus Chromatography system and a Superdex 75 10/300GL column (Cytiva), which was run at 4° C. using Tris-NaCl (5 mM Tris, 20 mM NaCl, pH 8.0) buffer as the eluent. The scFv-α-cbtx complex was up-concentrated to 14 mg/ml using 3.0 kDa MWCO ultracentrifugation units (Fisher Scientific) prior to plating.

Crystallization was performed using the sitting drop vapor diffusion method at 21° C. Drops were set up either at a 1:1 or 1:2 molar ratio of reservoir to protein in a total volume of 0.3 μL in a 96-well drop format on SWISSCI MRC 2 Well Crystallization Plates (JENA). The wells were sealed with crystal clear tape and equilibrated against 50 μL of reservoir solution at 21° C. Small crystals appeared in the 1:2 condition (0.1 M Bis-Tris, 0.2 M ammonium sulfate, 25% PEG3350, pH 6.5) in under a week. An optimization screen was performed around this condition in 1.2 μL drops and 100 μL reservoir. Crystals developed after two weeks and were harvested using mounted CryoLoops (Hampton Research) with cryoprotection performed by adding glycerol to a neighbour drop with no crystals to a final concentration of 20%. A 300 μm loop was used to fish several crystals. The loop edge was kept in contact with the cryosolution for approx. 5 s to equilibrate before flash freezing the crystal in liquid nitrogen and shipping these to the beamline for remote data collection.

Diffraction data collection was carried out at the P13 beamline (PETRAIII, EMBL, Germany) (PMID: 28009574). The beamline features a 6M PILATUS detector. Data were collected at 100 K for a full sweep of 360° with an oscillation degree of 0.1°, with 0.050 s exposure time, at 12700 eV. The complete data set was processed from 360° (3600 images) with the x-ray beam reduced to 50% intensity.

The structure of 2555_01_A01 bound to α-cbtx was determined by molecular replacement with Phaser-MR (PMID: 17164524) using an AlphaFold2 (PMID: 34265844) model of the expected scFv structure and the toxin PDB ID 4AEA as a search model. Model building and refinement were performed with phenix.refine (PMID: 20124702) and Coot (PMID: 20383002). Data collection and refinement statistics are summarized in Table 1. Molecular graphics were presented with PyMOL Molecular Graphics System (Version 2.2r7pre, Schrödinger, LLC).

### Results

To first understand the structural basis for the broad recognition of long chain α-neurotoxins by 2555_01_A01 and related clones, the inventors determined the crystal structure of the 2555_01_A01 antibody in scFv format bound to α-cbtx at 1.6 Å resolution (FIG. 2A, Table 5). Two scFv molecules and two α-cbtx molecules were present in the asymmetric unit and exhibited P212121 space group symmetry. Each α-cbtx molecule was bound by two scFv molecules, forming an interface with the heavy chain from one scFv and the light chain from the other in a 1:2 (α-cbtx: scFv) stoichiometry. A clear preference for a 1:1 stoichiometry was observed during the preparation of the complex by gel filtration (FIG. 2B), indicating that one of these interfaces was a crystallographic-driven interface. To assess whether either the heavy chain or light chain interfaces were crystallographic-driven interfaces, the Complexation Significance Scores (CSS) for the heavy chain and light chain-α-cbtx interfaces were compared in PISA. The light chain α-cbtx CSS score was 0, which meant that this interface was not significant in forming the complex, and 0.41 for the heavy chain interface. Accordingly, the α-cbtx molecule forming an interface with the heavy chain was treated as the principal molecule involved in the interaction for that scFv, and was chosen to characterize the interaction.

TABLE 5
Data collection and refinement statistics for
2555_01_A01 bound to α-cbtx

| | pH 6.0 | pH 5.5 | pH 4.5 |
|---|---|---|---|
| Wavelength | 0.95 | 0.95 | 0.98 |
| (Å) | | | |
| Resolution | 60.57-1.90 | 41.58-1.60 | 42.52-1.55 |
| range (Å) | (1.96-1.9) | (1.66-1.6) | (1.61-1.55) |
| Space group | P 21 21 21 | P 21 21 21 | P 21 21 21 |
| Unit cell | 76.95 82.65 98.19 | 76.87 83.15 98.34 | 76.86 83.86 98.65 |
| (Å) (*) | 90 90 90 | 90 90 90 | 90 90 90 |
| Total | 99496 (9810) | 419481 (28766) | 486522 (47473) |
| reflections | | | |
| Unique | 49774 (4905) | 83281 (8097) | 92830 (9113) |
| reflections | | | |
| Multiplicity | 2.0 (2.0) | 5.0 (3.6) | 5.2 (5.2) |
| Completeness | 99.25 (99.15) | 99.36 (98.42) | 99.65 (98.86) |
| (%) | | | |
| Mean | 12.62 (2.75) | 10.90 (1.38) | 10.86 (1.20) |
| I/sigma(I) | | | |
| R-work | 0.19 (0.30) | 0.18 (0.38) | 0.20 (0.42) |
| R-free | 0.25 (0.36) | 0.23 (0.40) | 0.22 (0.45) |
| ligands | 64 | 131 | 65 |
| solvent | 502 | 554 | 519 |
| Protein | 634 | 634 | 634 |
| residues | | | |

### Conclusions

The inventors were able to observe paratope-epitope interactions between the 2555_01_A01 scFv and α-cbtx at high resolution, which revealed the recognition of a highly conserved epitope by the antibody CDRH3 loop. At 24 residues long, the CDRH3 was stabilized by an intramolecular disulfide bridge and extended between finger I and II of α-cbtx, recognizing a conformational epitope in the three-finger neurotoxin fold. The complex revealed a tight interface, resulting in a total buried surface area of 952.4 Å2, 864 Å2 contributed by the CDRH3, and 87.8 Å2 by the CDRL3 loop.

### Example 3: Heavy Chain CDR3 Contains Determinants for Cross-Reactivity

### Material and Methods

Antibody formats were expressed and prepared as described in Example 1. Cristallography was performed as described in the previous Example 2.

### Results

Long chain α-neurotoxins induce paralysis by inhibiting nicotinic acetylcholine receptor (nAChR)-acetylcholine interaction. A loop in the nAChR, loop C, is a key responsive element required for neurotransmitter binding and is targeted by R33 and R36 residues on the tip of finger II on long chain α-neurotoxins. Both arginine residues engage aromatic residues on loop C, which coordinate with the acetylcholine molecule. A structural alignment of the Torpedo AChR and bound 2555_01_A01 structures revealed that aromatic Y190nAChR and Y198nAChR residues on loop C were superimposable with Y99HC and Y100eHC on the CDRH3 of 2555_01_A01, engaging R33cbtx and R36cbtx on α-cbtx (FIG. 3A). The CDRH3 loop registers 11 hydrogen bonds and a salt bridge to R33cbtx, R36cbtx, and D26cbtx residues, in addition to forming numerous hydrophobic interactions and hydrogen bonds to the backbone of α-cbtx (FIG. 3B). The hydroxyl group of Y100eHC on CDHR3 forms a hydrogen bond to D26cbtx and is oriented between R36cbtx and R33cbtx residues, forming cation-TT interactions to both R33cbtx and R36cbtx. The guanidinium group of R33cbtx is engaged in further cation-IT interactions with Y99HC, and a salt-bridge to D95HC on CDRH3, forming the core of the interaction. The interaction with R36cbtx is supported by 2 hydrogen bonds from S100cHC residue on CDRH3. As R36 is a valine in α-bgtx, the reduced capacity of α-bgtx to form hydrogen bonds and cation-TT interactions with Y100eHC and S100cHC may explain the lower affinity of all antibodies to α-bgtx.

Facing the C-terminal side of α-cbtx (FIG. 3C) reveals the contribution of the light chain to the interaction. D95aLC on CDRL3 forms a salt bridge to R70cbtx and a hydrogen bond with Y100fHC, buttressing the cation-TT interaction between Y100fHC and R70cbtx. Position 95aLC on CDRL3 was variant in all the affinity matured light chains and may explain the increase in affinity of 2555_01_A01 and related antibodies to α-cbtx compared to the parent antibody, which has glycine at this position. Overall, the CDRH3 loop in 2555_01_A01 and related clones engages long-chain α-neurotoxins through receptor mimicry to achieve cross-reactivity, and as a result, neutralize long-chain α-neurotoxins by inhibiting key residues important for their inhibition of nAChRs. Although these results rationalise the cross-reactivity of 2555_01_A01 and related clones through the CDRH3 interaction, it does not explain the broad pH-dependent binding of 2555_01_A01, as the paratope of 2555_01_A01 is shared with clones that bind pH-independently. Therefore, the pH sensitivity of 2555_01_A01 must lie outside the paratope-epitope interface.

### Conclusions

By determining the bound structure of 2555_01_A01 to α-cbtx, the inventors found that the antibody utilized conserved functional constraints in long-chain α-neurotoxins that are required for their inhibition of the nAChR, by mimicking the conserved interactions that long-chain α-neurotoxins make with the nAChR through receptor mimicry. The inventors thus showed that specificity was primarily determined by the paratope which interacted with the neurotoxin in a manner that mimicked the acetylcholine receptor-neurotoxin interface. This led to broad reactivity against multiple-antigens via binding trough the CDRH3 loop of the antibody. This mechanism of neutralization may be useful in optimizing this and other antibodies for improved cross-reactivity, e.g., through structure-guided engineering to present residues in the antibody paratope to further mimic the nAChR and form interactions with other conserved residues on long-chain α-neurotoxins important for function.

### Example 4: Broad pH-Sensitivity Determinants Located at the Light Chain Interface

### Material and Methods

Antibody formats were expressed and prepared as described in Example 1.

Cristallography was performed as described in the previous Example 2.

### Results

As the light chain was the variable component between each antibody, the inventors investigated the role of the light chain in the observed pH-dependent binding of 2555_01_A01. A crystallography optimization screen enabled to generate crystals and determine structures of 2555_01_A01 bound to α-cbtx at different pH levels (Table 5). The space group and asymmetric unit were the same across all three structures, with no apparent differences in crystal packing, allowing us to identify potential determinants for pH-dependent binding. We initially focused on charged residues in the 2555_01_A01 light chain and identified two charged residues, G95aLCD and S95bLCH, that had been introduced into the CDRL3 loop following light chain shuffling. Analyzing their role in binding, the inventors observed that H95bLC forms a hydrogen bond to S95LC, located near the apex of the β-hairpin loop in CDRL3, which positions D95aLC to interact with R70cbtx on α-cbtx (FIG. 4A). Comparing the conformation of H95bLC in structures determined at each pH showed that H95bLC was pH-responsive, exhibiting a change in rotamer position and hydrogen bond network at pH 4.5 (FIG. 4B), where the H95bLC indole ring switches hydrogen bonds from the S95LC main chain to the D95aLC main chain, destabilizing the interaction between D95aLC and R70cbtx, effectively lowering the affinity to α-cbtx. The effect of H95bLC and D95aLC (which may also be protonated) on the interaction to α-cbtx was tested by the double mutation, D95aLCH and H95bLCE (2555_01_A01-HE), as these were the residues observed in the equivalent positions of the non-pH-dependent 2554_01_D11 antibody. This resulted in a 2-3 fold reduction in pH-dependent binding to α-cbtx (FIG. 4C) and explained the elevated level of pH-dependent binding that 2555_01_A01 has to α-cbtx in comparison to α-bgtx and α-eptx long-chain α-neurotoxins. The α-bgtx and α-eptx long-chain α-neurotoxins feature a P70 as opposed to an R70 seen in α-cbtx, and would not be able to form a salt bridge with D95aLC to lower the affinity of the 2555_01_A01 antibody to these long-chain α-neurotoxins through the CDRL3 loop. However, this did not fully account for the pH-dependent binding of 2555_01_A01, as 2555_01_A01-HE was still 10-fold more pH-sensitive than the non-pH-dependent 2554_01_D11 antibody (FIG. 4C). 2555_01_A01 was also more pH-sensitive than antibodies that had both the D95ALC and H95bLC residues (FIG. 1A). Therefore, H95bLC and D95aLC residues alone do not explain the broad pH-dependent binding behavior of 2555_01_A01.

The 2555_01_A01 antibody contains another histidine residue, H34LC, which is located in the CDRL1 loop and conserved between all antibodies (FIG. 1A).

The inventors speculated that despite being conserved, the surrounding amino acid residue environment of this histidine may be unique to 2555_01_A01 and contribute to its broad pH-dependent binding.

To investigate this, the inventors compared the structures of 2555_01_A01 bound to α-cbtx at pH 5.5 and pH 4.5, which were nearly identical in resolution limits and statistics, to identify any differences in the environment of H34LC. Analysis of the structure of 2555_01_A01 bound to α-cbtx at pH 5.5 revealed that H34LC is solvent-exposed and forms side chain-mediated hydrogen bonds to a water molecule and the main chain of S50LC on CDRL2. H34LC is located at the interface between the heavy chain and light chain, packed beneath Y100IHC in CDRH3, where it forms TT-IT interactions (FIG. 4D). At pH 5.5, Y100IHC occupies a pocket at the interface above H34LC, with the phenol ring of Y100IHC positioned towards the LCDR2 loop and the Y100IHC main chain towards the CDRH3 loop. The Y100IHC main chain forms a hydrogen bond with the side chain carboxyl group of D95HC in CDRH3, stabilizing the interaction between D95HC and R33cbtx on α-cbtx (FIG. 4D). At pH 4.5, the Y100LHC side chain forms a new hydrogen bond to S50LC, and S50LC forms weaker intramolecular and intermolecular hydrogen bonds to itself and H34LC to accommodate the new hydrogen bond to Y100IHC, (FIG. 4E). The error associated with these hydrogen bonds was checked in the web server (http://cluster.physics.iisc.ernet.in/dpi/), which confirmed the hydrogen bond differences between pH 5.5 and pH 4.5 were greater than the associated error, and the lengths had changed between the two pH. Next, compared differences in electron density in these residues were compared by subtracting the X-ray crystal diffraction patterns between the pH 5.5 and pH 4.5 data sets, which is more sensitive to detecting changes in the co-ordinate position of atoms than when using the refined models and removes model bias. This revealed positive electron density above the plane of the phenol ring of Y100IHC, meaning that the phenol ring had moved downwards towards H34LC at pH 4.5 due to a more favourable cation-IT interaction with the positively charged H34LC (FIG. 4F). Positive electron density was also observed along the main chain of Y100IHC and above the side chain carboxyl group of D95HC, indicating that the change in position of Y100IHC had translated through to the interaction between D95HC and R33cbtx. The electron density of S50 had also changed, as expected due to the change in the hydrogen bond network already observed for this residue. Other electron density map differences were mainly observed in cysteine bridges, possibly due to radiation damage during data collection. Collectively, these findings indicate that S50, Y100IHC and D95HC all responded to the change in pH between pH 5.5 and pH 4.5 as a consequence of H34 protonation, which affects the interaction between D95HC and R33cbtx by shifting the Y100IHC residue position and weakening the hydrogen bond Y100IHC makes to stabilize D95HC. Because R33cbtx is highly conserved, this explains why pH-dependent binding is observed to all long-chain α-neurotoxins.

Notably, although all the non-pH-dependent antibodies have D95HC, Y100IHC and H34LC residues, they differ at position S50LC, containing either D, E or H (FIG. 1A). S50LC does not form a hydrogen bond to Y100IHC at pH 5.5 (or pH 6.0, data not shown), but D, E and H residues have hydrogen bonding capability and have a longer side chain than S50LC, which may stabilize Y100LHC at the chain interface at neutral pH by forming a more stable hydrogen bond than S50. Preventing any effects of H34LC protonation on Y100IHC position and binding.

### Conclusions

In contrast to the specificity primarily determined by the paratope, as exemplified in the previous Example, the inventors showed that determinants of pH-dependent binding are located away from the toxin interface. The pH-dependent antibodies are functional, and can neutralize long-chain alpha-neurotoxin in vivo.

Specifically, in relation to the origin of the pH-dependent antigen binding properties observed for 2555_01_A01, the inventors found that the light chain is responsible for equipping the antibody with these properties, independently of the antibody paratope.

The inventors describe a model where the hydrogen bonding network around H34LC, as opposed to the protonation of H34LC itself, allows H34LC to affect binding by modulating the position of residues on the CDRH3 important for the interactions with conserved residues on long-chain α-neurotoxins.

Fully elucidating the basis of the paratope-independent, pH-dependent antigen binding mechanisms would be useful for engineering purposes, which currently rely heavily on the single use of histidine residues. Moreover, in antibodies, many more amino acid residues are present outside the paratope (i.e., in the framework region and any CDRs not interacting with the antigen), and these constitute a larger sequence space for engineering pH-dependent binding properties than that of the paratope. Engineering pH-dependent antigen binding properties outside the antibody paratope are thus be advantageous for some antibody-antigen interfaces, where engineering the paratope might be detrimental to the specificity and function of the antibody.

In this work, the inventors discovered that a paratope-independent mechanism was robust in facilitating pH-dependent binding to a range of long-chain α-neurotoxins, consistently delivering nearly an order of magnitude difference in affinity between pH of pH 5.5 and pH 7.4. Notably, the fold difference in pH-dependent binding allows for the release of long-chain α-neurotoxins within the duration of the antibody recycling pathway.

The inventors propose that the chain interface environment is the stage for this paratope-independent, pH-dependent binding mechanism for the 2555_01_A01 antibody, specifically conferred by the histidine residue in the CDRL1 loop of the antibody. In a similar setting, Kolmar et al. (Ref. 9) identified histidine residues in the CDRL1 and CDRL3 loops that were important in conferring paratope-independent, pH-dependent antigen binding properties to the carcinoembryonic antigen-related cell adhesion 5 molecule. The light chain in their approach was chosen because it was precluded from the binding interaction to enable pairing with two different heavy chains in a bispecific antibody format, without affecting their binding, called “common light chain technology.” The identified histidine mutations that conferred pH-dependent antigen binding properties by the common light chain accumulated in the anchoring position of the CDR1 and CDR3 loops, in comparable sites to the histidine residues located in the 2555_01_A01 light chain. As the light chain employed in the bispecific antibody developed by Kolmar et al. was not involved in the interaction with the antigen, the introduction of pH-dependent antigen binding properties by the common light chain was, like 2555_01_A01, also independent of the paratope. This observation suggests that a similar pH-dependent binding mechanism was introduced to the antibody paratope by both sets of antibody light chains, which was conferred to heavy chains that had very different target specificities.

However, in their study, Kolmar et al. did not elucidate the mechanism of the light chain in conferring pH-dependent binding. In this study, the inventors observed structural changes in residues located at the chain interface, surrounding the histidine residue located in the 2555_01_A01 CDRL1 loop between structures determined at pH 5.5 and pH 4.5. The authors hypothesized that these residues are important in facilitating the pH-dependent antigen binding properties of 2555_01_A01 by affecting the CDRH3 loop structure and paratope.

However, surprisingly, this histidine residue was conserved among all the antibodies in the present work. Without being bound by theory, the inventors believe that the hydrogen bond network in 2555_01_A01 enables this antibody to bind pH-dependently by allowing histidine to affect the antibody CDRH3 loop structure and binding.

The invention provides a paratope-independent approach in conferring pH-dependent antigen binding properties. Antibodies that can release antigens for lysosomal degradation during antibody recycling in vivo have dissociation rates between Oct. 2, 2010-3/s at pH≤6.0 and are characterized by slow dissociation rates at neutral pH, typically ≤10-4/s. It was verified here that 2555_01_A01 was able to release α-cbtx in a human FcRn cellular antibody recycling assay within the duration of the recycling pathway, as no bound 2555_01_A01 antibody was detected following recycling (Data not shown). This was in contrast to the non-pH-dependent 2554_01_D11 antibody, which remained complexed to α-cbtx upon recycling, indicating that 2555_01_A01 may have kinetics amenable to facilitating the antibody-mediated lysosomal degradation of α-cbtx. Although the fold-difference in dissociation rate between pH 7.4 and pH 5.5 was sufficient for 2555_01_A01 to release α-cbtx in this assay, a greater fold difference would be required for antibodies that have slower dissociation rates than 2555_01_A01, and may necessitate the introduction of pH-dependent determinants in the paratope-epitope interface. Nevertheless, the inventors showed that a pH-dependent antigen binding mechanism driven independently of the antibody paratope was accommodated in a cross-neutralizing antibody against long-chain α-neurotoxins. pH-dependent antigen binding via this mechanism did not negatively affect any parameters important for the antibody function and could thus be a useful approach to engineer this property into antibodies without detriment to antibody specificity.

Overall, the structure presented here in the previous Examples outline the mechanism for the broad cross-reactivity and neutralization of antibodies discovered using phage display from a naïve antibody repertoire, which will guide engineering for improved neutralization potency. For instance, the antibody could be engineered to further mimic conserved interactions between nAChR and long-chain α-neurotoxins important for function. Lastly, evidence for an allosteric pH-sensitive binding mechanism is presented for the broad pH-dependent binding of 2555_01_A01, conferred by the antibody light chain, and enabled pH-sensitivity to be coupled with cross-reactivity.

### Example 5: Framework Region Library Design and Validation

### Material and Methods

### Expression of Fab and IgG

Both antibody formats were produced using mammalian expression as described previously (Ref. 10) In brief, the VH and VL domains were subcloned from the scFv-pSANG10-3F vector into a pINT12 vector for Fab expression and pINT3 vector for IgG expression. Individual VH and VL domains were PCR amplified using pSang10_pelB (CGCTGCCCAGCCGGCCATGG, SEQ. ID NO; and 48) HLINK3_R (CTGAACCGCCTCCACCACTCGA, SEQ. ID NO: 49) primers for the VH domain and LLINK2_F (CTCTGGCGGTGGCGCTAGC, SEQ. ID NO: 50) and 2097_R (GATGGTGATGATGATGTGCGGATGCG, SEQ. ID NO: 51) primers for the VL domain. The VH was subsequently cut using Ncol and Xhol endonucleases and the VL with Nhel and Notl. The respective domains were assembled into either the pINT3 or pINT12 vector in a four-component ligation using T4 DNA ligase (Roche, 10481220001). The vector contained the respective heavy chain constant domains for the Fab and IgG in addition to a stuffer region containing the CL and CMV promoter, also cut with Ncol and Notl. The resulting Fab and IgG formats were produced using transient mammalian expression using Expi293™ cells with ExpiFectamine™ 293 (ThermoFisher, A14525) as per the manufacturers guidelines. The cells were harvested and the resulting supernatants containing the IgGs purified using protein A resin (Neo Biotech, NB-45-00036-100) and the Fabs with anti-CH1 resin (Thermo Scientific, 194320010). The purified antibodies were then desalted into PBS and snap frozen for long term storage.

### SPR

The measurement of the antibody affinity at variable pH was performed by immobilizing long neurotoxin to a CM5 dextran chip by amine coupling, and flowing antibody Fab fragments in HEPES-MES (10 mM HEPES 50 mM MES-NaCl 0.05% P20, pH7.4) buffered at either pH 7.4 or pH 5.1 as described previously (Ref. 7). A double background subtraction was performed using a no-protein reference flow cell and a buffer only injection. The affinity was determined as a product of the koff/kon rates using a 1:1 model and a global fit.

### Creation of Phage Display Library by Golden Gate Assembly

The framework library was derived from the 2554_01_D11 scFv sequence, purchased codon optimized from Eurofins in a pEX-K168 vector. A pSANG4 phagemid was sequence verified and used to obtain the phagemid backbone. Individual inserts containing the framework and CDR regions of 2554_01_D11 were PCR amplified using either mutagenic or wild-type oligonucleotides (included to lower the average number of mutations per clone). The phagemid backbone was amplified using pSANG4Myc_Bbsl_For and pSANG4M13_Bbsl_Rev primers. All primers (Table 6) were ordered from TAG Copenhagen, and contained Bpil type-IIS restriction sites for golden gate assembly. The overhang fidelity was checked in the NEB fidelity tool and in the case of mutagenic oligonucleotides, one framework position was diversified per oligo using the NNK codon.

All PCR's were performed using the Q5 Hot Start HF polymerase (NEB, M0494S) in a 2-step PCR program, using the following template unless otherwise stated: Reagents were prepared in a volume of 25 μL, consisting of 0.5 μM of each primer, 1× diluted Q5 High-Fidelity Master Mix, 40 μg of DNA and made up to 25 μL with nuclease-free water (Thermo Scientific, 10977035). DNA was amplified using the following program: Initial denaturation, 98° C., 30 s; amplification for 30 cycles, 98° C., 10 s, and 72° C., 15 s; final extension, 72° C., 5 min. Four PCRs were performed for each insert and cleaned using a Genejet PCR Purification Kit (Thermo Scientific, K0702). For the preparation of the phagemid backbone, twenty PCRs were performed with an extended amplification time of 98° C., 10 s, and 72° C., 110 s, with 1 ng of pSANG4 phagemid used as the template. The pSANG4 backbone amplicon had the DNA template removed by adding 0.5 μL of FastDigest Dpnl (Thermo Scientific, FD1704) to each PCR and was incubated for 15 min at 37° C. Lastly, the phagemid amplicon was gel purified using a GeneJET Gel Extraction Kit (Thermo Scientific, K0692). The purity of all amplicons was confirmed by agarose gel electrophoresis and quantified by Nanodrop (Thermo Scientific, NanodropOne).

Sequential golden gate assembly reactions were used to construct the library. Firstly, individual VH and VL domains were assembled from 2.4 μg of inserts, this equated to 2700 fmoles (roughly 300 ng) of each of the three mutagenic inserts and their competing wild-type counterparts, added at an equal molar ratio to lower the average mutational frequency of each clone. To improve scalability, the amount of enzyme for each assembly was lowered below the manufacturers' guidelines to 0.6 L of Bpil (Fisher Scientific, FD1014), 90 U of T4 DNA Ligase and 2.8 μL of 10× T4 Ligase buffer (NEB, M0202T) per 1000 fmoles of DNA and made up to a final volume of 600 μL with nuclease-free water. The reaction was left overnight at 37° C. in a water bath, without an obvious detriment to the assembly efficiency (FIG. 7). After extraction from a 1.2% agarose gel, 4 ng (more than 100 fold higher than the theoretical diversity) of each assembled domain was divided between four separate PCRs, amplified, and cleaned using the GeneJET PCR Purification Kit (Thermo Scientific, K0702) before assembling the scFv. This was done to scale up each domain whilst maintaining diversity. The scFv was assembled using equal molar ratios of each VH and VL domain using the same enzyme and incubation conditions as described for the VH and VL sub-assemblies, and extracted from a 1.2% agarose gel, yielding 21 ng (2×1010) scFv molecules. Lastly, twenty-one PCRs were performed with the extracted scFv to obtain a sufficient amount of DNA to make a large library. An input of 1 ng extracted scFv and 0.1 μM of Insert_1_For and pSANG4_Myc_M13_Rev primers (FIG. 7) were used in each PCR. The number of cycles was reduced to fifteen to lower the prospect of off-site mutations and the amplicon was cleaned using the GeneJET PCR Purification Kit, yielding 2.6 μg of DNA.

Lastly, golden gate incorporation of library DNA into the phagemid backbone was performed using 4.4 μg of phagemid backbone and 2.2 μg of scFv DNA in a 1:3 molar ratio of phagemid:scFv. To drive an efficient assembly, the amount of enzyme was increased to 1400 U of T4 DNA Ligase, 12.5 μL Ligase buffer, and 2.8 μL Bpil per 1000 fmoles of DNA, made up to a final volume of 800 μL with nuclease-free water and incubated overnight at 37° C. The library was cleaned using the MiniElute PCR Purification Kit (Qiagen, 28006) and eluted using 40 μL of nuclease-free water pre-heated to 60° C. to give a total of 3.2 μg of DNA. In total twenty electroporations were performed using 2 μL of library for each aliquot of electrocompetent TG1 cells (Lucigen, 60000-PQ763-F) as described previously (Ref. 7).

To confirm the presence of inserts and correct assembly, individual transformants were picked for colony PCR screening using phagemid backbone-specific primers (pSANG_5th_For and pSANG_seq_Rev) and submitted for sequencing using the gpll_Rev primer.

Generation of scFv Library and Cloning into pSANG4 Phagemid Vector

Golden gate assembly of the scFv library was performed in two separate assemblies as described in the section Creation of phage display library by Golden Gate assembly. First, by assembling the individual light and heavy chains, and second, by assembling the full-length scFv. Between each assembly step the amount of DNA was scaled-up by PCR in order to maintain diversity and generate a large library. Amplification of the assembled heavy and light chains by PCR gave clear dominant bands of the expected size around 400 bp for the HC and 500 bp for the LC (FIG. 7A). An initial scaling of the subsequently assembled scFv showed some non-specific products, which was reduced when lowering the primer and gel extracted scFv DNA amounts (FIG. 7B).

After scaling up the assembled scFv and electroporating into TG1 cells, colony PCRs were carried out on randomly selected transformants using pSANG4 backbone specific primers to assess the percentage of clones that had an scFv insert. The expected 2 kb amplicon size was present in 56/57 clones (FIG. 8A). To check that the inserts originated from the golden gate assembly, and not from the phagemid template DNA used to generate the phagemid backbone, five colony PCR products were treated with Bpil enzyme. None of the transformants were cut, confirming that the scFv insert was assembled DNA. The template phagemid scFv amplicon containined two Bpil restriction sites, which led to multiple bands after incubation with Bpil (FIG. 8B).

### Results

In order to engineer a conserved pH-dependent determinant into the antibody, the inventors hypothesized that a structural change located at the centre of the antibody variable region, within the light-heavy chain framework interface, could provide a robust means of lowering binding affinity at acidic pH independent of the antigen identity. To identify framework mutations that could confer such pH-sensitive antigen binding properties, the inventors chose a light chain shuffled, anti-long chain α-neurotoxin IgG, 2554_01_D11, previously discovered using phage display selection (Ref. 10). The affinity of 2554_01_D11 to α-cobratoxin was high at both pH 7.4 and pH 5.1, with dissociation constant (KD) values of 2.3 nM and 10.4 nM, respectively (FIGS. 5A and B). The dissociation rate was two orders of magnitude lower than previously reported pH-sensitive clones discovered using in vitro display technologies, (Refs. 3, 9) and were therefore chosen for framework engineering.

For the creation of a binding framework interface library based on the single-chain variable fragment (scFv) format, the inventors chose residues located at homologous positions in the heavy and light chains, as residues in this region have led to structural changes important for neutralization for antibodies generated using in vivo discovery approaches (FIGS. 5C and D). The inventors also took precedence to avoid residues located in the framework interface region that remove light chain pairing, such as those found in single-domain antibodies.

In total, 8 residues were selected for randomization with the NNK codon, 4 heavy chain: Q39, G44, V89, Q105, and 4 light chain: Q38, S43, D85, and G100. The library size was estimated to have a diversity of 4.5×1010 unique clones based on the number of individual transformants and approached the theoretical size of the library. Colony PCR screening showed that 56/57 inserts (98%) were full-length (FIG. 8). Sequencing 27 colonies revealed that 19 (70%) had an open reading frame, were unique, and possessed mutations observed at each position. A parsimonious approach was used to maintain wild-type residues in each clone during the creation of the library using Golden Gate assembly. This was approached by spiking competing wild-type assembly fragments to lower the mutational level of each clone. In accordance with this approach, there were no clones that contained mutations in all 8 positions chosen in the library design, with the number of mutations ranging from 2 and 7 for each clone.

### Conclusions

The inventors thus validated the design and utility of the library created to introduce pH-dependence determinants and to be used as the basis for the identification of pH-dependent antigen-binding scaffolds.

### Example 6: Selection of Framework Mutations Providing Universal pH-Sensitive Antigen Binding Properties to scFvs Using Phage Display

### Material and Methods

Phage Display Phage display selections were performed in solution using a 50 nM concentration of biotinylated α-cobratoxin. Phages were rescued from the library by seeding 200 ml of cells to an OD600=0.1 in 2TY Glucose Ampicillin (100 μg/mL) media (2TYGA), this number of cells equated to the theoretical size of the library. Cells were incubated at 37° C., 280 rpm until OD600=0.5, then a 10-fold excess of proteolytic sensitive helper phage was added for 1 hr at 37° C., 150 rpm to allow for infection. The cultures were spun at 3,200 rpm for 2 minutes, the supernatant was discarded, and the cells were resuspended in 2TYKA media (2TYGA+50 μg/mL Kanamycin). The phages were then propagated overnight at 25° C. and 280 rpm. A TG1 colony was selected from a pre-prepared plate and used to inoculate 5 mL of 2TY media and incubated overnight at 30° C., 280 rpm. The next day, the supernatant was obtained by centrifuging the overnight culture for 10 minutes at 10,500×g and 4° C. Phages were precipitated by adding 1/10 volume of PEG/NaCl (20% PEG 6000/2.5 M NaCl) and incubated on ice for 1 hr before centrifugation at 4,500 rpm for 10 minutes at 4° C. to pellet the phages. The phage pellet was re-suspended in 1 mL of PBS, transferred to an Eppendorf tube, and centrifuged at 14,000 rpm for 10 min at 4° C. to remove any residual cells. The supernatant was transferred to an Eppendorf tube containing 250 μL PEG/NaCl and spun at 14,000 rpm for 10 min at 4° C. The supernatant was discarded, and the phage pellet was re-suspended in 1 mL PBS. The solution was spun down at 14,000 rpm for 10 min at 4° C. until no cell pellet was observed. Phages were immediately used for the phage display selections, for long-term storage, phages were stored in 20% (w/v) glycerol at −80° C.

Two selections were performed in parallel with and without α-cobratoxin using 212 phages. Phages, biotinylated α-cobratoxin (100 nM) and 2×80 μL streptavidin-coated Dynabeads (Fisher Scientific, M-280) were blocked in 3% MPBS (PBS+3% milk: VWR, A0830.100) with end-over-end rotation for 1 hr at room temperature. Equal volumes of blocked α-cobratoxin and phages were mixed and phages were permitted to bind for 1 hr with end-over-end rotation. Blocked streptavidin beads were then added to the phage-α-cobratoxin solution for 5 min with end-over-end rotation to capture phages bound to the biotinylated x-cobratoxin. The captured streptavidin Dynabeads were washed 3 times with PBST (PBS+Tween20) and 3 times with PBS before phages were eluted by adding 100 μL trypsin (1 mg/mL, Sigma-Aldrich, T9201-500 MG) and incubating for 15 min with end-over-end rotation. The phage eluent was used to infect TG1 cells grown to an OD600=0.5 and incubated for 1 hr at 37° C., 150 rpm. Cells were prepared for plating by spinning at 2000 g, for 10 min at room temperature. Dilution plates were prepared ranging from 5 to 500,000-fold dilution of the supernatant on 2TYGA plates to determine the background and enrichment of antigen-specific phages. The following day, colonies on the output plate were scraped and resuspended in 2TYGA media with 25% glycerol, then homogenized for several hours with end-over-end rotation. The OD600 was measured, and the cells were stored at −80° C. for subsequent rescue and the next selection round. The enrichment was determined by dividing the number of colony-forming units on the test plate by the number of colony-forming units from the no-antigen selection.

### Results

To validate the utility of the library, two rounds of phage display selection were performed using 50 nM of α-cobratoxin for both rounds, upon which selected clones were sent for DNA sequencing. The concentration of α-cobratoxin was kept the same for both rounds to minimize selection bias for the parent antibody, which has a K_{D} of 2 nM (FIG. 5A), which could have been favoured if stringent selection conditions (lowering of the concentration of α-cobratoxin) had been employed.

Enrichment of phages was observed between both rounds (FIG. 6). Sequencing 10 clones confirmed that these clones were a full-length variable domain and unique, and that full coverage of each position in the library had been achieved (Table. 7). There were no wild-type sequences, and most clones had 2-3 mutations. Off-site mutations were also observed, and the binding capability of these individual clones has not yet been verified.

TABLE 6
Primers used for golden gate assembly, cloning and sequencing

| Primer name | Sequence Bpil recognition sites are underlined |
|---|---|
| Insert 1_For | Ggctacgaagacaccccagccggccatggctc |
| H2_39Rev | ggctacgaagactaccatccactccaaaccttggcccggtgcknnacgaacccagctaatcg |
| H2_42Rev | ggctacgaagactaccatccactccaaaccknngcccggtgcctgacgaacccagctaatcg |
| H2_43Rev | ggctacgaagactaccatccactccaaknnttggcccggtgcctgacgaacccagctaatcg |
| H2_WTRev | ggctacgaagactaccatccactccaaaccttggcccggtgcctgacgaacccagctaatcg |
| H2_45Rev | ggctacgaagactaccatccactcknnaccttggcccggtgcctgacgaacccagctaatcg |
| H2_For | Ggctacgaagactaatgggtggtattatcccgatttttggtactgctaattatgcgc |
| H3_89Rev | Ggctacgaagactatcacgggcgcagtaataknnagcggtatcatcgctacg |
| H3_WTRev | Ggctacgaagactatcacgggcgcagtaatacacagcggtatcatcgctacg |
| H3_For | Ggctacgaagactagtgataatctgggttattgcagcggcggctcc |
| H4_105Rev | Ggctacgaagactaagacggtgactaaggtaccknnaccccaaacatcc |
| H4_WTRev | Ggctacgaagactaagacggtgactaaggtaccttgaccccaaacatcc |
| H4_For | Ggctacgaagactagtctcgagcggtggtggcggctccgg |
| L2_37Rev | ggctacgaagactagacggtcgtcggtgacgagcccgggcgknnctgataccaatgcac |
| L2_43Rev | Ggctacgaagactagacggtcgtcggknncgagcccgggcgctgctgatacc |
| L2_WTRev | ggctacgaagactagacggtcgtcggtgacgagcccgggcgctgctgataccaatgcac |
| L2_For | Ggctacgaagactacgtcatctatgaggacaaccagcgtccgagcggggtgc |
| L3_85Rev | Ggctacgaagactagctttggcagtagtaknncgcctcgtcctcg |
| L3_WTRev | Ggctacgaagactagctttggcagtagtagtccgcctcgtcctcg |
| L4_100For | Ggctacgaagactaaagctacgaccgctctaaccacgaagttgtttttggcnnkggtacgaag |
| | ctgac |
| pSANG4_Myc_ | Ggctacgaagactacaactttcaacagtttctgcggccccattcagatcctcttc |
| M13_Rev v2 | |
| pSANG4Myc_ | ggctacgaagactagttgtttagcaaaacctcatacagaaaattcatttactaacgtctgg |
| BbsI_For | |
| pSANG4_ | Ggctacgaagaccatgggccgcatagaaaggaacaac |
| Vector_Rev | |
| pSANG5th_For | Tggaaaaacgccagcaacgc |
| -96gIII | ccctcatagtt gcgtaacg |

TABLE 7
Sequences of random clones picked after round 2 of phage display
selection from the framework chain interface library.
VH

| | |
|---|---|
| WT | QVQLVQSGAEVKKPGSSVKVSCKASGGTFSSYAISWVRQAPGQGLEWMG |
| | GIIPIFGTANYAQKFQGRVTITADESTSTAYMELRSLRSDDTAVYYCARDNL |
| | GYCSGGSCYSDYYYYYMDVWGQGTLVTV |
| C1 | HVQLVQSGAEVKKPGSSVKVSCKASGGTFSSYAISWVRQAPGQTLEWMG |
| | GIIPIFGTANYAQKFQGRVTITADESTSTAYMELRSLRSDDTAVYYCARDNL |
| | GYCSGGSCYSDYYYYYMDVWGQGTLVTV |
| C2 | QVQLVQSGAEVKKPGSSVKVSCKASGGTFSSYAISWVRSAPGQGLEWMG |
| | GIIPIFGTANYAQKFQGRVTITADESTSTAYMELRSLRSDDTAVYYCARDNL |
| | GYCSGGSCYSDYYYYYMDVWGVGTLVTV |
| C3 | QVQLVQSGAEVKKPGSSVKVSCKASGGTFSSYAISWVRQAPGQGLEWMG |
| | GIIPIFGTANYAQKFKGRVTITADESTSTAYMELRSLRSDDTADYYCARDNL |
| | GYCSGGSCYSDYYYYYMDVWGQGTLVTV |
| C4 | QVQLVQSGAEVKKPGSSVKVSCKASGGTFSSYAISWVRQAPGQRLEWMG |
| | GIIPIFGTANYAQKFQGRVTITADESTSTAYMELRSLRSDDTAQYYCARDNL |
| | GYCSGGSCYSDYYYYYMDVWGQGTLVTV |
| C5 | QVQLVQSGAEVKKPGSSVKVSCKASGGTFSSYAISWVRRAPGQGLEWMG |
| | GIIPIFGTANYAQKFQGRVTITADESTSTAYMELRSLRSDDTAQYYCARDNL |
| | GYCSGGSCYSDYYYYYMDVWGQGTLVTV |
| C6 | QVQLVQSGAEVKKPGSSVKVSCKASGGTFSSYAISWVRQAPGQNLEWMG |
| | GIIPIFGTANYAQKFQGRVTITADESTSTAYMELRSLRSDDTAVYYCARDNL |
| | GYCSGGSCYSDYYYYYMDVWGQGTLVTV |
| C7 | QVQLVQSGAEVKKPGSSVKVSCKASGGTFSSYAISWVRTAPGQGLEWMG |
| | GIIPIFGTANYAQKFQGRVTITADESTSTAYMELRSLRSDDTATYYCARDNL |
| | GYCSGGSCYSDYYYYYMDVWGQGTLVTV |
| C8 | QVQLVQSGAEVKKPGSSVKVSCKASGGTFSSYAISWVRQAPGQYLEWMG |
| | GIIPIFGTANYAQKFQGRVTITADESTSTAYMELRSLRSDDTAVYYCARDNL |
| | GYCSGGSCYSDYYYYYMDVWGQGTLVTV |
| C9 | QFQLVQSGAEVKKPGSSVKVSCKASGGTFSSYAISWVRQAPGQGLEWMG |
| | GIIPIFGTANYAQKFQGRVTITADESTSTAYMELRSLRSDDTATYYCARDNL |
| | GYCSGGSCYSDYYYYYMDVWGQGTLVTV |
| C10 | QVQLVQSGAEVKKPGSSVKVSCKASGGTFSSYAISWVREAPGQGLEWMG |
| | GIIPIFGTANYAQKFQGRVTITADESTSTAYMELRSLRSDDTAIYYCARDN |
| | LGYCSGGSCYSDYYYYYMDVWGQGTLVTV |

VL

| | |
|---|---|
| WT | NFMLTQPRSVSESPGKTVTISCTRSSGSIGSDYVHWYQQRPGSSPTTVIYE |
| | DNQRPSGVPDRFSGSIDSSSNSASLTISGLKTEDEADYYCQSYDRSNHEVV |
| | FGGGTKLTVL |
| C1 | NFMLTQPRSVSESPGKTVTISCTRSSGSIGSDYVHWYQQRPGSSPTTVIYE |
| | DNQRPSGVPDRFSGSIDSSSNSASLTISGLKTEDEADYYCQSYDRSNHEVV |
| | FGGGTKLTVL |
| C2 | NFMLTQPRSVSESPGKTVTISCTRSSGSIGSDYVHWYQQRPGSSPTTVIYE |
| | DNQRPSGVPDRFSGSIDSSSNSASLTISGLKTEDEADYYCQSYDRSNHEVV |
| | FGGGTKLTVL |
| C3 | NFMLTQPRSVSESPGKTVTISCTRSSGSIGSDYVHWYQQRPGSRPTTVIYE |
| | DNQRPSGVPDRFSGSIDSSSNSASLTISGLKTEDEADYYCQSYDRSNHEVV |
| | FGGGTKLTVL |
| C4 | NFMLTQPRSVSESPGKTVTISCTRSSGSIGSDYVHWYQQRPGSQPTTVIYE |
| | DNQRPSGVPDRFSGSIDSSSNSASLTISGLKTEDEATYYCQSYDRSNHEVV |
| | FGVGTKLTVL |
| C5 | NFMLTQPRSVSESPGKTVTISCTRSSGSIGSDYVHWYQQRPGSKPTTVIYE |
| | DNQRPSGVPDRFSGSIDSSSNSASLTISGLKTEDEADYYCQSYDRSNHEVV |
| | FGGGTKLTVL |
| C6 | NFMLTQPRSVSESPGKTVTISCTRSSGSIGSDYVHWYQQRPGSSPTTVIYE |
| | DNQRPSGVPDRFSGSIDSSSNSASLTISGLKTEDEADYYCQSYDRSNHEVV |
| | FGWGTKLTVL |
| C7 | NFMLTQPRSVSESPGKTVTISCTRSSGSIGSDYVHWYQFRPGSSPTTVIYE |
| | DNQRPSGVPDRFSGSIDSSSNSASLTISGLKTEDEADYYCQSYDRSNHEVV |
| | FGGGTKLTVL |
| C8 | NFMLTQPRSVSESPGKTVTISCTRSSGSIGSDYVHWYQQRPGSSPTTVIYE |
| | DNQRPSGVPDRFSGSIDSSSNSASLTISGLKTEDEAAYYCQSYDRSNHEVV |
| | FGGGTKLTVL |
| C9 | NFMLTQPRSVSESPGKTVTISCTRSSGSIGSDYVHWYQQRPGSSPTTVIYE |
| | DNQRPSGVPDRFSGSIDSSSNSASLTISGLKTEDEADYYCQSYDRSNHEVV |
| | FGGGTKLTVL |
| C10 | NFMLTQPRSVSESPGKTVTISCTRYSGSIGSDYVHWYQVRPGSSPTTVIYE |
| | DNQRPSGVPDRFSGSIDSSSNSASLTISGLKTEDEAHYYCQSYDRSNHEVV |
| | FGHGTKLTVL |

VH: Variable heavy chain, VL: Variable light chain, Bold sites in WT sequences: Sites chosen for library creation, Bold sites in C-sequences: Observed mutations

### Conclusions

In this work, a framework chain-interface library from a non-pH-dependent antibody was generated to serve as basis for the discovery of antigen-binding proteins with the rationale that pH-dependent binding conferred by the antibody framework itself, specifically mediated by amino acid residues located in the light-heavy chain interface, can provide a universal method for introducing pH-dependent antigen binding properties into antibodies.

### Example 7: Discovery of pH-Dependent Antigen-Binding Proteins from the Chain-Interface Library

### Material and Methods

### ScFv Selection

From the phage display selection output of Example 6, 92 individual scFv-expressing monoclonal colonies were picked randomly for testing in DELFIA immunoassays. To express soluble scFvs, the scFv genes from the third selection round were sub-cloned from the phage display vector (plONTAS1) into the pSANG10-3F expression vector using Ncol and Notl restriction endonucleases. The expression vectors were then transformed into E. coli BL21 (DE3) cells (New England Biolabs), following protocols from Martin et al. (Ref. 13). Individual scFv-producing monoclonal colonies (276 colonies) were picked and expressed in 96-well format using autoinduction media.

### DELFIA Immunoassay

DELFIA immunoassay was performed as described herein in Example 8.

### Sequence Alignment

Sequences were aligned and the mutated residues in the frame-work mutated libraries were highlighted.

### Results

2554_01_D11, the antibody used as parent for the generation of the chain-interface phage display library was confirmed to display no pH sensitivity.

scFVs with as similar affinity to α-cobratoxin as the parent were identified, further showing up to the range of 2-2.5 fold higher pH sensitivity by DELFIA immunoassay as described herein (FIG. 9, Table 8). Alignments of the variable heavy and light chains of the scFv clones picked displaying pH sensitivity above 1.25, as measured by the ratio of DELFIA signal at neutral pH 7.4 over the DELFIA signal at acidic pH 5.8, showed specific mutations in the mutated residues of the frame-work in variable heavy chains and variable light chains (FIG. 10 and FIG. 11).

TABLE 8
DELFIA measurements of the clones showing pH sensitivity
(pH 7.4 DELFIA/pH 5.8 DELFIA) above 1.25.

| | | | pH |
|---|---|---|---|
| | | | sensitivity |
| Clones | pH 7.4 | pH 5.8 | (7.4/5.8) |
| Post CTR | 67836 | 6437 | 10.5 |
| (scFv-A01) | | | |
| A08 | 226635 | 101180 | 2.2 |
| B03 | 365354 | 193391 | 1.9 |
| F07 | 225550 | 123744 | 1.8 |
| C04 | 238424 | 140342 | 1.7 |
| B10 | 303535 | 181341 | 1.7 |
| H03 | 221566 | 135433 | 1.6 |
| F05 | 359214 | 237633 | 1.5 |
| B04 | 538642 | 359351 | 1.5 |
| E05 | 286747 | 193283 | 1.5 |
| B05 | 369181 | 254874 | 1.4 |
| C07 | 457029 | 319667 | 1.4 |
| D01 | 318495 | 223150 | 1.4 |
| C09 | 470384 | 330595 | 1.4 |
| D08 | 337409 | 241264 | 1.4 |
| D05 | 265256 | 191683 | 1.4 |
| D12 | 313506 | 227956 | 1.4 |
| G05 | 338958 | 251327 | 1.3 |
| E08 | 519175 | 389566 | 1.3 |
| H02 | 372469 | 281761 | 1.3 |
| F03 | 351377 | 266831 | 1.3 |
| F09 | 310333 | 239180 | 1.3 |
| B02 | 488599 | 377507 | 1.3 |
| G02 | 346177 | 268194 | 1.3 |

The clones are as in the alignments of FIGS. 10 and 11 (e.g. clone F09_F09_R of FIGS. 10 and 11 is clone “F09” in Table 8 above), and are also comprised in the 92 clones plotted on FIG. 9. pH sensitivity is calculated as described herein based on DELFIA values measured at pH 7.4 and pH 5.8. Post CTR: positive control.

### Conclusions

The chain-interface library designed by introducing the mutations as described herein successfully enabled to identify pH dependent antigen binding proteins with similar affinity as the parent antigen binding protein used to generate the library, and having 2-2.5 fold higher pH sensitivity.

### Example 8: Identification of Additional Residues Involved in pH-Dependence of Antigen-Binding Proteins by Point Mutations

### Material and Methods

### Point Mutation Approach

For point mutations the in vivo assembly (IVA) cloning technique was used, with unique primers to introduce the individual mutations as described in Ref. 12.

DELFIA assay

### Day 1

2YTGK medium was inoculated with glycerol stocks and grown overnight (O/N) at 800 revolutions per minute (rpm) at 30 degrees Celsius.

### Day 2

Autoinduction media was inoculated with O/N cultures and grow/express O/N at 800 rpm at 30 degrees Celsius

Black Maxisorp plates were coated with 2.5 μg/mL anti-FLAG M2 antibody (Sigma) O/N at 4 degrees Celsius

### Day 3

Plates were washed 3 times with Phosphate-Buffered Saline (PBS). Wells were blocked in 200 uL 3% (w/v) skimmed milk in PBS (MPBS) 1 hour with shaking. scFv cultures were centrifuged at 3000 g for 10-20 minutes. Plates were washed with PBS. Then 25 μL of 6% MPBS was added to each well, followed by 25 L of supernatant to each well.

Plates were incubated at room temperature for 1 hour and the plates were washed 3 times with PBS pH 7.4, and 3 times with PBST pH 7.4.

50 μL of 50 nM of biotinylated α-cobratoxin prepared in 3% MPBS pH 7.4 was then added and left to incubate for 1 hour.

The plates were washed three times with 200 μL PBS and 3 times with 200 μL PBST at either pH 7.4 or pH 5.8 to prime those wells for antigen release in the next step. 200 μL of 3% MPBS pH 7.4 or pH 5.8 was added for 1 hour to dissociate the α-cobratoxin followed by wash with PBS at either pH 5.8 or pH 7.4 for the different conditions, then all the wells were washed with 200 μL PBST pH 7.4.

50 μL of a 1/500 dilution of Streptavidin conjugated Europium in DELFIA assay buffer was added to each well (=10 ng of Streptavidin-Europium/well) and incubated for 30 minutes at room temperature.

DELFIA enhancement solution was aliquoted for detection of all the plates, and plates left in the dark to reach room temperature.

Plates were washed 3 times with PBS+0.1% Tween and 3 times with PBS in the plate washer. 50 μL of Enhancement solution was then added. Plates were then incubated for 15 minutes with shaking and the top plate covered.

Switch on Victor Nivo Plate Reader at least 30 min before to let the lamp warm up. The plates were read on Victor Nivo Plate Reader set at 25 degrees Celsius and equipped with the right filters for DELFIA (excitation 320-340 nm, emission 615 nm).

### Results

Measurements by DELFIA immunoassays point mutations providing in the range of 1.25 and up to 6 fold increased pH sensitivity were identified.

### Conclusions

By introducing point mutations to the parent antigen-binding protein used to generate the chain-interface library, (2554_01_D11), further residue positions conferring pH-dependence to antigen-binding proteins were identified, as measured by DELFIA assay.

### Example 9: Identification of Additional Residues Involved in pH-Dependence of Antigen-Binding Proteins by TheraSAb-Dab Analysis

### Material and Methods

### Therapeutic Antibody Alignment

Antibody sequences were downloaded from the Therapeutic Structural Antibody Database (Thera-SAbDab) (https://opig.stats.ox.ac.uk/webapps/sabdab-sabpred/therasabdab/search/(accessed 23rd of October 2023)). From the Thera-SAbDab, all sequences were downloaded using the “get all therapeutics” button. From the downloaded sequences, only sequences with the “Genetically Human” trait in the “Genetics (Bispecifics delimited with semicolon)” were kept for analysis. The heavy chains of the resulting 328 antibody sequences were aligned using Jalview (version 2.11.2.7) with the “muscle with defaults” alignment option. The light chains of the 328 antibody sequences were divided into kappa and lambda and aligned using the same parameters described above.

### Interface Analysis:

human antibody structures (8AHN, 8BSF, 8DCC, 7U8E, 3NPS) were picked by random from the SAb-Dab website (https://opig.stats.ox.ac.uk/webapps/sabdab-sabpred/sabdab/search/?all=true) and downloaded. Heavy-light chain interfaces were denoted using the ChimeraX-function “interface” followed by clicking the network line between the heavy and light chain and selecting the “Select contact residues of X and X”. The interface command calculates the buried solvent-accessible surface area (SASA) for each pair of chains in the specified set of atoms, based on chain IDs, and generates a network diagram of the interchain interfaces. The function was used with the default parameters as used by ChimeraX (https://www.rbvi.ucsf.edu/chimerax/docs/user/commands/interfaces.html).

### Results

The 5 sequences of the antibody structures were aligned using Jalview as described above and the residue positions which were shown to interact with the opposing chain in all 5 structures were transferred to the Thera-SAbDab alignment to generate FIG. 13.

### Conclusions

By comparing interaction points of heavy and light chain interfaces denoted using the Chimera-x function “interface” with the conservation of amino acids (determined by alignment) of 328 therapeutic antibodies, several additional residue positions were selected, which were found to carry potential for introducing pH-sensitivity.

### Example 10: Site-directed mutagenesis of Bevacizumab, Adalimumab and 2554_01_D11

### Material and Methods

Site-directed mutagenesis was carried out using wildtype Bevacizumab and Adalimumab, the sequences for which was found using https://opig.stats.ox.ac.uk/webapps/sabdab-sabpred/therasabdab/search/. The amino acid sequence for heavy and light chain was found which was back translated and linked together using a flexible linker peptide (Gly4 Ser Gly4 Ser Gly3 Ala Ser) to generate scFv versions of the antibodies. The resulting DNA sequences were ordered from: https://www.twistbioscience.com. Additionally, the wildtype gene for 2554_01_D11 was PCR amplified using GTAACCACCACACCCGCCGCGCTTAATG (SEQ. ID NO: 221) and CACCATACCCACGCCGAAACAAGCGC (SEQ. ID NO: 222) prior to site-directed mutagenesis.

Using the sequences as template, site-directed mutagenesis was carried out using primers carrying NNK/NNM motifs (Table 11) with the following materials (Table 9):

TABLE 9

| Material | Amount (μL) |
|---|---|
| Ultrapure H₂O (10977035) | To 50 μL |
| 5x Phusion HF buffer (B0518SVIAL) | 10 |
| 10 mM dNTP ( ) | 1 |
| 10 μM forward primer | 2.5 |
| 10 μM reverse primer | 2.5 |
| 10 ng template DNA | Concentration |
| | dependent |
| Phusion HF DNA polymerase | 0.5 |
| (M0530SVIAL) | |

The below PCR setup (Table 10) was used:

TABLE 10

| | | |
|---|---|---|
| Temper- | | |
| ature ° C. | Time | Cycles |

| | | | |
|---|---|---|---|
| 98 | 30 | sec | 1 |
| 98 | 10 | sec | 30 |
| 53 | 20 | sec | |
| 72 | 30 | sec | |
| 72 | 5 | min | 1 |

Following PCR mutagenesis and purification using GeneJET PCR purification kit (K0701), the fragments were combined and digested with Eco31I (FD0294) for 30 minutes at 37° C. The digestion was carried out in 100 μL volume containing 35 μL Ultrapure H₂O, 10 μL FastDigest buffer, 50 μL DNA (6 μg), and 5 μL FastDigest enzyme (Eco31I). Following restriction, the restricted overlapping DNA sequences were purified and ligated in 80 μL volume using T4-ligase (4 μL), 10× ligation buffer (8 μL), 900 μg DNA fragments (10 μL) and Ultrapure H₂O to 80 μL. Next, a PCR was carried out using the above materials and temperature, but only with the outer primers to ensure only full length scFv sequences were amplified. These full-length scFvs were digested using 1 μL of FastDigest enzymes Ncol and Notl, 10 UL PCR solution, 2 μL 10× FD buffer and 16 μL Ultrapure H2O. Additionally, the 1 μg pSANG10-3F expression vector was digested using the same setup.

The digested vector (50 ng) and scFv genes (111 ng) were purified using GeneJET PCR purification kit (K0701) and ligated using 50 ng vector DNA, 111 ng scFv genes (1:15 vector: insert ratio), 1 μL T4 ligase, 2 μL 10× T4 ligase buffer, and Ultrapure H₂O to 20 μL. The ligation was carried out at 37° C. for 30-60 minutes.

The ligated vector+scFv was transformed into BL21 (DE3) competent E. coli (NEB, C2527H) by first thawing the cells on ice for 10 minutes, then mixing the 50 μL cells with 5 μL ligation product and incubating on ice for 30 minutes. Next, the cells were heat shocked in a water bath at 42° C. for 10 seconds, incubated on ice for 5 minutes, and transferred to 950 μL SOC medium. The cells were then incubated at 37° C. with 250 rpm shaking for 1 hour, after which 100 μL cells were plated out on 2YTGK plates (2YT+2% glucose+50 μg/mL kanamycin). The remaining 900 μL cell culture were centrifuged at 2400×g, resuspended in 100-200 μL volume and plate onto a second 2TYGK plate. The plates were incubated overnight at 37° C.

The following day the plates were picked into 96-well polypropylene plates ( ) containing 150 μL 2TYGK media and grown overnight, after which 50 μL of 50% glycerol was added and the plates were stored at −80° C.

TABLE 11
Mutagenesis primers

| Primer name | Primer sequence |
|---|---|
| Ada_1_Forward | GGTCTGCTGCTCCTCGCT |
| Ada_1_Reverse | TATGGTCTCACTAACCCATTCCAAKNNTTTACCTG |
| Ada_2_Forward | TATGGTCTCATTAGTGCAATTACCTGGAACTCTGG |
| Ada_2_Reverse | TATGGTCTCACCTTGGCACAGTAGTAKNNAGC |
| Ada_3_Forward | TATGGTCTCAAAGGTTTCTTACTTGTCCACCGC |
| Ada_3_Reverse | TATGGTCTCACAGTGACCAAAGTACCKNNACCC |
| Ada_4_Forward | TATGGTCTCAACTGTCAGCAGTGGTGGTGGTGGTAGC |
| Ada_4_Reverse | TATGGTCTCATGGAGCCTTACCCGGTTTKNNTTG |
| Ada_5_Forward | TATGGTCTCATCCAAAGCTGCTGATTTACGCT |
| Ada_5_Reverse | GTGATGGTGATGATGATGTGCGG |
| Bev_1_Forward | GGTCTGCTGCTCCTCGCT |
| Bev_1_Reverse | TATGGTCTCAGACCCATTCCAAKNNCTTACCCG |
| Bev_2_Forward | TATGGTCTCAGGTCGGTTGGATTAATACCTACACCG |
| Bev_2_Reverse | TATGGTCTCAACTTAGCACAATAGTAKNNAGCAGTGT |
| Bev_3_Forward | TATGGTCTCAAAGTACCCACACTATTACGGTTCC |
| Bev_3_Reverse | TATGGTCTCACCAAAGTACCKNNACCCCAA |
| Bev_4_Forward | TATGGTCTCATTGGTTACTGTGAGTTCCGGT |
| Bev_4_Reverse | TATGGTCTCACTTTACCTGGCTTKNNTTGGTACCA |
| Bev_5_Forward | TATGGTCTCAAAAGCCCCGAAAGTTTTGATTTATTTCAC |
| Bev_5_Reverse | TGATGGTGATGATGATGTGCGG |
| Fragment 1 | TATGGTCTCAGGGAGGGATCATCCCTATCTTTG |
| (1).FOR | |
| Fragment 1 | TATGGTCTCACTCTCGCACAMNNATACACGG |
| (1).REV | |
| Fragment 2 | TATGGTCTCAAGAGACAACCTAGGATATTGTAGTGGTG |
| (1).FOR | |
| Fragment 2 | TATGGTCTCACGCTGCCAATGCTGCCACTGCTGCG |
| (1).REV | |
| Fragment 3 | TATGGTCTAGCGACTATGTGCATTGGNNKCAGC |
| (1).FOR | |
| Fragment 3 | TATGGTCTCAGATGACMNNGGTMNNGGAGC |
| (1).REV | |
| Fragment 4 | TATGGTCTCACATCNNKGAGGATAACCAAAGACC |
| (1).FOR | |
| Fragment 4 | TATGGTCTCACTGCGATCATAAGACTGACAMNNGTAGTC |
| (1).REV | |
| Fragment 5 | TATGGTCTCAGCAGCAATCATGAAGTGGTGNNKGG |
| (1).FOR | |
| Fragment 5 | TGCGGATGCGGCCGCGGGCTGA |
| (1).REV | |
| Vector (1). FOR | CACCATACCCACGCCGAAACAAGCGC |
| Vector (1).REV | TATGGTCTCATCCCATMNNCTCMNNCCCT |

Sequencing of scFv Genes

Sequencing was carried out using the eurofins Mix2Seq service with s10b primer (GGCTTTGTTAGCAGCCGGATCTCA) (SEQ. ID NO: 253) and analyzed using snapgene.

### Results

Site-directed mutagenesis of four residue positions found to induce pH-dependence in 2554_01_D11 (FIG. 15.B) induced pH-dependence in other (blockbuster) mAbs Bevacizumab and Adalimumab (FIG. 16). The top 5 mutants showing the highest pH-dependence were sequenced (FIG. 17).

### Conclusion

Frame mutations that provide 2554_01_D11 with pH-dependent antigen binding properties are conserved in other (blockbuster) mAbs.

Sequence overview

SEQ. ID NO: 1
Heavy chain CDR1 of antibody 2555_01_A01
GGTFSSYA
SEQ. ID NO: 2
Heavy chain CDR2 of antibody 2555_01_A01
IIPIFGTA
SEQ. ID NO: 3
Heavy chain CDR3 of antibody 2555_01_A01
DNLGYCSGGSCYSDYYYYYMDV
SEQ. ID NO: 4
Light chain CDR1 of antibody 2555_01_A01
NIGQQI
SEQ. ID NO: 5
Light chain CDR2 of antibody 2555_01_A01
SDS
SEQ. ID NO: 6
Light chain CDR3 of antibody 2555_01_A01
QVWDSGSDHVV
SEQ. ID NO: 7
Light chain CDR1 of Parent antibody
TRSSGSIASTYVH
SEQ. ID NO: 8
Light chain CDR2 of Parent, 2554_01_D11, 2551_01_A12 antibodies
EDNQRPS
SEQ. ID NO: 9
Light chain CDR3 of Parent antibody
QSYDSSNGSVV
SEQ. ID NO: 10
Light chain CDR1 of 2554_01_D11 and 2554_01_E01 antibodies
TRSSGSIGSDYVH
SEQ. ID NO: 11
Light chain CDR3 of 2554_01_D11 antibody
QSYDRSNHEVV
SEQ. ID NO: 12
Light chain CDR2 of 2554_01_E01 antibody
EDNRRPS
SEQ. ID NO: 13
Light chain CDR3 of 2554_01_E01 antibody
QSYDSTTNHVV
SEQ. ID NO: 14
Light chain CDR1 of 2551_01_A12 antibody
TRSSGRIVSDYVH
SEQ. ID NO: 15
Light chain CDR3 of 2551_01_A12 antibody
QSYDSSNAYVV
SEQ. ID NO: 16
Light chain CDR1 of 2558_02_G09, 2555_01_A01 antibodies
EGDNIGQQIVH
SEQ. ID NO: 17
Light chain CDR2 of 2558_02_G09 antibody
DGSRRPS
SEQ. ID NO: 18
Light chain CDR3 of 2558_02_G09 antibody
QVWDITSDHVV
SEQ. ID NO: 19
Light chain CDR2 of 2555_01_A01 antibody
SDSDRPS
SEQ. ID NO: 20
Light chain CDR1 of 2555_01_A04 antibody
GGDYIGGESVH
SEQ. ID NO: 21
Light chain CDR2 of 2555_01_A04 antibody
DDTHRPS
SEQ. ID NO: 22
Light chain CDR3 of 2555_01_A04 antibody
QVWDVSSDHVV
SEQ. ID NO: 23
Light chain CDR1 of 2551_01_B11 antibody
GGHNIGSNIVH
SEQ. ID NO: 24
Light chain CDR2 of 2551_01_B11 antibody
HNTNRPS
SEQ. ID NO: 25
Light chain CDR3 of 2551_01_B11 antibody
QVWDSSSEHVV
SEQ. ID NO: 26
Parental VL FWR1 of the library
NFMLTQPRSVSESPGKTVTISC
SEQ. ID NO: 27
Parental VL FWR2 of the library
WYQQRPGSSPTTVIY
SEQ. ID NO: 28
Parental VL FWR3 of the library
GVPDRFSGSIDSSSNSASLTISGLKTEDEADYYC
SEQ. ID NO: 29
Parental VL FWR4 of the library
FGGGTKLTVL
SEQ. ID NO: 30
Parental VH FWR 1 of the library
QVQLVQSGAEVKKPGSSVKVSCKASGGTFS
SEQ. ID NO: 31
Parental VH FWR2 of the library
WRQAPGQGLEWMG
SEQ. ID NO: 32
Parental VH FWR3 of the library
RVTITADESTSTAYMELRSLRSDDTAVYYCAR
SEQ. ID NO: 33
Parental VH FWR4 of the library, Heavy chain FWR4 of 2555_01_A01 antibody,
Adalimumab, Bevacizumab and Fasinumab
WGQGTLVTVSS
SEQ. ID NO: 34
Library VH primary sequence
QVQLVQSGAEVKKPGSSVKVSCKASGGTFSSYAISWVRQAPGQGLEWMGGIIPIFGT
ANYAQKFQGRVTITADESTSTAYMELRSLRSDDTAVYYCARDNLGYCSGGSCYSDYY
YYYMDVWGQGTLVTVSS
SEQ. ID NO: 35
Library VL primary sequence and VL of WT, and C1, C2 and C9 clones picked after
round 2 of phage display selection from the framework chain interface library.
NFMLTQPRSVSESPGKTVTISCTRSSGSIGSDYVHWYQQRPGSSPTTVIYEDNQRPS
GVPDRFSGSIDSSSNSASLTISGLKTEDEADYYCQSYDRSNHEVVFGGGTKLTVL
SEQ. ID NO: 36
Library VH polynucleotide sequence
CAGGTGCAGCTGGTGCAATCTGGGGCTGAGGTGAAGAAGCCTGGGTCCTCGGTG
AAGGTCTCCTGCAAGGCTTCTGGAGGCACCTTCAGCAGCTATGCTATCAGCTGGG
TGCGACAGGCCCCTGGACAAGGGCTTGAGTGGATGGGAGGGATCATCCCTATCT
TTGGTACAGCAAACTACGCACAGAAGTTCCAGGGCAGAGTCACGATTACCGCGGA
CGAATCCACGAGCACAGCCTACATGGAGCTGAGGAGCCTGAGATCTGACGACAC
GGCCGTGTATTACTGTGCGAGAGACAACCTAGGATATTGTAGTGGTGGTAGCTGC
TACTCTGACTACTACTACTACTACATGGACGTCTGGGGCCAGGGCACCCTGGTCA
CCGTCTCGAGT
SEQ. ID NO: 37
Library VL polynucleotide sequence
AATTTTATGCTGACTCAGCCCCGCTCTGTGTCGGAGTCTCCGGGGAAGACGGTAA
CCATCTCCTGCACCCGCAGCAGTGGCAGCATTGGCAGCGACTATGTGCATTGGTA
CCAGCAGCGCCCGGGCAGCTCCCCCACCACTGTCATCTATGAGGATAACCAAAG
ACCCTCTGGGGTCCCTGATCGGTTCTCTGGCTCCATCGACAGCTCCTCCAACTCT
GCCTCCCTCACCATCTCTGGACTGAAGACTGAGGACGAGGCTGACTACTACTGTC
AGTCTTATGATCGCAGCAATCATGAAGTGGTGTTCGGCGGAGGGACCAAGCTGAC
CGTCCTA
SEQ. ID NO: 38
Heavy chain FWR1 of 2555_01_A01 antibody
QVQLVQSGAEVKKPGSSVKVSCKAS
SEQ ID NO: 39
Heavy chain FWR2 of 2555_01_A01 antibody
ISWVRQAPGQGLEWMGG
SEQ. ID NO: 40
Heavy chain FWR3 of 2555_01_A01 antibody
NYAQKFQGRVTITADESTSTAYMELRSLRSDDTAVYYCAR
SEQ. ID NO: 41
Light chain FWR1 of 2555_01_A01 antibody
SYELTQPPSVSVAPGRTATITC
SEQ. ID NO: 42
Alternative light chain FWR1 of 2555_01_A01 antibody
SYELTQPPSVSVAPGRTATITCEGD
SEQ. ID NO: 43
Light chain FWR2 of 2555_01_A01 antibody
WYQQKPGQAPVAVIS
SEQ. ID NO: 44
Alternative light chain FWR2 of 2555_01_A01 antibody
VHWYQQKPGQAPVAVIS
SEQ. ID NO: 45
Light chain FWR3 of 2555_01_A01 antibody
GIPERFSGSNSGNTATLTISRVEAGDEADYYC
SEQ. ID NO: 46
Alternative light chain FWR3 of 2555_01_A01 antibody
DRPSGIPERFSGSNSGNTATLTISRVEAGDEADYYC
SEQ. ID NO: 47
Light chain FWR4 of 2555_01_A01 antibody
FGGGTKVTVL
SEQ. ID NO: 48
pSang10_pelB primer used for PCR amplification of VH domain
CGCTGCCCAGCCGGCCATGG
SEQ. ID NO: 49
HLINK3_R primer used for PCR amplification of VH domain
CTGAACCGCCTCCACCACTCGA
SEQ. ID NO: 50
LLINK2_F primer used for PCR amplification of VL domain
CTCTGGCGGTGGCGCTAGC
SEQ. ID NO: 51
2097_R primer used for PCR amplification for the VL domain
GATGGTGATGATGATGTGCGGATGCG
SEQ. ID NO: 52
Primer Insert 1_For
GGCTACGAAGACACCCCAGCCGGCCATGGCTC
SEQ. ID NO: 53
Primer H2_39Rev
GGCTACGAAGACTACCATCCACTCCAAACCTTGGCCCGGTGCKNNACGAACCCA
GCTAATCG
SEQ. ID NO: 54
Primer H2_42Rev
GGCTACGAAGACTACCATCCACTCCAAACCKNNGCCCGGTGCCTGACGAACCCA
GCTAATCG
SEQ. ID NO: 55
Primer H2_43Rev
GGCTACGAAGACTACCATCCACTCCAAKNNTTGGCCCGGTGCCTGACGAACCCA
GCTAATCG
SEQ. ID NO: 56
Primer H2_WTRev
GGCTACGAAGACTACCATCCACTCCAAACCTTGGCCCGGTGCCTGACGAACCCA
GCTAATCG
SEQ. ID NO: 57
Primer H2_45Rev
GGCTACGAAGACTACCATCCACTCKNNACCTTGGCCCGGTGCCTGACGAACCCA
GCTAATCG
SEQ. ID NO: 58
Primer H2_For
GGCTACGAAGACTAATGGGTGGTATTATCCCGATTTTTGGTACTGCTAATTATGCG
C
SEQ. ID NO: 59
Primer H3_89Rev
GGCTACGAAGACTATCACGGGCGCAGTAATAKNNAGCGGTATCATCGCTACG
SEQ. ID NO: 60
Primer H3_WTRev
GGCTACGAAGACTATCACGGGCGCAGTAATACACAGCGGTATCATCGCTACG
SEQ. ID NO: 61
Primer H3_For
GGCTACGAAGACTAGTGATAATCTGGGTTATTGCAGCGGCGGCTCC
SEQ. ID NO: 62
Primer H4_105Rev
GGCTACGAAGACTAAGACGGTGACTAAGGTACCKNNACCCCAAACATCC
SEQ. ID NO: 63
Primer H4_WTRev
GGCTACGAAGACTAAGACGGTGACTAAGGTACCTTGACCCCAAACATCC
SEQ. ID NO: 64
Primer H4_For
GGCTACGAAGACTAGTCTCGAGCGGTGGTGGCGGCTCCGG
SEQ. ID NO: 65
Primer L2_37Rev
GGCTACGAAGACTAGACGGTCGTCGGTGACGAGCCCGGGCGKNNCTGATACCAA
TGCAC
SEQ. ID NO: 66
Primer L2_43Rev
GGCTACGAAGACTAGACGGTCGTCGGKNNCGAGCCCGGGCGCTGCTGATACC
SEQ. ID NO: 67
Primer L2_WTRev
GGCTACGAAGACTAGACGGTCGTCGGTGACGAGCCCGGGCGCTGCTGATACCAA
TGCAC
SEQ. ID NO: 68
Primer L2_For
GGCTACGAAGACTACGTCATCTATGAGGACAACCAGCGTCCGAGCGGGGTGC
SEQ. ID NO: 69
Primer L3_85Rev
GGCTACGAAGACTAGCTTTGGCAGTAGTAKNNCGCCTCGTCCTCG
SEQ. ID NO: 70
Primer L3_WTRev
GGCTACGAAGACTAGCTTTGGCAGTAGTAGTCCGCCTCGTCCTCG
SEQ. ID NO: 71
Primer L4_100For
GGCTACGAAGACTAAAGCTACGACCGCTCTAACCACGAAGTTGTTTTTGGCNNKG
GTACGAAGCTGAC
SEQ. ID NO: 72
Primer pSANG4_Myc_M13_Rev v2
GGCTACGAAGACTACAACTTTCAACAGTTTCTGCGGCCCCATTCAGATCCTCTTC
SEQ. ID NO: 73
Primer pSANG4Myc_BbsI_For
GGCTACGAAGACTAGTTGTTTAGCAAAACCTCATACAGAAAATTCATTTACTAACG
TCTGG
SEQ. ID NO: 74
Primer pSANG4_Vector_Rev
GGCTACGAAGACCATGGGCCGCATAGAAAGGAACAAC
SEQ. ID NO: 75
Primer pSANG5th_For
TGGAAAAACGCCAGCAACGC
SEQ. ID NO: 76
Primer-96gIII
CCCTCATAGTTGCGTAACG
SEQ. ID NO: 77
VH WT clone picked after round 2 of phage display selection from the framework chain
interface library.
QVQLVQSGAEVKKPGSSVKVSCKASGGTFSSYAISWVRQAPGQGLEWMGGIIPIFGT
ANYAQKFQGRVTITADESTSTAYMELRSLRSDDTAVYYCARDNLGYCSGGSCYSDYY
YYYMDVWGQGTLVTV
SEQ. ID NO: 78
VH C1 clone picked after round 2 of phage display selection from the framework chain
interface library.
HVQLVQSGAEVKKPGSSVKVSCKASGGTFSSYAISWVRQAPGQTLEWMGGIIPIFGT
ANYAQKFQGRVTITADESTSTAYMELRSLRSDDTAVYYCARDNLGYCSGGSCYSDYY
YYYMDVWGQGTLVTV
SEQ. ID NO: 79
VH C2 clone picked after round 2 of phage display selection from the framework chain
interface library.
QVQLVQSGAEVKKPGSSVKVSCKASGGTFSSYAISWVRSAPGQGLEWMGGIIPIFGT
ANYAQKFQGRVTITADESTSTAYMELRSLRSDDTAVYYCARDNLGYCSGGSCYSDYY
YYYMDVWGVGTLVTV
SEQ. ID NO: 80
VH C3 clone picked after round 2 of phage display selection from the framework chain
interface library.
QVQLVQSGAEVKKPGSSVKVSCKASGGTFSSYAISWVRQAPGQGLEWMGGIIPIFGT
ANYAQKFKGRVTITADESTSTAYMELRSLRSDDTADYYCARDNLGYCSGGSCYSDYY
YYYMDVWGQGTLVTV
SEQ. ID NO: 81
VH C4 clone picked after round 2 of phage display selection from the framework chain
interface library.
QVQLVQSGAEVKKPGSSVKVSCKASGGTFSSYAISWVRQAPGQRLEWMGGIIPIFGT
ANYAQKFQGRVTITADESTSTAYMELRSLRSDDTAQYYCARDNLGYCSGGSCYSDY
YYYYMDVWGQGTLVTV
SEQ. ID NO: 82
VH C5 clone picked after round 2 of phage display selection from the framework chain
interface library.
QVQLVQSGAEVKKPGSSVKVSCKASGGTFSSYAISWVRRAPGQGLEWMGGIIPIFGT
ANYAQKFQGRVTITADESTSTAYMELRSLRSDDTAQYYCARDNLGYCSGGSCYSDY
YYYYMDVWGQGTLVTV
SEQ. ID NO: 83
VH C6 clone picked after round 2 of phage display selection from the framework chain
interface library.
QVQLVQSGAEVKKPGSSVKVSCKASGGTFSSYAISWVRQAPGQNLEWMGGIIPIFGT
ANYAQKFQGRVTITADESTSTAYMELRSLRSDDTAVYYCARDNLGYCSGGSCYSDYY
YYYMDVWGQGTLVTV
SEQ. ID NO: 84
VH C7 clone picked after round 2 of phage display selection from the framework chain
interface library.
QVQLVQSGAEVKKPGSSVKVSCKASGGTFSSYAISWVRTAPGQGLEWMGGIIPIFGT
ANYAQKFQGRVTITADESTSTAYMELRSLRSDDTATYYCARDNLGYCSGGSCYSDYY
YYYMDVWGQGTLVTV
SEQ. ID NO: 85
VH C8 clone picked after round 2 of phage display selection from the framework chain
interface library.
QVQLVQSGAEVKKPGSSVKVSCKASGGTFSSYAISWVRQAPGQYLEWMGGIIPIFGT
ANYAQKFQGRVTITADESTSTAYMELRSLRSDDTAVYYCARDNLGYCSGGSCYSDYY
YYYMDVWGQGTLVTV
SEQ. ID NO: 86
VH C9 clone picked after round 2 of phage display selection from the framework chain
interface library.
QFQLVQSGAEVKKPGSSVKVSCKASGGTFSSYAISWVRQAPGQGLEWMGGIIPIFGT
ANYAQKFQGRVTITADESTSTAYMELRSLRSDDTATYYCARDNLGYCSGGSCYSDYY
YYYMDVWGQGTLVTV
SEQ. ID NO: 87
VH C10 clone picked after round 2 of phage display selection from the framework chain
interface library.
QVQLVQSGAEVKKPGSSVKVSCKASGGTFSSYAISWVREAPGQGLEWMGGIIPIFGT
ANYAQKFQGRVTITADESTSTAYMELRSLRSDDTAIYYCARDNLGYCSGGSCYSDYY
YYYMDVWGQGTLVTV
SEQ. ID NO: 88
VL C3 clone picked after round 2 of phage display selection from the framework chain
interface library.
NFMLTQPRSVSESPGKTVTISCTRSSGSIGSDYVHWYQQRPGSRPTTVIYEDNQRPS
GVPDRFSGSIDSSSNSASLTISGLKTEDEADYYCQSYDRSNHEVVFGGGTKLTVL
SEQ. ID NO: 89
VL C4 clone picked after round 2 of phage display selection from the framework chain
interface library.
NFMLTQPRSVSESPGKTVTISCTRSSGSIGSDYVHWYQQRPGSQPTTVIYEDNQRPS
GVPDRFSGSIDSSSNSASLTISGLKTEDEATYYCQSYDRSNHEVVFGVGTKLTVL
SEQ. ID NO: 90
VL C5 clone picked after round 2 of phage display selection from the framework chain
interface library.
NFMLTQPRSVSESPGKTVTISCTRSSGSIGSDYVHWYQQRPGSKPTTVIYEDNQRPS
GVPDRFSGSIDSSSNSASLTISGLKTEDEADYYCQSYDRSNHEVVFGGGTKLTVL
SEQ. ID NO: 91
VL C6 clone picked after round 2 of phage display selection from the framework chain
interface library.
NFMLTQPRSVSESPGKTVTISCTRSSGSIGSDYVHWYQQRPGSSPTTVIYEDNQRPS
GVPDRFSGSIDSSSNSASLTISGLKTEDEADYYCQSYDRSNHEVVFGWGTKLTVL
SEQ. ID NO: 92
VL C7 clone picked after round 2 of phage display selection from the framework chain
interface library.
NFMLTQPRSVSESPGKTVTISCTRSSGSIGSDYVHWYQFRPGSSPTTVIYEDNQRPS
GVPDRFSGSIDSSSNSASLTISGLKTEDEADYYCQSYDRSNHEVVFGGGTKLTVL
SEQ. ID NO: 93
VL C8 clone picked after round 2 of phage display selection from the framework chain
interface library.
NFMLTQPRSVSESPGKTVTISCTRSSGSIGSDYVHWYQQRPGSSPTTVIYEDNQRPS
GVPDRFSGSIDSSSNSASLTISGLKTEDEAAYYCQSYDRSNHEVVFGGGTKLTVL
SEQ. ID NO: 94
VL C10 clone picked after round 2 of phage display selection from the framework chain
interface library.
NFMLTQPRSVSESPGKTVTISCTRYSGSIGSDYVHWYQVRPGSSPTTVIYEDNQRPS
GVPDRFSGSIDSSSNSASLTISGLKTEDEAHYYCQSYDRSNHEVVFGHGTKLTVL
SEQ. ID NO: 95
Consensus VH sequence of randomly picked scFVs from 2^{nd} round with increased pH
sensitivity
XVQLVQSXAEVKKPGSXVKVSCKASGGTFSSYAISWVRXAPGQXXEXMGGIIPIFGTA
NYAQKFQGRVTITADXSTSTAYMXLXSLRSDDTAXYXCARDNLGYCSGGSCYSDYYY
YYMDVXGXGTLVTVSS
SEQ. ID NO: 96
Consensus VL sequence of randomly picked scFVs from 2^{nd} round with increased pH
sensitivity
SSGGGGSGGGGSGGGASNFMLTQPRSVSESPGKTVTISCTRSXGSIGSDXVHWXQ
XRPGSXXTXVIXEDNQRPSGVPDRFSGSIDSSSNSASLTISGLKTEDEAXYXCQSYDR
SNHEVVXGXGTKLTVXSSGGGGSGGGGSGGGASNFMLTQPRSVSESPGKTVTISCT
RSXGSIGSDXVHWXQXRPGSXXTXVIXEDNQRPSGVPDRFSGSIDSSSNSASLTISGL
KTEDEAXYXCQSYDRSNHEVVXGXGTKLTVX
SEQ. ID NO: 97
Consensus VH FWR1 of randomly picked scFVs from 2^{nd} round with increased pH
sensitivity
XVQLVQSXAEVKKPGSXVKVSCKASGGTFSXVQLVQSXAEVKKPGSXVKVSCKASG
GTFS
SEQ. ID NO: 98
Consensus VH FWR2 of randomly picked scFVs from 2^{nd} round with increased pH
sensitivity
WRXAPGQXXEXMG
SEQ. ID NO: 99
Consensus VH FWR3 of randomly picked scFVs from 2^{nd} round with increased pH
sensitivity
RVTITADXSTSTAYMXLXSLRSDDTAXYXCAR
SEQ. ID NO: 100
Consensus VH FWR4 of randomly picked scFvs from 2^{nd} round with increased pH
sensitivity and Adalimumab, Bevacizumab and Fasinumab
XGXGTLVTVSS
SEQ. ID NO: 101
Consensus VL FWR2 of randomly picked scFvs from 2^{nd} round with increased pH
sensitivity
WXQXRPGSXXTXVIX
SEQ. ID NO: 102
Consensus VL FWR3 of randomly picked scFvs from 2^{nd} round with increased pH
sensitivity
GVPDRFSGSIDSSSNSASLTISGLKTEDEAXYXC
SEQ. ID NO: 103
Consensus VL FWR4 of randomly picked scFvs from 2^{nd} round with increased pH
sensitivity
XGXGTKLTVX
SEQ. ID NO: 104
Consensus Light chain CDR1 from point mutation approach
TRSXGSIGSDXVH
SEQ. ID NO: 105
VH sequence of 2554_01_D11 (+1), VH sequence of B04_F_B04_R (+1), VH
sequence of B02_F_B02_R (+1)
QVQLVQSGAEVKKPGSSVKVSCKASGGTFSSYAISWVRQAPGQGLEWMGGIIPIFGT
ANYAQKFQGRVTITADESTSTAYMELRSLRSDDTAVYYCARDNLGYCSGGSCYSDYY
YYYMDVWGQGTLVTVSSGG
SEQ ID NO: 106
VH sequence of F09_F_09_R (+1)
QVQLVQSGAEVKKPGSYVKVSCKASGGTFSSYAISWVRSAPGQGLEWMGGIIPIFGT
ANYAQKFQGRVTITADESTSTAYMELRSLRSDDTAVYYCARDNLGYCSGGSCYSDYY
YYYMDVWGQGTLVTVSSGG
SEQ ID NO: 107
VH sequence of F05_F_F05_R (+1)
QVQLVQSGAEVKKPGSSVKVSCKASGGTFSSYAISWVRSAPGQGLEWMGGIIPIFGT
ANYAQKFQGRVTITADESTSTAYMELRSLRSDDTAVYYCARDNLGYCSGGSCYSDYY
YYYMDVWGQGTLVTVSSGG
SEQ ID NO: 108
VH sequence of G02_F_G02_R (+1)
QVQLVQSGAEVKKPGSSVKVSCKASGGTFSSYAISWVRQAPGQGLEWMGGIIPIFGT
ANYAQKFQGRVTITADESTSTAYMLRSLRSDDTAVYYCARDNLGYCSGGSCYSDYYY
YYMDVWGQGTLVTVSSGG
SEQ ID NO: 109
VH sequence of D01_F_D01_R (+1), VH sequence of H03_F_H03_R (+1)
QVQLVQSGAEVKKPGSSVKVSCKASGGTFSSYAISWVRQAPGQGLEWMGGIIPIFGT
ANYAQKFQGRVTITADESTSTAYMELRSLRSDDTAVYYCARDNLGYCSGGSCYSDYY
YYYMDVWGTGTLVTVSSGG
SEQ ID NO: 110
VH sequence of A08_F_A08_R (+1)
QVQLVQSAEVKKPGSSVKVSCKASGGTFSSYAISWVRQAPGQGLEWMGGIIPIFGTA
NYAQKFQGRVTITADESTSTAYMELRSLRSDDTAVYYCARDNLGYCSGGSCYSDYYY
YYMDVWGRGTLVTVSSGG
SEQ ID NO: 111
VH sequence of D05_F_D05_R (+1)
QVQLVQSGAEVKKPGSSVKVSCKASGGTFSSYAISWVRQAPGQGLEWMGGIIPIFGT
ANYAQKFQGRVTITADESTSTAYMELRSLRSDDTATYYCARDNLGYCSGGSCYSDYY
YYYMDVWGTGTLVTVSSGG
SEQ ID NO: 112
VH sequence of E08_F_E08_R (+1)
QVQLVQSGAEVKKPGSSVKVSCKASGGTFSSYAISWVRQAPGQPLEWMGGIIPIFGT
ANYAQKFQGRVTITADESTSTAYMELRSLRSDDTAVYYCARDNLGYCSGGSCYSDYY
YYYMDVWGRGTLVTVSSGG
SEQ ID NO: 113
VH sequence of D12_F_D12_R (+1)
QVQLVQSGAEVKKPGSSVKVSCKASGGTFSSYAISWVRQAPGQRLEWMGGIIPIFGT
ANYAQKFQGRVTITADESTSTAYMELRSLRSDDTANYYCARDNLGYCSGGSCYSDYY
YYYMDVWGQGTLVTVSSGG
SEQ ID NO: 114
VH sequence of F07_F_F07_R (+1)
QVQLVQSGAEVKKPGSSVKVSCKASGGTFSSYAISWVRQAPGQNLEWMGGIIPIFGT
ANYAQKFQGRVTITADESTSTAYMELRSLRSDDTAIYYCARDNLGYCSGGSCYSDYY
YYYMDVWGQGTLVTVSSGG
SEQ ID NO: 115
VH sequence of G05_F_G05_R (+1)
QVQLVQSGAEVKKPGSSVKVSCKASGGTFSSYAISWVRQAPGQSLEWMGGIIPIFGT
ANYAQKFQGRVTITADSTSTAYMELRSLRSDDTAQYYCARDNLGYCSGGSCYSDYY
YYYMDVWGQGTLVTVSSGG
SEQ ID NO: 116
VH sequence of B05_F_B05_R (+1)
QVQLVQSGAEVKKPGSSVKVSCKASGGTFSSYAISWVRQAPGQGLEWMGGIIPIFGT
ANYAQKFQGRVTITADESTSTAYMELRSLRSDDTAVYYCARDNLGYCSGGSCYSDYY
YYYMDVWGKGTLVTVSSGG
SEQ ID NO: 117
VH sequence of C07_F_C07_R (+1), VH sequence of C09_F_C09_R (+1)
QVQLVQSGAEVKKPGSSVKVSCKASGGTFSSYAISWVRQAPGQKLEWMGGIIPIFGT
ANYAQKFQGRVTITADESTSTAYMELRSLRSDDTAVYYCARDNLGYCSGGSCYSDYY
YYYMDVWGPGTLVTVSSGG
SEQ ID NO: 118
VH sequence of E05_F_E05_R (+1)
HVQLVQSGAEVKKPGSSVKVSCKASGGTFSSYAISWVREAPGQGLEWMGGIIPIFGT
ANYAQKFQGRVTITADESTSTAYMELRSLRSDDTAVYYCARDNLGYCSGGSCYSDYY
YYYMDVWGDGTLVTVSSGG
SEQ ID NO: 119
VH sequence of H02_F_H02_R (+1)
HVQLVQSGAEVKKPGSSVKVSCKASGGTFSSYAISWVRQAPGQQLEWMGGIIPIFGT
ANYAQKFQGRVTITADESTSTAYMELRSLRSDDTATYYCARDNLGYCSGGSCYSDYY
YYYMDVWGPGTLVTVSSGG
SEQ ID NO: 120
VH sequence of D08_F_D08_R (+1)
HVQLVQSGAEVKKPGSSVKVSCKASGGTFSSYAISWVRQAPGQQLEWMGGIIPIFGT
ANYAQKFQGRVTITADESTSTAYMELRSLRSDDTALYYCARDNLGYCSGGSCYSDYY
YYYMDVWGKGTLVTVSSGG
SEQ ID NO: 121
VH sequence of C04_F_C04_R (+1)
QVQLVQSGAEVKKPGSSVKVSCKASGGTFSSYAISWVRQAPGQALEWMGGIIPIFGT
ANYAQKFQGRVTITADESTSTAYMELRSLRSDDTAAYYCARDNLGYCSGGSCYSDYY
YYYMDVWGPGTLVTVSSGG
SEQ ID NO: 122
VH sequence of F03_F_F03_R (+1)
QVQLVQSGAEVKKPGSSVKVSCKASGGTFSSYAISWVRQAPGQKLEWMGGIIPIFGT
ANYAQKFQGRVTITADESTSTAYMELRSLRSDDTAKYYCARDNLGYCSGGSCYSDYY
YYYMDVWGIGTLVTVSSGG
SEQ ID NO: 123
VH sequence of B03_F_B03_R (+1)
QVQLVQSGAEVKKPGSSVKVSCKASGGTFSSYAISWVRQAPGQTLEWMGGIIPIFGT
ANYAQKFQGRVTITADESTSTAYMELRSLRSDDTAVYYCARDNLGYCSGGSCYSDYY
YYYMDVWGTGTLVTVSSGG
SEQ ID NO: 124
VH sequence of B10_F_B10_R (+1)
QVQLVQSGAEVKKPGSSVKVSCKASGGTFSSYAISWVRAAPGQGLEWMGGIIPIFGT
ANYAQKFQGRVTITADESTSTAYMELKSLRSDDTAVYYCARDNLGYCSGGSCYSDYY
YYYMDVWGPGTLVTVSSGG
SEQ ID NO: 125
VL sequence of 2554_01_D11 (+1), VL sequence of F05_F_F05_R (+1), VL sequence
of G02_F_G02_R (+1)
ASNFMLTQPRSVSESPGKTVTISCTRSSGSIGSDYVHWYQQRPGSSPTTVIYEDNQR
PSGVPDRFSGSIDSSSNSASLTISGLKTEDEADYYCQSYDRSNHEVVFGGGTKLTVL
SEQ ID NO: 126
VL sequence of F09_F_09_R (+1)
ASNFMLTQPRSVSESPGKTVTISCTRSSGSIGSDYVHWYQLRPGSSPTTVIYEDNQR
PSGVPDRFSGSIDSSSNSASLTISGLKTEDEADYYCQSYDRSNHEVVFGGGTKLTVL
SEQ ID NO: 127
VL sequence of B04_F_B04_R (+1)
ASNFMLTQPRSVSESPGKTVTISCTRSSGSIGSDYVHWYQYRPGSSPTTVIYEDNQR
PSGVPDRFSGSIDSSSNSASLTISGLKTEDEADYYCQSYDRSNHEVVFGGGTKLTVL
SEQ ID NO: 128
VL sequence of B02_F_B02_R (+1)
ASNFMLTQPRSVSESPGKTVTISCTRSSGSIGSDYVHWYQSRPGSSPTTVIYEDNQR
PSGVPDRFSGSIDSSSNSASLTISGLKTEDEASYYCQSYDRSNHEVVFGGGTKLTVL
SEQ ID NO: 129
VL sequence of D01_F_D01_R (+1)
ASNFMLTQPRSVSESPGKTVTISCTRSSGSIGSDYVHWYQQRPGSSPTTVIYEDNQR
PSGVPDRFSGSIDSSSNSASLTISGLKTEDEANYYCQSYDRSNHEVVFGGGTKLTVL
SEQ ID NO: 130
VL sequence of H03_F_H03_R (+1)
ASNFMLTQPRSVSESPGKTVTISCTRSSGSIGSDYVHWYQIRPGSSPTTVIYEDNQRP
SGVPDRFSGSIDSSSNSASLTISGLKTEDEATYYCQSYDRSNHEVVFGGGTKLTVL
SEQ ID NO: 131
VL sequence of A08_F_A08_R (+1)
ASNFMLTQPRSVSESPGKTVTISCTRSSGSIGSDYVHWYQTRPGSSPTTVIYEDNQR
PSGVPDRFSGSIDSSSNSASLTISGLKTEDEAFYYCQSYDRSNHEVVFGGGTKLTVL
SEQ ID NO: 132
VL sequence of D05_F_D05_R (+1)
ASNFMLTQPRSVSESPGKTVTISCTRSSGSIGSDYVHWYQQRPGSSPTTVIYEDNQR
PSGVPDRFSGSIDSSSNSASLTISGLKTEDEADYYCQSYDRSNHEVVFGSGTKLTVL
SEQ ID NO: 133
VL sequence of E08_F_E08_R (+1)
ASNFMLTQPRSVSESPGKTVTISCTRSSGSIGSDYVHWYQQRPGSSPTTVIYEDNQR
PSGVPDRFSGSIDSSSNSASLTISGLKTEDEADYYCQSYDRSNHEVVFGGGTKLTVI
SEQ ID NO: 134
VL sequence of D12_F_D12_R (+1)
ASNFMLTQPRSVSESPGKTVTISCTRSSGSIGSDYVHWYQARPGSSPTTVIYEDNQR
PSGVPDRFSGSIDSSSNSASLTISGLKTEDEADYYCQSYDRSNHEVVFGGGTKLTVL
SEQ ID NO: 135
VL sequence of F07_F_F07_R (+1)
ASNFMLTQPRSVSESPGKTVTISCTRSSGSIGSDYVHWYQRRPGSSPTTVIYEDNQR
PSGVPDRFSGSIDSSSNSASLTISGLKTEDEADYYCQSYDRSNHEVVFGGGTKLTVL
SEQ ID NO: 136
VL sequence of G05_F_G05_R (+1)
ASNFMLTQPRSVSESPGKTVTISCTRSSGSIGSDYVHWYQQRPGSPPTTVIYEDNQR
PSGVPDRFSGSIDSSSNSASLTISGLKTEDEADYYCQSYDRSNHEVVFGGGTKLTVL
SEQ ID NO: 137
VL sequence of B05_F_B05_R (+1)
ASNFMLTQPRSVSESPGKTVTISCTRSYGSIGSDYVHWYQQRPGSTPTTVIYEDNQR
PSGVPDRFSGSIDSSSNSASLTISGLKTEDEADYYCQSYDRSNHEVVFGGGTKLTVL
SEQ ID NO: 138
VL sequence of C07_F_C07_R (+1), VL sequence of C09_F_C09_R (+1)
ASNFMLTQPRSVSESPGKTVTISCTRSSGSIGSDYVHWYQQRPGSTPTTVIYEDNQR
PSGVPDRFSGSIDSSSNSASLTISGLKTEDEADYYCQSYDRSNHEVVFGYGTKLTVL
SEQ ID NO: 139
VL sequence of E05_F_E05_R (+1)
ASNFMLTQPRSVSESPGKTVTISCTRSSGSIGSDYVHWYQQRPGSSPTTVIYEDNQR
PSGVPDRFSGSIDSSSNSASLTISGLKTEDEAVYYCQSYDRSNHEVVFGGGTKLTVL
SEQ ID NO: 140
VL sequence of H02_F_H02_R (+1)
ASNFMLTQPRSVSESPGKTVTISCTRSSGSIGSDYVHWYQQRPGSSPTTVIYEDNQR
PSGVPDRFSGSIDSSSNSASLTISGLKTEDEALYYCQSYDRSNHEVVFGGGTKLTVL
SEQ ID NO: 141
VL sequence of D08_F_D08_R (+1), VL sequence of C04_F_C04_R (+1)
ASNFMLTQPRSVSESPGKTVTISCTRSSGSIGSDYVHWYQQRPGSSPTTVIYEDNQR
PSGVPDRFSGSIDSSSNSASLTISGLKTEDEASYYCQSYDRSNHEVVFGGGTKLTVL
SEQ ID NO: 142
VL sequence of F03_F_F03_R (+1)
ASNFMLTQPRSVSESPGKTVTISCTRSSGSIGSDYVHWYQQRPGSAPTTVIYEDNQR
PSGVPDRFSGSIDSSSNSASLTISGLKTEDEASYYCQSYDRSNHEVVFGGGTKLTVL
SEQ ID NO: 143
VL sequence of B03_F_B03_R (+1)
ASNFMLTQPRSVSESPGKTVTISCTRSSGSIGSDYVHWYQQRPGSVPTTVIYEDNQR
PSGVPDRFSGSIDSSSNSASLTISGLKTEDEAAYYCQSYDRSNHEVVFGLGTKLTVL
SEQ ID NO: 144
VL sequence of B10_F_B10_R (+1)
ASNFMLTQPRSVSESPGKTVTISCTRSSGSIGSDYVHWYQVRPGSSPTTVIYEDNQR
PSGVPDRFSGSIDSSSNSASLTISGLKTEDEARYYCQSYDRSNHEVVFGGGTKLTVL
SEQ ID NO: 145
Heavy chain
XXQVQLVESGGGLVQPGGSLRLSCAASGFTFSSGSYAMSWVRQAPGKGLEWVGGI
SPKTSGGWNTYYADSVKGRFTISRDNSKNFDTFTLYLQMNSLRAEDTAVYYCATXRR
DQLYGXXPLXYGGGGGGGGYYYYGSGXEXLYDAFDYDPQYSWGQGTLVTVSS
SEQ ID NO: 146
Lambda light chain
QSVLTQPPSVSGAPGQTVTISCXGSSSNIGSYYDVSWYQQLPGTAPKLLIYDDSNRPS
GVPDRFSGSXDXKSGNTASLTISGLQAEDEADYYCQSWDSSLSGXVVFGGGTKLTVL
SEQ ID NO: 147
Kappa light chain
DIVMTQSPSSLSASVGDRVTITCRASQSILYSSSSKSYLAWYQQKPGQAPKLLIYGAS
SRASGVPSRFSGSGSGTDFTLTISSLQPEDFATYYCQQYNSWVPPLXFTFGQGTKVE
IK
SEQ ID NO: 148
Adalimumab
MAEVQLVESGGGLVQPGRSLRLSCAASGFTFDDYAMHWVRQAPGKGLEWVSAITW
NSGHIDYADSVEGRFTISRDNAKNSLYLQMNSLRAEDTAVYYCAKVSYLSTASSLDY
WGQGTLVTVSSGGGGSGGGGSGGGASDIQMTQSPSSLSASVGDRVTITCRASQGIR
NYLAWYQQKPGKAPKLLIYAASTLQSGVPSRFSGSGSGTDFTLTISSLQPEDVATYYC
QRYNRAPYTFGQGTKVEIKAAASAHHHHHH
SEQ ID NO: 149
Bevacizumab
MAEVQLVESGGGLVQPGGSLRLSCAASGYTFTNYGMNWVRQAPGKGLEWVGWINT
YTGEPTYAADFKRRFTFSLDTSKSTAYLQMNSLRAEDTAVYYCAKYPHYYGSSHWYF
DVWGQGTLVTVSSGGGGSGGGGSGGGASDIQMTQSPSSLSASVGDRVTITCSASQ
DISNYLNWYQQKPGKAPKVLIYFTSSLHSGVPSRFSGSGSGTDFTLTISSLQPEDFAT
YYCQQYSTVPWTFGQGTKVEIKAAASAHHHHHH
SEQ ID NO: 150
Fasinumab
MAQVQLVQSGAEVKKPGASVKVSCKVSGFTLTELSIHWVRQAPGKGLEWMGGFDPE
DGETIYAQKFQGRVTMTEDTSTDTAYMELTSLRSEDTAVYYCSTIFGVVTNFDNWGQ
GTLVTVSSGGGGSGGGGSGGGASDIQMTQSPSSLSASAGDRVTITCRASQAIRNDL
GWYQQKPGKAPKRLIYAAFNLQSGVPSRFSGSGSGTEFTLTISSLQPEDLASYYCQQ
YNRYPWTFGQGTKVEIKAAASAHHHHHH
SEQ. ID NO: 151
VL FWR 1 of Adalimumab and Bevacizumab
DIQMTQSPSSLSASVGDRVTITC
SEQ. ID NO: 152
VL FWR2 of Adalimumab
WYQQKPGKAPKLLIY
SEQ. ID NO: 153
VL FWR3 of Adalimumab
GVPSRFSGSGSGTDFTLTISSLQPEDVATYYC
SEQ. ID NO: 154
VL FWR4 of Adalimumab, Bevacizumab and Fasinumab
FGQGTKVEIK
SEQ. ID NO: 155
VH FWR1 of Adalimumab
EVQLVESGGGLVQPGRSLRLSCAASGFTFD
SEQ. ID NO: 156
VH FWR2 of Adalimumab
WVRQAPGKGLEWVS
SEQ. ID NO: 157
VH FWR3 of Adalimumab
RFTISRDNAKNSLYLQMNSLRAEDTAVYYCAK
SEQ. ID NO: 158
VL FWR2 of Bevacizumab
WYQQKPGKAPKVLIY
SEQ. ID NO: 159
VL FWR3 of Bevacizumab
GVPSRFSGSGSGTDFTLTISSLQPEDFATYYC
SEQ. ID NO: 160
VH FWR1 of Bevacizumab
EVQLVESGGGLVQPGGSLRLSCAASGYTFT
SEQ. ID NO: 161
VH FWR2 of Bevacizumab
WVRQAPGKGLEWVG
SEQ. ID NO: 162
VH FWR3 of Bevacizumab
RFTFSLDTSKSTAYLQMNSLRAEDTAVYYCAK
SEQ. ID NO: 163
VL FWR1 of Fasinumab
DIQMTQSPSSLSASAGDRVTITC
SEQ. ID NO: 164
VL FWR2 of Fasinumab
WYQQKPGKAPKRLIY
SEQ. ID NO: 165
VL FWR3 of Fasinumab
GVPSRFSGSGSGTEFTLTISSLQPEDLASYYC
SEQ. ID NO: 166
VH FWR1 of Fasinumab
QVQLVQSGAEVKKPGASVKVSCKVSGFTLT
SEQ. ID NO: 167
VH FWR2 of Fasinumab
WRQAPGKGLEWMG
SEQ. ID NO: 168
VH FWR3 of Fasinumab
RVTMTEDTSTDTAYMELTSLRSEDTAVYYCST
SEQ. ID NO: 169
Light Chain CDR1 of Adalimumab
RASQGIRNYLA
SEQ. ID NO: 170
Light Chain CDR2 of Adalimumab
AASTLQS
SEQ. ID NO: 171
Light Chain CDR3 of Adalimumab
QRYNRAPYT
SEQ. ID NO: 172
Light Chain CDR1 of Bevacizumab
SASQDISNYLN
SEQ. ID NO: 173
Light Chain CDR2 of Bevacizumab
FTSSLHS
SEQ. ID NO: 174
Light Chain CDR3 of Bevacizumab
QQYSTVPWT
SEQ. ID NO: 175
Light Chain CDR1 of Fasinumab
RASQAIRNDLG
SEQ. ID NO: 176
Light Chain CDR2 of Fasinumab
AAFNLQS
SEQ. ID NO: 177
Light Chain CDR3 of Fasinumab
QQYNRYPWT
SEQ. ID NO: 178
Heavy Chain CDR1 of Adalimumab
DYAMH
SEQ. ID NO: 179
Heavy Chain CDR2 of Adalimumab
AITWNSGHIDYADSVEG
SEQ. ID NO: 180
Heavy Chain CDR3 of Adalimumab
VSYLSTASSLDY
SEQ. ID NO: 181
Heavy Chain CDR 1 of Bevacizumab
NYGMN
SEQ. ID NO: 182
Heavy Chain CDR2 of Bevacizumab
WINTYTGEPTYAADFKR
SEQ. ID NO: 183
Heavy Chain CDR3 of Bevacizumab
YPHYYGSSHWYFDV
SEQ. ID NO: 184
Heavy Chain CDR1 of Fasinumab
ELSIH
SEQ. ID NO: 185
Heavy Chain CDR2 of Fasinumab
GFDPEDGETIYAQKFQG
SEQ. ID NO: 186
Heavy Chain CDR3 of Fasinumab
IFGVVTNFDN
SEQ. ID NO: 187
Heavy Chain FWR2 of Adalimumab consensus 1
WVRXAPGKXXEXVS
SEQ. ID NO: 188
Heavy Chain FWR3 of Adalimumab consensus 1
RFTISRDXAKNSLYLXMXSLRAEDTAXYXCAK
SEQ. ID NO: 189
Heavy Chain FWR4 of Adalimumab, Bevacizumab and Fasinumab consensus 1
XGXGTLVTVSS
SEQ. ID NO: 190
Light Chain FWR2 of Adalimumab, Bevacizumab and Fasinumab consensus 1
WXQXKPGKXXKXLIX
SEQ. ID NO: 191
Light Chain FWR3 of Adalimumab consensus 1
GVPSRFSGSGSGTDFTLTISSLQPEDVAXYXC
SEQ. ID NO: 192
Light Chain FWR4 of Adalimumab, Bevacizumab and Fasinumab consensus 1
XGXGTKVEIX
SEQ. ID NO: 193
Heavy Chain FWR2 of Adalimumab consensus 2
WVRQAPGKXLEWVS
SEQ. ID NO: 194
Heavy Chain FWR3 of Adalimumab consensus 2
RFTISRDNAKNSLYLQMNSLRAEDTAXYYCAK
SEQ. ID NO: 195
Heavy Chain FWR4 of Adalimumab, Bevacizumab and Fasinumab consensus 2
WGXGTLVTVSS
SEQ. ID NO: 196
Light Chain FWR2 of Adalimumab consensus 2
WYQXKPGKAPKLLIY
SEQ. ID NO: 197
Heavy Chain FWR2 of Bevacizumab consensus 1
WVRXAPGKXXEXVG
SEQ. ID NO: 198
Heavy Chain FWR3 of Bevacizumab consensus 1
RFTFSLDXSKSTAYLXMXSLRXEDTAXYXCAK
SEQ. ID NO: 199
Light Chain FWR3 of Bevacizumab consensus 1
GVPSRFSGSGSGTDFTLTISSLQPEDFAXYXC
SEQ. ID NO: 200
Heavy Chain FWR2 of Bevacizumab consensus 2
WRQAPGKXLEWVG
SEQ. ID NO: 201
Heavy Chain FWR3 of Bevacizumab consensus 2
RFTFSLDTSKSTAYLQMNSLRXEDTAXYYCAK
SEQ. ID NO: 202
Light Chain FWR2 of Bevacizumab consensus 2
WYQXKPGKAPKVLIY
SEQ. ID NO: 203
Heavy Chain FWR2 of Fasinumab consensus 1
WVRXAPGKXXEXMG
SEQ. ID NO: 204
Heavy Chain FWR3 of Fasinumab consensus 1
RVTMTEDXSTDTAYMXLXSLRSEDTAXYXCST
SEQ. ID NO: 205
Light Chain FWR3 of Fasinumab consensus 1
GVPSRFSGSGSGTEFTLTISSLQPEDLAXYXC
SEQ. ID NO: 206
Heavy Chain FWR2 of Fasinumab consensus 2
WRQAPGKXLEWMG
SEQ. ID NO: 207
Heavy Chain FWR3 of Fasinumab consensus 2
RVTMTEDTSTDTAYMELTSLRSEDTAXYYCST
SEQ. ID NO: 208
Light Chain FWR2 of Fasinumab consensus 2
WYQXKPGKAPKRLIY
SEQ. ID NO: 209
Parent consensus sequence of Bevacizumab
MKYLLPTAAAGLLLLAAQPAMAEVQLVESGGGLVQPGGSLRLSCAASGYTFTNYGMN
WVRQAPGKXLEWVGWINTYTGEPTYAADFKRRFTFSLDTSKSTAYLQMNSLRAEDT
AXYYCAKYPHYYGSSHWYFDVWGXGTLVTVSSGGGGSGGGGSGGGASDIQMTQS
PSSLSASVGDRVTITCSASQDISNYLNWYQXKPGKAPKVLIYFTSSLHSGVPSRFSGS
GSGTDFTLTISSLQPEDFATYYCQQYSTVPWTFGQGTKVEIKAAASAHHHHHHKLDY
KDHDGDYKDHDIDYKDDDDK
SEQ. ID NO: 210
Bevacizumab mutant 1
MKYLLPTAAAGLLLLAAQPAMAEVQLVESGGGLVQPGGSLRLSCAASGYTFTNYGMN
WVRQAPGKALEWVGWINTYTGEPTYAADFKRRFTFSLDTSKSTAYLQMNSLRAEDT
AAYYCAKYPHYYGSSHWYFDVWGTGTLVTVSSGGGGSGGGGSGGGASDIQMTQS
PSSLSASVGDRVTITCSASQDISNYLNWYQFKPGKAPKVLIYFTSSLHSGVPSRFSGS
GSGTDFTLTISSLQPEDFATYYCQQYSTVPWTFGQGTKVEIKAAASAHHHHHHKLDY
KDHDGDYKDHDIDYKDDDDK
SEQ. ID NO: 211
Bevacizumab mutant 2
MKYLLPTAAAGLLLLAAQPAMAEVQLVESGGGLVQPGGSLRLSCAASGYTFTNYGMN
WRQAPGKALEWVGWINTYTGEPTYAADFKRRFTFSLDTSKSTAYLQMNSLRAEDT
ATYYCAKYPHYYGSSHWYFDVWGDGTLVTVSSGGGGSGGGGSGGGASDIQMTQS
PSSLSASVGDRVTITCSASQDISNYLNWYQLKPGKAPKVLIYFTSSLHSGVPSRFSGS
GSGTDFTLTISSLQPEDFATYYCQQYSTVPWTFGQGTKVEIKAAASAHHHHHHKLDY
KDHDGDYKDHDIDYKDDDDK
SEQ. ID NO: 212
Bevacizumab mutant 3
MKYLLPTAAAGLLLLAAQPAMAEVQLVESGGGLVQPGGSLRLSCAASGYTFTNYGMN
WRQAPGKPLEWVGWINTYTGEPTYAADFKRRFTFSLDTSKSTAYLQMNSLRAEDT
AIYYCAKYPHYYGSSHWYFDVWGSGTLVTVSSGGGGSGGGGSGGGASDIQMTQSP
SSLSASVGDRVTITCSASQDISNYLNWYQSKPGKAPKVLIYFTSSLHSGVPSRFSGSG
SGTDFTLTISSLQPEDFATYYCQQYSTVPWTFGQGTKVEIKAAASAHHHHHHKLDYK
DHDGDYKDHDIDYKDDDDK
SEQ. ID NO: 213
Bevacizumab mutant 4
MKYLLPTAAAGLLLLAAQPAMAEVQLVESGGGLVQPGGSLRLSCAASGYTFTNYGMN
WVRQAPGKSLEWVGWINTYTGEPTYAADFKRRFTFSLDTSKSTAYLQMNSLRTEDTA
TYYCAKYPHYYGSSHWYFDVWGSGTLVTVSSGGGGSGGGGSGGGASDIQMTQSP
SSLSASVGDRVTITCSASQDISNYLNWYQTKPGKAPKVLIYFTSSLHSGVPSRFSGSG
SGTDFTLTISSLQPEDFATYYCQQYSTVPWTFGQGTKVEIKAAASAHHHHHHKLDYK
DHDGDYKDHDIDYKDDDDK
SEQ. ID NO: 214
Bevacizumab mutant 5
MKYLLPTAAAGLLLLAAQPAMAEVQLVESGGGLVQPGGSLRLSCAASGYTFTNYGMN
WRQAPGKTLEWVGWINTYTGEPTYAADFKRRFTFSLDTSKSTAYLQMNSLRAEDTA
LYYCAKYPHYYGSSHWYFDVWGPGTLVTVSSGGGGSGGGGSGGGASDIQMTQSPS
SLSASVGDRVTITCSASQDISNYLNWYQIKPGKAPKVLIYFTSSLHSGVPSRFSGSGS
GTDFTLTISSLQPEDFATYYCQQYSTVPWTFGQGTKVEIKAAASAHHHHHHKLDYKD
HDGDYKDHDIDYKDDDDK
SEQ. ID NO: 215
Parent consensus sequence of Adalimumab
MKYLLPTAAAGLLLLAAQPAMAEVQLVESGGGLVQPGRSLRLSCAASGFTFDDYAMH
WRQAPGKXLEWVSAITWNSGHIDYADSVEGRFTISRDNAKNSLYLQMNSLRAEDTA
XYYCAKVSYLSTASSLDYWGXGTLVTVSSGGGGSGGGGSGGGASDIQMTQSPSSLS
ASVGDRVTITCRASQGIRNYLAWYQXKPGKAPKLLIYAASTLQSGVPSRFSGSGSGTD
FTLTISSLQPEDVATYYCQRYNRAPYTFGQGTKVEIKAAASAHHHHHHKLDYKDHDG
DYKDHDIDYKDDDDK
SEQ. ID NO: 216
Adalimumab mutant 1
MKYLLPTAAAGLLLLAAQPAMAEVQLVESGGGLVQPGRSLRLSCAASGFTFDDYAMH
WRQAPGKYLEWVSAITWNSGHIDYADSVEGRFTISRDNAKNSLYLQMNSLRAEDTA
YYYCAKVSYLSTASSLDYWGSGTLVTVSSGGGGSGGGGSGGGASDIQMTQSPSSLS
ASVGDRVTITCRASQGIRNYLAWYQSKPGKAPKLLIYAASTLQSGVPSRFSGSGSGTD
FTLTISSLQPEDVATYYCQRYNRAPYTFGQGTKVEIKAAASAHHHHHHKLDYKDHDG
DYKDHDIDYKDDDDK
SEQ. ID NO: 217
Adalimumab mutant 2
MKYLLPTAAAGLLLLAAQPAMAEVQLVESGGGLVQPGRSLRLSCAASGFTFDDYAMH
WRQAPGKGLEWVSAITWNSGHIDYADSVEGRFTISRDNAKNSLYLQMNSLRAEDTA
DYYCAKVSYLSTASSLDYWGQGTLVTVSSGGGGSGGGGSGGGASDIQMTQSPSSL
SASVGDRVTITCRASQGIRNYLAWYQRKPGKAPKLLIYAASTLQSGVPSRFSGSGSGT
DFTLTISSLQPEDVATYYCQRYNRAPYTFGQGTKVEIKAAASAHHHHHHKLDYKDHD
GDYKDHDIDYKDDDDK
SEQ. ID NO: 218
Adalimumab mutant 3
MKYLLPTAAAGLLLLAAQPAMAEVQLVESGGGLVQPGRSLRLSCAASGFTFDDYAMH
WVRQAPGKRLEWVSAITWNSGHIDYADSVEGRFTISRDNAKNSLYLQMNSLRAEDTA
FYYCAKVSYLSTASSLDYWGTGTLVTVSSGGGGSGGGGSGGGASDIQMTQSPSSLS
ASVGDRVTITCRASQGIRNYLAWYQLKPGKAPKLLIYAASTLQSGVPSRFSGSGSGTD
FTLTISSLQPEDVATYYCQRYNRAPYTFGQGTKVEIKAAASAHHHHHHKLDYKDHDG
DYKDHDIDYKDDDDK
SEQ. ID NO: 219
Adalimumab mutant 4
MKYLLPTAAAGLLLLAAQPAMAEVQLVESGGGLVQPGRSLRLSCAASGFTFDDYAMH
WVRQAPGKRLEWVSAITWNSGHIDYADSVEGRFTISRDNAKNSLYLQMNSLRAEDTA
FYYCAKVSYLSTASSLDYWGTGTLVTVSSGGGGSGGGGSGGGASDIQMTQSPSSLS
ASVGDRVTITCRASQGIRNYLAWYQLKPGKAPKLLIYAASTLQSGVPSRFSGSGSGTD
FTLTISSLQPEDVATYYCQRYNRAPYTFGQGTKVEIKAAASAHHHHHHKLDYKDHDG
DYKDHDIDYKDDDDK
SEQ. ID NO: 220
Adalimumab mutant 5
MKYLLPTAAAGLLLLAAQPAMAEVQLVESGGGLVQPGRSLRLSCAASGFTFDDYAMH
WRQAPGKTLEWVSAITWNSGHIDYADSVEGRFTISRDNAKNSLYLQMNSLRAEDTA
SYYCAKVSYLSTASSLDYWGPGTLVTVSSGGGGSGGGGSGGGASDIQMTQSPSSLS
ASVGDRVTITCRASQGIRNYLAWYQQKPGKAPKLLIYAASTLQSGVPSRFSGSGSGT
DFTLTISSLQPEDVATYYCQRYNRAPYTFGQGTKVEIKAAASAHHHHHHKLDYKDHD
GDYKDHDIDYKDDDDK
SEQ. ID NO: 221
2554_01_D11 PCR primer 1
GTAACCACCACACCCGCCGCGCTTAATG
SEQ. ID NO: 222
2554_01_D11 PCR primer 2 and Vector (1).FOR
CACCATACCCACGCCGAAACAAGCGC
SEQ. ID NO: 223
Ada_1_Forward and Bev_1_Forward
GGTCTGCTGCTCCTCGCT
SEQ. ID NO: 224
Ada_1_Reverse
TATGGTCTCACTAACCCATTCCAAKNNTTTACCTG
SEQ. ID NO: 225
Ada_2_Forward
TATGGTCTCATTAGTGCAATTACCTGGAACTCTGG
SEQ. ID NO: 226
Ada_2_Reverse
TATGGTCTCACCTTGGCACAGTAGTAKNNAGC
SEQ. ID NO: 227
Ada_3_Forward
TATGGTCTCAAAGGTTTCTTACTTGTCCACCGC
SEQ. ID NO: 228
Ada_3_Reverse
TATGGTCTCACAGTGACCAAAGTACCKNNACCC
SEQ. ID NO: 229
Ada_4_Forward
TATGGTCTCAACTGTCAGCAGTGGTGGTGGTGGTAGC
SEQ. ID NO: 230
Ada_4_Reverse
TATGGTCTCATGGAGCCTTACCCGGTTTKNNTTG
SEQ. ID NO: 231
Ada_5_Forward
TATGGTCTCATCCAAAGCTGCTGATTTACGCT
SEQ. ID NO: 232
Ada_5_Reverse
GTGATGGTGATGATGATGTGCGG
SEQ. ID NO: 233
Bev_1_Reverse
TATGGTCTCAGACCCATTCCAAKNNCTTACCCG
SEQ. ID NO: 234
Bev_2_Forward
TATGGTCTCAGGTCGGTTGGATTAATACCTACACCG
SEQ. ID NO: 235
Bev_2_Reverse
TATGGTCTCAACTTAGCACAATAGTAKNNAGCAGTGT
SEQ. ID NO: 236
Bev_3_Forward
TATGGTCTCAAAGTACCCACACTATTACGGTTCC
SEQ. ID NO: 237
Bev_3_Reverse
TATGGTCTCACCAAAGTACCKNNACCCCAA
SEQ. ID NO: 238
Bev_4_Forward
TATGGTCTCATTGGTTACTGTGAGTTCCGGT
SEQ. ID NO: 239
Bev_4_Reverse
TATGGTCTCACTTTACCTGGCTTKNNTTGGTACCA
SEQ. ID NO: 240
Bev_5_Forward
TATGGTCTCAAAAGCCCCGAAAGTTTTGATTTATTTCAC
SEQ. ID NO: 241
Bev_5_Reverse
TGATGGTGATGATGATGTGCGG
SEQ. ID NO: 242
Fragment 1 (1).FOR
TATGGTCTCAGGGAGGGATCATCCCTATCTTTG
SEQ. ID NO: 243
Fragment 1 (1).REV
TATGGTCTCACTCTCGCACAMNNATACACGG
SEQ. ID NO: 244
Fragment 2 (1).FOR
TATGGTCTCAAGAGACAACCTAGGATATTGTAGTGGTG
SEQ. ID NO: 245
Fragment 2 (1).REV
TATGGTCTCACGCTGCCAATGCTGCCACTGCTGCG
SEQ. ID NO: 246
Fragment 3 (1).FOR
TATGGTCTAGCGACTATGTGCATTGGNNKCAGC
SEQ. ID NO: 247
Fragment 3 (1).REV
TATGGTCTCAGATGACMNNGGTMNNGGAGC
SEQ. ID NO: 248
Fragment 4 (1).FOR
TATGGTCTCACATCNNKGAGGATAACCAAAGACC
SEQ. ID NO: 249
Fragment 4 (1).REV
TATGGTCTCACTGCGATCATAAGACTGACAMNNGTAGTC
SEQ. ID NO: 250
Fragment 5 (1).FOR
TATGGTCTCAGCAGCAATCATGAAGTGGTGNNKGG
SEQ. ID NO: 251
Fragment 5 (1).REV
TGCGGATGCGGCCGCGGGCTGA
SEQ. ID NO: 252
Vector (1). REV
TATGGTCTCATCCCATMNNCTCMNNCCCT
SEQ. ID NO: 253
s10b primer
GGCTTTGTTAGCAGCCGGATCTCA
SEQ. ID NO: 254
Sequence of D11 scFv clone ″*″ from FIG. 18
MAQVQLVQSGAEVKKPGSSVKVSCKASGGTFSSYAISWVRQAPGQGLEWMGGIIPIF
GTANYAQKFQGRVTITADESTSTAYMELRSLRSDDTAVYYCARDNLGYCSGGSCYSD
YYYYYMDVWGQGTLVTVSSGGGGSGGGGSGGGASNFMLTQPRSVSESPGKTVTIS
CTRSSGSIGSDYVHWNQQRPGSSPTTVIYEDNQRPSGVPDRFSGSIDSSSNSASLTIS
GLKTEDEADYYCQSYDRSNHEVVFGGGTKLTVL

### REFERENCES

1. M. L. Murtaugh, S. W. Fanning, T. M. Sharma, A. M. Terry, J. R. Horn, A combinatorial histidine scanning library approach to engineer highly pH-dependent protein switches: Engineering pH-Sensitive Protein Switches. Protein Science. 20, 1619-1631 (2011).
2. B. C. Mackness, J. A. Jaworski, E. Boudanova, A. Park, D. Valente, C. Mauriac, O. Pasquier, T. Schmidt, M. Kabiri, A. Kandira, K. Radošević, H. Qiu, Antibody Fc engineering for enhanced neonatal Fc receptor binding and prolonged circulation half-life. mAbs. 11, 1276-1288 (2019).
3. C. Schröter, R. Günther, L. Rhiel, S. Becker, L. Toleikis, A. Doerner, J. Becker, A. Schönemann, D. Nasu, B. Neuteboom, H. Kolmar, B. Hock, A generic approach to engineer antibody pH-switches using combinatorial histidine scanning libraries and yeast display. mAbs. 7, 138-151 (2015).
4. L. Ledsgaard, A. Ljungars, C. Rimbault, C. V. Sørensen, T. Tulika, J. Wade, Y. Wouters, J. McCafferty, A. H. Laustsen, Advances in antibody phage display technology. Drug Discovery Today. 27, 2151-2169 (2022).
5. T. Igawa, S. Ishii, T. Tachibana, A. Maeda, Y. Higuchi, S. Shimaoka, C. Moriyama, T. Watanabe, R. Takubo, Y. Doi, T. Wakabayashi, A. Hayasaka, S. Kadono, T. Miyazaki, K. Haraya, Y. Sekimori, T. Kojima, Y. Nabuchi, Y. Aso, Y. Kawabe, K. Hattori, Antibody recycling by engineered pH-dependent antigen binding improves the duration of antigen neutralization. Nat Biotechnol. 28, 1203-1207 (2010).
6. J. Chaparro-Riggers, H. Liang, R. M. DeVay, L. Bai, J. E. Sutton, W. Chen, T. Geng, K. Lindquist, M. G. Casas, L. M. Boustany, C. L. Brown, J. Chabot, B. Gomes, P. Garzone, A. Rossi, P. Strop, D. Shelton, J. Pons, A. Rajpal, Increasing Serum Half-life and Extending Cholesterol Lowering in Vivo by Engineering Antibody with pH-sensitive Binding to PCSK9. Journal of Biological Chemistry. 287, 11090-11097 (2012).
7. L. Ledsgaard, A. H. Laustsen, U. Pus, J. Wade, P. Villar, K. Boddum, P. Slavny, E. W. Masters, A. S. Arias, S. Oscoz, D. T. Griffiths, A. M. Luther, M. Lindholm, R. A. Leah, M. S. Møller, H. Ali, J. McCafferty, B. Lomonte, J. M. Gutiérrez, A. Karatt-Vellatt, In vitro discovery of a human monoclonal antibody that neutralizes lethality of cobra snake venom. mAbs. 14, 2085536 (2022).
8. L. Ledsgaard, J. Wade, K. Boddum, I. Oganesyan, J. Harrison, T. P. Jenkins, P. Villar, R. A. Leah, R. Zenobi, J. McCafferty, B. Lomonte, J. M. Gutiérrez, A. H. Laustsen, A. Karatt-Vellatt, “Discovery of a broadly-neutralizing human antibody that can rescue mice challenged with neurotoxin-rich snake venoms” (preprint, Bioengineering, 2022), doi: 10.1101/2022.06.17.496531.
9. J. P. Bogen, S. C. Hinz, J. Grzeschik, A. Ebenig, S. Krah, S. Zielonka, H. Kolmar, Dual Function pH Responsive Bispecific Antibodies for Tumor Targeting and Antigen Depletion in Plasma. Front. Immunol. 10, 1892 (2019).
10. L. Ledsgaard, J. Wade, T. P. Jenkins, K. Boddum, I. Oganesyan, J. A. Harrison, P. Villar, R. A. Leah, R. Zenobi, S. Schoffelen, B. Voldborg, A. Ljungars, J. McCafferty, B. Lomonte, J. M. Gutiérrez, A. H. Laustsen, A. Karatt-Vellatt, Discovery and optimization of a broadly-neutralizing human monoclonal antibody against long-chain α-neurotoxins from snakes. Nat Commun. 14, 682 (2023).
11. C. Rimbault, P. D. Knudsen, A. Damsbo, K. Boddum, H. Ali, C. M. Hackney, L. Ellgaard, M.-F. Bohn, A. H. Laustsen, A single-chain variable fragment selected against a conformational epitope of a recombinantly produced snake toxin using phage display. New Biotechnology. 76, 23-32 (2023).
12. J. García-Nafría, J. F. Watson, I. H. Greger, IVA cloning: A single-tube universal cloning system exploiting bacterial In Vivo Assembly. Sci Rep 6, 27459 (2016).
13. C. D. Martin, G. Rojas, J. N. Mitchell, K. J. Vincent, J. Wu, J. McCafferty, D. J. Schofield, A simple vector system to improve performance and utilisation of recombinant antibodies. BMC Biotechnol 6, 46 (2006).

### Items

The invention may further be defined by any one of the following items:

1. A method for isolating antigen-binding proteins having a pH-dependent scaffold, wherein the scaffold consists of the regions of the variable regions not being part of the paratope, said method comprising the steps of:
providing a library comprising antigen-binding proteins, each comprising an antibody light chain variable region (VL), wherein said VLs comprise one or more mutations positioned outside of the paratope of the antigen-binding protein, wherein said library comprises a plurality of antigen-binding proteins containing different mutations in the VL;
selecting antigen-binding proteins that display higher binding affinity in acidic pH than neutral pH, or lower binding affinity in acidic pH than neutral pH from said library,
thereby isolating antigen-binding proteins having a pH-dependent scaffold.

2. A method of generating a pH-dependent antigen-binding protein binding a specific epitope, said method comprising the steps of:
isolating an antigen-binding protein having a pH dependent scaffold according to the method of item 1;
providing an antigen-binding protein binding said specific epitope;
exchanging the paratope of said antigen-binding protein having a pH dependent scaffold for the paratope of said antigen-binding protein binding said epitope,
   thereby generating a pH dependent antigen-binding protein binding said specific epitope.

3. The method according to any one of the preceding items, wherein one or more antigen-binding proteins of the library further comprises a heavy chain comprising a variable region (VH), wherein said VHs comprise one or more mutations positioned outside of the paratope of the antigen-binding protein.

4. The method according to any one of the preceding items, wherein the one or more mutations are in residue positions located at the interface between the VL and the VH of said antigen binding protein.

5. The method according to any one of the preceding items, wherein the parental sequence of the VLs and/or the parental sequence of the VHs are sequences of an antigen binding protein, which does not have pH-dependent antigen-binding.

6. The method according to any one of the preceding items, wherein the one or more mutations are not positioned in the complementarity-determining-regions (CDRs).

7. The method according to any one of the preceding items, wherein the one or more mutations are in one or more of the frameworks regions (FWRs).

8. The method according to any one of the preceding items, wherein the antigen-binding proteins of the library comprise an antibody light chain variable region (VL), wherein said VL comprises 3 complementarity-determining regions, CDR1, CDR2 and CDR3 surrounded by 4 framework regions FWR1, FWR2, FWR3 and FWR4, wherein at least one of said FWR1, FWR2, FWR3 and FWR4 contains one or more mutations compared to a parental FWR1, FWR2, FWR3 and FWR4.

9. The method according to item 8, wherein the antigen-binding proteins of the library comprise a VL FWR1 of SEQ. ID NO: 26, SEQ. ID NO: 151, or SEQ. ID NO: 163, except that 1 to 2 amino acids are mutated.

10. The method according to any one of items 8 to 9, wherein the antigen-binding proteins of the library comprise a VL FWR2 of SEQ. ID NO: 27, SEQ. ID NO: 152, SEQ. ID NO: 158 or SEQ. ID NO: 164, except that 1 to 2 amino acids are mutated.

11. The method according to item 8, wherein the antigen-binding proteins of the library comprise a VL FWR2 of SEQ. ID NO: 27except that 1 to 2 amino acids are mutated.

12. The method according to any one of items 8 to 11, wherein the antigen-binding proteins of the library comprise a VL FWR3 of SEQ. ID NO: 28, SEQ. ID NO: 153, SEQ. ID NO: 159 or SEQ. ID NO: 165, except that 1 to 2 amino acids are mutated.

13. The method according to item 8 or 11, wherein the antigen-binding proteins of the library comprise a VL FWR3 of SEQ. ID NO: 28 except that 1 to 2 amino acids are mutated.

14. The method according to any one of items 8 to 13, wherein the antigen-binding proteins of the library comprise a VL FWR4 of SEQ. ID NO: 29 or SEQ. ID NO: 154, except that 1 to 2 amino acids are mutated.

15. The method according to item 8,12 or 12, wherein the antigen-binding proteins of the library comprise a VL FWR4 of SEQ. ID NO: 29 except that 1 to 2 amino acids are mutated.

16. The method according to any one of the preceding items, wherein the antigen-binding proteins of the library comprise an antibody heavy chain variable region (VH), wherein said VH comprises 3 complementarity-determining regions, CDR1, CDR2 and CDR3 surrounded by 4 framework regions FWR1, FWR2, FWR3 and FWR4, wherein at least one of said FWR1, FWR2, FWR3 and FWR4 contains one or more mutations compared to a parental FWR1, FWR2, FWR3 and FWR4.

17. The method according to item 16, wherein the antigen-binding proteins of the library comprise a VH FWR1 of SEQ. ID NO: 30, SEQ. ID NO: 155, SEQ. ID NO: 160 or SEQ. ID NO: 166, except that 1 to 2 amino acids are mutated.

18. The method according to any one of items 16 to 17, wherein the antigen-binding proteins of the library comprise a VH FWR2 of SEQ. ID NO: 31, SEQ. ID NO: 156, SEQ. ID NO: 161 or SEQ. ID NO: 167, except that 1 to 2 amino acids are mutated.

19. The method according to item 16, wherein the antigen-binding proteins of the library comprise a VH FWR2 of SEQ. ID NO: 31 except that 1 to 2 amino acids are mutated.

20. The method according to any one of items 16 to 19, wherein the antigen-binding proteins of the library comprise a VH FWR3 of SEQ. ID NO: 32, SEQ. ID NO: 157, SEQ. ID NO: 162 or SEQ. ID NO: 168, except that 1 to 2 amino acids are mutated.

21. The method according to item 16 or 19, wherein the antigen-binding proteins of the library comprise a VH FWR3 of SEQ. ID NO: 32 except that 1 to 2 amino acids are mutated.

22. The method according to any one of items 16 to 21, wherein the antigen-binding proteins of the library comprise a VH FWR4 of SEQ. ID NO: 33 except that 1 to 2 amino acids are mutated.

23. The method according to any one of the preceding items, wherein the one or more mutations are not at residue positions occupied by Histidine residues.

24. The method according to any one of the preceding items, wherein the one or more mutations are not mutations to Histidine residues.

25. The method according to any one of the preceding items, wherein the one or more mutations are at residue positions at least 1 amino acid away from an histidine residue, such as at least 2 amino acids, for example at least 5 amino acids, such as at least 8 amino acids, for example at least 10 amino acids, such as at least 15 amino acids, for example at least 20 amino acids, such as at least 25 amino acids, for example at least 50 amino acids away from an histidine residue.

26. The method according to any one of the preceding items, wherein the one or more mutations are at VH residues 39, 44, 45, 47, 89, 91, 103 or 105 according to Kabat numbering.

27. The method according to any one of the preceding items, wherein the one or more mutations are at VH residues selected from the group consisting of: 39, 44, 89, and 105 according to Kabat numbering.

28. The method according to any one of the preceding items, wherein the one or more mutations are at VH residues selected from the group consisting of: Q39, G44, V89, and Q105 according to Kabat numbering.

29. The method according to any one of the preceding items, wherein the one or more mutations are at VL residues 32, 36, 38, 43, 44, 46, 49, 50, 85, 87, 98 or 100 according to Kabat numbering.

30. The method according to any one of the preceding items, wherein the one or more mutations are at VL residue positions selected from the group consisting of: 38, 43, 85, and 100, according to Kabat numbering.

31. The method according to any one of the preceding items, wherein the one or more mutations are at VL residues selected from the group consisting of: Q38, S43, D85, and G100 according to Kabat numbering.

32. The method according to any one of the preceding items, wherein the antigen-binding proteins of the library comprise:
a) a VH FWR2 of SEQ ID NO:31
b) a VH FWR3 of SEQ ID NO:32
c) a VH FWR4 of SEQ ID NO:33
and
d) a VL FWR2 of SEQ. ID NO: 27
e) a VL FWR3 of SEQ. ID NO: 28
f) a VL FWR4 of SEQ. ID NO: 29,
wherein one or more mutations are introduced in residue position VH 39, VH 44, VH 89, VH 105, VL 38, VL 43, VL 85 and/or VL 100 according to Kabat numbering.

33. The method according to any one of the preceding items, wherein the antigen-binding proteins of the library comprise:
a) a VH FWR2 of SEQ ID NO: 156
b) a VH FWR3 of SEQ ID NO: 157
c) a VH FWR4 of SEQ ID NO:33
and
d) a VL FWR2 of SEQ. ID NO: 152
e) a VL FWR3 of SEQ. ID NO: 153
f) a VL FWR4 of SEQ. ID NO: 154,
wherein one or more mutations are introduced in residue position VH 39, VH 44, VH 45, VH 47, VH 89, VH 91, VH 103, VH 105, VL 32, VL 36, VL 38, VL 43, VL 44, VL 46, VL49, VL 50, VL 85 VL 87, VL 98 and/or VL 100 according to Kabat numbering, preferably wherein one or more mutations are introduced in residue position VH 44, VH 89, VH 105, and/or VL 38 according to Kabat numbering.

34. The method according to any one of the preceding items, wherein the antigen-binding proteins of the library comprise:
a) a VH FWR2 of SEQ ID NO: 161
b) a VH FWR3 of SEQ ID NO: 162
c) a VH FWR4 of SEQ ID NO:33
and
d) a VL FWR2 of SEQ. ID NO: 158
e) a VL FWR3 of SEQ. ID NO: 159
f) a VL FWR4 of SEQ. ID NO: 154,
wherein one or more mutations are introduced in residue position VH 39, VH 44, VH 45, VH 47, VH 89, VH 91, VH 103, VH 105, VL 32, VL 36, VL 38, VL 43, VL 44, VL 46, VL49, VL 50, VL 85 VL 87, VL 98 and/or VL 100 according to Kabat numbering, preferably wherein one or more mutations are introduced in residue position VH 44, VH 89, VH 105, and/or VL 38 according to Kabat numbering.

35. The method according to any one of the preceding items, wherein the antigen-binding proteins of the library comprise:
a) a VH FWR2 of SEQ ID NO: 164
b) a VH FWR3 of SEQ ID NO: 165
c) a VH FWR4 of SEQ ID NO:33
and
d) a VL FWR2 of SEQ. ID NO: 167
e) a VL FWR3 of SEQ. ID NO: 168
f) a VL FWR4 of SEQ. ID NO: 154,
wherein one or more mutations are introduced in residue position VH 39, VH 44, VH 45, VH 47, VH 89, VH 91, VH 103, VH 105, VL 32, VL 36, VL 38, VL 43, VL 44, VL 46, VL49, VL 50, VL 85 VL 87, VL 98 and/or VL 100 according to Kabat numbering, preferably wherein one or more mutations are introduced in residue position VH 44, VH 89, VH 105, and/or VL 38 according to Kabat numbering.

36. The method according to any one of the preceding items, wherein the step of selecting antigen-binding proteins that display lower KD in acidic pH than neutral pH, or higher KD in acidic pH than neutral pH, from said library comprises using an in vitro display technology.

37. The method according to item 36, wherein the in vitro display technology is selected from the group consisting of: phage display, ribosome display, yeast display, bacterial display, mammalian display, and CIS display.

38. A pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein the pH-dependent antigen-binding protein has higher binding affinity in acidic pH than neutral pH, or lower binding affinity in acidic pH than neutral pH, and wherein said pH-dependent antigen-binding protein does not comprise heavy chain complementarity-determining regions 1, 2 and 3 of SEQ. ID NOs 1, 2, 3 respectively and light chain complementarity-determining region 1, 2 and 3 of SEQ. ID NOs 4, 5, 6 respectively.

39. A pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein the pH-dependent antigen-binding protein has higher binding affinity in acidic pH than neutral pH, or lower binding affinity in acidic pH than neutral pH, and wherein said pH-dependent antigen-binding protein does not comprise heavy chain complementarity-determining regions 1, 2 and 3 of any of:
SEQ. ID NOs 1, 2, 3 respectively,
SEQ. ID NOs 178, 179, 180 respectively,
SEQ. ID NOs 181, 182, 183 respectively, and
SEQ. ID NOs 184, 185, 186 respectively,
and light chain complementarity-determining region 1, 2 and 3 of any of:
SEQ. ID NOs 4, 5, 6 respectively,
SEQ. ID NOs 169, 170, 171 respectively,
SEQ. ID NOs 172, 173, 174 respectively and
SEQ. ID NOs 175, 176, 177 respectively.

40. A pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein the pH-dependent antigen-binding protein has higher binding affinity in acidic pH than neutral pH, and wherein said pH-dependent antigen-binding protein does not comprise heavy chain complementarity-determining regions 1, 2 and 3 of any of:
SEQ. ID NOs 1, 2, 3 respectively,
SEQ. ID NOs 178, 179, 180 respectively,
SEQ. ID NOs 181, 182, 183 respectively, and
SEQ. ID NOs 184, 185, 186 respectively,
and light chain complementarity-determining region 1, 2 and 3 of any of:
SEQ. ID NOs 4, 5, 6 respectively,
SEQ. ID NOs 169, 170, 171 respectively,
SEQ. ID NOs 172, 173, 174 respectively and
SEQ. ID NOs 175, 176, 177 respectively.

41. The pH-dependent antigen-binding protein according to any one of items 38 to 40, wherein the pH-dependent antigen-binding protein is as defined in any one of items 1 to 37.

42. The pH-dependent binding protein according to any one of items 38 to 41, wherein the one or more mutations are at residue positions located at the interface between the VL and the VH of the pH-dependent antigen binding protein.

43. The pH-dependent antigen-binding protein according to any one of items 38 to 42, wherein the mutations are not at residue positions occupied by Histidine residues.

44. The pH-dependent antigen-binding protein according to any one of items 38 to 43, wherein the one or more mutations are not mutations to Histidine residues.

45. The pH-dependent antigen-binding protein according to any one of items 38 to 44 wherein the one or more mutations are at residue positions at least 1 amino acid away from an histidine residue, such as at least 2 amino acids, for example at least 5 amino acids, such as at least 8 amino acids, for example at least 10 amino acids, such as at least 15 amino acids, for example at least 20 amino acids, such as at least 25 amino acids, for example at least 50 amino acids away from an histidine residue.

46. The pH-dependent antigen-binding protein according to any one of items 38 to 45, wherein the one or more mutations are at VH residues selected from the group consisting of: 39, 44, 89, and 105 according to Kabat numbering, preferably wherein the mutations are at VH residues selected from the group consisting of 44, 89, and 105.

47. The pH-dependent antigen-binding protein according to any one of items 38 to 46, wherein the one or more mutations are at VH residues selected from the group consisting of: 45, 47, 91, and 103 according to Kabat numbering.

48. The pH-dependent antigen-binding protein according to any one of items 38 to 47, wherein the one or more mutations are at VH residues selected from the group consisting of: L45, W47, Y91, and W103

49. The pH-dependent antigen-binding protein according to any one of items 38 to 48, wherein the one or more mutations are at VL residue positions selected from the group consisting of: 38, 43, 85, and 100, according to Kabat numbering, preferably wherein the mutation is at VL residue position 38.

50. The pH-dependent antigen-binding protein according to any one of items 38 to 49 wherein the one or more mutations are at VL residue positions selected from the group consisting of: 32, 46, and 49 according to Kabat numbering.

51. The pH-dependent antigen-binding protein according to any one of items 38 to 50, wherein the one or more mutations are at VL residue positions selected from the group consisting of: 46 and 49 according to Kabat numbering.

52. The pH-dependent antigen-binding protein according to any one of items 38 to 51, wherein the one or more mutations are at VL residue positions selected from the group consisting of: 36, 44, 87 and 98 according to Kabat numbering.

53. The pH-dependent antigen-binding protein according to any one of items 38 to 52, wherein the one or more mutations are at VL residues selected from the group consisting of: Y32, T46, and Y49 according to Kabat numbering.

54. The pH-dependent antigen-binding protein according to any one of items 38 to 53, wherein the one or more mutations are at VL residues selected from the group consisting of: T46, and Y49 according to Kabat numbering.

55. The pH-dependent antigen-binding protein according to any one of items 38 to 54, wherein the one or more mutations are at VL residues selected from the group consisting of: Y36, P44, Y87, and F98 according to Kabat numbering.

56. The pH-dependent antigen-binding protein according to any one of items 38 to 55, wherein the one or more mutations are at VH residues selected from the group consisting of: 39, 44, 45, 47, 89, 91, 103 and 105 according to Kabat numbering.

57. The pH-dependent antigen-binding protein according to any one of items 38 to 56, wherein the VH contains one or more, such as two, such as three, such as four, of the following amino acid residues:
an S, E, R, T, or A at position 39,
a P, R, N, S, K, Q, A, Y, or T at position 44,
a T, N, I, Q, A, L, Y, D, F, S, or K at position 89, and/or
a T, R, K, P, D, I, S, or T at position 105, wherein all positions are indicated according to Kabat numbering.

58. The pH-dependent antigen-binding protein according to any one of items 38 to 57, wherein the VH contains one or more, such as two, such as three, such as four, of the following amino acid residues:
an S, E, or A at position 39,
a P, R, N, S, K, Q, A, or T at position 44,
a T, N, I, Q, A, L, Y, D, F, S, or K at position 89, and/or
a T, R, K, P, D, I, S, or T at position 105, wherein all positions are indicated according to Kabat numbering.

59. The pH-dependent antigen-binding protein according to any one of items 38 to 58, wherein the one or more mutations are at VL residues selected from the group consisting of: 32, 36, 38, 43, 44, 46, 49, 85, 87, 98 and 100 according to Kabat numbering.

60. The pH-dependent antigen-binding protein according to any one of items 38 to 59, wherein the one or more mutations are at VL residues selected from the group consisting of: 36, 38, 43, 44, 46, 49, 85, 87, 98 and 100 according to Kabat numbering.

61. The pH-dependent antigen-binding protein according to any one of items 38 to 60, wherein the VL contains one or more, such as two, such as three, such as four, such as five, such as six, such as seven, of the following amino acid residues:
an N at position 36,
a L, Y, S, I, T, A, R, F, or V at position 38,
a P, T, A, R, Q, K, or V at position 43
an A, or S at position 46,
an A, H, or S at position 49,
an S, N, T, F, V, L, S, A, H, or R at position 85, and/or
an S, Y, W, or L at position 100, wherein all positions are indicated according to Kabat numbering.

62. The pH-dependent antigen-binding protein according to any one of items 38 to 61, wherein the VL contains one or more, such as two, such as three, such as four, such as five, such as six, such as seven, of the following amino acid residues:
an N at position 36,
a L, Y, S, I, T, A, R, F, or V at position 38,
a P, T, A, or V at position 43
an A, or S at position 46,
an A, H, or S at position 49,
an S, N, T, F, V, L, S, A, or R at position 85, and/or
an S, Y, or L at position 100, wherein all positions are indicated according to Kabat numbering.

63. The pH-dependent antigen-binding protein according to any one of items 38 to 62, wherein the one or more mutations are at VH residues selected from the group consisting of: Q39, G44, L45, W47, V89, Y91, W103 and Q105 according to Kabat numbering.

64. The pH-dependent antigen-binding protein according to any one of items 38 to 63, wherein the one or more mutations are at VL residues selected from the group consisting of: Y32, Y36, Q38, D43, P44, T46, Y49, D85, Y87, F98 and G100 according to Kabat numbering.

65. The pH-dependent antigen-binding protein according to any one of items 38 to 64, wherein the one or more mutations are at VL residues selected from the group consisting of: Y36, Q38, D43, P44, T46, Y49, D85, Y87, F98 and G100 according to Kabat numbering.

66. The pH-dependent antigen-binding protein according to any one of items 38 to 65, wherein the antigen-binding protein comprises:
a) a VH FWR2 of SEQ ID NO:31
b) a VH FWR3 of SEQ ID NO:32
c) a VH FWR4 of SEQ ID NO:33
and
d) a VL FWR2 of SEQ. ID NO: 27
e) a VL FWR3 of SEQ. ID NO: 28
f) a VL FWR4 of SEQ. ID NO: 29 wherein one or more mutations are introduced in residue positions selected from the group consisting of: VH 39, VH 44, VH 89, VH 105, VL 38, VL 43, VL 85 and VL 100 according to Kabat numbering.

67. The pH-dependent antigen-binding protein according to any one of items 38 to 66, wherein the antigen-binding protein comprises:
a) a VH FWR2 of SEQ ID NO:31
b) a VH FWR3 of SEQ ID NO:32
c) a VH FWR4 of SEQ ID NO:33
and
d) a VL FWR2 of SEQ. ID NO: 27
e) a VL FWR3 of SEQ. ID NO: 28
f) a VL FWR4 of SEQ. ID NO: 29
wherein one or more mutations are introduced in residue positions selected from the group consisting of: VH 45, VH 47, VH 91, VH 103, VL 36, VL 44, VL 87, VL 98 according to Kabat numbering.

68. The pH-dependent antigen-binding protein according to any one of items 38 to 67, wherein the antigen-binding protein comprises:
a) a VH FWR2 of SEQ ID NO:31
b) a VH FWR3 of SEQ ID NO:32
c) a VH FWR4 of SEQ ID NO:33
and
d) a VL FWR2 of SEQ. ID NO: 27
e) a VL FWR3 of SEQ. ID NO: 28
f) a VL FWR4 of SEQ. ID NO: 29
wherein one or more mutations are introduced in residue positions selected from the group consisting of: VL 32, VL 46, VL 49 according to Kabat numbering.

69. The pH-dependent antigen-binding protein according to any one of items 38 to 68, wherein the antigen-binding protein comprises:
a) a VH FWR2 of SEQ ID NO:31
b) a VH FWR3 of SEQ ID NO:32
c) a VH FWR4 of SEQ ID NO:33
and
d) a VL FWR2 of SEQ. ID NO: 27
e) a VL FWR3 of SEQ. ID NO: 28
f) a VL FWR4 of SEQ. ID NO: 29
wherein one or more mutations are introduced in residue positions selected from the group consisting of: VL 46, VL 49 according to Kabat numbering.

70. The pH-dependent antigen-binding protein according to any one of items 38 to 69, wherein the antigen-binding protein comprises:
a) a VH FWR2 of SEQ ID NO:31
b) a VH FWR3 of SEQ ID NO:32
c) a VH FWR4 of SEQ ID NO:33
and
d) a VL FWR2 of SEQ. ID NO: 27
e) a VL FWR3 of SEQ. ID NO: 28
f) a VL FWR4 of SEQ. ID NO: 29
wherein one or more mutations are introduced in residue positions selected from the group consisting of: VH 39, VH 44, VH 45, VH 89, VH 91, VH 103, VH 105, VL 32, VL 36, VL 38, VL 43, VL 46, VL 49, VL 85, VL 87, VL 98 and VL 100 according to Kabat numbering.

71. The pH-dependent antigen-binding protein according to any one of items 38 to 70, wherein the antigen-binding protein comprises:
a) a VH FWR2 of SEQ ID NO:31
b) a VH FWR3 of SEQ ID NO:32
c) a VH FWR4 of SEQ ID NO:33
and
d) a VL FWR2 of SEQ. ID NO: 27
e) a VL FWR3 of SEQ. ID NO: 28
f) a VL FWR4 of SEQ. ID NO: 29
wherein one or more mutations are introduced in residue positions selected from the group consisting of: VH 39, VH 44, VH 45, VH 89, VH 91, VH 103, VH 105, VL 36, VL 38, VL 43, VL 46, VL 49, VL 85, VL 87, VL 98 and VL 100 according to Kabat numbering.

72. The pH-dependent antigen-binding protein according to any one of items 38 to 71, wherein the antigen-binding protein comprises:
a) a VH FWR2 of SEQ ID NO: 156
b) a VH FWR3 of SEQ ID NO: 157
c) a VH FWR4 of SEQ ID NO:33
and
d) a VL FWR2 of SEQ. ID NO: 152
e) a VL FWR3 of SEQ. ID NO: 153
f) a VL FWR4 of SEQ. ID NO: 154,
wherein one or more mutations are introduced in residue position VH 39, VH 44, VH 45, VH 47, VH 89, VH 91, VH 103, VH 105, VL 32, VL 36, VL 38, VL 43, VL 44, VL 46, VL49, VL 50, VL 85 VL 87, VL 98 and/or VL 100 according to Kabat numbering, preferably wherein one or more mutations are introduced in residue position VH 44, VH 89, VH 105, and/or VL 38 according to Kabat numbering.

73. The pH-dependent antigen-binding protein according to any one of items 38 to 72, wherein the antigen-binding protein comprises:
a) a VH FWR2 of SEQ ID NO: 161
b) a VH FWR3 of SEQ ID NO: 162
c) a VH FWR4 of SEQ ID NO:33
and
d) a VL FWR2 of SEQ. ID NO: 158
e) a VL FWR3 of SEQ. ID NO: 159
f) a VL FWR4 of SEQ. ID NO: 154,
wherein one or more mutations are introduced in residue position VH 39, VH 44, VH 45, VH 47, VH 89, VH 91, VH 103, VH 105, VL 32, VL 36, VL 38, VL 43, VL 44, VL 46, VL49, VL 50, VL 85 VL 87, VL 98 and/or VL 100 according to Kabat numbering, preferably wherein one or more mutations are introduced in residue position VH 44, VH 89, VH 105, and/or VL 38 according to Kabat numbering.

74. The pH-dependent antigen-binding protein according to any one of items 38 to 73, wherein the antigen-binding protein comprises:
a) a VH FWR2 of SEQ ID NO: 164
b) a VH FWR3 of SEQ ID NO: 165
c) a VH FWR4 of SEQ ID NO:33
and
d) a VL FWR2 of SEQ. ID NO: 167
e) a VL FWR3 of SEQ. ID NO: 168
f) a VL FWR4 of SEQ. ID NO: 154,
wherein one or more mutations are introduced in residue position VH 39, VH 44, VH 45, VH 47, VH 89, VH 91, VH 103, VH 105, VL 32, VL 36, VL 38, VL 43, VL 44, VL 46, VL49, VL 50, VL 85 VL 87, VL 98 and/or VL 100 according to Kabat numbering, preferably wherein one or more mutations are introduced in residue position VH 44, VH 89, VH 105, and/or VL 38 according to Kabat numbering.

75. The pH-dependent antigen-binding protein according to any one of items 38 to 74, wherein the antigen-binding protein comprises:
a) a VH FWR2 of WRX₁APGQX₂X₃EX₄MG (SEQ ID NO:98)
b) a VH FWR3 of RVTITADX₅STSTAYMX₆LX₇SLRSDDTAX₈YX₉CAR (SEQ ID NO:99)
c) a VH FWR4 of X₁₀GX₁₁GTLVTVSS (SEQ ID NO: 100),
wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
X₁ is any amino acid except Q, preferably wherein X₁ is S, E, or A, and/or
X₂ is any amino acid except G, preferably wherein X₂ is P, R, N, S, K, Q, A, or T, and/or
X₈ is any amino acid except V, preferably wherein X₈ is T, N, I, Q, A, L, or K, and/or
X₁₁ is any amino acid except Q, more preferably wherein X₁₁ is T, R, K, P, D, I, or T;
and
d) a light chain CDR1 of TRSX₁GSIGSDX₂VH (SEQ. ID NO: 104)
e) a VL FWR2 of WX₃QX₄RPGSX₅X₆TX₇VIX₈ (SEQ. ID NO: 101)
f) a VL FWR3 of GVPDRFSGSIDSSSNSASLTISGLKTEDEAX₉YX₁₀C (SEQ. ID NO: 102)
g) a VL FWR4 of X₁₁GX₁₂GTKLTVX₁₃ (SEQ. ID NO: 103),
wherein each of X₁, X₂, X₃, X₄, X₅, X₆, X₇, X₈, X₉, X₁₀, X₁₁, X₁₂, X₁₃ may be any amino acid, with the proviso that at least one of X₂, X₄, X₅, X₇, X₈, X₉, X₁₂ is selected from:
X₂ is any amino acid except Y, preferably wherein X₂ is A, S, T, or H, and/or
X₄ is any amino acid except Q, preferably wherein X₄ is L, Y, S, I, T, A, R, or V, and/or
X₅ is any amino acid except S, preferably wherein X₅ is P, T, A, or V, and/or
X₇ is any amino acid except T preferably wherein X₇ is A, or S, and/or
X₈ is any amino acid except Y, preferably wherein X₈ is A, H, or S, and/or
X₉ is any amino acid except D, preferably wherein X₆ is S, N, T, F, V, L, S, A or R, and/or
X₁₂ is any amino acid except G, preferably wherein X₁₂ is S, Y, or L
76. The pH-dependent antigen-binding protein according to any one of items 38 to 75, wherein the antigen-binding protein comprises:
   a) a VH FWR2 of WVRX₁APGQX₂X₃EX₄MG (SEQ ID NO:98)
   b) a VH FWR3 of RVTITADX₅STSTAYMX₆LX₇SLRSDDTAX₈YX₉CAR (SEQ ID NO:99)
   c) a VH FWR4 of X₁₀GX₁₁GTLVTVSS (SEQ ID NO: 100),
   wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
   X₁ is any amino acid except Q, preferably wherein X₁ is S, E, or A, and/or
   X₂ is any amino acid except G, preferably wherein X₂ is P, R, N, S, K, Q, A, or T, and/or
   X₈ is any amino acid except V, preferably wherein X₈ is T, N, I, Q, A, L, or K, and/or
   X₁₁ is any amino acid except Q, more preferably wherein X₁₁ is T, R, K, P, D, I, or T;
   and
   d) a light chain CDR1 of TRSX₁GSIGSDX₂VH (SEQ. ID NO: 104)
   e) a VL FWR2 of WX₃QX₄RPGSX₅X₆TX₇VIX₈ (SEQ. ID NO: 101)
   f) a VL FWR3 of GVPDRFSGSIDSSSNSASLTISGLKTEDEAX₉YX₁₀C (SEQ. ID NO: 102)
   g) a VL FWR4 of X₁₁GX₁₂GTKLTVX₁₃ (SEQ. ID NO: 103),
   wherein each of X₁, X₂, X₃, X₄, X₅, X₆, X₇, X₈, X₉, X₁₀, X₁₁, X₁₂, X₁₃ may be any amino acid, with the proviso that at least one of X₂, X₃, X₄, X₅, X₇, X₈, X₉, X₁₂ is selected from:
   X₂ is any amino acid except Y, preferably wherein X₂ is A, S, T, or H, and/or
   X₃ is any amino acid except Y, preferably wherein X₃ is N, and/or
   X₄ is any amino acid except Q, preferably wherein X₄ is L, Y, S, I, T, A, R, or V, and/or
   X₅ is any amino acid except S, preferably wherein X is P, T, A, or V, and/or
   X₇ is any amino acid except T preferably wherein X₇ is A, or S, and/or
   X₈ is any amino acid except Y, preferably wherein X₈ is A, H, or S, and/or
   X₆ is any amino acid except D, preferably wherein X₆ is S, N, T, F, V, L, S, A or R, and/or
   X₁₂ is any amino acid except G, preferably wherein X₁₂ is S, Y, or L.

77. The pH-dependent antigen-binding protein according to any one of items 38 to 76, wherein the antigen-binding protein comprises:
a) a VH FWR2 of WRX APGQX₂X₃EX₄MG (SEQ ID NO:98)
b) a VH FWR3 of RVTITADX₅STSTAYMX₆LX₇SLRSDDTAX₈YX₉CAR (SEQ ID NO:99)
c) a VH FWR4 of X₁₀GX₁₁GTLVTVSS (SEQ ID NO:100),
wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
X₁ is any amino acid except Q, preferably wherein X₁ is S, E, or A, and/or
X₂ is any amino acid except G, preferably wherein X₂ is P, R, N, S, K, Q, A, or T, and/or
X₈ is any amino acid except V, preferably wherein X₈ is T, N, I, Q, A, L, or K, and/or
X₁₁ is any amino acid except Q, preferably wherein X₁₁ is T, R, K, P, D, I, or T;
and
d) a VL FWR2 of WX₁QX₂RPGSX₃X₄TX₅VIX₆ (SEQ. ID NO: 101)
e) a VL FWR3 of GVPDRFSGSIDSSSNSASLTISGLKTEDEAX₇YX₈C (SEQ. ID NO: 102)
f) a VL FWR4 of X₉GX₁₀GTKLTVX₁₁ (SEQ. ID NO: 103), wherein each of X₁, X₂, X₃, X₄, X₅, X₆, X₇, X₈, X₉, X₁₀, X₁₁ may be any amino acid, with the proviso that at least one of X₂, X₃, X₅, X₆, X₇, X₁₀ is selected from:
X₂ is any amino acid except Q, preferably wherein X₂ is L, Y, S, I, T, A, R, or V, and/or
X₃ is any amino acid except S, preferably wherein X₃ is P, T, A, or V, and/or
X₅ is any amino acid except T preferably wherein X₅ is A, or S, and/or
X₆ is any amino acid except Y, preferably wherein X₆ is A, H, or S, and/or
X₇ is any amino acid except D, preferably wherein X₇ is S, N, T, F, V, L, S, A or R, and/or
X₁₀ is any amino acid except G, preferably wherein X₁₀ is S, Y, or L.

78. The pH-dependent antigen-binding protein according to any one of items 38 to 77, wherein the antigen-binding protein comprises:
a) a VH FWR2 of WVRXIAPGQX₂X₃EX₄MG (SEQ ID NO:98)
b) a VH FWR3 of RVTITADX₅STSTAYMX₆LX₇SLRSDDTAX₈YX₉CAR (SEQ ID NO:99)
c) a VH FWR4 of X₁₀GX₁₁GTLVTVSS (SEQ ID NO: 100), wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
X₁ is any amino acid except Q, preferably wherein X₁ is S, E, or A, and/or
X₂ is any amino acid except G, preferably wherein X₂ is P, R, N, S, K, Q, A, or T, and/or
X₈ is any amino acid except V, preferably wherein X₈ is T, N, I, Q, A, L, or K, and/or
X₁₁ is any amino acid except Q, preferably wherein X₁₁ is T, R, K, P, D, I, or T;
and
d) a VL FWR2 of WX₁QX₂RPGSX₃X₄TX₅VIX₆ (SEQ. ID NO: 101)
e) a VL FWR3 of GVPDRFSGSIDSSSNSASLTISGLKTEDEAX₇YX₈C (SEQ. ID NO: 102)
f) a VL FWR4 of X₉GX₁₀GTKLTVX₁₁ (SEQ. ID NO: 103), wherein each of X₁, X₂, X₃, X₄, X₅, X₆, X₇, X₈, X₉, X₁₀, X₁₁ may be any amino acid, with the proviso that at least one of X₁, X₂, X₃, X₅, X₆, X₇, X₁₀ is selected from:
X₁ is any amino acid except Y, preferably wherein X₁ is N, and/or
X₂ is any amino acid except Q, preferably wherein X₂ is L, Y, S, I, T, A, R, or V, and/or
X₃ is any amino acid except S, preferably wherein X₃ is P, T, A, or V, and/or
X₅ is any amino acid except T preferably wherein X₅ is A, or S, and/or
X₆ is any amino acid except Y, preferably wherein X₆ is A, H, or S, and/or
X₇ is any amino acid except D, preferably wherein X₇ is S, N, T, F, V, L, S, A or R, and/or
X₁₀ is any amino acid except G, preferably wherein X₁₀ is S, Y, or L.

79. The pH-dependent antigen-binding protein according to any one of items 38 to 78, wherein the antigen-binding protein comprises:
a) a VH FWR2 of WVRX₁APGKX₂X₃EX₄VS (SEQ ID NO:187)
b) a VH FWR3 of RFTISRDX₅AKNSLYLX₆MX₇SLRAEDTAX₈YX₉CAK (SEQ ID NO: 188)
c) a VH FWR4 of X₁₀GX₁₁GTLVTVSS (SEQ ID NO: 100),
wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
X₁ is any amino acid except Q, preferably wherein X₁ is S, E, or A, and/or
X₂ is any amino acid except G, preferably wherein X₂ is P, R, N, S, K, Q, A, Y or T, even more preferably wherein X₂ is Y, R, or T, and/or
X₈ is any amino acid except V, preferably wherein X₈ is T, N, I, Q, A, L, Y, D, F, S or K, even more preferably wherein X₈ is Y, D, F, or S and/or
X₁₁ is any amino acid except Q, preferably wherein X₁₁ is T, R, K, P, D, I, S or T, even more preferably wherein X₁₁ is T, S, or P;
and
d) a VL FWR2 of WX₁QX₂KPGKX₃X₄KX₅LIX₆ (SEQ. ID NO: 190)
e) a VL FWR3 of GVPSRFSGSGSGTDFTLTISSLQPEDVAX₇YX₈C (SEQ. ID NO: 191)
f) a VL FWR4 of X₉GX₁₀GTKVEIX₁₁ (SEQ. ID NO: 192),
wherein each of X₁, X₂, X₃, X₄, X₅, X₆, X₇, X₈, X₉, X₁₀, X₁₁ may be any amino acid, with the proviso that at least one of X₁, X₂, X₃, X₅, X₆, X₇, X₁₀ is selected from:
X₁ is any amino acid except Y, preferably wherein X₁ is N, and/or
X₂ is any amino acid except Q, preferably wherein X₂ is L, Y, S, I, T, A, R, F, or V, even more preferably wherein X₂ is S, R, or L, and/or
X₃ is any amino acid except A, preferably wherein X₃ is P, T, or V, and/or
X₅ is any amino acid except L preferably wherein X₅ is A, or S, and/or
X₆ is any amino acid except Y, preferably wherein X₆ is A, H, or S, and/or
X₇ is any amino acid except T, preferably wherein X₇ is N, F, V, L, S, A or R, and/or
X₁₀ is any amino acid except Q, preferably wherein X₁₀ is S, Y, or L.

80. The pH-dependent antigen-binding protein according to any one of items 38 to 79, wherein the antigen-binding protein comprises:
a) a VH FWR2 of WVRQAPGKX₁LEWVS (SEQ ID NO: 193)
b) a VH FWR3 of RFTISRDNAKNSLYLQMNSLRAEDTAX₂YYCAK (SEQ ID NO:194)
c) a VH FWR4 of WGX₃GTLVTVSS (SEQ ID NO:195),
and
d) a VL FWR2 of WYQX₄KPGKAPKLLIY (SEQ. ID NO: 196)
wherein each of X₁, X₂, X₃, X₄ may be any amino acid, with the proviso that at least one of X₁, X₂, X₃, X₄ is selected from:
X₁ is any amino acid except G, preferably wherein X₁ is P, R, N, S, K, Q, A, Y or T, even more preferably wherein X₁ is Y, R, or T, and/or
X₂ is any amino acid except V, preferably wherein X₂ is preferably wherein X₂ is T, N, I, Q, A, L, Y, D, F, S or K, even more preferably wherein X₂ is Y, D, F or S, and/or
X₃ is any amino acid except Q, preferably wherein X₃ is T, R, K, P, D, I, S or T, even more preferably wherein X₃ is S, T or P, and/or
X₄ is any amino acid except Q, preferably wherein X₄ is L, Y, S, I, T, A, R, F, or V, even more preferably wherein X₄ is R, S, or L.

81. The pH-dependent antigen-binding protein according to any one of items 38 to 80, wherein the antigen-binding protein comprises:
a) a VH FWR2 of WVRX₁APGKX₂X₃EX₄VG (SEQ ID NO:197)
b) a VH FWR3 of RFTFSLDX₅SKSTAYLX₆MX₇SLRX₈EDTAX₉YX₁₀CAK (SEQ ID NO: 198)
c) a VH FWR4 of X₁₁GX₁₂GTLVTVSS (SEQ ID NO:189),
wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
X₁ is any amino acid except Q, preferably wherein X₁ is S, E, or A, and/or
X₂ is any amino acid except G, preferably wherein X₂ is P, R, N, S, K, Q, A, or T, even more preferably wherein X₂ is A, P, S, or T, and/or
X₈ is any amino acid except A, preferably wherein X₈ is T, and/or
X₉ is any amino acid except V, preferably wherein X₈ is T, N, I, Q, A, L, or K, even more preferably wherein X₈ is A, T, I, or L, and/or
X₁₂ is any amino acid except Q, preferably wherein X₁₁ is T, R, K, P, D, I, S, or T, even more preferably wherein X₁₁ is T, D, S, or P;
and
d) a VL FWR2 of WX₁QX₂KPGKX₃X₄KX₅LIX₆ (SEQ. ID NO: 190)
e) a VL FWR3 of GVPSRFSGSGSGTDFTLTISSLQPEDFAX₇YX₈C (SEQ. ID NO: 199)
f) a VL FWR4 of X₉GX₁₀GTKVEIX₁₁ (SEQ. ID NO: 192), wherein each of X₁, X₂, X₃, X₄, X₅, X₆, X₇, X₈, X₉, X₁₀, X₁₁ may be any amino acid, with the proviso that at least one of X₁, X₂, X₃, X₅, X₆, X₇, X₁₀ is selected from:
X₁ is any amino acid except Y, preferably wherein X₁ is N, and/or
X₂ is any amino acid except Q, preferably wherein X₂ is L, Y, S, I, T, A, R, F, or V, even more preferably wherein X₂ is F, L, S, T, or I, and/or
X₃ is any amino acid except A, preferably wherein X₃ is P, T, or V, and/or
X₅ is any amino acid except V preferably wherein X₅ is A, or S, and/or
X₆ is any amino acid except Y, preferably wherein X₆ is A, H, or S, and/or
X₇ is any amino acid except T, preferably wherein X₇ is S, N, F, V, L, A or R, and/or
X₁₀ is any amino acid except Q, preferably wherein X₁₀ is S, Y, or L.

82. The pH-dependent antigen-binding protein according to any one of items 38 to 81, wherein the antigen-binding protein comprises:
a) a VH FWR2 of WVRQAPGKX₁LEWVG (SEQ ID NO:200)
b) a VH FWR3 of RFTFSLDTSKSTAYLQMNSLRX₂EDTAX₃YYCAK (SEQ ID NO:201)
c) a VH FWR4 of WGX₄GTLVTVSS (SEQ ID NO:195),
and
d) a VL FWR2 of WYQX₅KPGKAPKVLIY (SEQ. ID NO: 202)
wherein each of X₁, X₂, X₃, X₄, may be any amino acid, with the proviso that at least one of X₁, X₂, X₃, X₄, is selected from:
X₁ is any amino acid except G, preferably wherein X₁ is P, R, N, S, K, Q, A, Y or T, even more preferably wherein X₁ is A, P, S, or T, and/or
X₂ is any amino acid except A, preferably wherein X₈ is T, and/or
X₃ is any amino acid except V, preferably wherein X₈ is T, N, I, Q, A, L, Y, D, F, S or K preferably wherein X₂ is A, T, I or L, and/or
X₄ is any amino acid except Q, preferably wherein X₃ is T, R, K, P, D, I, S or T preferably wherein X₃ is T, D, S, or P, and/or
X₅ is any amino acid except Q, preferably wherein X₄ is L, Y, S, I, T, A, R, F or V, even more preferably wherein X₄ is F, L, S, T or I.

83. The pH-dependent antigen-binding protein according to any one of items 38 to 82, wherein the antigen-binding protein comprises:
a) a VH FWR2 of WVRX₁APGKX₂X₃EX₄MG (SEQ ID NO:203)
b) a VH FWR3 of RVTMTEDX₅STDTAYMX₆LX₇SLRSEDTAX₈YX₉CST (SEQ ID NO:204)
c) a VH FWR4 of X₁₀GX₁₁GTLVTVSS (SEQ ID NO: 189),
wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
X₁ is any amino acid except Q, preferably wherein X₁ is S, E, or A, and/or
X₂ is any amino acid except G, preferably wherein X₂ is P, R, N, S, K, Q, A, or T, and/or
X₈ is any amino acid except V, preferably wherein X₈ is T, N, I, Q, A, L, or K, and/or
X₁₁ is any amino acid except Q, preferably wherein X₁₁ is T, R, K, P, D, I, S, or T;
and
d) a VL FWR2 of WX₁QX₂KPGKX₃X₄KX₅LIX₆ (SEQ. ID NO: 190)
e) a VL FWR3 of GVPSRFSGSGSGTEFTLTISSLQPEDLAX₇YX₈C (SEQ. ID NO: 205)
f) a VL FWR4 of X₉GX₁₀GTKVEIX₁₁ (SEQ. ID NO: 192),
wherein each of X₁, X₂, X₃, X₄, X₅, X₆, X₇, X₈, X₉, X₁₀, X₁₁ may be any amino acid, with the proviso that at least one of X₁, X₂, X₃, X₅, X₆, X₇, X₁₀ is selected from:
X₁ is any amino acid except Y, preferably wherein X₁ is N, and/or
X₂ is any amino acid except Q, preferably wherein X₂ is L, Y, S, I, T, A, R, F, or V, and/or
X₃ is any amino acid except A, preferably wherein X₃ is P, T, or V, and/or
X₅ is any amino acid except R, preferably wherein X₅ is A, or S, and/or
X₆ is any amino acid except Y, preferably wherein X₆ is A, H, or S, and/or
X₇ is any amino acid except S, preferably wherein X₇ is N, F, V, L, A or R, and/or
X₁₀ is any amino acid except Q, preferably wherein Xie is S, Y, or L.

84. The pH-dependent antigen-binding protein according to any one of items 38 to 83, wherein the antigen-binding protein comprises:

a) a VH FWR2 of SEQ ID NO:39
b) a VH FWR3 of SEQ ID NO:40
c) a VH FWR4 of SEQ ID NO:33
and
d) a VL FWR2 of SEQ. ID NO: 43
e) FWR2′ is S50 according to Kabat numbering
f) a VL FWR3 of SEQ. ID NO: 45
g) a VL FWR4 of SEQ. ID NO: 47

85. An antigen-binding protein comprising an antibody light chain variable region (VL) and an antibody heavy chain variable region (VH), wherein one or more mutations are introduced in residue positions selected from the group consisting of: VH 39, VH 44, VH 45, VH 47, VH 89, VH 91, VH 103, VH 105, VL 32, VL36, VL 38, VL 43, VL 44, VL 46, VL49, VL 50, VL 85, VL 87, VL 98 and VL 100 according to Kabat numbering.

86. An antigen-binding protein comprising an antibody light chain variable region (VL) and an antibody heavy chain variable region (VH), wherein one or more mutations are introduced at residue positions selected from the group consisting of: VH 39, VH 44, VH 45, VH 47, VH 89, VH 91, VH 103, VH 105, VL 32, VL36, VL 38, VL 43, VL 44, VL 46, VL49, VL 50, VL 85, VL 87, VL 98 and VL 100 according to Kabat numbering, wherein the VH contains one or more, such as two, such as three, such as four, of the following amino acid residues:
an S, E, or A at position 39,
a P, R, N, S, K, Q, A, or T at position 44,
a T, N, I, Q, A, L, Y, D, F, S, or K at position 89, and/or
a T, R, K, P, D, I, S, or T at position 105.
and/or
wherein the VL contains one or more, such as two, such as three, such as four, such as five, such as six, such as seven, of the following amino acid residues:
an N at position 36,
a L, Y, S, I, T, A, R, F, or V at position 38,
a P, T, A or V at position 43
an A, or S at position 46,
an A, H, or S at position 49,
an S, N, T, F, V, L, S, A, or R at position 85, and/or
an S, Y, or L at position 100,
wherein all positions are indicated according to Kabat numbering.

87. The antigen-binding protein according to any one of items 85 and 86 wherein, the one or more mutations are introduced in a VL region of the antigen-binding protein comprising the framework regions:
i. FWR1 of SEQ. ID NO: 26,
ii. FWR2 of SEQ. ID NO: 27,
iii. FWR3 of SEQ. ID NO: 28, and
iv. FWR4 of SEQ. ID NO: 29;
   or
i. FWR1 of SEQ. ID NO: 151,
ii. FWR2 of SEQ. ID NO: 152,
iii. FWR3 of SEQ. ID NO: 153, and
iv. FWR4 of SEQ. ID NO: 154;
   or
i. FWR1 of SEQ. ID NO: 151,
ii. FWR2 of SEQ. ID NO: 158,
iii. FWR3 of SEQ. ID NO: 159, and
iv. FWR4 of SEQ. ID NO: 154;
   or
i. FWR1 of SEQ. ID NO: 163,
ii. FWR2 of SEQ. ID NO: 164,
iii. FWR3 of SEQ. ID NO: 165, and
iv. FWR4 of SEQ. ID NO: 154;
and in a VH region of the antigen-binding protein comprising the framework regions:
i. FWR1 of SEQ. ID NO: 30,
ii. FWR2 of SEQ. ID NO: 31,
iii. FWR3 of SEQ. ID NO: 32, and
iv. FWR4 of SEQ. ID NO: 33;
   or
i. FWR1 of SEQ. ID NO: 155,
ii. FWR2 of SEQ. ID NO: 156,
iii. FWR3 of SEQ. ID NO: 157, and
iv. FWR4 of SEQ. ID NO: 33;
   or
i. FWR1 of SEQ. ID NO: 160,
ii. FWR2 of SEQ. ID NO: 161,
iii. FWR3 of SEQ. ID NO: 162, and
iv. FWR4 of SEQ. ID NO: 33;
   or
i. FWR1 of SEQ. ID NO: 166,
ii. FWR2 of SEQ. ID NO: 167,
iii. FWR3 of SEQ. ID NO: 168, and
iv. FWR4 of SEQ. ID NO: 33.

88. The antigen-binding protein according to any one of items 85 to 87, wherein the one or more mutations are introduced in residue positions selected from the group consisting of: VH 39, VH 44, VH 45, VH 47, VH 89, VH 91, VH 103, VH 105, VL 36, VL 38, VL 43, VL 44, VL 46, VL 49, VL 50, VL 85, VL 87, VL 98 and VL 100 according to Kabat numbering

89. The antigen-binding protein according to any one of items 85 to 88, wherein the one or more mutations are substitution of an amino acid selected from the group consisting of: VH Q39, VH G44, VH L45, VH W47, VH V89, VH Y91, VH W103, VH Q105, VL Y32, VL Y36, VL Q38, VL S43, VL P44, VL T46, VL Y49, VL E50, VL D85, VL Y87, VL F98, VL G100 according to Kabat numbering for another amino acid.

90. The antigen-binding protein according to any one of items 85 to 89, wherein the one or more mutations are substitution of an amino acid selected from the group consisting of: VH Q39, VH G44, VH L45, VH W47, VH V89, VH Y91, VH W103, VH Q105, VL Y36, VL Q38, VL S43, VL P44, VL T46, VL Y49, VL E50, VL D85, VL Y87, VL F98, VL G100 according to Kabat numbering for another amino acid.

91. The antigen-binding protein according to any one of items 85 to 90, wherein the one or more mutations are substitution of an amino acid selected from the group consisting of: VH 39, VH 44, VH 89, VH 105, VL 38, VL 43, VL 50, VL 85 and VL 100 according to Kabat numbering for another amino acid.

92. The antigen-binding protein according to any one of items 85 to 89, wherein the one or more mutations are substitution of an amino acid selected from the group consisting of: VH Q39, VH G44, VH V89, VH Q105, VL Q38, VL S43, VL E50, VL D85, VL G100 according to Kabat numbering for another amino acid.

93. The antigen-binding protein according to any one of items 85 to 92, wherein the one or more mutations are introduced in residue positions selected from the group consisting of VH 39, VH 44, VH 89, VH 105, VL 38, VL 43, VL 85 and VL 105 according to Kabat numbering.

94. The antigen-binding protein according to any one of items 85 to 93, wherein the one or more mutations are substitution of an amino acid selected from the group consisting of: VH Q39, VH G44, VH V89, VH Q105, VL Q38, VL S43, VL D85, VL G100 according to Kabat numbering for another amino acid.

95. The antigen-binding protein according to any one of items 85 to 94, wherein the one or more mutations are introduced in residue positions selected from the group consisting of VH 45, VH 47, VH 91, and VH 103 according to Kabat numbering.

96. The antigen-binding protein according to any one of items 85 to 95, wherein the one or more mutations are introduced in residue positions selected from the group consisting of VH L45, VH W47, VH Y91, and VH W103 according to Kabat numbering.

97. The antigen-binding protein according to any one of items 85 to 96, wherein the one or more mutations are introduced in residue positions selected from the group consisting of VL 32, VL 46 and VL 49 according to Kabat numbering.

98. The antigen-binding protein according to any one of items 85 to 97, wherein the one or more mutations are introduced in residue positions selected from the group consisting of VL 46 and VL 49 according to Kabat numbering.

99. The antigen-binding protein according to any one of items 85 to 98, wherein the one or more mutations are introduced in residue positions selected from the group consisting of VL Y32, VL T46 and VL Y49 according to Kabat numbering.

100. The antigen-binding protein according to any one of items 85 to 99, wherein the one or more mutations are introduced in residue positions selected from the group consisting of VL T46 and VL Y49 according to Kabat numbering.

101. The antigen-binding protein according to any one of items 85 to 100, wherein the one or more mutations are introduced in residue positions selected from the group consisting of VL 36, VL 44, VL 87 and VL 98 according to Kabat numbering.

102. The antigen-binding protein according to any one of items 85 to 101, wherein the one or more mutations are introduced in residue positions selected from the group consisting of VL Y36, VL P44, VL Y87 and VL F98 according to Kabat numbering

103. The antigen-binding protein according to any one of items 85 to 102, wherein the antigen-binding protein is a pH-dependent antigen-binding protein having higher binding affinity in acidic pH than neutral pH, or lower binding affinity in acidic pH than neutral pH.

104. The antigen-binding protein according to any one of items 85 to 103, wherein the antigen-binding protein does not comprise heavy chain complementarity-determining regions 1, 2 and 3 of any of:
SEQ. ID NOs 1, 2, 3 respectively,
SEQ. ID NOs 178, 179, 180 respectively,
SEQ. ID NOs 181, 182, 183 respectively, and
SEQ. ID NOs 184, 185, 186 respectively,
and light chain complementarity-determining region 1, 2 and 3 of any of:
SEQ. ID NOs 4, 5, 6 respectively,
SEQ. ID NOs 169, 170, 171 respectively,
SEQ. ID NOs 172, 173, 174 respectively, and
SEQ. ID NOs 175, 176, 177 respectively.

105. The antigen-binding protein according to any one of items 85 to 104, wherein the antigen-binding protein does not comprise heavy chain complementarity-determining regions 1, 2 and 3 of SEQ. ID NOs 1, 2, 3 respectively and light chain complementarity-determining region 1, 2 and 3 of SEQ. ID NOs 4, 5, 6 respectively.

106. The pH-dependent antigen binding protein according to any one of items 38 to 84 or the antigen-binding protein according to any one of items 85 to 105 comprising a VH FWR1 of SEQ. ID NO: 30, SEQ. ID NO: 155, SEQ. ID NO: 160 or SEQ. ID NO: 166, except that 1 to 2 amino acids are mutated.

107. The pH-dependent antigen binding protein according to any one of items 38 to 84 or the antigen-binding protein according to any one of items 85 to 106 comprising a VH FWR1 of SEQ. ID NO: 30, SEQ. ID NO: 155, SEQ. ID NO: 160 or SEQ. ID NO: 166, except that 1 to 2 amino acids are mutated comprising a VH FWR2 of SEQ. ID NO: 31, SEQ. ID NO: 156, SEQ. ID NO: 161 or SEQ. ID NO: 167, except that 1 to 2 amino acids are mutated.

108. The pH-dependent antigen binding protein according to any one of items 38 to 84 or the antigen-binding protein according to any one of items 85 to 107 comprising a VH FWR1 of SEQ. ID NO: 30, SEQ. ID NO: 155, SEQ. ID NO: 160 or SEQ. ID NO: 166, except that 1 to 2 amino acids are mutated comprising a VH FWR2 of SEQ. ID NO: 31 except that 1 to 2 amino acids are mutated.

109. The pH-dependent antigen binding protein according to any one of items 38 to 84 or the antigen-binding protein according to any one of items 85 to 108 comprising a VH FWR1 of SEQ. ID NO: 30, SEQ. ID NO: 155, SEQ. ID NO: 160 or SEQ. ID NO: 166, except that 1 to 2 amino acids are mutated comprising a VH FWR3 of SEQ. ID NO: 32, SEQ. ID NO: 157, SEQ. ID NO: 162 or SEQ. ID NO: 168, except that 1 to 2 amino acids are mutated.

110. The pH-dependent antigen binding protein according to any one of items 38 to 84 or the antigen-binding protein according to any one of items 85 to 109 comprising a VH FWR1 of SEQ. ID NO: 30, SEQ. ID NO: 155, SEQ. ID NO: 160 or SEQ. ID NO: 166, except that 1 to 2 amino acids are mutated comprising a VH FWR3 of SEQ. ID NO: 32 except that 1 to 2 amino acids are mutated.

111. The pH-dependent antigen binding protein according to any one of items 38 to 84 or the antigen-binding protein according to any one of items 85 to 110 comprising a VH FWR1 of SEQ. ID NO: 30, SEQ. ID NO: 155, SEQ. ID NO: 160 or SEQ. ID NO: 166, except that 1 to 2 amino acids are mutated comprising a VH FWR4 of SEQ. ID NO: 33 except that 1 to 2 amino acids are mutated.

112. The antigen-binding protein according to any one of items 85 to 111, wherein the antigen-binding protein is a pH-dependent antigen-binding protein as defined in any one of items 38 to 84.

113. The method according to any one of items 1 to 37, wherein the antigen-binding proteins of the library comprise or consist of the pH-dependent antigen-binding proteins according to any one of items 38 to 84 or the antigen binding proteins according to any one of items 85 to 112.

114. A method for producing a pH-dependent antigen-binding protein directed to an antigen of interest, said method comprising the steps of:
providing an antigen-binding protein binding the antigen of interest;
introducing in said antigen-binding protein one or more mutations in residue positions selected from the group consisting of: VH 39, VH 44, VH 45, VH 47, VH 89, VH 91, VH 103, VH 105, VL 32, VL 36, VL 38, VL 43, VL 44, VL 46, VL49, VL 50, VL 85 VL 87, VL 98 and VL 100 according to Kabat numbering,
thereby producing a pH-dependent antigen-binding protein targeted to said antigen of interest.

115. The method according item 114, wherein the one or more mutations are introduced in residue positions selected from the group consisting of: VH 39, VH 44, VH 45, VH 47, VH 89, VH 91, VH 103, VH 105, VL 36, VL 38, VL 43, VL 44, VL 46, VL 49, VL 50, VL 85, VL 87, VL 98 and VL 100 according to Kabat numbering

116. The method according to any one of items 114 and 115, wherein the one or more mutations are substitution of an amino acid selected from the group consisting of: VH Q39, VH G44, VH L45, VH W47, VH V89, VH Y91, VH W103, VH Q105, VL Y32, VL Y36, VL Q38, VL S43, VL P44, VL T46, VL Y49, VL E50, VL D85, VL Y87, VL F98, VL G100 according to Kabat numbering for another amino acid.

117. The method according to any one of items 114 to 116, wherein the one or more mutations are substitution of an amino acid selected from the group consisting of: VH Q39, VH G44, VH L45, VH W47, VH V89, VH Y91, VH W103, VH Q105, VL Y36, VL Q38, VL S43, VL P44, VL T46, VL Y49, VL E50, VL D85, VL Y87, VL F98, VL G100 according to Kabat numbering for another amino acid.

118. The method according to any one of items 114 to 117, wherein the one or more mutations are introduced in residue positions from the group consisting of: VH 39, VH 44, VH 89, VH 105, VL 38, VL 43, VL 50, VL 85 and VL 100 according to Kabat numbering for another amino acid.

119. The method according to any one of items 114 to 118, wherein the one or more mutations are introduced in residue positions selected from the group consisting of: VH 39, VH 44, VH 89, VH 105, VL 38, VL 43, VL 85 and VL 100 according to Kabat numbering.

120. The method according to any one of items 114 to 119, wherein the one or more mutations are substitution of an amino acid selected from the group consisting of: VH Q39, VH G44, VH V89, VH Q105, VL Q38, VL S43, VL E50, VL D85 and VL G100 according to Kabat numbering for another amino acid.

121. The method according to any one of items 114 to 120, wherein the one or more mutations are substitution of an amino acid selected from the group consisting of: VH Q39, VH G44, VH V89, VH Q105, VL Q38, VL S43, VL D85 and VL G100 according to Kabat numbering for another amino acid.

122. The method according to any one of items 114 to 121, wherein the one or more mutations are introduced in residue positions selected from the group consisting of VH 45, VH 47, VH 91, and VH 103 according to Kabat numbering.

123. The method according to any one of items 114 to 122, wherein the one or more mutations are introduced in residue positions selected from the group consisting of VH L45, VH W47, VH Y91, and VH W103 according to Kabat numbering.

124. The method according to any one of items 114 to 123, wherein the one or more mutations are introduced in residue positions selected from the group consisting of VL 32, VL 46 and VL 49 according to Kabat numbering.

125. The method according to any one of items 114 to 124, wherein the one or more mutations are introduced in residue positions selected from the group consisting of VL 46 and VL 49 according to Kabat numbering.

126. The method according to any one of items 114 to 125, wherein the one or more mutations are introduced in residue positions selected from the group consisting of VL Y32, VL T46 and VL Y49 according to Kabat numbering.

127. The method according to any one of items 114 to 126, wherein the one or more mutations are introduced in residue positions selected from the group consisting of VL T46 and VL Y49 according to Kabat numbering.

128. The method according to any one of items 114 to 127, wherein the one or more mutations are introduced in residue positions selected from the group consisting of VL 36, VL 44, VL 87 and VL 98 according to Kabat numbering.

129. The method according to any one of items 114 to 128, wherein the one or more mutations are introduced in residue positions selected from the group consisting of VL Y36, VL P44, VL Y87 and VL F98 according to Kabat numbering

130. The method according to any one of items 114 to 129, wherein the method further comprises a step of testing that the antigen-binding protein comprising the one or more mutations has a higher binding affinity in acidic pH than neutral pH, or a lower binding affinity in acidic pH than neutral pH.

131. The method according to any one of items 114 to 130, wherein the method further comprises a step of selecting antigen-binding proteins having a higher binding affinity in acidic pH than neutral pH, or a lower binding affinity in acidic pH than neutral pH.

132. The method according to any one of items 114 to 131, wherein the method further comprises a step of selecting the antibodies displaying highest thermal stability, such as the highest Fab fragment melting temperature.

133. The method or the pH-dependent antigen-binding protein according to any one of the preceding items, wherein the one or more mutations are at residue positions occupied by charged residues, and wherein the mutation for example is substitution to a non-charged amino acid or to an amino acid, which cannot engage in hydrogen bonding.

134. The method or the pH-dependent antigen-binding protein according to any one of the preceding items, wherein the one or more mutations are mutations to charged residues.

135. The method or the pH-dependent antigen-binding protein according to any one of the preceding items, wherein the one or more mutations are at residue positions occupied by residues which can engage in hydrogen bonding, and wherein the mutation for example is substitution to an amino acid, which cannot engage in hydrogen bonding.

136. The method or the pH-dependent antigen-binding protein according to any one of the preceding items wherein the one or more mutations are mutations to residues which can engage in hydrogen bonding.

137. The method or the pH-dependent antigen-binding protein according to any one of the preceding items wherein the one or more mutations are mutations to residues which cannot engage in hydrogen bonding.

138. The method or the pH-dependent antigen-binding protein according to any one of the preceding items, wherein the one or more mutations are at residue positions occupied by residues selected from the group consisting of: Arginine, Asparagine, Aspartic acid, Glutamine, Glutamic acid, Histidine, Lysine, Serine, Threonine, Tryptophan, and Tyrosine, and wherein the mutation for example is substitution to a non-charged amino acid or to an amino acid, which cannot engage in hydrogen bonding.

139. The method or the pH-dependent antigen-binding protein according to any one of the preceding items wherein the one or more mutations are mutations to residues selected from the group consisting of: Arginine, Asparagine, Aspartic acid, Glutamine, Glutamic acid, Histidine, Lysine, Serine, Threonine, Tryptophan, and Tyrosine.

140. The method or the pH-dependent antigen-binding protein according to any one of the preceding items, wherein the one or more mutations are at residue positions occupied by residues selected from the group consisting of: Arginine, Asparagine, Aspartic acid, Glutamine, Glutamic acid, Lysine, Serine, Threonine, Tryptophan, and Tyrosine, and wherein the mutation for example is substitution to a non-charged amino acid or to an amino acid, which cannot engage in hydrogen bonding.

141. The method or the pH-dependent antigen-binding protein according to any one of the preceding items wherein the one or more mutations are mutations to residues selected from the group consisting of: Arginine, Asparagine, Aspartic acid, Glutamine, Glutamic acid, Lysine, Serine, Threonine, Tryptophan, and Tyrosine.

142. The method or the pH-dependent antigen-binding protein according to any one of the preceding items, wherein the one or more mutations are at residue positions occupied by residues selected from the group consisting of: Arginine, Aspartic acid, Glutamic acid, Histidine, and Lysine, and wherein the mutation for example is substitution to a non-charged amino acid or to an amino acid, which cannot engage in hydrogen bonding.

143. The method or the pH-dependent antigen-binding protein according to any one of the preceding items wherein the one or more mutations are mutations to residues selected from the group consisting of: Arginine, Aspartic acid, Glutamic acid, Histidine, and Lysine.

144. The method or the pH-dependent antigen-binding protein according to any one of the preceding items, wherein the one or more mutations are at residue positions occupied by residues selected from the group consisting of: Arginine, Aspartic acid, Glutamic acid, Histidine, and Lysine, and wherein the mutation for example is substitution to a non-charged amino acid or to an amino acid, which cannot engage in hydrogen bonding.

145. The method or the pH-dependent antigen-binding protein according to any one of the preceding items wherein the one or more mutations are mutations to residues selected from the group consisting of: Arginine, Aspartic acid, Glutamic acid, Histidine, and Lysine.

146. The method or the pH-dependent antigen-binding protein according to anyone of the preceding items, wherein the pH-dependent antigen-binding protein has a lower Kd value to its antigen at acidic pH compared to the Kd value at neutral pH.

147. The method or the pH-dependent antigen-binding protein according to item 146, wherein the Kd value at acidic pH is decreased by a factor 2, such as a factor 5, for instance a factor 10, such as a factor 25, for instance a factor 50, such as a factor 75, for instance a factor 100, such as a factor 125, for instance a factor 250, such as a factor 500, for instance a factor 750, such as a factor 1000 compared to neutral pH.

148. The method or the pH-dependent antigen-binding protein according to any one of the preceding items, wherein the pH-dependent antigen-binding protein has a higher Kd value to its antigen at acidic pH compared to the Kd value at neutral pH.

149. The method or the pH-dependent antigen-binding protein according to item 148, wherein the Kd value at acidic pH is increased by a factor 2, such as a factor 5, for instance a factor 10, such as a factor 25, for instance a factor 50, such as a factor 75, for instance a factor 100, such as a factor 125, for instance a factor 250, such as a factor 500, for instance a factor 750, such as a factor 1000 compared to neutral pH.

150. The method or the pH-dependent antigen-binding protein according to any one of the preceding items, wherein the pH-dependent antigen-binding protein is selected from the group consisting of a full-length antibody, a Fab fragment, a F(ab′) fragment, a F(ab′)2 fragment, an scFv, a diabody, and a triabody.

151. The method or the pH-dependent antigen-binding protein according to any one of the preceding items, wherein the pH-dependent antigen-binding protein comprises an immunoglobulin constant region.

152. The method or the pH-dependent antigen-binding protein according to any one of the preceding items, wherein the pH-dependent antigen-binding protein immunoglobulin constant region is selected from the group consisting of IgG, IgM, IgA, IgD, and IgE.

153. The method or the pH-dependent antigen-binding protein according to any one of the preceding items, wherein the pH-dependent antigen-binding protein immunoglobulin constant region is selected from the group consisting of IgG and IgA.

154. The method or the pH-dependent antigen-binding protein according to any one of the preceding items, wherein the pH-dependent antigen-binding protein immunoglobulin constant region is selected from the group consisting of IgG1, IgA1, and IgA2.

155. The method or the pH-dependent antigen-binding protein according to any one of the preceding items, wherein the pH-dependent antigen-binding protein immunoglobulin constant region is selected from the group consisting of IgG1, IgG2, IgG3, and IgG4.

156. The method or the pH-dependent antigen-binding protein according to any one of the preceding items, wherein the pH-dependent antigen-binding protein is a monoclonal antibody.

157. The method or the pH-dependent antigen-binding protein according to any one of the preceding items, wherein the pH-dependent antigen-binding protein is a human antibody or a chimeric antibody.

158. The method or the pH-dependent antigen-binding protein according to any one of the preceding items, wherein the pH-dependent antigen-binding protein has one or more modification(s) selected from the group consisting of PEGylation, polysialylation, Fc region mutation and N-glycosylation.

159. The method or the pH-dependent antigen-binding protein according to any one of the preceding items, wherein the pH-dependent antigen-binding protein comprises a detection label.

160. The method or the pH-dependent antigen-binding protein according to item 159, wherein the detection label is selected from the group consisting of a colorimetric, a fluorescent, a luminescent, a magnetic, and a paramagnetic label.

161. The method or the pH-dependent antigen-binding protein according to item 159, wherein the detection label is biotin.

162. The method or the pH-dependent antigen-binding protein according to item 159, wherein the detection label is a gold nanoparticle.

163. A composition comprising the pH-dependent antigen-binding protein of any one of items 38 to 113 and a pharmaceutically acceptable excipient.

164. A pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein the pH-dependent antigen-binding protein has higher binding affinity in acidic pH than neutral pH, or lower binding affinity in acidic pH than neutral pH, or the composition according to item 163, for use in a method of treatment of cancer, autoimmune diseases, metabolic diseases, or haematological diseases in a patient in need thereof.

165. The pH-dependent antigen-binding protein according to item 164, wherein the cancer is a solid-tumor cancer.

166. The pH-dependent antigen-binding protein according to any one of items 164 to 165, wherein the pH-dependent antigen-binding protein is the protein according to any one of items 38 to 113.

167. A method of treating cancer, autoimmune diseases, metabolic diseases, or haematological diseases, comprising administering to a patient in need thereof a pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein the pH-dependent antigen-binding protein has higher binding affinity in acidic pH than neutral pH, or lower binding affinity in acidic pH than neutral pH, or the composition according to item 163.

168. The method according to item 167 wherein the cancer is a solid-tumor cancer.

169. The method according to any one of items 167 to 168, wherein the pH-dependent antigen-binding protein is the protein according to any one of items 38 to 113.

170. Use of a pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, wherein the parental and wherein the pH-dependent antigen-binding protein has higher binding affinity in acidic pH than neutral pH, or lower binding affinity in acidic pH than neutral pH, or the composition according to item 163 in the manufacture of a medicament for the treatment of cancer, autoimmune diseases, metabolic diseases, or haematological diseases.

171. The use according to item 170, wherein the cancer is a solid-tumor cancer.

172. The use according to any one of items 170 and 171, wherein the pH-dependent antigen-binding protein is the protein according to any one of items 38 to 113.

173. Use of a pH-dependent antigen-binding protein comprising an antibody light chain variable region (VL), wherein said VL comprises 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein the pH-dependent antigen-binding protein has higher binding affinity in acidic pH than neutral pH, or lower binding affinity in acidic pH than neutral pH, or the composition according to item 163 in an in vitro method for detection and/or diagnosis of cancer.

174. The method according to any one of items 167 to 169, wherein the plasma recycling of the pH-dependent antigen-binding protein is improved compared to the antigen-binding protein comprising the parental FWR1, FWR2, FWR2′, FWR3 and FWR4.

175. The method according to any one of items 167 to 169, 174, wherein the pH-dependent antigen-binding improves the clearance of its antigen from the plasma compared to the antigen-binding protein comprising the parental FWR1, FWR2, FWR2′, FWR3 and FWR4.

176. The method according to any one of items 167 to 169, 174 to 175, wherein the pH-dependent antigen-binding improves the release from its antigen in endosomes compared to the antigen-binding protein comprising the parental FWR1, FWR2, FWR2′, FWR3 and FWR4.

177. The method according to any one of items 167 to 169, 174 to 175, wherein the intracellular uptake of the pH-dependent antigen-binding protein is improved in acidic microenvironments compared to the antigen-binding protein comprising the parental FWR1, FWR2, FWR2′, FWR3 and FWR4.

178. An in-vitro antigen detection method comprising:
providing a pH-dependent antigen-binding protein according to any one of items 38 to 113;
contacting the pH-dependent antigen-binding protein with the antigen it binds to; and
detecting the contact between the pH-dependent antigen-binding protein and its antigen,
thereby detecting the antigen.

179. An in-vitro antigen purification method comprising:
providing a pH-dependent antigen-binding protein according to any one of items 38 to 113;
contacting the pH-dependent antigen-binding protein with the antigen it binds to in a complex mixture; and
separating the pH-dependent antigen-binding protein/antigen complex from the complex mixture,
thereby purifying the antigen.

180. The antigen-binding protein according to any one of items 85 to 113, wherein the each of the VL and VH comprise 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein said pH-dependent antigen-binding protein does not comprise heavy chain complementarity-determining regions 1, 2 and 3 of any of:
SEQ. ID NOs 1, 2, 3 respectively,
SEQ. ID NOs 178, 179, 180 respectively,
SEQ. ID NOs 181, 182, 183 respectively, and
SEQ. ID NOs 184, 185, 186 respectively,
and light chain complementarity-determining region 1, 2 and 3 of any of:
SEQ. ID NOs 4, 5, 6 respectively,
SEQ. ID NOs 169, 170, 171 respectively,
SEQ. ID NOs 172, 173, 174 respectively, and
SEQ. ID NOs 175, 176, 177 respectively.

181. The antigen-binding protein according to any one of items 85 to 113, wherein the each of the VL and VH comprise 3 complementarity-determining regions, CDR1, CDR2 and CDR3 surrounded by 4 framework regions FWR1, FWR2, FWR3 and FWR4, wherein the FWR2 and the adjacent amino acid of CDR2 is referred to as FWR2′, and wherein at least one of said FWR1, FWR2, FWR2′, FWR3 and FWR4 contains at least one mutation compared to a parental

FWR1, FWR2, FWR2′, FWR3 and FWR4, and wherein said pH-dependent antigen-binding protein does not comprise heavy chain complementarity-determining regions 1, 2 and 3 of SEQ. ID NOs 1, 2, 3 respectively and light chain complementarity-determining region 1, 2 and 3 of SEQ. ID NOs 4, 5, 6 respectively

182. The antigen binding protein according to any one of items 180 and 181, wherein the 3 complementarity-determining regions, CDR1, CDR2 and CDR3, the 4 framework regions FWR1, FWR2, FWR3 and FWR4, and the FWR2′ are as described in any one of items 38 to 84.

183. The method according to any one of items 114 to 162, wherein the one or more mutations are introduced in residue positions selected from the group consisting of: VH 39, VH 44, VH 45, VH 47, VH 89, VH 91, VH 103, VH 105, VL 36, VL 38, VL 43, VL 44, VL 46, VL 49, VL 85, VL 87, VL 98 and VL 100 according to Kabat numbering

184. The method according to any one of items 114 to 162 and 183, wherein the one or more mutations are substitution of an amino acid selected from the group consisting of: VH Q39, VH G44, VH L45, VH W47, VH V89, VH Y91, VH W103, VH Q105, VL Y32, VL Y36, VL Q38, VL S43, VL P44, VL T46, VL Y49, VL D85, VL Y87, VL F98, VL G100 according to Kabat numbering for another amino acid.

185. The method according to any one of items 114 to 162, and 183 to 184 wherein the one or more mutations are substitution of an amino acid selected from the group consisting of: VH Q39, VH G44, VH L45, VH W47, VH V89, VH Y91, VH W103, VH Q105, VL Y36, VL Q38, VL S43, VL P44, VL T46, VL Y49, VL D85, VL Y87, VL F98, VL G100 according to Kabat numbering for another amino acid.

186. The method according to any one of items 114 to 162, and 183 to 185, wherein the one or more mutations are introduced in residue positions from the group consisting of: VH 39, VH 44, VH 89, VH 105, VL 38, VL 43, VL 85 and VL 100 according to Kabat numbering for another amino acid.

187. The method, the pH-dependent antigen-binding protein, the antigen binding protein, the composition or the use according to any one of the preceding items, wherein said higher binding affinity in acidic pH than neutral pH, or lower binding affinity in acidic pH than neutral pH, is a lower KD or a higher KD, respectively.

188. The method, the pH-dependent antigen-binding protein, the antigen binding protein, the composition or the use according to any one of the preceding items, wherein said higher binding affinity in acidic pH than neutral pH, or lower binding affinity in acidic pH than neutral pH is measured as a ratio of DELFIA signals in acidic pH and neutral pH.

189. The method, the pH-dependent antigen-binding protein, the antigen binding protein, the composition or the use according to any one of the preceding items, wherein said higher binding affinity in acidic pH than neutral pH, or lower binding affinity in acidic pH than neutral pH is by at least 1.25-fold, such as at least 1.3-fold, such as at least 1.5-fold, such as at least 1.6-fold, such as at least 1.7-fold, such as at least 1.8-fold, such as at least 1.9-fold, such as at least 2-fold, such as at least 2.2-fold, such as at least 2.5-fold, such as at least 5-fold, such as at least 6-fold, such as at least 10-fold, such as at least 10.5-fold.

1-23. (canceled)

24. An antigen-binding protein comprising an antibody light chain variable region (VL) and an antibody heavy chain variable region (VH), wherein one or more mutations are introduced at residue positions VH 39, VH 44, VH 45, VH 47, VH 89, VH 91, VH 103, VH 105, VL36, VL 38, VL 43, VL 44, VL 46, VL 85, VL 98 or VL 100 according to Kabat numbering.

25. The antigen-binding protein according to claim 24, wherein:
the VH contains one or more of the following amino acid residues:
an S, E, R, T, or A at position 39,
a P, R, N, S, K, Q, A, Y, or T at position 44,
a T, N, I, Q, A, L, Y, D, F, S, or K at position 89, or
a T, R, K, P, D, I, S, or T at position 105,
or
the VL contains one or more of the following amino acid residues:
an N at position 36,
a L, Y, S, I, T, A, R, F, or V at position 38,
a P, T, A, R, Q, K, or V at position 43
an A or S at position 46,
an S, N, T, F, V, L, S, A, or R at position 85, or
an S, Y, W, or L at position 100,
wherein all positions are indicated according to Kabat numbering.

26. The antigen-binding protein according to claim 24, wherein, the one or more mutations are introduced in a VL region of the antigen-binding protein comprising the framework regions:
a) FWR1 of SEQ ID NO: 26,
b) FWR2 of SEQ ID NO: 27,
c) FWR3 of SEQ ID NO: 28, and
d) FWR4 of SEQ ID NO: 29;
or
a) FWR1 of SEQ ID NO: 151,
b) FWR2 of SEQ ID NO: 152,
c) FWR3 of SEQ ID NO: 153, and
d) FWR4 of SEQ ID NO: 154;
or
a) FWR1 of SEQ ID NO: 151,
b) FWR2 of SEQ ID NO: 158,
c) FWR3 of SEQ ID NO: 159, and
d) FWR4 of SEQ ID NO: 154;
or
a) FWR1 of SEQ ID NO: 163,
b) FWR2 of SEQ ID NO: 164,
c) FWR3 of SEQ ID NO: 165, and
d) FWR4 of SEQ ID NO: 154;
and in a VH region of the antigen-binding protein comprising the framework regions:
a) FWR1 of SEQ ID NO: 30,
b) FWR2 of SEQ ID NO: 31,
c) FWR3 of SEQ ID NO: 32, and
d) FWR4 of SEQ ID NO: 33;
or
a) FWR1 of SEQ ID NO: 155,
b) FWR2 of SEQ ID NO: 156,
c) FWR3 of SEQ ID NO: 157, and
d) FWR4 of SEQ ID NO: 33;
or
a) FWR1 of SEQ ID NO: 160,
b) FWR2 of SEQ ID NO: 161,
c) FWR3 of SEQ ID NO: 162, and
d) FWR4 of SEQ ID NO: 33;
or
a) FWR1 of SEQ ID NO: 166,
b) FWR2 of SEQ ID NO: 167,
c) FWR3 of SEQ ID NO: 168, and
d) FWR4 of SEQ ID NO: 33.

27. The antigen-binding protein according to claim 26, wherein the one or more mutations are not mutations to Histidine residues.

28. The antigen-binding protein according to claim 24, wherein the antigen-binding protein is a pH-dependent antigen-binding protein having higher binding affinity in acidic pH than neutral pH, or lower binding affinity in acidic pH than neutral pH.

29. The antigen-binding protein according to claim 24, wherein the each of the VL and VH comprise three complementarity-determining regions, CDR1, CDR2 and CDR3 surrounded by four framework regions FWR1, FWR2, FWR3 and FWR4, wherein the FWR2 and an adjacent amino acid of CDR2 is referred to as FWR2′, and wherein at least one of said FWR1, FWR2, FWR2′, FWR3 and FWR4 contains at least one mutation compared to a parental FWR1, FWR2, FWR2′, FWR3 and FWR4, and wherein said pH-dependent antigen-binding protein does not comprise heavy chain complementarity-determining regions 1, 2 and 3 of any of:
SEQ ID NOs 1, 2, 3 respectively,
SEQ ID NOs 178, 179, 180 respectively,
SEQ ID NOs 181, 182, 183 respectively, and
SEQ ID NOs 184, 185, 186 respectively,
and light chain complementarity-determining region 1, 2 and 3 of any of:
SEQ ID NOs 4, 5, 6 respectively,
SEQ ID NOs 169, 170, 171 respectively,
SEQ ID NOs 172, 173, 174 respectively and
SEQ ID NOs 175, 176, 177 respectively.

30. The antigen binding protein according to claim 29, wherein the antigen-binding protein comprises:
a) a VH FWR2 of
(SEQ ID NO: 98)
WVRX₁APGQX₂X₃EX₄MG
b) a VH FWR3 of
(SEQ ID NO: 99)
RVTITADX₅STSTAYMX₆LX₇SLRSDDTAX₈YX₉CAR
c) a VH FWR4 of
(SEQ ID NO: 100)
X₁₀GX₁₁GTLVTVSS,
wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
X₁ is any amino acid except Q, or
X₂ is any amino acid except G, or
X₈ is any amino acid except V, or
X₁₁ is any amino acid except Q;
and
d) a light chain CDR1 of
(SEQ ID NO: 104)
TRSX₁GSIGSDX₂VH
e) a VL FWR2 of
(SEQ ID NO: 101)
WX₃QX₄RPGSX₅X₆TX₇VIX₈
f) a VL FWR3 of
(SEQ ID NO: 102)
GVPDRFSGSIDSSSNSASLTISGLKTEDEAX₉YX₁₀C
g) a VL FWR4 of
(SEQ ID NO: 103)
X₁₁GX₁₂GTKLTVX₁₃,
wherein each of X₁, X₂, X₃, X₄, X₅, X₆, X₇, X₈, X₉, X₁₀, X₁₁, X₁₂, X₁₃ may be any amino acid, with the proviso that at least one of X₂, X₃, X₄, X₅, X₇, X₈, X₉, X₁₂ is selected from:
X₂ is any amino acid except Y, or
X₃ is any amino acid except Y, or
X₄ is any amino acid except Q, or
X₅ is any amino acid except S, or
X₇ is any amino acid except T, or
X₈ is any amino acid except Y, or
X₉ is any amino acid except D, or
X₁₂ is any amino acid except G.

31. The antigen binding protein according to claim 29, wherein the antigen-binding protein comprises:
a) a VH FWR2 of
(SEQ ID NO: 98)
WVRX₁APGQX₂X₃EX₄MG
b) a VH FWR3 of
(SEQ ID NO: 99)
RVTITADX ₅STSTAYMX₆LX₇SLRSDDTAX₈YX₉CAR
c) a VH FWR4 of
(SEQ ID NO: 100)
X₁₀GX₁₁GTLVTVSS,
wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
X₁ is S, E, or A, or
X₂ is P, R, N, S, K, Q, A, or T, or
X₈ is T, N, I, Q, A, L, or K, or
X₁₁ is T, R, K, P, D, I, or T;
and
d) a light chain CDR1 of
(SEQ ID NO: 104)
TRSX₁GSIGSDX₂VH
e) a VL FWR2 of
(SEQ ID NO: 101)
WX₃QX₄RPGSX₅X₆TX₇VIX₈
f) a VL FWR3 of
(SEQ ID NO: 102)
GVPDRFSGSIDSSSNSASLTISGLKTEDEAX₉YX₁₀C
g) a VL FWR4 of
(SEQ ID NO: 103)
X₁₁GX₁₂GTKLTVX₁₃,
wherein each of X₁, X₂, X₃, X₄, X₅, X₆, X₇, X₈, X₉, X₁₀, X₁₁, X₁₂, X₁₃ may be any amino acid, with the proviso that at least one of X₂, X₃, X₄, X₅, X₇, X₈, X₉, X₁₂ is selected from:
X₂ is A, S, or T, or
X₃ is N, or
X₄ is L, Y, S, I, T, A, R, or V, or
X₅ is P, T, A, or V, or
X₇ is A, or S, or
X₈ is A, or S, or
X₉ is S, N, T, F, V, L, S, A or R, or
X₁₂ is S, Y, or L.

32. The antigen-binding protein according to claim 29, wherein the antigen-binding protein comprises:
a) a VH FWR2 of
(SEQ ID NO: 187)
WVRX₁APGKX₂X₃EX₄VS
b) a VH FWR3 of
(SEQ ID NO: 188)
RFTISRDX₅AKNSLYLX₆MX₇SLRAEDTAX₈YX₉CAK
c) a VH FWR4 of
(SEQ ID NO: 100)
X₁₀GX₁₁GTLVTVSS,
wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
X₁ is any amino acid except Q, or
X₂ is any amino acid except G, or
X₈ is any amino acid except V, or
X₁₁ is any amino acid except Q;
and
d) a VL FWR2 of
(SEQ ID NO: 190)
WX₁QX₂KPGKX₃X₄KX₅LIX₆
e) a VL FWR3 of
(SEQ ID NO: 191)
GVPSRFSGSGSGTDFTLTISSLQPEDVAX₇YX₈C
f) a VL FWR4 of
(SEQ ID NO: 192)
X₉GX₁₀GTKVEIX₁₁,
wherein each of X₁, X₂, X₃, X₄, X₅, X₆, X₇, X₈, X₉, X₁₀, X₁₁ may be any amino acid, with the proviso that at least one of X₁, X₂, X₃, X₅, X₆, X₇, X₁₀ is selected from:
X₁ is any amino acid except Y, or
X₂ is any amino acid except Q, or
X₃ is any amino acid except A, or
X₅ is any amino acid except L, or
X₆ is any amino acid except Y, or
X₇ is any amino acid except T, or
X₁₀ is any amino acid except Q.

33. The antigen-binding protein according to claim 29, wherein the antigen-binding protein comprises:
a) a VH FWR2 of
(SEQ ID NO: 187)
WVRX₁APGKX₂X₃EX₄VS
b) a VH FWR3 of
(SEQ ID NO: 188)
RFTISRDX₅AKNSLYLX₆MX₇SLRAEDTAX₈YX₉CAK
c) a VH FWR4 of
(SEQ ID NO: 100)
X₁₀GX₁₁GTLVTVSS,
wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
X₁ is S, E, or A, or
X₂ is P, R, N, S, K, Q, A, Y or T, or
X₈ is T, N, I, Q, A, L, Y, D, F, S or K, or
X₁₁ is T, R, K, P, D, I, S or T;
and
d) a VL FWR2 of
(SEQ ID NO: 190)
WX₁QX₂KPGKX₃X₄KX₅LIX₆
e) a VL FWR3 of
(SEQ ID NO: 191)
GVPSRFSGSGSGTDFTLTISSLQPEDVAX₇YX₈C
f) a VL FWR4 of
(SEQ ID NO: 192)
X₉GX₁₀GTKVEIX₁₁,
wherein each of X₁, X₂, X₃, X₄, X₅, X₆, X₇, X₈, X₉, X₁₀, X₁₁ may be any amino acid, with the proviso that at least one of X₁, X₂, X₃, X₅, X₆, X₇, X₁₀ is selected from:
X₁ is N, or
X₂ is L, Y, S, I, T, A, R, F, or V, or
X₃ is P, T, or V, or
X₅ is A, or S, or
X₆ is A, or S, or
X₇ is N, F, V, L, S, A or R, or
X₁₀ is S, Y, or L.

34. The antigen-binding protein according to claim 29, wherein the antigen-binding protein comprises:
a) a VH FWR2 of
(SEQ ID NO: 197)
WVRX₁APGKX₂X₃EX₄VG
b) a VH FWR3 of
(SEQ ID NO: 198)
RFTFSLDX₅SKSTAYLX₆MX₇SLRX₈EDTAX₉YX₁₀CAK
c) a VH FWR4 of
(SEQ ID NO: 189)
X₁₁GX₁₂GTLVTVSS,
wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
X₁ is any amino acid except Q, or
X₂ is any amino acid except G, or
X₈ is any amino acid except A, or
X₉ is any amino acid except V, or
X₁₂ is any amino acid except Q;
and
d) a VL FWR2 of
(SEQ ID NO: 190)
WX₁QX₂KPGKX₃X₄KX₅LIX₆
e) a VL FWR3 of
(SEQ ID NO: 199)
GVPSRFSGSGSGTDFTLTISSLQPEDFAX₇YX₈C
f) a VL FWR4 of
(SEQ ID NO: 192)
X₉GX₁₀GTKVEIX₁₁,
wherein each of X₁, X₂, X₃, X₄, X₅, X₆, X₇, X₈, X₉, X₁₀, X₁₁ may be any amino acid, with the proviso that at least one of X₁, X₂, X₃, X₅, X₆, X₇, X₁₀ is selected from:
X₁ is any amino acid except Y, or
X₂ is any amino acid except Q, or
X₃ is any amino acid except A, or
X₅ is any amino acid except V, or
X₆ is any amino acid except Y, or
X₇ is any amino acid except T, or
X₁₀ is any amino acid except Q.

35. The antigen-binding protein according to claim 29, wherein the antigen-binding protein comprises:
a) a VH FWR2 of
(SEQ ID NO: 197)
WVRX₁APGKX₂X₃EX₄VG
b) a VH FWR3 of
(SEQ ID NO: 198)
RFTFSLDX₅SKSTAYLX₆MX₇SLRX₈EDTAX₉YX₁₀CAK
c) a VH FWR4 of
(SEQ ID NO: 189)
X₁₁GX₁₂GTLVTVSS,
wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
X₁ is S, E, or A, or
X₂ is P, R, N, S, K, Q, A, or T, or
X₈ is T, or
X₉ is T, N, I, Q, A, L, or K, or
X₁₂ is T, R, K, P, D, I, S, or T;
and
d) a VL FWR2 of
(SEQ ID NO: 190)
WX₁QX₂KPGKX₃X₄KX₅LIX₆
e) a VL FWR3 of
(SEQ ID NO: 199)
GVPSRFSGSGSGTDFTLTISSLQPEDFAX₇YX₈C
f) a VL FWR4 of
(SEQ ID NO: 192)
X₉GX₁₀GTKVEIX₁₁,
wherein each of X₁, X₂, X₃, X₄, X₅, X₆, X₇, X₈, X₉, X₁₀, X₁₁ may be any amino acid, with the proviso that at least one of X₁, X₂, X₃, X₅, X₆, X₇, X₁₀ is selected from:
X₁ is N, or
X₂ is L, Y, S, I, T, A, R, F, or V, or
X₃ is P, T, or V, or
X₅ is A, or S, or
X₆ is A, or S, or
X₇ is S, N, F, V, L, A, or R, or
X₁₀ is S, Y, or L.

36. The antigen-binding protein according to claim 29, wherein the antigen-binding protein comprises:
a) a VH FWR2 of
(SEQ ID NO: 203)
WVRX₁APGKX₂X₃EX₄MG
b) a VH FWR3 of
(SEQ ID NO: 204)
RVTMTEDX₅STDTAYMX₆LX₇SLRSEDTAX₈YX₉CST
c) a VH FWR4 of
(SEQ ID NO: 189)
X₁₀GX₁₁GTLVTVSS,
wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
X₁ is any amino acid except Q, or
X₂ is any amino acid except G, or
X₈ is any amino acid except V, or
X₁₁ is any amino acid except Q;
and
d) a VL FWR2 of
(SEQ ID NO: 190)
WX₁QX₂KPGKX₃X₄KX₅LIX₆
e) a VL FWR3 of
(SEQ ID NO: 205)
GVPSRFSGSGSGTEFTLTISSLQPEDLAX₇YX₈C
f) a VL FWR4 of
(SEQ ID NO: 192)
X₉GX₁₀GTKVEIX₁₁,
wherein each of X₁, X₂, X₃, X₄, X₅, X₆, X₇, X₈, X₉, X₁₀, X₁₁ may be any amino acid, with the proviso that at least one of X₁, X₂, X₃, X₅, X₆, X₇, X₁₀ is selected from:
X₁ is any amino acid except Y, or
X₂ is any amino acid except Q, or
X₃ is any amino acid except A, or
X₅ is any amino acid except R, or
X₆ is any amino acid except Y, or
X₇ is any amino acid except S, or
X₁₀ is any amino acid except Q.

37. The antigen-binding protein according to claim 29, wherein the antigen-binding protein comprises:
a) a VH FWR2 of
(SEQ ID NO: 203)
WVRX₁APGKX₂X₃EX₄MG
b) a VH FWR3 of
(SEQ ID NO: 204)
RVTMTEDX₅STDTAYMX₆LX₇SLRSEDTAX₈YX₉CST
c) a VH FWR4 of
(SEQ ID NO: 189)
X₁₀GX₁₁GTLVTVSS,
wherein each of X₁, X₂, X₃, X₄, X₅, X₆, X₇, X₈, X₉, X₁₀, X₁₁ may be any amino acid, with the proviso that at least one of X₁, X₂, X₈, X₁₁ is selected from:
X₁ is S, E, or A, or
X₂ is P, R, N, S, K, Q, A, or T, or
X₈ is T, N, I, Q, A, L, or K, or
X₁₁ is T, R, K, P, D, I, S, or T;
and
d) a VL FWR2 of
(SEQ ID NO: 190)
WX₁QX₂KPGKX₃X₄KX₅LIX₆
e) a VL FWR3 of
(SEQ ID NO: 205)
GVPSRFSGSGSGTEFTLTISSLQPEDLAX₇YX₈C
f) a VL FWR4 of
(SEQ ID NO: 192)
X₉GX₁₀GTKVEIX₁₁,
wherein each of X₁, X₂, X₃, X₄, X₅, X₆, X₇, X₈, X₉, X₁₀, X₁₁ may be any amino acid, with the proviso that at least one of X₁, X₂, X₃, X₅, X₆, X₇, X₁₀ is selected from:
X₁ is N, or
X₂ is L, Y, S, I, T, A, R, F, or V, or
X₃ is P, T, or V, or
X₅ is A, or S, or
X₆ is A, or S, or
X₇ is N, F, V, L, A or R, or
X₁₀ is S, Y, or L.

38. The pH-dependent antigen-binding protein according to claim 28, wherein the K_{D} value at acidic pH is increased or decreased by at least a factor 2 compared to neutral pH.

39. A composition comprising:
an antigen-binding protein comprising an antibody light chain variable region (VL) and an antibody heavy chain variable region (VH), wherein one or more mutations are introduced at residue positions VH 39, VH 44, VH 45, VH 47, VH 89, VH 91, VH 103, VH 105, VL36, VL 38, VL 43, VL 44, VL 46, VL 85, VL 98 and VL 100 according to Kabat numbering; and
a pharmaceutically acceptable excipient.

40. A method for isolating antigen-binding proteins having a pH-dependent scaffold, wherein the scaffold consists of the regions of the variable regions not being part of a paratope, said method comprising the steps of:
providing a library comprising antigen-binding proteins, each comprising an antibody light chain variable region (VL), wherein said VLs comprise one or more mutations positioned outside of a paratope of the antigen-binding protein, wherein said library comprises a plurality of antigen-binding proteins containing different mutations in the VL;
and
selecting antigen-binding proteins that display higher binding affinity in acidic pH than neutral pH, or lower binding affinity in acidic pH than neutral pH from said library, thereby isolating antigen-binding proteins having a pH-dependent scaffold.

41. The method according to claim 40, wherein said method further comprises the steps of:
providing an antigen-binding protein binding said specific epitope;
exchanging the paratope of said antigen-binding protein having a pH dependent scaffold for the paratope of said antigen-binding protein binding said epitope.
